SEQUENCE LISTING (441 sequences)

SEQ ID NO: 1 - DNA, 116 bp, Homo sapiens
gttcccggaacggcctcttgggggcgttccagccccacggacccgcagggagtccccgccgcaatttgcatggggctcatttgcatgaccccgccccgcgcgggagtcgggggcgc

SEQ ID NO: 2 - DNA, 116 bp, Artificial sequence
  Synthetic
gttttcggaacggttttttgggggcgttttagttttacggattcgtagggagttttcgtcgtaatttgtatggggtttatttgtatgatttcgtttcgcgcgggagtcgggggcgt

SEQ ID NO: 3 - DNA, 23 bp, Artificial sequence
  Synthetic
ggcgttttagttttacggattcg

SEQ ID NO: 4 - DNA, 27 bp, Artificial sequence
  Synthetic
acaaataaaccccatacaaattacgac

SEQ ID NO: 5 - DNA, 20 bp, Artificial sequence
  Synthetic
cgccgaggcgaaaactccct

SEQ ID NO: 6 - DNA, 126 bp, Homo sapiens
cggattcaacatgggcaatgtgcctacactttcattcttccagaacacgatggcaactgtcgtgagagtacgacagaccagtacaacacaaacgctctgcagagagatgctccacacgtggaaccg

SEQ ID NO: 7 - DNA, 126 bp, Artificial sequence
  Synthetic
cggatttaatatgggtaatgtgtttatatttttatttttttagaatacgatggtaattgtcgtgagagtacgatagattagtataatataaacgttttgtagagagatgttttatacgtggaatcg

SEQ ID NO: 8 - DNA, 26 bp, Artificial sequence
  Synthetic
ttttagaatacgatggtaattgtcgt

SEQ ID NO: 9 - DNA, 35 bp, Artificial sequence
  Synthetic
acatctctctacaaaacgtttatattatactaatc

SEQ ID NO: 10 - DNA, 20 bp, Artificial sequence
  Synthetic
cgccgaggctatcgtactct

SEQ ID NO: 11 - DNA, 109 bp, Homo sapiens
ggagctacgacgagcagctgcggctggcgatggaactgtcggcgcaggagcaggaggagaggcggcggcgcgcgcgccaggaggaggaggagctggagcgcatcctgag

SEQ ID NO: 12 - DNA, 109 bp, Artificial sequence
  Synthetic
ggagttacgacgagtagttgcggttggcgatggaattgtcggcgtaggagtaggaggagaggcggcggcgcgcgcgttaggaggaggaggagttggagcgtattttgag

SEQ ID NO: 13 - DNA, 20 bp, Artificial sequence
  Synthetic
agttacgacgagtagttgcg

SEQ ID NO: 14 - DNA, 18 bp, Artificial sequence
  Synthetic
tcctcctactcctacgcc

SEQ ID NO: 15 - DNA, 21 bp, Artificial sequence
  Synthetic
ccacggacgcgacaattccat

SEQ ID NO: 16 - DNA, 143 bp, Homo sapiens
ggtggcaacggctggagtgccgtcgcccgcgccactcaccccggcgcggcgccctgcgcggccgctcagcggaaggccagcaggaagatcagtacgacgttgatgagaaccaggagcgccagcacggcggagaccaccacgcg

SEQ ID NO: 17 - DNA, 143 bp, Artificial sequence
  Synthetic
ggtggtaacggttggagtgtcgtcgttcgcgttatttatttcggcgcggcgttttgcgcggtcgtttagcggaaggttagtaggaagattagtacgacgttgatgagaattaggagcgttagtacggcggagattattacgcg

SEQ ID NO: 18 - DNA, 23 bp, Artificial sequence
  Synthetic
cgttcgcgttatttatttcggcg

SEQ ID NO: 19 - DNA, 24 bp, Artificial sequence
  Synthetic
gctcctaattctcatcaacgtcgt

SEQ ID NO: 20 - DNA, 20 bp, Artificial sequence
  Synthetic
cgccgagggcggcgttttgc

SEQ ID NO: 21 - DNA, 100 bp, Homo sapiens
ggcccggggccgcctgggcccctaggggctggacgtcaacctgttagatagagggcgtgggaccccccgcaggcggctgctcggacgaccgcatccggag

SEQ ID NO: 22 - DNA, 100 bp, Artificial sequence
  Synthetic
ggttcggggtcgtttgggtttttaggggttggacgttaatttgttagatagagggcgtgggatttttcgtaggcggttgttcggacgatcgtattcggag

SEQ ID NO: 23 - DNA, 24 bp, Artificial sequence
  Synthetic
cgttaatttgttagatagagggcg

SEQ ID NO: 24 - DNA, 18 bp, Artificial sequence
  Synthetic
acgatcgtccgaacaacc

SEQ ID NO: 25 - DNA, 21 bp, Artificial sequence
  Synthetic
ccacggacgcgcctacgaaaa

SEQ ID NO: 26 - DNA, 17 bp, Artificial sequence
  Synthetic
tccgaacaaccgcctac

SEQ ID NO: 27 - DNA, 21 bp, Artificial sequence
  Synthetic
ccacggacgcgaaaaatccca

SEQ ID NO: 28 - DNA, 119 bp, Homo sapiens
gcttccagccgcgcgctccgtgccactgccgctctctgcagccccgcgtccccgcagcctccccatggccagcccgcttcgctccgctgcggcccttgcccgccaggtacctcgaaccc

SEQ ID NO: 29 - DNA, 119 bp, Artificial sequence
  Synthetic
gtttttagtcgcgcgtttcgtgttattgtcgttttttgtagtttcgcgttttcgtagtttttttatggttagttcgtttcgtttcgttgcggtttttgttcgttaggtatttcgaattt

SEQ ID NO: 30 - DNA, 27 bp, Artificial sequence
  Synthetic
gtgttattgtcgttttttgtagtttcg

SEQ ID NO: 31 - DNA, 18 bp, Artificial sequence
  Synthetic
cgcaacgaaacgaaacga

SEQ ID NO: 32 - DNA, 20 bp, Artificial sequence
  Synthetic
cgccgagggcgttttcgtag

SEQ ID NO: 33 - DNA, 140 bp, Homo sapiens
gcccgccgcacgccgcaatgctccgcgctccccgcggggtcgggcgactcagacagggaccggaaaagaaccacgcagaagaaagccctatttcttgtcgtctgttcctgtgcagccttgcagcctcgccgcccccgcgt

SEQ ID NO: 34 - DNA, 140 bp, Artificial sequence
  Synthetic
gttcgtcgtacgtcgtaatgtttcgcgtttttcgcggggtcgggcgatttagatagggatcggaaaagaattacgtagaagaaagttttattttttgtcgtttgtttttgtgtagttttgtagtttcgtcgttttcgcgt

SEQ ID NO: 35 - DNA, 21 bp, Artificial sequence
  Synthetic
cgtaatgtttcgcgtttttcg

SEQ ID NO: 36 - DNA, 27 bp, Artificial sequence
  Synthetic
actttcttctacgtaattcttttccga

SEQ ID NO: 37 - DNA, 20 bp, Artificial sequence
  Synthetic
cgccgagggcggggtcgggc

SEQ ID NO: 38 - DNA, 85 bp, Homo sapiens
gccggggagtcgagaagcaagtactagcgctccaggaccgcgcgcgccgccccgcgccgccccgcgccgcccctcggtccagagc

SEQ ID NO: 39 - DNA, 85 bp, Artificial sequence
  Synthetic
gtcggggagtcgagaagtaagtattagcgttttaggatcgcgcgcgtcgtttcgcgtcgtttcgcgtcgtttttcggtttagagt

SEQ ID NO: 40 - DNA, 23 bp, Artificial sequence
  Synthetic
agtattagcgttttaggatcgcg

SEQ ID NO: 41 - DNA, 21 bp, Artificial sequence
  Synthetic
actctaaaccgaaaaacgacg

SEQ ID NO: 42 - DNA, 21 bp, Artificial sequence
  Synthetic
ccacggacggcgaaacgacgc

SEQ ID NO: 43 - DNA, 74 bp, Homo sapiens
gccgggagcccgcacttcctcctcgggggcctcagaaaaccacagggcgcggggccagggcggcggcccccagg

SEQ ID NO: 44 - DNA, 74 bp, Artificial sequence
  Synthetic
gtcgggagttcgtatttttttttcgggggttttagaaaattatagggcgcggggttagggcggcggtttttagg

SEQ ID NO: 45 - DNA, 25 bp, Artificial sequence
  Synthetic
tcgggagttcgtatttttttttcgg

SEQ ID NO: 46 - DNA, 17 bp, Artificial sequence
  Synthetic
aaaaccgccgccctaac

SEQ ID NO: 47 - DNA, 20 bp, Artificial sequence
  Synthetic
cgccgaggccccgcgcccta

SEQ ID NO: 48 - DNA, 78 bp, Homo sapiens
cggggcctaccctcaggcagcgctcgctcgaggccagcttccgagctccaacccctgcccgaaacctcggcctcactg

SEQ ID NO: 49 - DNA, 78 bp, Artificial sequence
  Synthetic
cggggtttatttttaggtagcgttcgttcgaggttagttttcgagttttaatttttgttcgaaatttcggttttattg

SEQ ID NO: 50 - DNA, 24 bp, Artificial sequence
  Synthetic
gggtttatttttaggtagcgttcg

SEQ ID NO: 51 - DNA, 29 bp, Artificial sequence
  Synthetic
cgaaatttcgaacaaaaattaaaactcga

SEQ ID NO: 52 - DNA, 21 bp, Artificial sequence
  Synthetic
ccacggacggttcgaggttag

SEQ ID NO: 53 - DNA, 141 bp, Homo sapiens
tgtcctgacacgatggccacaggcacagtttgtggtgatgcccaggggcccgcgcggccccacggtggtccagtttacactcgggccccgcactcctgaagttccgcgcgggaggagaagggcgtccctttcgcagctcgg

SEQ ID NO: 54 - DNA, 141 bp, Artificial sequence
  Synthetic
tgttttgatacgatggttataggtatagtttgtggtgatgtttaggggttcgcgcggttttacggtggtttagtttatattcgggtttcgtatttttgaagtttcgcgcgggaggagaagggcgtttttttcgtagttcgg

SEQ ID NO: 55 - DNA, 19 bp, Artificial sequence
  Synthetic
tgatgtttaggggttcgcg

SEQ ID NO: 56 - DNA, 26 bp, Artificial sequence
  Synthetic
cgaaacttcaaaaatacgaaacccga

SEQ ID NO: 57 - DNA, 20 bp, Artificial sequence
  Synthetic
cgccgagggcggttttacgg

SEQ ID NO: 58 - DNA, 112 bp, Homo sapiens
ccggagcactcgccgctgcgcgccctgaagccgctggcggtaggcggccctcgaggccggcgggctgggcggctcggcagcctgcgccgcggcctccgcctcggccgccagc

SEQ ID NO: 59 - DNA, 112 bp, Artificial sequence
  Synthetic
tcggagtattcgtcgttgcgcgttttgaagtcgttggcggtaggcggttttcgaggtcggcgggttgggcggttcggtagtttgcgtcgcggttttcgtttcggtcgttagt

SEQ ID NO: 60 - DNA, 17 bp, Artificial sequence
  Synthetic
gtattcgtcgttgcgcg

SEQ ID NO: 61 - DNA, 18 bp, Artificial sequence
  Synthetic
cctcgaaaaccgcctacc

SEQ ID NO: 62 - DNA, 21 bp, Artificial sequence
  Synthetic
ccacggacgcgccaacgactt

SEQ ID NO: 63 - DNA, 134 bp, Homo sapiens
cgccgtgagtgttatagttcttaaaggcggcgtgtccggagtttcttccttctggtggggttcgtggtctcgccggctcaggagtgaagctgcagatcttcgcggtgagtgttacagctcctaaggcggcgcat

SEQ ID NO: 64 - DNA, 134 bp, Artificial sequence
  Synthetic
cgtcgtgagtgttatagtttttaaaggcggcgtgttcggagttttttttttttggtggggttcgtggtttcgtcggtttaggagtgaagttgtagattttcgcggtgagtgttatagtttttaaggcggcgtat

SEQ ID NO: 65 - DNA, 17 bp, Artificial sequence
  Synthetic
taaaggcggcgtgttcg

SEQ ID NO: 66 - DNA, 21 bp, Artificial sequence
  Synthetic
caacttcactcctaaaccgac

SEQ ID NO: 67 - DNA, 21 bp, Artificial sequence
  Synthetic
ccacggacgcgaaaccacgaa

SEQ ID NO: 68 - DNA, 107 bp, Homo sapiens
aactggccttctggctactccggaatcgccaagcagatgaggccagaccgccgccagcgctgatcacgcgcgctcccacaggtcctggcgcgcgtgttcagccgcgc

SEQ ID NO: 69 - DNA, 107 bp, Artificial sequence
  Synthetic
aattggttttttggttatttcggaatcgttaagtagatgaggttagatcgtcgttagcgttgattacgcgcgtttttataggttttggcgcgcgtgtttagtcgcgt

SEQ ID NO: 70 - DNA, 26 bp, Artificial sequence
  Synthetic
tggttttttggttatttcggaatcgt

SEQ ID NO: 71 - DNA, 19 bp, Artificial sequence
  Synthetic
gcgcgtaatcaacgctaac

SEQ ID NO: 72 - DNA, 20 bp, Artificial sequence
  Synthetic
cgccgaggcgacgatctaac

SEQ ID NO: 73 - DNA, 85 bp, Homo sapiens
ggagcgggcagaggaggagcccagcgccgaggcccaggcgcgccccgccctcgcccctccccgtgcccctcccccgctgctcccc

SEQ ID NO: 74 - DNA, 85 bp, Artificial sequence
  Synthetic
ggagcgggtagaggaggagtttagcgtcgaggtttaggcgcgtttcgttttcgttttttttcgtgtttttttttcgttgtttttt

SEQ ID NO: 75 - DNA, 19 bp, Artificial sequence
  Synthetic
gaggaggagtttagcgtcg

SEQ ID NO: 76 - DNA, 24 bp, Artificial sequence
  Synthetic
cacgaaaaaaaacgaaaacgaaac

SEQ ID NO: 77 - DNA, 20 bp, Artificial sequence
  Synthetic
cgccgaggcgcgcctaaacc

SEQ ID NO: 78 - DNA, 107 bp, Homo sapiens
gcggtctatcacgggcacccctaacacttggtgagtgcgcagtgctctcggcagtctctgggctccatacgatgcctaccgcacgccctagcagaggaggtctctgt

SEQ ID NO: 79 - DNA, 107 bp, Artificial sequence
  Synthetic
gcggtttattacgggtatttttaatatttggtgagtgcgtagtgttttcggtagtttttgggttttatacgatgtttatcgtacgttttagtagaggaggtttttgt

SEQ ID NO: 80 - DNA, 20 bp, Artificial sequence
  Synthetic
tgagtgcgtagtgttttcgg

SEQ ID NO: 81 - DNA, 27 bp, Artificial sequence
  Synthetic
ctcctctactaaaacgtacgataaaca

SEQ ID NO: 82 - DNA, 20 bp, Artificial sequence
  Synthetic
cgccgaggatcgtataaaac

SEQ ID NO: 83 - DNA, 21 bp, Artificial sequence
  Synthetic
atatttggtgagtgcgtagtg

SEQ ID NO: 84 - DNA, 26 bp, Artificial sequence
  Synthetic
acgtacgataaacatcgtataaaacc

SEQ ID NO: 85 - DNA, 20 bp, Artificial sequence
  Synthetic
cgccgagggttttcggtagt

SEQ ID NO: 86 - DNA, 121 bp, Homo sapiens
tactccactgccggcttggtgcccacgctcggcttccgcccacccatggactacgcctttagcgatctcatgcgtgaccgctcggccgccgctgctgcggcggtgcacaaggagccgacct

SEQ ID NO: 87 - DNA, 121 bp, Artificial sequence
  Synthetic
tattttattgtcggtttggtgtttacgttcggttttcgtttatttatggattacgtttttagcgattttatgcgtgatcgttcggtcgtcgttgttgcggcggtgtataaggagtcgattt

SEQ ID NO: 88 - DNA, 23 bp, Artificial sequence
  Synthetic
tggtgtttacgttcggttttcgt

SEQ ID NO: 89 - DNA, 17 bp, Artificial sequence
  Synthetic
ccgcaacaacgacgacc

SEQ ID NO: 90 - DNA, 20 bp, Artificial sequence
  Synthetic
cgccgaggcgaacgatcacg

SEQ ID NO: 91 - DNA, 106 bp, Homo sapiens
aggccggtcacgaacaaagcgctggcgagtgcgcgcccgcccacgcgcacaggtgcccgcgacaagacgccccgtccccgcccacgcggcccccgcgggctgagcc

SEQ ID NO: 92 - DNA, 106 bp, Artificial sequence
  Synthetic
aggtcggttacgaataaagcgttggcgagtgcgcgttcgtttacgcgtataggtgttcgcgataagacgtttcgttttcgtttacgcggttttcgcgggttgagtt

SEQ ID NO: 93 - DNA, 21 bp, Artificial sequence
  Synthetic
gttacgaataaagcgttggcg

SEQ ID NO: 94 - DNA, 20 bp, Artificial sequence
  Synthetic
aacgaaacgtcttatcgcga

SEQ ID NO: 95 - DNA, 21 bp, Artificial sequence
  Synthetic
ccacggacggagtgcgcgttc

SEQ ID NO: 96 - DNA, 85 bp, Homo sapiens
gccggccccgcagcatcctcctgctcgcggctctcccgccacctgtcccgctccctgccgcgccctggggcccgcacctacccac

SEQ ID NO: 97 - DNA, 85 bp, Artificial sequence
  Synthetic
gtcggtttcgtagtatttttttgttcgcggttttttcgttatttgtttcgttttttgtcgcgttttggggttcgtatttatttat

SEQ ID NO: 98 - DNA, 25 bp, Artificial sequence
  Synthetic
cggtttcgtagtatttttttgttcg

SEQ ID NO: 99 - DNA, 17 bp, Artificial sequence
  Synthetic
gaaccccaaaacgcgac

SEQ ID NO: 100 - DNA, 20 bp, Artificial sequence
  Synthetic
cgccgagggcggttttttcg

SEQ ID NO: 101 - DNA, 134 bp, Homo sapiens
cgcctcctgggctccccccggagtgggagggagccgcggtcccgcctccgcgcccgttccctcccaggcccctcggccgccgcgccgagctttccgcgcgtggacagactgcccggccgacggacggacgcagg

SEQ ID NO: 102 - DNA, 134 bp, Artificial sequence
  Synthetic
cgttttttgggtttttttcggagtgggagggagtcgcggtttcgttttcgcgttcgtttttttttaggtttttcggtcgtcgcgtcgagtttttcgcgcgtggatagattgttcggtcgacggacggacgtagg

SEQ ID NO: 103 - DNA, 19 bp, Artificial sequence
  Synthetic
gagtcgcggtttcgttttc

SEQ ID NO: 104 - DNA, 19 bp, Artificial sequence
  Synthetic
gacgcgacgaccgaaaaac

SEQ ID NO: 105 - DNA, 20 bp, Artificial sequence
  Synthetic
cgccgaggcgcgttcgtttt

SEQ ID NO: 106 - DNA, 108 bp, Homo sapiens
tccggcgccgcgttttctagagaaccgggtctcagcgatgctcatttcagccccgtcttaatgcaacaaacgaaaccccacacgaacgaaaaggaacatgtctgcgct

SEQ ID NO: 107 - DNA, 107 bp, Artificial sequence
  Synthetic
tcggcgtcgcgttttttagagaatcgggttttagcgatgtttattttagtttcgttttaatgtaataaacgaaattttatacgaacgaaaaggaatatgtttgcgtt

SEQ ID NO: 108 - DNA, 21 bp, Artificial sequence
  Synthetic
ggcgtcgcgttttttagagaa

SEQ ID NO: 109 - DNA, 28 bp, Artificial sequence
  Synthetic
ttccttttcgttcgtataaaatttcgtt

SEQ ID NO: 110 - DNA, 21 bp, Artificial sequence
  Synthetic
ccacggacgatcgggttttag

SEQ ID NO: 111 - DNA, 128 bp, Homo sapiens
gggcctgctggccggggacccgcgcgtcgagcgcctggtgcgcgacagcgcctcctactgccgcgagcgcttcgaccccgacgagtactccacggccgtgcgcgaggcgccagcggagctcgccgaag

SEQ ID NO: 112 - DNA, 128 bp, Artificial sequence
  Synthetic
gggtttgttggtcggggattcgcgcgtcgagcgtttggtgcgcgatagcgttttttattgtcgcgagcgtttcgatttcgacgagtattttacggtcgtgcgcgaggcgttagcggagttcgtcgaag

SEQ ID NO: 113 - DNA, 23 bp, Artificial sequence
  Synthetic
cgatagcgttttttattgtcgcg

SEQ ID NO: 114 - DNA, 22 bp, Artificial sequence
  Synthetic
gcacgaccgtaaaatactcgtc

SEQ ID NO: 115 - DNA, 21 bp, Artificial sequence
  Synthetic
ccacggacgcgaaatcgaaac

SEQ ID NO: 116 - DNA, 140 bp, Homo sapiens
tagcagcagccgcagccatggcggggatgaagacagcctccggggactacatcgactcgtcatgggagctgcgggtgtttgtgggagaggaggacccagaggccgagtcggtcaccctgcgggtcactggggagtcgcac

SEQ ID NO: 117 - DNA, 140 bp, Artificial sequence
  Synthetic
tagtagtagtcgtagttatggcggggatgaagatagttttcggggattatatcgattcgttatgggagttgcgggtgtttgtgggagaggaggatttagaggtcgagtcggttattttgcgggttattggggagtcgtat

SEQ ID NO: 118 - DNA, 24 bp, Artificial sequence
  Synthetic
gttttcggggattatatcgattcg

SEQ ID NO: 119 - DNA, 22 bp, Artificial sequence
  Synthetic
cccaataacccgcaaaataacc

SEQ ID NO: 120 - DNA, 20 bp, Artificial sequence
  Synthetic
cgccgaggcgactcgacctc

SEQ ID NO: 121 - DNA, 104 bp, Homo sapiens
gtcccagagacgccctagggtcagaggtcatctccgtggcaacggaaacttcccgcgctacggcggctccaacgggccgcttccgccgcattgcgtagcgaagc

SEQ ID NO: 122 - DNA, 104 bp, Artificial sequence
  Synthetic
gttttagagacgttttagggttagaggttattttcgtggtaacggaaatttttcgcgttacggcggttttaacgggtcgttttcgtcgtattgcgtagcgaagt

SEQ ID NO: 123 - DNA, 24 bp, Artificial sequence
  Synthetic
tttcgtggtaacggaaatttttcg

SEQ ID NO: 124 - DNA, 17 bp, Artificial sequence
  Synthetic
cgacgaaaacgacccgt

SEQ ID NO: 125 - DNA, 20 bp, Artificial sequence
  Synthetic
cgccgagggcgttacggcgg

SEQ ID NO: 126 - DNA, 107 bp, Homo sapiens
gcgccccggccgcaggcggaggacagggaggagcgcacacgagaaagctcccacgcgcccgcgcctcgcctccgacgggaaggcgcctcttccgaccgtcctggatg

SEQ ID NO: 127 - DNA, 107 bp, Artificial sequence
  Synthetic
gcgtttcggtcgtaggcggaggatagggaggagcgtatacgagaaagtttttacgcgttcgcgtttcgttttcgacgggaaggcgtttttttcgatcgttttggatg

SEQ ID NO: 128 - DNA, 25 bp, Artificial sequence
  Synthetic
gagcgtatacgagaaagtttttacg

SEQ ID NO: 129 - DNA, 17 bp, Artificial sequence
  Synthetic
aacgccttcccgtcgaa

SEQ ID NO: 130 - DNA, 21 bp, Artificial sequence
  Synthetic
ccacggacggcgttcgcgttt

SEQ ID NO: 131 - DNA, 108 bp, Homo sapiens
cgagagggcgcgagcacagccgaggccatggaggtgacggcggaccagccgcgctgggtgagccaccaccaccccgccgtgctcaacgggcagcacccggacacgcac

SEQ ID NO: 132 - DNA, 108 bp, Artificial sequence
  Synthetic
cgagagggcgcgagtatagtcgaggttatggaggtgacggcggattagtcgcgttgggtgagttattattatttcgtcgtgtttaacgggtagtattcggatacgtat

SEQ ID NO: 133 - DNA, 17 bp, Artificial sequence
  Synthetic
gggcgcgagtatagtcg

SEQ ID NO: 134 - DNA, 17 bp, Artificial sequence
  Synthetic
caacgcgactaatccgc

SEQ ID NO: 135 - DNA, 20 bp, Artificial sequence
  Synthetic
cgccgaggccgtcacctcca

SEQ ID NO: 136 - DNA, 141 bp, Homo sapiens
cgccccctcacctccccgatcatgccgttccagacgccatcgatcttctttccgtgcttgccattggtgaccaggtagaggtcgtagctgaagccgatggtatgcgccagccgcttcagaatgtcgatgcagaaacccttg

SEQ ID NO: 137 - DNA, 141 bp, Artificial sequence
  Synthetic
cgtttttttattttttcgattatgtcgttttagacgttatcgatttttttttcgtgtttgttattggtgattaggtagaggtcgtagttgaagtcgatggtatgcgttagtcgttttagaatgtcgatgtagaaatttttg

SEQ ID NO: 138 - DNA, 27 bp, Artificial sequence
  Synthetic
tcgattatgtcgttttagacgttatcg

SEQ ID NO: 139 - DNA, 28 bp, Artificial sequence
  Synthetic
tctacatcgacattctaaaacgactaac

SEQ ID NO: 140 - DNA, 21 bp, Artificial sequence
  Synthetic
ccacggacgcgcataccatcg

SEQ ID NO: 141 - DNA, 93 bp, Homo sapiens
cggcgaggcttcccgcctggcgcattacaacaagcgctcgaccatcacctccagggagatccagacggccgtgcgcctgctgcttcccgggga

SEQ ID NO: 142 - DNA, 93 bp, Artificial sequence
  Synthetic
cggcgaggtttttcgtttggcgtattataataagcgttcgattattatttttagggagatttagacggtcgtgcgtttgttgtttttcgggga

SEQ ID NO: 143 - DNA, 23 bp, Artificial sequence
  Synthetic
tggcgtattataataagcgttcg

SEQ ID NO: 144 - DNA, 18 bp, Artificial sequence
  Synthetic
aacaacaaacgcacgacc

SEQ ID NO: 145 - DNA, 21 bp, Artificial sequence
  Synthetic
ccacggacgcgtctaaatctc

SEQ ID NO: 146 - DNA, 101 bp, Homo sapiens
gggcgggccaggcgctgggcacggtgatggccaccactggggccctgggcaactactacgtggactcgttcctgctgggcgccgacgccgcggatgagctg

SEQ ID NO: 147 - DNA, 101 bp, Artificial sequence
  Synthetic
gggcgggttaggcgttgggtacggtgatggttattattggggttttgggtaattattacgtggattcgtttttgttgggcgtcgacgtcgcggatgagttg

SEQ ID NO: 148 - DNA, 24 bp, Artificial sequence
  Synthetic
ttgggtaattattacgtggattcg

SEQ ID NO: 149 - DNA, 16 bp, Artificial sequence
  Synthetic
actcatccgcgacgtc

SEQ ID NO: 150 - DNA, 21 bp, Artificial sequence
  Synthetic
ccacggacgcgacgcccaaca

SEQ ID NO: 151 - DNA, 95 bp, Homo sapiens
gggccattgccagaagacgtcttctcggggcgccaggattcacctttccttcccgacctcaacttcttcgcggccgactcctgtctccagctatc

SEQ ID NO: 152 - DNA, 95 bp, Artificial sequence
  Synthetic
gggttattgttagaagacgttttttcggggcgttaggatttatttttttttttcgattttaattttttcgcggtcgatttttgtttttagttatt

SEQ ID NO: 153 - DNA, 22 bp, Artificial sequence
  Synthetic
gttagaagacgttttttcgggg

SEQ ID NO: 154 - DNA, 20 bp, Artificial sequence
  Synthetic
aaaacaaaaatcgaccgcga

SEQ ID NO: 155 - DNA, 20 bp, Artificial sequence
  Synthetic
cgccgagggcgttaggattt

SEQ ID NO: 156 - DNA, 106 bp, Homo sapiens
ggccccggaagcccagctcccgggccctggagcccgccacggcggcagccctgcggcggcggctggacctgggcagttgcctggacgtgctggcctttgcccagca

SEQ ID NO: 157 - DNA, 106 bp, Artificial sequence
  Synthetic
ggtttcggaagtttagttttcgggttttggagttcgttacggcggtagttttgcggcggcggttggatttgggtagttgtttggacgtgttggtttttgtttagta

SEQ ID NO: 158 - DNA, 25 bp, Artificial sequence
  Synthetic
agttttcgggttttggagttcgtta

SEQ ID NO: 159 - DNA, 17 bp, Artificial sequence
  Synthetic
ccaaatccaaccgccgc

SEQ ID NO: 160 - DNA, 20 bp, Artificial sequence
  Synthetic
cgccgaggacggcggtagtt

SEQ ID NO: 161 - DNA, 106 bp, Homo sapiens
ggcggcgccggcggctgcgcggggggcgccaggccctgctgctgctgctgctgctgactgcggtagtaggcggcggcggccacggcggcaaagttgtgggtctgga

SEQ ID NO: 162 - DNA, 106 bp, Artificial sequence
  Synthetic
ggcggcgtcggcggttgcgcggggggcgttaggttttgttgttgttgttgttgttgattgcggtagtaggcggcggcggttacggcggtaaagttgtgggtttgga

SEQ ID NO: 163 - DNA, 19 bp, Artificial sequence
  Synthetic
ttgattgcggtagtaggcg

SEQ ID NO: 164 - DNA, 19 bp, Artificial sequence
  Synthetic
aacccacaactttaccgcc

SEQ ID NO: 165 - DNA, 20 bp, Artificial sequence
  Synthetic
cgccgaggcgtaaccgccgc

SEQ ID NO: 166 - DNA, 154 bp, Homo sapiens
ggtttccccccaccccggcctcggggtctctccacgtctccccgccgacgtgctcacctgctcagggggcgcccccgagccgcgccccgcgcccgcccccaggagggcctccgcgagccggctgcacaccccgaggcggtcccggctgcacaac

SEQ ID NO: 167 - DNA, 154 bp, Artificial sequence
  Synthetic
ggtttttttttatttcggtttcggggtttttttacgttttttcgtcgacgtgtttatttgtttagggggcgttttcgagtcgcgtttcgcgttcgtttttaggagggttttcgcgagtcggttgtatatttcgaggcggtttcggttgtataat

SEQ ID NO: 168 - DNA, 27 bp, Artificial sequence
  Synthetic
gtttcggggtttttttacgttttttcg

SEQ ID NO: 169 - DNA, 19 bp, Artificial sequence
  Synthetic
aaacgcgactcgaaaacgc

SEQ ID NO: 170 - DNA, 20 bp, Artificial sequence
  Synthetic
cgccgagggtcgacgtgttt

SEQ ID NO: 171 - DNA, 95 bp, Homo sapiens
ctccggttttcgcggttctcagcgatattaggcgcggccagtgtctgaaagctcctcggggttacgtcctggggcgactggaggcggctcacgac

SEQ ID NO: 172 - DNA, 95 bp, Artificial sequence
  Synthetic
tttcggttttcgcggtttttagcgatattaggcgcggttagtgtttgaaagtttttcggggttacgttttggggcgattggaggcggtttacgat

SEQ ID NO: 173 - DNA, 22 bp, Artificial sequence
  Synthetic
cggtttttagcgatattaggcg

SEQ ID NO: 174 - DNA, 18 bp, Artificial sequence
  Synthetic
cccaaaacgtaaccccga

SEQ ID NO: 175 - DNA, 20 bp, Artificial sequence
  Synthetic
cgccgagggcggttagtgtt

SEQ ID NO: 176 - DNA, 143 bp, Homo sapiens
cgacggccgcggaggaggaaggccagggggaaatttgcatttcgtaaaaccgcggttaagaaatgacgatgccacgtagacaagccagttgtgacgttcagcacaacgtgctactgaactaccgagatccgccaccaaatggc

SEQ ID NO: 177 - DNA, 143 bp, Artificial sequence
  Synthetic
cgacggtcgcggaggaggaaggttagggggaaatttgtatttcgtaaaatcgcggttaagaaatgacgatgttacgtagataagttagttgtgacgtttagtataacgtgttattgaattatcgagattcgttattaaatggt

SEQ ID NO: 178 - DNA, 25 bp, Artificial sequence
  Synthetic
gggaaatttgtatttcgtaaaatcg

SEQ ID NO: 179 - DNA, 27 bp, Artificial sequence
  Synthetic
acaactaacttatctacgtaacatcgt

SEQ ID NO: 180 - DNA, 21 bp, Artificial sequence
  Synthetic
ccacggacggcggttaagaaa

SEQ ID NO: 181 - DNA, 116 bp, Homo sapiens
ggcttggggtccagccgcccgcccctgccgccaccgcaccatgtcctgcctctactcccgcctcagcgccccctgcggggtccgcgccttcagctgcatctcggcctgcgggcccc

SEQ ID NO: 182 - DNA, 116 bp, Artificial sequence
  Synthetic
ggtttggggtttagtcgttcgtttttgtcgttatcgtattatgttttgtttttattttcgttttagcgttttttgcggggttcgcgtttttagttgtatttcggtttgcgggtttt

SEQ ID NO: 183 - DNA, 22 bp, Artificial sequence
  Synthetic
tcgttcgtttttgtcgttatcg

SEQ ID NO: 184 - DNA, 23 bp, Artificial sequence
  Synthetic
aaccgaaatacaactaaaaacgc

SEQ ID NO: 185 - DNA, 21 bp, Artificial sequence
  Synthetic
ccacggacgcgaaccccgcaa

SEQ ID NO: 186 - DNA, 108 bp, Homo sapiens
ggaaggctgcagcgagagatttacatattcatccgagcttaaggaagccgcgataatgcaggtacagcccgaaacccacgcccccagaccttatctgcgcgccccgcc

SEQ ID NO: 187 - DNA, 108 bp, Artificial sequence
  Synthetic
ggaaggttgtagcgagagatttatatatttattcgagtttaaggaagtcgcgataatgtaggtatagttcgaaatttacgtttttagattttatttgcgcgtttcgtt

SEQ ID NO: 188 - DNA, 19 bp, Artificial sequence
  Synthetic
ttcgagtttaaggaagtcg

SEQ ID NO: 189 - DNA, 23 bp, Artificial sequence
  Synthetic
tctaaaaacgtaaatttcgaact

SEQ ID NO: 190 - DNA, 21 bp, Artificial sequence
  Synthetic
ccacggacggcgataatgtag

SEQ ID NO: 191 - DNA, 137 bp, Homo sapiens
ggagttatttttaaccatcgcctcccagaacattacggagcttcctctctccaacacgcaggaaaccctacttggctgtgcttcctgctaacacgaggccctgcgattgctgagaacaacagccccgagactgcgcg

SEQ ID NO: 192 - DNA, 137 bp, Artificial sequence
  Synthetic
ggagttatttttaattatcgttttttagaatattacggagttttttttttttaatacgtaggaaattttatttggttgtgttttttgttaatacgaggttttgcgattgttgagaataatagtttcgagattgcgcg

SEQ ID NO: 193 - DNA, 30 bp, Artificial sequence
  Synthetic
tttaattatcgttttttagaatattacgga

SEQ ID NO: 194 - DNA, 26 bp, Artificial sequence
  Synthetic
actattattctcaacaatcgcaaaac

SEQ ID NO: 195 - DNA, 21 bp, Artificial sequence
  Synthetic
ccacggacgcctcgtattaac

SEQ ID NO: 196 - DNA, 117 bp, Homo sapiens
ggcgggcgcttggccaaacagcccaagactgcggaatcacactcgccactgtgtacctggacgccatctgcagacccagcgcctgcggggattccggaaacgggagagcgggcttcc

SEQ ID NO: 197 - DNA, 117 bp, Artificial sequence
  Synthetic
ggcgggcgtttggttaaatagtttaagattgcggaattatattcgttattgtgtatttggacgttatttgtagatttagcgtttgcggggatttcggaaacgggagagcgggttttt

SEQ ID NO: 198 - DNA, 27 bp, Artificial sequence
  Synthetic
agtttaagattgcggaattatattcgt

SEQ ID NO: 199 - DNA, 17 bp, Artificial sequence
  Synthetic
ttccgaaatccccgcaa

SEQ ID NO: 200 - DNA, 20 bp, Artificial sequence
  Synthetic
cgccgaggaacgctaaatct

SEQ ID NO: 201 - DNA, 156 bp, Homo sapiens
cgcaggctgaggccctcgggtccccagcgggtcctcgccatcagtcactctctacgggccaggcctgggggtcacggcctgcaggagcctccctgcgcggccccactccctcatctgcgaccccgtggggaggcgaccctgaccaccctcgttccg

SEQ ID NO: 202 - DNA, 156 bp, Artificial sequence
  Synthetic
cgtaggttgaggttttcgggtttttagcgggttttcgttattagttattttttacgggttaggtttgggggttacggtttgtaggagtttttttgcgcggttttatttttttatttgcgatttcgtggggaggcgattttgattattttcgtttcg

SEQ ID NO: 203 - DNA, 23 bp, Artificial sequence
  Synthetic
ggttgaggttttcgggtttttag

SEQ ID NO: 204 - DNA, 17 bp, Artificial sequence
  Synthetic
cctccccacgaaatcgc

SEQ ID NO: 205 - DNA, 20 bp, Artificial sequence
  Synthetic
cgccgagggcgggttttcgt

SEQ ID NO: 206 - DNA, 18 bp, Artificial sequence
  Synthetic
aggagtttttttgcgcgg

SEQ ID NO: 207 - DNA, 24 bp, Artificial sequence
  Synthetic
acgaaaataatcaaaatcgcctcc

SEQ ID NO: 208 - DNA, 20 bp, Artificial sequence
  Synthetic
cgccgaggcccacgaaatcg

SEQ ID NO: 209 - DNA, 114 bp, Homo sapiens
cgggggagggcggcatcagccagagcctcagccgacggcgctccccaggtccacttcccgctccgataccctccccctaagcacgatacccagggcccagggctgctcttggcg

SEQ ID NO: 210 - DNA, 114 bp, Artificial sequence
  Synthetic
cgggggagggcggtattagttagagttttagtcgacggcgttttttaggtttatttttcgtttcgatattttttttttaagtacgatatttagggtttagggttgtttttggcg

SEQ ID NO: 211 - DNA, 25 bp, Artificial sequence
  Synthetic
gcggtattagttagagttttagtcg

SEQ ID NO: 212 - DNA, 24 bp, Artificial sequence
  Synthetic
acaaccctaaaccctaaatatcgt

SEQ ID NO: 213 - DNA, 21 bp, Artificial sequence
  Synthetic
ccacggacggacggcgttttt

SEQ ID NO: 214 - DNA, 107 bp, Homo sapiens
ctccgctccccgcaggcctggccgcgcgacgggcacccagcgggttgttatcaattattcaggccccaagttcacgggcactgcatccatttccctcgcgtgcgccc

SEQ ID NO: 215 - DNA, 107 bp, Artificial sequence
  Synthetic
tttcgtttttcgtaggtttggtcgcgcgacgggtatttagcgggttgttattaattatttaggttttaagtttacgggtattgtatttatttttttcgcgtgcgttt

SEQ ID NO: 216 - DNA, 19 bp, Artificial sequence
  Synthetic
tttcgtaggtttggtcgcg

SEQ ID NO: 217 - DNA, 26 bp, Artificial sequence
  Synthetic
aacctaaataattaataacaacccgc

SEQ ID NO: 218 - DNA, 21 bp, Artificial sequence
  Synthetic
ccacggacggcgacgggtatt

SEQ ID NO: 219 - DNA, 88 bp, Homo sapiens
gtgggccgggcgtgacgcgcggtcaaagtgcaatgatttttcagttcggttggctaaacagggtcagagctgagagcgaagcagaagg

SEQ ID NO: 220 - DNA, 88 bp, Artificial sequence
  Synthetic
gtgggtcgggcgtgacgcgcggttaaagtgtaatgattttttagttcggttggttaaatagggttagagttgagagcgaagtagaagg

SEQ ID NO: 221 - DNA, 18 bp, Artificial sequence
  Synthetic
tggttcgggcgtgacgcg

SEQ ID NO: 222 - DNA, 23 bp, Artificial sequence
  Synthetic
tctaaccctatttaaccaaccga

SEQ ID NO: 223 - DNA, 20 bp, Artificial sequence
  Synthetic
cgccgagggcggttaaagtg

SEQ ID NO: 224 - DNA, 94 bp, Homo sapiens
ggacctcctcggccccgccccatccgccttcgggatgctgctgagccccgtcacctccacccccttctcggtcaaggacatcctgcgactggag

SEQ ID NO: 225 - DNA, 94 bp, Artificial sequence
  Synthetic
ggattttttcggtttcgttttattcgttttcgggatgttgttgagtttcgttatttttatttttttttcggttaaggatattttgcgattggag

SEQ ID NO: 226 - DNA, 25 bp, Artificial sequence
  Synthetic
gattttttcggtttcgttttattcg

SEQ ID NO: 227 - DNA, 24 bp, Artificial sequence
  Synthetic
caatcgcaaaatatccttaaccga

SEQ ID NO: 228 - DNA, 21 bp, Artificial sequence
  Synthetic
ccacggacggttttcgggatg

SEQ ID NO: 229 - DNA, 89 bp, Homo sapiens
ctgtcagtgctgaccgagcgccgcgccttccggccatacgggctccacggtgcgcggttccccagccctcgcggccctccccgcccccg

SEQ ID NO: 230 - DNA, 89 bp, Artificial sequence
  Synthetic
ttgttagtgttgatcgagcgtcgcgtttttcggttatacgggttttacggtgcgcggttttttagttttcgcggtttttttcgttttcg

SEQ ID NO: 231 - DNA, 22 bp, Artificial sequence
  Synthetic
cgtcgcgtttttcggttatacg

SEQ ID NO: 232 - DNA, 21 bp, Artificial sequence
  Synthetic
cgcgaaaactaaaaaaccgcg

SEQ ID NO: 233 - DNA, 21 bp, Artificial sequence
  Synthetic
ccacggacggcaccgtaaaac

SEQ ID NO: 234 - DNA, 114 bp, Homo sapiens
cggagtatggtgaggagcgcgggggacgggtgcgggaaggggacagcagggctgagcctggggcccgcaagacccagcagcccgagcgggcgcagagaccccacgccacgcaca

SEQ ID NO: 235 - DNA, 114 bp, Artificial sequence
  Synthetic
cggagtatggtgaggagcgcgggggacgggtgcgggaaggggatagtagggttgagtttggggttcgtaagatttagtagttcgagcgggcgtagagattttacgttacgtata

SEQ ID NO: 236 - DNA, 18 bp, Artificial sequence
  Synthetic
ggttgagtttggggttcg

SEQ ID NO: 237 - DNA, 23 bp, Artificial sequence
  Synthetic
cgtaacgtaaaatctctacgccc

SEQ ID NO: 238 - DNA, 21 bp, Artificial sequence
  Synthetic
ccacggacgcgctcgaactac

SEQ ID NO: 239 - DNA, 95 bp, Homo sapiens
ggagagcagcccgcagaacctggccgcgtactacacgcctttcccgtcctatggacactacagaaacagcctggccaccgtggaggaagacttcc

SEQ ID NO: 240 - DNA, 95 bp, Artificial sequence
  Synthetic
ggagagtagttcgtagaatttggtcgcgtattatacgtttttttcgttttatggatattatagaaatagtttggttatcgtggaggaagattttt

SEQ ID NO: 241 - DNA, 23 bp, Artificial sequence
  Synthetic
gagtagttcgtagaatttggtcg

SEQ ID NO: 242 - DNA, 31 bp, Artificial sequence
  Synthetic
ccacgataaccaaactatttctataatatcc

SEQ ID NO: 243 - DNA, 21 bp, Artificial sequence
  Synthetic
ccacggacggcgtattatacg

SEQ ID NO: 244 - DNA, 23 bp, Artificial sequence
  Synthetic
ggagagtagttcgtagaatttgg

SEQ ID NO: 245 - DNA, 35 bp, Artificial sequence
  Synthetic
ctatttctataatatccataaaacgaaaaaaacgt

SEQ ID NO: 246 - DNA, 21 bp, Artificial sequence
  Synthetic
ccacggacggtcgcgtattat

SEQ ID NO: 247 - DNA, 92 bp, Homo sapiens
gggaaggtgccctgcgcgcgcgcgctcaccagatgaagtcggtgcagtggctgcagaaggtgggctgcttgaagaagcgggcggtgaatttg

SEQ ID NO: 248 - DNA, 92 bp, Artificial sequence
  Synthetic
gggaaggtgttttgcgcgcgcgcgtttattagatgaagtcggtgtagtggttgtagaaggtgggttgtttgaagaagcgggcggtgaatttg

SEQ ID NO: 249 - DNA, 17 bp, Artificial sequence
  Synthetic
ggaaggtgttttgcgcg

SEQ ID NO: 250 - DNA, 21 bp, Artificial sequence
  Synthetic
cttctacaaccactacaccga

SEQ ID NO: 251 - DNA, 21 bp, Artificial sequence
  Synthetic
ccacggacggcgcgcgtttat

SEQ ID NO: 252 - DNA, 107 bp, Homo sapiens
gcctcggggcccggggactcacaattacgggcagagaacacatagtgaagagcacggtcatcagcgccagcagcaggaggtgatccagctcctccaggggctgaggg

SEQ ID NO: 253 - DNA, 107 bp, Artificial sequence
  Synthetic
gtttcggggttcggggatttataattacgggtagagaatatatagtgaagagtacggttattagcgttagtagtaggaggtgatttagtttttttaggggttgaggg

SEQ ID NO: 254 - DNA, 24 bp, Artificial sequence
  Synthetic
gggttcggggatttataattacgg

SEQ ID NO: 255 - DNA, 25 bp, Artificial sequence
  Synthetic
cctcctactactaacgctaataacc

SEQ ID NO: 256 - DNA, 21 bp, Artificial sequence
  Synthetic
ccacggacgcgtactcttcac

SEQ ID NO: 257 - DNA, 80 bp, Homo sapiens
ggcggctgcagcggcacccgcgctcctgcaccagggactgtgccgagccgcgcgcggacgggagggaagcgtcccctcag

SEQ ID NO: 258 - DNA, 80 bp, Artificial sequence
  Synthetic
ggcggttgtagcggtattcgcgtttttgtattagggattgtgtcgagtcgcgcgcggacgggagggaagcgtttttttag

SEQ ID NO: 259 - DNA, 18 bp, Artificial sequence
  Synthetic
gttgtagcggtattcgcg

SEQ ID NO: 260 - DNA, 18 bp, Artificial sequence
  Synthetic
cttctctcccgtccgcgc

SEQ ID NO: 261 - DNA, 20 bp, Artificial sequence
  Synthetic
cgccgaggcgcgactcgaca

SEQ ID NO: 262 - DNA, 143 bp, Homo sapiens
tccagaaacacgggtatctccgcgtggtgctttgcggtcgccgtcgttgtggccgtccggggtggggtgtgaggaggggacgaaggagggaaggaagggcaaggcggggggggctctgcgagagcgcgcccagccccgccttc

SEQ ID NO: 263 - DNA, 143 bp, Artificial sequence
  Synthetic
tttagaaatacgggtattttcgcgtggtgttttgcggtcgtcgtcgttgtggtcgttcggggtggggtgtgaggaggggacgaaggagggaaggaagggtaaggcggggggggttttgcgagagcgcgtttagtttcgttttt

SEQ ID NO: 264 - DNA, 21 bp, Artificial sequence
  Synthetic
agaaatacgggtattttcgcg

SEQ ID NO: 265 - DNA, 17 bp, Artificial sequence
  Synthetic
ccacaacgacgacgacc

SEQ ID NO: 266 - DNA, 21 bp, Artificial sequence
  Synthetic
ccacggacgcgcaaaacacca

SEQ ID NO: 267 - DNA, 120 bp, Homo sapiens
cggtcgggcaggcgggacggagattacctggctgtccaggggaccttatgcagggtttggcccgagcccaggggcagcgaggggcgtctgcggatgcggctccctgtgcggcacaacacc

SEQ ID NO: 268 - DNA, 120 bp, Artificial sequence
  Synthetic
cggtcgggtaggcgggacggagattatttggttgtttaggggattttatgtagggtttggttcgagtttaggggtagcgaggggcgtttgcggatgcggttttttgtgcggtataatatt

SEQ ID NO: 269 - DNA, 20 bp, Artificial sequence
  Synthetic
gttcgagtttaggggtagcg

SEQ ID NO: 270 - DNA, 17 bp, Artificial sequence
  Synthetic
ccgcacaaaaaaccgca

SEQ ID NO: 271 - DNA, 21 bp, Artificial sequence
  Synthetic
ccacggacgatccgcaaacgc

SEQ ID NO: 272 - DNA, 55 bp, Homo sapiens
ccggagcactcgccgctgcgcgccctgaagccgctggcggtaggcggccctcgag

SEQ ID NO: 273 - DNA, 55 bp, Artificial sequence
  Synthetic
tcggagtattcgtcgttgcgcgttttgaagtcgttggcggtaggcggttttcgag

SEQ ID NO: 274 - DNA, 18 bp, Artificial sequence
  Synthetic
ggagtattcgtcgttgcg

SEQ ID NO: 275 - DNA, 17 bp, Artificial sequence
  Synthetic
cgaaaaccgcctaccgc

SEQ ID NO: 276 - DNA, 20 bp, Artificial sequence
  Synthetic
cgccgagggcgttttgaagt

SEQ ID NO: 277 - DNA, 96 bp, Homo sapiens
cccgggcctacggtcctcccgccacctccacggggcggctgttggggccccaccaggcagagccgtgttctcaggcgttggctctcatggaggtgg

SEQ ID NO: 278 - DNA, 96 bp, Artificial sequence
  Synthetic
ttcgggtttacggttttttcgttatttttacggggcggttgttggggttttattaggtagagtcgtgtttttaggcgttggtttttatggaggtgg

SEQ ID NO: 279 - DNA, 25 bp, Artificial sequence
  Synthetic
acggttttttcgttatttttacggg

SEQ ID NO: 280 - DNA, 21 bp, Artificial sequence
  Synthetic
caacgcctaaaaacacgactc

SEQ ID NO: 281 - DNA, 20 bp, Artificial sequence
  Synthetic
cgccgaggggcggttgttgg

SEQ ID NO: 282 - DNA, 148 bp, Homo sapiens
gggcctgtcccgttccctgctccccatacaggcgaggctgcgtgcacacagcttcctgtaccccaggagggcctgcctggcacgcacccggtggctgcaccatccacacgcaagactgcaacttcagatgctccgcacgctggagatg

SEQ ID NO: 283 - DNA, 148 bp, Artificial sequence
  Synthetic
gggtttgtttcgttttttgttttttatataggcgaggttgcgtgtatatagttttttgtattttaggagggtttgtttggtacgtattcggtggttgtattatttatacgtaagattgtaattttagatgtttcgtacgttggagatg

SEQ ID NO: 284 - DNA, 20 bp, Artificial sequence
  Synthetic
ttatataggcgaggttgcgt

SEQ ID NO: 285 - DNA, 30 bp, Artificial sequence
  Synthetic
cttacgtataaataatacaaccaccgaata

SEQ ID NO: 286 - DNA, 21 bp, Artificial sequence
  Synthetic
ccacggacgacgtaccaaaca

SEQ ID NO: 287 - DNA, 88 bp, Homo sapiens
cggagctaggagggtggggctcggagggcgcaggaagagcggctctgcgaggaaagggaaaggagaggccgcttctgggaagggaccc

SEQ ID NO: 288 - DNA, 88 bp, Artificial sequence
  Synthetic
cggagttaggagggtggggttcggagggcgtaggaagagcggttttgcgaggaaagggaaaggagaggtcgtttttgggaagggattt

SEQ ID NO: 289 - DNA, 18 bp, Artificial sequence
  Synthetic
ttaggagggtggggttcg

SEQ ID NO: 290 - DNA, 18 bp, Artificial sequence
  Synthetic
ctttcctcgcaaaaccgc

SEQ ID NO: 291 - DNA, 21 bp, Artificial sequence
  Synthetic
ccacggacgggagggcgtagg

SEQ ID NO: 292 - DNA, 59 bp, Homo sapiens
gccccggcgggccccgaggcggccgcggcctgcaacgtcatcgtgaacggcacgcgcgg

SEQ ID NO: 293 - DNA, 59 bp, Artificial sequence
  Synthetic
gtttcggcgggtttcgaggcggtcgcggtttgtaacgttatcgtgaacggtacgcgcgg

SEQ ID NO: 294 - DNA, 17 bp, Artificial sequence
  Synthetic
tttcggcgggtttcgag

SEQ ID NO: 295 - DNA, 20 bp, Artificial sequence
  Synthetic
cgtaccgttcacgataacgt

SEQ ID NO: 296 - DNA, 20 bp, Artificial sequence
  Synthetic
cgccgaggggcggtcgcggt

SEQ ID NO: 297 - DNA, 20 bp, Artificial sequence
  Synthetic
cgccgaggttacaaaccgcg

SEQ ID NO: 298 - DNA, 89 bp, Homo sapiens
ctaggcgagatggtggaaggcgtgtccgtacgggggtgggctggggtccccgtgcagaagggcgcgcgaggacccaggctggttttccc

SEQ ID NO: 299 - DNA, 89 bp, Artificial sequence
  Synthetic
ttaggcgagatggtggaaggcgtgttcgtacgggggtgggttggggttttcgtgtagaagggcgcgcgaggatttaggttggttttttt

SEQ ID NO: 300 - DNA, 17 bp, Artificial sequence
  Synthetic
cgagatggtggaaggcg

SEQ ID NO: 301 - DNA, 17 bp, Artificial sequence
  Synthetic
gcgcccttctacacgaa

SEQ ID NO: 302 - DNA, 21 bp, Artificial sequence
  Synthetic
ccacggacggtgttcgtacgg

SEQ ID NO: 303 - DNA, 145 bp, Homo sapiens
gcgctgctgcgccgccaggcaaggcgagggtccgggagaaggctcggctccctcctaaacatgtggcccgtggcgtccccttgtcccctccgagcgatgctcctgcgcccttcgccgcctcccgcgctgctgcgccgccaggcaa

SEQ ID NO: 304 - DNA, 123 bp, Artificial sequence
  Synthetic
ggcgagggttcgggagaaggttcggtttttttttaaatatgtggttcgtggcgtttttttgtttttttcgagcgatgtttttgcgtttttcgtcgtttttcgcgttgttgcgtcgttaggtaa

SEQ ID NO: 305 - DNA, 21 bp, Artificial sequence
  Synthetic
aaatatgtggttcgtggcgtt

SEQ ID NO: 306 - DNA, 19 bp, Artificial sequence
  Synthetic
acgcaacaacgcgaaaaac

SEQ ID NO: 307 - DNA, 20 bp, Artificial sequence
  Synthetic
cgccgaggcgacgaaaaacg

SEQ ID NO: 308 - DNA, 137 bp, Homo sapiens
acgagaaagagatcgtgcagggggtgctgcaacagggcacggcgtggaggaggaaccagaccgcggccagagcgttcaggtactcctgccctcgcggctcctcccctctagcgtcctttcctccccgagtgcagagg

SEQ ID NO: 309 - DNA, 137 bp, Artificial sequence
  Synthetic
acgagaaagagatcgtgtagggggtgttgtaatagggtacggcgtggaggaggaattagatcgcggttagagcgtttaggtatttttgttttcgcggttttttttttttagcgttttttttttttcgagtgtagagg

SEQ ID NO: 310 - DNA, 21 bp, Artificial sequence
  Synthetic
ggggtgttgtaatagggtacg

SEQ ID NO: 311 - DNA, 19 bp, Artificial sequence
  Synthetic
ctaaacgctctaaccgcga

SEQ ID NO: 312 - DNA, 21 bp, Artificial sequence
  Synthetic
ccacggacgggcgtggaggag

SEQ ID NO: 313 - DNA, 117 bp, Homo sapiens
cgcgccgttggtcacctcgccggccgccagcgtcgaatggaagcccgacttgtaccaggactcgtacgggtgcgccatgcccacgcgcgggtacagcccgtcggctgccgtcgtgtg

SEQ ID NO: 314 - DNA, 117 bp, Artificial sequence
  Synthetic
cgcgtcgttggttatttcgtcggtcgttagcgtcgaatggaagttcgatttgtattaggattcgtacgggtgcgttatgtttacgcgcgggtatagttcgtcggttgtcgtcgtgtg

SEQ ID NO: 315 - DNA, 21 bp, Artificial sequence
  Synthetic
tagcgtcgaatggaagttcga

SEQ ID NO: 316 - DNA, 18 bp, Artificial sequence
  Synthetic
ggtcgttagcgtcgaatg

SEQ ID NO: 317 - DNA, 20 bp, Artificial sequence
  Synthetic
gcgcgtaaacataacgcacc

SEQ ID NO: 318 - DNA, 21 bp, Artificial sequence
  Synthetic
ccacggacgccgtacgaatcc

SEQ ID NO: 319 - DNA, 85 bp, Homo sapiens
ggttccttcccgtgggttcttaatcgtctcgctgacttccagaatgaaactgcagaccctcgcggtaaagatggcgtgaccagaa

SEQ ID NO: 320 - DNA, 85 bp, Artificial sequence
  Synthetic
ggttttttttcgtgggtttttaatcgtttcgttgatttttagaatgaaattgtagattttcgcggtaaagatggcgtgattagaa

SEQ ID NO: 321 - DNA, 22 bp, Artificial sequence
  Synthetic
tcgtgggtttttaatcgtttcg

SEQ ID NO: 322 - DNA, 18 bp, Artificial sequence
  Synthetic
tcacgccatctttaccgc

SEQ ID NO: 323 - DNA, 21 bp, Artificial sequence
  Synthetic
ccacggacgcgaaaatctaca

SEQ ID NO: 324 - DNA, 26 bp, Artificial sequence
  Synthetic
ggttttttttcgtgggtttttaatcg

SEQ ID NO: 325 - DNA, 21 bp, Artificial sequence
  Synthetic
ctaatcacgccatctttaccg

SEQ ID NO: 326 - DNA, 21 bp, Artificial sequence
  Synthetic
ccacggacggtttcgttgatt

SEQ ID NO: 327 - DNA, 115 bp, Homo sapiens
ggagtgagtgcctacaacgcgcaggccggactgatcccccgttgctgcaggttggtgccccaagctgcgggtgctcgggcgccaactaaagccagctctgtccagacgcggaaag

SEQ ID NO: 328 - DNA, 115 bp, Artificial sequence
  Synthetic
ggagtgagtgtttataacgcgtaggtcggattgatttttcgttgttgtaggttggtgttttaagttgcgggtgttcgggcgttaattaaagttagttttgtttagacgcggaaag

SEQ ID NO: 329 - DNA, 23 bp, Artificial sequence
  Synthetic
cgtaggtcggattgatttttcgt

SEQ ID NO: 330 - DNA, 30 bp, Artificial sequence
  Synthetic
tctaaacaaaactaactttaattaacgccc

SEQ ID NO: 331 - DNA, 21 bp, Artificial sequence
  Synthetic
ccacggacgcgaacacccgca

SEQ ID NO: 332 - DNA, 19 bp, Artificial sequence
  Synthetic
aggaaattgcgggttttcg

SEQ ID NO: 333 - DNA, 21 bp, Artificial sequence
  Synthetic
ggaaggaaattgcgggttttc

SEQ ID NO: 334 - DNA, 25 bp, Artificial sequence
  Synthetic
ccaaaaatcgtcgctaaaaatcaac

SEQ ID NO: 335 - DNA, 21 bp, Artificial sequence
  Synthetic
ccacggacgcgcgcattcact

SEQ ID NO: 336 - DNA, 100 bp, Homo sapiens
ggcggccgcgacccctccccgctgacctcactcgagccgccgcctggcgcagatataagcggcggcccatctgaagagggctcggcaggcgcccggggtc

SEQ ID NO: 337 - DNA, 100 bp, Artificial sequence
  Synthetic
ggcggtcgcgattttttttcgttgattttattcgagtcgtcgtttggcgtagatataagcggcggtttatttgaagagggttcggtaggcgttcggggtt

SEQ ID NO: 338 - DNA, 23 bp, Artificial sequence
  Synthetic
tttcgttgattttattcgagtcg

SEQ ID NO: 339 - DNA, 19 bp, Artificial sequence
  Synthetic
tcttcaaataaaccgccgc

SEQ ID NO: 340 - DNA, 20 bp, Artificial sequence
  Synthetic
cgccgagggtcgtttggcgt

SEQ ID NO: 341 - DNA, 118 bp, Homo sapiens
cgggtggtgaagctgccccacggcctgggagagccttatcgccgcggtcgctggacgtgtgtggatgtttatgagcgagacctggagccccacagcttcggcggactcctggagggaa

SEQ ID NO: 342 - DNA, 118 bp, Artificial sequence
  Synthetic
cgggtggtgaagttgttttacggtttgggagagttttatcgtcgcggtcgttggacgtgtgtggatgtttatgagcgagatttggagttttatagtttcggcggatttttggagggaa

SEQ ID NO: 343 - DNA, 22 bp, Artificial sequence
  Synthetic
gtttgggagagttttatcgtcg

SEQ ID NO: 344 - DNA, 18 bp, Artificial sequence
  Synthetic
cctccaaaaatccgccga

SEQ ID NO: 345 - DNA, 20 bp, Artificial sequence
  Synthetic
cgccgagggcggtcgttgga

SEQ ID NO: 346 - DNA, 70 bp, Homo sapiens
ggggcgggggccgacagcccacgctggcgcggcaggcgcgtgcgcccgccgttttcgtgagcccgagcag

SEQ ID NO: 347 - DNA, 70 bp, Artificial sequence
  Synthetic
ggggtcggggtcgatagtttacgttggcgcggtaggcgcgtgcgttcgtcgttttcgtgagttcgagtag

SEQ ID NO: 348 - DNA, 20 bp, Artificial sequence
  Synthetic
gtcggggtcgatagtttacg

SEQ ID NO: 349 - DNA, 19 bp, Artificial sequence
  Synthetic
actcgaactcacgaaaacg

SEQ ID NO: 350 - DNA, 20 bp, Artificial sequence
  Synthetic
cgccgagggacgaacgcacg

SEQ ID NO: 351 - DNA, 96 bp, Homo sapiens
ggagcccccagccccacgcgggcacacgcagggtgggtggtcacgcccgcagggtccgcgagcgcggcgcagagcgcgggccgtgggaagtttctc

SEQ ID NO: 352 - DNA, 96 bp, Artificial sequence
  Synthetic
ggagtttttagttttacgcgggtatacgtagggtgggtggttacgttcgtagggttcgcgagcgcggcgtagagcgcgggtcgtgggaagtttttt

SEQ ID NO: 353 - DNA, 18 bp, Artificial sequence
  Synthetic
gtagggtgggtggttacg

SEQ ID NO: 354 - DNA, 17 bp, Artificial sequence
  Synthetic
aacttcccacgacccgc

SEQ ID NO: 355 - DNA, 20 bp, Artificial sequence
  Synthetic
cgccgagggttcgtagggtt

SEQ ID NO: 356 - DNA, 127 bp, Homo sapiens
ggcgccgccattgcggtcctcattttgctgctggtgggttgggctacagcaggcctctggagccacaccagggcacgggagtgggtgcagggaccgtcaccgcgccttcacacgcaccatagtgccc

SEQ ID NO: 357 - DNA, 127 bp, Artificial sequence
  Synthetic
ggcgtcgttattgcggtttttattttgttgttggtgggttgggttatagtaggtttttggagttatattagggtacgggagtgggtgtagggatcgttatcgcgtttttatacgtattatagtgttt

SEQ ID NO: 358 - DNA, 23 bp, Artificial sequence
  Synthetic
tggagttatattagggtacggga

SEQ ID NO: 359 - DNA, 28 bp, Artificial sequence
  Synthetic
acactataatacgtataaaaacgcgata

SEQ ID NO: 360 - DNA, 21 bp, Artificial sequence
  Synthetic
ccacggacgaacgatccctac

SEQ ID NO: 361 - DNA, 140 bp, Homo sapiens
ggcggcgaggggcgcgtccgcgggtgggtttcacctgggtggtgggcatgtcgggcccgctagggcgagggtctggccaggggcgtagttctcctggtgggtggggacgctccgtggcgattggggtcactcctctgagg

SEQ ID NO: 362 - DNA, 140 bp, Artificial sequence
  Synthetic
ggcggcgaggggcgcgttcgcgggtgggttttatttgggtggtgggtatgtcgggttcgttagggcgagggtttggttaggggcgtagtttttttggtgggtggggacgtttcgtggcgattggggttatttttttgagg

SEQ ID NO: 363 - DNA, 17 bp, Artificial sequence
  Synthetic
ggtggtgggtatgtcgg

SEQ ID NO: 364 - DNA, 17 bp, Artificial sequence
  Synthetic
ccaatcgccacgaaacg

SEQ ID NO: 365 - DNA, 21 bp, Artificial sequence
  Synthetic
ccacggacgggttcgttaggg

SEQ ID NO: 366 - DNA, 117 bp, Homo sapiens
ccgtgggcgcggacagctgccgggagcggcaggcgtctcgatcggggacgcaggcacttccgtccctgcagagcatcagacgcgtctcgggacactggggacaacatctcctccgcg

SEQ ID NO: 367 - DNA, 117 bp, Artificial sequence
  Synthetic
tcgtgggcgcggatagttgtcgggagcggtaggcgtttcgatcggggacgtaggtattttcgtttttgtagagtattagacgcgtttcgggatattggggataatatttttttcgcg

SEQ ID NO: 368 - DNA, 18 bp, Artificial sequence
  Synthetic
gttgtcgggagcggtagg

SEQ ID NO: 369 - DNA, 21 bp, Artificial sequence
  Synthetic
ccaatatcccgaaacgcgtct

SEQ ID NO: 370 - DNA, 21 bp, Artificial sequence
  Synthetic
ccacggacggcgtttcgatcg

SEQ ID NO: 371 - DNA, 120 bp, Homo sapiens
aagctgcgcccggagacgtgggagcgttctcttgttttccgagtgcgcggactcatcgggtcacagtttatgcttttatgacgcggtgagtccagccactgattcctaacggtttagagt

SEQ ID NO: 372 - DNA, 120 bp, Artificial sequence
  Synthetic
aagttgcgttcggagacgtgggagcgtttttttgtttttcgagtgcgcggatttatcgggttatagtttatgtttttatgacgcggtgagtttagttattgatttttaacggtttagagt

SEQ ID NO: 373 - DNA, 23 bp, Artificial sequence
  Synthetic
cgtttttttgtttttcgagtgcg

SEQ ID NO: 374 - DNA, 23 bp, Artificial sequence
  Synthetic
tcaataactaaactcaccgcgtc

SEQ ID NO: 375 - DNA, 21 bp, Artificial sequence
  Synthetic
ccacggacggcggatttatcg

SEQ ID NO: 376 - DNA, 224 bp, Homo sapiens
ctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaaga

SEQ ID NO: 377 - DNA, 224 bp, Artificial sequence
  Synthetic
ttttgatttgagttttttttggaattttgtaggttttatttgtttttttttagatgagttttttttttggtgtttgtttttttgattaggtgtttaagatagtgttgtgggtgtaggtattaatattggtttgtgtgataaggttatgaggttggtgtaaagtggttttggagtgtgtattaagtaggtgtatagtaggtttgaatagattttttattttaaga

SEQ ID NO: 378 - DNA, 19 bp, Artificial sequence
  Synthetic
ccatgaggctggtgtaaag

SEQ ID NO: 379 - DNA, 24 bp, Artificial sequence
  Synthetic
ctactgtgcacctacttaatacac

SEQ ID NO: 380 - DNA, 20 bp, Artificial sequence
  Synthetic
cgccgagggcggccttggag

SEQ ID NO: 381 - DNA, 30 bp, Artificial sequence
  Synthetic
gtgtttgtttttttgattaggtgtttaaga

SEQ ID NO: 382 - DNA, 26 bp, Artificial sequence
  Synthetic
ctttacaccaacctcataaccttatc

SEQ ID NO: 383 - DNA, 21 bp, Artificial sequence
  Synthetic
gacgcggagatagtgttgtgg

SEQ ID NO: 384 - DNA, 139 bp, Homo sapiens
ggccacacaggcccactctggccctctgagcccccggcggacccagggcattcaaggagcggctctgggctgccagcgcaggcctccgcgcaaacacagcaggctggaagtggcgctcatcaccggcacgtcttcccag

SEQ ID NO: 385 - DNA, 139 bp, Artificial sequence
  Synthetic
ggttatataggtttattttggttttttgagttttcggcggatttagggtatttaaggagcggttttgggttgttagcgtaggttttcgcgtaaatatagtaggttggaagtggcgtttattatcggtacgtttttttag

SEQ ID NO: 386 - DNA, 28 bp, Artificial sequence
  Synthetic
ggtttattttggttttttgagttttcgg

SEQ ID NO: 387 - DNA, 24 bp, Artificial sequence
  Synthetic
tccaacctactatatttacgcgaa

SEQ ID NO: 388 - DNA, 21 bp, Artificial sequence
  Synthetic
ccacggacggcggatttaggg

SEQ ID NO: 389 - DNA, 171 bp, Artificial sequence
  Synthetic
tccacgtggtgcccactctggacaggtggagcagagggaaggtggtggcatggtggggagggtggcctggaggacccgattggctgagtgtaaaccaggagaggacatgactttcagccctgcagccagacacagctgagctggtgtgacctgtgtggagagttcatctgg

SEQ ID NO: 390 - DNA, 180 bp, Artificial sequence
  Synthetic
tttatcgtggtgtttattttggataggtggagtagagggaaggtggtgcgtatggtgggcgagcgcgtgcgtttggaggatttcgattggttgacgtgtaaattaggacgaggatatgatttttagttttgtagttagatatagttgagttggtgtgatttgtgtggagagtttatttgg

SEQ ID NO: 391 - DNA, 15 bp, Artificial sequence
  Synthetic
cgcatggtgggcgag

SEQ ID NO: 392 - DNA, 18 bp, Artificial sequence
  Synthetic
acacgtcagccaatcggg

SEQ ID NO: 393 - DNA, 20 bp, Artificial sequence
  Synthetic
gacgcggaggcgcgtgcgcc

SEQ ID NO: 394 - DNA, 17 bp, Artificial sequence
  Synthetic
tgcgtatggtgggcgag

SEQ ID NO: 395 - DNA, 26 bp, Artificial sequence
  Synthetic
cctaatttacacgtcaaccaatcgaa

SEQ ID NO: 396 - DNA, 21 bp, Artificial sequence
  Synthetic
gacgcggaggcgcgtgcgttt

SEQ ID NO: 397 - DNA, 21 bp, Artificial sequence
  Synthetic
ccacggacggcgcgtgcgttt

SEQ ID NO: 398 - DNA, 28 bp, Artificial sequence
  Synthetic
agccggttttccggctgagacctcggcg

SEQ ID NO: 399 - DNA, 28 bp, Artificial sequence
  Synthetic
agccggttttccggctgagacctcggcg

SEQ ID NO: 400 - DNA, 29 bp, Artificial sequence
  Synthetic
agccggttttccggctgagactccgcgtc

SEQ ID NO: 401 - DNA, 29 bp, Artificial sequence
  Synthetic
agccggttttccggctgagacgtccgtgg

SEQ ID NO: 402 - DNA, 28 bp, Artificial sequence
  Synthetic
agccggttttccggctgagaggacgcgc

SEQ ID NO: 403 - DNA, 108 bp, Homo sapiens
ggaaggaaattgcgggttcccgtctgccttgtctccagcttctctgctgaagcccggtagcagtgaatgcgcgctgactttcagcgacgactcctggaagcaacgcca

SEQ ID NO: 404 - DNA, 108 bp, Artificial sequence
  Synthetic
ggaaggaaattgcgggttttcgtttgttttgtttttagtttttttgttgaagttcggtagtagtgaatgcgcgttgatttttagcgacgatttttggaagtaacgtta

SEQ ID NO: 405 - DNA, 27 bp, Artificial sequence
  Synthetic
aggccacggacgcgaaaaatcccacgc

SEQ ID NO: 406 - DNA, 17 bp, Artificial sequence
  Synthetic
gtcgagcgtttggtgcg

SEQ ID NO: 407 - DNA, 19 bp, Artificial sequence
  Synthetic
ctcgtcgaaatcgaaacgc

SEQ ID NO: 408 - DNA, 32 bp, Artificial sequence
  Synthetic
cgcgccgagggcgatagcgttttttattgtcg

SEQ ID NO: 409 - DNA, 19 bp, Artificial sequence
  Synthetic
cgaggttatggaggtgacg

SEQ ID NO: 410 - DNA, 22 bp, Artificial sequence
  Synthetic
cgaatactacccgttaaacacg

SEQ ID NO: 411 - DNA, 27 bp, Artificial sequence
  Synthetic
aggccacggacgggcggattagtcgcg

SEQ ID NO: 412 - DNA, 330 bp, Homo sapiens
ggcggcgccgcgaccgccttccttcgctgcgtcccgcccgctccacgcctcgctcaccgccgccgcttctccctgccccgcagcgcgcagggaccatgtcggcggagaccgcgagcggccccacagaggaccaggtggaaatcctggagtacaacttcaacaaggtcgacaagcacccggattccaccacgctgtgcctcatcgcggccgaggcaggcctttccgaggaggagacccaggtgcgtccccacacgcgcccagcgcgccccgacccctgcctgggctgagccttctcgcggctgggcggtcctgttcgtccgcgcctcccgc

SEQ ID NO: 413 - DNA, 330 bp, Artificial sequence
  Synthetic
ggcggcgtcgcgatcgttttttttcgttgcgtttcgttcgttttacgtttcgtttatcgtcgtcgtttttttttgtttcgtagcgcgtagggattatgtcggcggagatcgcgagcggttttatagaggattaggtggaaattttggagtataattttaataaggtcgataagtattcggattttattacgttgtgttttatcgcggtcgaggtaggttttttcgaggaggagatttaggtgcgtttttatacgcgtttagcgcgtttcgatttttgtttgggttgagttttttcgcggttgggcggttttgttcgttcgcgtttttcgt

SEQ ID NO: 414 - DNA, 22 bp, Artificial sequence
  Synthetic
gcgtcgcgatcgttttttttcg

SEQ ID NO: 415 - DNA, 24 bp, Artificial sequence
  Synthetic
aacgacgacgataaacgaaacgta

SEQ ID NO: 416 - DNA, 30 bp, Artificial sequence
  Synthetic
aggccacggacggttgcgtttcgttcgttt

SEQ ID NO: 417 - DNA, 22 bp, Artificial sequence
  Synthetic
gtagcgcgtagggattatgtcg

SEQ ID NO: 418 - DNA, 27 bp, Artificial sequence
  Synthetic
tttccacctaatcctctataaaaccgc

SEQ ID NO: 419 - DNA, 26 bp, Artificial sequence
  Synthetic
aggccacggacgctcgcgatctccgc

SEQ ID NO: 420 - DNA, 19 bp, Artificial sequence
  Synthetic
acgttgtgttttatcgcgg

SEQ ID NO: 421 - DNA, 22 bp, Artificial sequence
  Synthetic
ctaaacgcgtataaaaacgcac

SEQ ID NO: 422 - DNA, 32 bp, Artificial sequence
  Synthetic
aggccacggacggtcgaggtaggttttttcga

SEQ ID NO: 423 - DNA, 16 bp, Artificial sequence
  Synthetic
caactcatccgcgacg

SEQ ID NO: 424 - DNA, 27 bp, Artificial sequence
  Synthetic
aggccacggacggtcgacgcccaacaa

SEQ ID NO: 425 - DNA, 37 bp, Artificial sequence
  Synthetic
cgcgccgagggcgttaggatttatttttttttttcga

SEQ ID NO: 426 - DNA, 72 bp, Homo sapiens
cgggacagagccgaccaatcaggcggctcggcagcggggcagaggtcagggggcgggccgaggggaagccaa

SEQ ID NO: 427 - DNA, 72 bp, Artificial sequence
  Synthetic
cgggatagagtcgattaattaggcggttcggtagcggggtagaggttagggggcgggtcgaggggaagttaa

SEQ ID NO: 428 - DNA, 24 bp, Artificial sequence
  Synthetic
cgggatagagtcgattaattaggc

SEQ ID NO: 429 - DNA, 18 bp, Artificial sequence
  Synthetic
taacttcccctcgacccg

SEQ ID NO: 430 - DNA, 24 bp, Artificial sequence
  Synthetic
cgcgccgaggcggttcggtagcgg

SEQ ID NO: 431 - DNA, 26 bp, Artificial sequence
  Synthetic
cgcgccgaggttacaaaccgcgaccg

SEQ ID NO: 432 - DNA, 26 bp, Artificial sequence
  Synthetic
ttttcgttgattttattcgagtcgtc

SEQ ID NO: 433 - DNA, 22 bp, Artificial sequence
  Synthetic
gaaccctcttcaaataaaccgc

SEQ ID NO: 434 - DNA, 30 bp, Artificial sequence
  Synthetic
cgcgccgaggcgtttggcgtagatataagc

SEQ ID NO: 435 - DNA, 33 bp, Artificial sequence
  Synthetic
aggccacggacggcggatttatcgggttatagt

SEQ ID NO: 436 - DNA, 35 bp, Artificial sequence
  Synthetic
acggacgcggaggcggatttagggtatttaaggag

SEQ ID NO: 437 - RNA, 1371 nt, Homo sapiens
aaucauuagagccugagucacucuccccaggagacccagaccuagaacuacccagagcaagaccacagcuggugaacaguccagccugucuccaguuggacuagccacaauucaagugcuugaaaaccacauguggagcagacaagauggagacaaauuccucucuccccacgaacaucucuggagggacaccugcuguaucugcuggcuaucucuuccuggauaucaucacuuaucugguauuugcagucaccuuuguccucgggguccugggcaacgggcuugugaucuggguggcuggauuccggaugacacacacagucaccaccaucaguuaccugaaccuggccguggcugacuucuguuucaccuccacuuugccauucuucauggucaggaaggccaugggaggacauuggccuuucggcugguuccugugcaaauucgucuuuaccauaguggacaucaacuuguucggaagugucuuccugaucgcccucauugcucuggaccgcuguguuugcguccugcauccagucuggacccagaaccaccgcaccgugagccuggccaagaaggugaucauugggcccugggugauggcucugcuccucacauugccaguuaucauucgugugacuacaguaccugguaaaacggggacaguagccugcacuuuuaacuuuucgcccuggaccaacgacccuaaagagaggauaaauguggccguugccauguugacggugagaggcaucauccgguucaucauuggcuucagcgcacccauguccaucguugcugucaguuaugggcuuauugccaccaagauccacaagcaaggcuugauuaaguccagucgucccuuacggguccucuccuuugucgcagcagccuuuuuucucugcugguccccauaucaggugguggcccuuauagccacagucagaauccgugaguuauugcaaggcauguacaaagaaauugguauugcaguggaugugacaagugcccuggccuucuucaacagcugccucaaccccaugcucuaugucuucaugggccaggacuuccgggagaggcugauccacgcccuucccgccagucuggagagggcccugaccgaggacucaacccaaaccagugacacagcuaccaauucuacuuuaccuucugcagagguggaguuacaggcaaagugaggagggagcugggggacacuuucgagcucccagcuccagcuucgucucaccuugaguuaggcugagccacaggcauuuccugcuuauuuuaggauuacccacucaucagaaaaaaaaaaaaaagccuuuguguccccugauuuggggagaauaaacagauaugaguuuauua

SEQ ID NO: 438 - RNA, 106 nt, Homo sapiens
guugccauguugacggugagaggcaucauccgguucaucauuggcuucagcgcacccauguccaucguugcugucaguuaugggcuuauugccaccaagauccaca

SEQ ID NO: 439 - DNA, 18 bp, Artificial sequence
  Synthetic
tgacggtgagaggcatca

SEQ ID NO: 440 - DNA, 22 bp, Artificial sequence
  Synthetic
ggtggcaataagcccataactg

SEQ ID NO: 441 - DNA, 23 bp, Artificial sequence
  Synthetic
cggttcatcattggcttcagcgc

(19) United States
(12) Patent Application Publication
(10) Pub. No.: US 2022/0136058 A1
(43) Pub. Date: May 5, 2022

(54) CHARACTERIZING METHYLATED DNA, RNA, AND PROTEINS IN THE DETECTION OF LUNG NEOPLASIA

(71) Applicants: Exact Sciences Development Company, LLC, Madison, WI (US); Mayo Foundation for Medical Education and Research, Rochester, MN (US)

(72) Inventors: Hatim Allawi, Middleton, WI (US); Graham P. Lidgard, Middleton, WI (US); Maria Giakoumopoulos, Middleton, WI (US); David A. Ahlquist, Rochester, MN (US); William R. Taylor, Lake City, MN (US); Douglas Mahoney, Rochester, MN (US)

(21) Appl. No.: 17/297,356

(22) PCT Filed: Nov. 26, 2019
(86) PCT No.: PCT/US2019/063401

§ 371 Date: May 26, 2021

Related U.S. Application Data
(60) Provisional application No. 62/771,965, filed on Nov. 27, 2018.

(51) Int. Cl. C12Q 1/6886; G01N 33/574

(52) U.S. Cl. CPC C12Q 1/6886; C12Q 2600/154; G01N 33/57423

(57) ABSTRACT

Provided herein is technology relating to detecting neoplasia and particularly, but not exclusively, to methods, compositions, and related uses for detecting neoplasms such as lung cancer.

The present application claims priority to U.S. Provisional Application Ser. No. 62/771,965, filed Nov. 27, 2018, which is incorporated herein by reference.

### FIELD OF THE INVENTION

Provided herein is technology relating to detecting neoplasia and particularly, but not exclusively, to methods, compositions, and related uses for detecting neoplasms such as lung cancer.

### BACKGROUND OF THE INVENTION

Lung cancer remains the number one cancer killer in the US, and effective screening approaches are desperately needed. Lung cancer alone accounts for 221,000 deaths annually.

DNA methylation profiling has shown unique patterns in DNA promoter regions with cancer and has potential application for detection of lung malignancies. However, optimally discriminant markers and marker panels are needed.

### SUMMARY OF THE INVENTION

Provided herein is a collection of methylation markers assayed on tissue or plasma that achieves extremely high discrimination for all types of lung cancer while remaining negative in normal lung tissue and benign nodules. Markers selected from the collection can be used alone or in a panel, for example, to characterize blood or bodily fluid, with applications in lung cancer screening and discrimination of malignant from benign nodules. In some embodiments, markers from the panel are used to distinguish one form of lung cancer from another, e.g., for distinguishing the presence of a lung adenocarcinoma or large cell carcinoma from the presence of a lung small cell carcinoma, or for detecting mixed pathology carcinomas. Provided herein is technology for screening markers that provide a high signal-to-noise ratio and a low background level when detected from samples taken from a subject.

Methylation markers and/or panels of markers (e.g., chromosomal region(s)) having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX were identified in studies by comparing the methylation state of methylation markers from lung cancer samples to the corresponding markers in normal (non-cancerous) samples.

As described herein, the technology provides a number of methylation markers and subsets thereof (e.g., sets of 2, 3, 4, 5, 6, 7, 8, 9, 10, 11, 12 or more markers) with high discrimination for lung cancer and, in some embodiments, with discrimination between lung cancer types. Experiments applied a selection filter to candidate markers to identify markers that provide a high signal to noise ratio and a low background level to provide high specificity and selectivity for purposes of characterizing biological samples, e.g., for cancer screening or diagnosis. For example, as described herein below, analysis of methylation of combination of 8 markers, SLC12A8, KLHDC7B, PARP15, OPLAH, BCL2L11, MAX.chr12.526, HOXB2, and EMX1, resulted in 98.5% sensitivity (134/136 cancers) for all of the cancer tissues tested, with 100% specificity. In another embodiment, a panel of 6 markers (SHOX2, SOBP, ZNF781, CYP26C1, SUCLG2, and SKI) resulted in a sensitivity of 92.2% at 93% specificity, and a panel of 4 markers (ZNF781, BARX1, EMX1, and HOXA9) resulted in an overall sensitivity of 96% and specificity of 94%.

Accordingly, provided herein is technology related to a method of processing a sample obtained from a subject, the method comprising assaying a methylation state of one or more marker genes in the sample. In preferred embodiments, the methylation state of the methylation marker is determined by measuring the amounts of a methylation marker and of a reference marker in the sample, and comparing the amount of the methylation marker to the amount of reference marker in the sample to determine a methylation state for the methylation marker in the sample. While not limiting the invention to any particular application or applications, the method finds use, e.g., in characterizing samples from a subject having or suspected of having lung cancer, when the methylation state of the methylation marker is different than a methylation state of that marker assayed in a subject that does not have a neoplasm. In preferred embodiments, the methylation marker comprises a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX. In some embodiments, the reference marker is selected from B3GALT6 DNA and β-actin DNA.

In some embodiments, the technology comprises assaying a plurality of markers, e.g., comprising assaying the methylation states of 2 to 21 markers, preferably 2 to 8 markers, preferably 4 to 6 markers. For example, in some embodiments, the method comprises analysis of the methylation status of two or more markers selected from SLC12A8, KLHDC7B, PARP15, OPLAH, BCL2L11, MAX.chr12.526, HOXB2, EMX1, CYP26C1, SOBP, SUCLG2, SHOX2, ZDHHC1, NFIX, FLJ45983, HOXA9, B3GALT6, ZNF781, SP9, BARX1, and SKI. In some preferred embodiments, the method comprises analysis of the methylation status of a set of markers comprising SLC12A8, KLHDC7B, PARP15, OPLAH, BCL2L11, MAX.chr12.526, HOXB2, and EMX1. In some embodiments, the method comprises analysis of the methylation status of a set of markers selected from: the group consisting of ZNF781, BARX1, and EMX1; the group consisting of SHOX2, SOBP, ZNF781, CYP26C1, SUCLG2, and SKI; the group consisting of SLC12A8, KLHDC7B, PARP15, OPLAH, BCL2L11, MAX.chr12.526, HOXB2, and EMX1; the group consisting of SHOX2, SOBP, ZNF781, BTACT, CYP26C1, and DLX4; and the group consisting of SHOX2, SOBP, ZNF781, CYP26C1, SUCLG2, and SKI. In certain embodiments, the at least one methylation marker comprises the group selected from ZNF781, BARX1, and EMX1, and further comprises SOBP and/or HOXA9. In other embodiments, the at least one methylation marker comprises a group selected from BARX1, HOXB2, FLJ45983, IFFO1, HOPX, TRH, HOXA9, SOBP, ZNF781, and FAM59B.

In some embodiments, the at least one methylation marker comprises one or both of IFFO1 and HOPX, and optionally further comprises one or more marker genes selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1 and ZNF32. In certain embodiments, the at least one methylation marker gene consists of at least one of IFFO1 and HOPX, and further comprises one or more of BARX1, FLJ45983, HOXA9, ZNF781, HOXB2, SOBP, TRH, and FAM59B, while in certain preferred embodiments, the at least one methylation marker gene consists of at least one of IFFO1 and HOPX, and the group BARX1, FLJ45983, HOXA9, ZNF781, HOXB2, SOBP, TRH, and FAM59B.

The technology is not limited in the methylation state assessed. In some embodiments assessing the methylation state of the methylation marker in the sample comprises determining the methylation state of one base. In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

In some embodiments, the technology provides a method of generating a record reporting a lung neoplasm in a subject, the method comprising the steps of:

a) assaying a sample from a subject for an amount of at least one methylated methylation marker gene selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZWIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX in a sample obtained from a subject;

b) assaying said sample for an amount of reference marker in said sample;

c) comparing the amount of said at least one methylated methylation marker to the amount of reference marker in said sample to determine a methylation state for said at least one methylation marker in said sample; and

d) generating a record reporting the methylation state for said at least one marker gene in said sample, wherein the methylation state of said methylation marker is indicative of the presence or absence of a lung neoplasm in said subject.

In some embodiments, the technology provides a method of characterizing a sample, comprising:
a) measuring an amount of at least one methylation marker gene in DNA selected from the group consisting of BARX1, LOC100129726, SPOCK2, TSC22D4, MAX_chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNFJ32, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMY1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX;
b) measuring the amount of at least one reference marker in the DNA; and
c) calculating a value for the amount of the at least one methylation marker gene measured in the DNA as a percentage of the amount of the reference marker measured in the DNA, wherein the value indicates the amount of the at least one methylation marker DNA measured in the sample.

In some preferred embodiments, the at least one methylation marker gene consists of one to fifteen methylation marker genes.

In some embodiments, amounts of at least two of the markers are measured, and preferably the at least two methylation marker genes are selected from the group consisting of SLC12A8, KLHDC7B, PARP15, OPLAH, BCL2L11, MAX.chr12.526, HOXB2, EMX1 CYP26C1, SOBP, SUCLG2, SHOX2, ZDHHC1, NFIX, FLJ45983, HOXA9, B3GALT6, ZNF781, SP9, BARX1, and SKI. In other embodiments, the methylation markers comprise a group selected from BARX1, HOXB2, FLJ45983, IFFO1, HOPX, TRH, HOXA9, SOBP, ZNF781, and FAM59B. In certain preferred embodiments, the method comprises analysis of the methylation status of a set of markers selected from: the group consisting of ZNF781, BARX1, and EMX1; the group consisting of SHOX2, SOBP, ZNF781, CYP26C1, SUCLG2, and SKI; the group consisting of SLC12A8, KLHDC7B, PARP15, OPLAH, BCL2L11, MAX.chr12.526, HOXB2, and EMX1; the group consisting of SHOX2, SOBP, ZNF781, BTACT, CYP26C1, and DLX4; and the group consisting of SHOX2, SOBP, ZNF781, CYP26C1, SUCLG2, and SKI. In certain embodiments, the at least one methylation marker comprises the group selected from ZNF781, BARX1, and EMX1, and further comprises SOBP and/or HOXA9. In some embodiments, methylation markers are selected such that the methylation status of said one or more markers is indicative of only one of lung adenocarcinoma, large cell carcinoma, squamous cell carcinoma, or small cell carcinoma. In other embodiments, methylation markers are selected such that the methylation status of said one or more markers is indicative of more than one of lung adenocarcinoma, large cell carcinoma, squamous cell carcinoma, and small cell carcinoma. In yet other embodiments, methylation markers are selected such that the methylation status of said one or more markers is indicative of any one of or combination of lung adenocarcinoma, large cell carcinoma, squamous cell carcinoma, small cell carcinoma, generic non-small cell lung cancer, and/or undefined lung carcinoma. In some embodiments assaying or measuring the methylation state of the methylation marker in the sample comprises determining the methylation state of one base, while in other embodiments the assay comprises determining the extent of methylation at a plurality of bases. In some embodiments the methylation state of the marker comprises an increased or decreased methylation of the marker relative to a normal methylation state of the marker, e.g., as the marker would appear in a non-cancerous sample, while in some embodiments the methylation state of the marker comprises a different pattern of methylation of the marker relative to a normal methylation state of the marker. In preferred embodiments the reference marker is a methylated reference marker. In some embodiments, the reference marker comprises a portion of a gene.

The technology is not limited to particular sample types. For example, in some embodiments the sample is a tissue sample, a blood sample, a plasma sample, a serum sample, or a sputum sample. In certain preferred embodiments a tissue sample comprises lung tissue. In certain preferred embodiments, the sample comprises DNA isolated from plasma.

The technology is not limited to any particular method of assaying DNA from samples. For example, in some embodiments the assaying comprises using polymerase chain reaction, nucleic acid sequencing, mass spectrometry, methylation specific nuclease, mass-based separation, and/or target capture. In certain preferred embodiments the assaying comprises using a flap endonuclease assay.

In some embodiments, the DNA is treated with a reagent that selectively modifies DNA in a manner specific to the methylation status of the DNA. For example, in in some embodiments, DNA is treated with a restriction enzyme that a methylation-sensitive restriction enzyme, or a methylation-dependent restriction enzyme.

In particularly preferred embodiments the sample DNA and/or reference marker DNA are bisulfite-converted and the assay for determining the methylation level of the DNA is achieved by a technique comprising the use of methylation-specific PCR, quantitative methylation-specific PCR, methylation-specific DNA restriction enzyme analysis, quantitative bisulfite pyrosequencing, flap endonuclease assay (e.g., a QUARTS flap endonuclease assay), and/or bisulfite genomic sequencing PCR.

The technology also provides methods of characterizing a sample or combination of samples from a subject comprising analyzing the sample(s) for a plurality of different types of marker molecules. For example, in some embodiments, the technology provides a method comprising measuring an amount of at least one methylation marker gene in DNA from a sample obtained from a subject, and further comprises one or more of measuring an amount of at least one RNA marker in a sample obtained from the subject, and assaying for the presence or absence of at least one protein marker in a sample obtained from the subject. In some embodiments, a single sample from a subject is analyzed for methylation marker DNA(s), marker RNA(s), and marker protein(s).

Analysis of DNA, RNA and protein markers are not limited to use of any particular technologies. Methods for analyzing DNA and RNA are well known, in include but are not limited to nucleic acid detection assays comprising amplification, probe hybridization, for example. Methods for analyzing proteins include but are not limited to enzyme-linked immunosorbent assay (ELISA) detection, protein immunoprecipitation, Western blot, immunostaining, etc.

The technology also provides kits. For example, in some embodiments the technology provides a kit, comprising a) at least one oligonucleotide, wherein at least a portion of the oligonucleotide specifically hybridizes to a marker selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERAMT3, NFIX, SIPR4, SKI, SUCLG2, TBX15, ZDHHC1, ZNF329, IFFO1, and HOPX. In preferred embodiments, the portion of the oligonucleotide that hybridizes to the marker specifically hybridizes to bisulfite-treated DNA comprising the methylation marker. In some embodiments, the kit comprises at least one additional oligonucleotide, wherein at least a portion of the additional oligonucleotide specifically hybridizes to a reference nucleic acid. In some embodiments the kit comprises at least two additional oligonucleotides and, in some embodiments, the kit further comprises a bisulfite reagent.

In certain embodiments at least a portion of the oligonucleotide specifically hybridizes to a least one the marker selected from the group consisting of SLC12A8, KLHDC7B, PARP15, OPLAH, BCL2L11, MAX.chr12.526, HOXB2, EMX1, CYP26C1, SOBP, SUCLG2, SHOX2, ZDHHC1, NFIX, FLJ45983, HOXA9, B3GALT6, ZNF781, SP9, BARX1, and SKI. In other embodiments, at least a portion of the oligonucleotide specifically hybridizes to a least one the marker selected from the group consisting of BARX1, HOXB2, FLJ45983, IFFO1, HOPX, TRH, HOXA9, SOBP, ZNF781, and FAM59B.

In preferred embodiments, the kit comprises a set of oligonucleotides, each of which hybridizes to one marker in a set of markers, the set of markers selected from: the group consisting of ZNF781, BARX1, and EMX1; the group consisting of SHOX2, SOBP, ZNF781, CYP26C1, SUCLG2, and SKI; the group consisting of SLC12A8, KLHDC7B, PARP15, OPLAH, BCL2L11, MAX.chr12.526, HOXB2, and EMX1; the group consisting of SHOX2, SOBP, ZNF781, BTACT, CYP26C1, and DLX4; and the group consisting of SHOX2, SOBP, ZNF781, CYP26C1, SUCLG2, and SKI. In certain embodiments, the set of methylation markers comprises the group selected from ZNF781, BARX1, and EMX1, and further comprises SOBP and/or HOXA9. In some embodiments, the set of markers comprises one or both of IFFO1 and HOPX, and further comprises one or more markers selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNFJ32, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1 and ZNF32. In other embodiments, the set of methylation markers comprises one or both of IFFO1 and HOPX, and further comprises one or more markers selected from BARX1, HOXB2, FLJ45983, IFFO1, HOPX, TRH, HOXA9, SOBP, ZNF781, and FAM59B. In certain embodiments, the set of methylation markers consists of one or both of IFFO1 and HOPX, and one or more markers selected from BARX1, HOXB2, FLJ45983, IFFO1, HOPX, TRH, HOXA9, SOBP, ZNF781, and FAM59B.

In some embodiments, the at least one oligonucleotide in the kit is selected to hybridize to methylation marker(s) that are indicative of only one of type of lung carcinoma, e.g., lung adenocarcinoma, large cell carcinoma, squamous cell carcinoma, or small cell carcinoma. In other embodiments, the at least one oligonucleotide is selected to hybridize to methylation marker(s) that are indicative of more than one of lung adenocarcinoma, large cell carcinoma, squamous cell carcinoma, and small cell carcinoma. In yet other embodiments, the at least one oligonucleotide is selected to hybridize to methylation marker(s) that are indicative of any one of, or any combination of lung adenocarcinoma, large cell carcinoma, squamous cell carcinoma, small cell carcinoma, and/or undefined lung carcinoma.

In preferred embodiments, oligonucleotide(s) provided in the kit are selected from one or more of a capture oligonucleotide, a pair of nucleic acid primers, a nucleic acid probe, and an invasive oligonucleotide. In preferred embodiments, oligonucleotide(s) specifically hybridize to bisulfite-treated DNA comprising said methylation marker(s).

In some embodiments the kit further comprises a solid support, such a magnetic bead or particle. In preferred embodiments, a solid support comprises one or more capture reagents, e.g., oligonucleotides complementary said one or more markers genes.

The technology also provides compositions. For example, in some embodiments the technology provides a composition comprising a mixture, e.g., a reaction mixture, that comprises a complex of a target nucleic acid selected from the group consisting of BARX1, LOC100129726, SPOCK2, TSC22D4, MAX.chr8.124, RASSF1, ZNF671, STA8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12a, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, and an oligonucleotide that specifically hybridizes to the target nucleic acid. In some embodiments, the target nucleic acid is bisulfite-converted target nucleic acid. In preferred embodiments, the mixture comprises a complex of a target nucleic acid selected from the group consisting of SLC12A8, KLHDC7B, PARP15, OPLAH, BCL2L11, MAX.chr12.526, HOXB2, EMX1, CYP26C1, SOBP, SUCLG2, SHOX2, ZDHHC1, NFIX, FLJ45983, HOXA9, B3GALT6, ZNF781, SP9, BARX1, and SKI, and an oligonucleotide that specifically hybridizes to the target nucleic acid (whether unconverted or bisulfite-converted). In other preferred embodiments, the mixture comprises a complex of a target nucleic acid selected from the group consisting of BARX1, HOXB2, FLJ45983, IFFO1, HOPX, TRH, HOXA9, SOBP, ZNF781, and FAM59B, and an oligonucleotide that specifically hybridizes to the target nucleic acid (whether unconverted or bisulfite-converted). Oligonucleotides in the mixture include but are not limited to one or more of a capture oligonucleotide, a pair of nucleic acid primers, a hybridization probe, a hydrolysis probe, a flap assay probe, and an invasive oligonucleotide.

In some embodiments, the target nucleic acid in the mixture comprises a nucleic acid sequence selected from the group consisting of SEQ ID NOS: 1, 6, 11, 16, 21, 28, 33, 38, 43, 48, 53, 58, 63, 68, 73, 78, 86, 91, 96, 101, 106, 111, 116, 121, 126, 131, 136, 141, 146, 151, 156, 161, 166, 171, 176, 181, 186, 191, 196, 201, 214, 219, 224, 229, 234, 239, 247, 252, 257, 262, 267, 272, 277, 282, 287, 292, 298, 303, 308, 313, 319, 327, 336, 341, 346, 351, 356, 361, 366, 371, 384, 403, 412, and 426, and complements thereof.

In some embodiments, the mixture comprises bisulfite-converted target nucleic acid that comprises a nucleic acid sequence selected from the group consisting of SEQ ID NOS: 2, 7, 12, 17, 22, 29, 34, 39, 44, 49, 54, 59, 64, 69, 74, 79, 87, 92, 97, 102, 107, 112, 117, 122, 127, 132, 137, 142, 147, 152, 157, 162, 167, 172, 177, 182, 187, 192, 197, 202, 210, 215, 220, 225, 230, 235, 240, 248, 253, 258, 263, 268, 273, 278, 283, 288, 293, 299, 304, 309, 314, 320, 328, 337, 342, 347, 352, 357, 362, 367, 372, 385, 404, 413, and 427, and complements thereof.

In some embodiments, a kit comprises reagents or materials for at least two assays, wherein the assays are selected from measuring an amount of, or the presence or absence of 1) at least one methylated DNA marker; 2) at least one RNA marker; and 3) at least one protein marker. In preferred embodiments, the at least one methylated DNA marker is selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12a, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX. In some embodiments, the at least one protein comprises an antigen, e.g., a cancer-associated antigen, while in some embodiments, the at least one protein comprises an antibody, e.g., an autoantibody to a cancer-associated antigen.

In some embodiments, an oligonucleotide in said mixture comprises a reporter molecule, and in preferred embodiments, the reporter molecule comprises a fluorophore. In some embodiments the oligonucleotide comprises a flap sequence. In some embodiments the mixture further comprises one or more of a FRET cassette; a FEN-1 endonuclease and/or a thermostable DNA polymerase, preferably a bacterial DNA polymerase.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz. 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated. In some embodiments, a nucleic acid may be characterized as “unmethylated” if it is not methylated at a specific locus (e.g., the locus of a specific single CpG dinucleotide) or specific combination of loci, even if it is methylated at other loci in the same gene or molecule.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs. As used herein, the terms “marker gene” and “marker” are used interchangeably to refer to DNA (or other sample components) that is associated with a condition, e.g., cancer, regardless of whether the marker region is in a coding region of DNA. Markers may include, e.g., regulatory regions, flanking regions, intergenic regions, etc. Similarly, the term “marker” used in reference to any component of a sample, e.g., protein, RNA, carbohydrate, small molecule, etc., refers to a component that can be assayed in a sample (e.g., measured or otherwise characterized) and that is associated with a condition of a subject, or of the sample from a subject. The term “methylation marker” refers to a gene or DNA in which the methylation state of the gene or DNA is associated with a condition, e.g., cancer.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides is the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera, et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. An exemplary reagent is a bisulfite reagent.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivatives thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

A change in the nucleic acid nucleotide sequence by a methylation-specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a nucleotide that is typically methylated and an unmethylated selected nucleotides refers specifically to a nucleotide that typically occurs in unmethylated form.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence. As used in reference to a double-stranded nucleic acid, the term “target” is not limited to a particular strand of the duplexed target, e.g., a coding strand, but may be used in reference to either or both strands of, for example, a double-stranded gene or reference DNA.

The term “marker”, as used herein, refers to a substance (e.g., a nucleic acid, or a region of a nucleic acid, or a protein) that may be used to distinguish non-normal cells (e.g., cancer cells) from normal cells (non-cancerous cells), e.g., based on presence, absence, or status (e.g., methylation state) of the marker substance. As used herein “normal” methylation of a marker refers to a degree of methylation typically found in normal cells, e.g., in non-cancerous cells.

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including race horses), and the like. The presently-disclosed subject matter further includes a system for diagnosing a lung cancer in a subject. The system can be provided, for example, as a commercial kit that can be used to screen for a risk of lung cancer or diagnose a lung cancer in a subject from whom a biological sample has been collected. An exemplary system provided in accordance with the present technology includes assessing the methylation state of a marker described herein.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic or other DNA or RNA, without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.” Those of skill in the art will understand the term “PCR” encompasses many variants of the originally described method using, e.g., real time PCR, nested PCR, reverse transcription PCR (RT-PCR), single primer and arbitrarily primed PCR, etc.

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety).

In some embodiments, target nucleic acid is amplified (e.g., by PCR) and amplified nucleic acid is detected simultaneously using an invasive cleavage assay. Assays configured for performing a detection assay (e.g., invasive cleavage assay) in combination with an amplification assay are described in U.S. Pat. No. 9,096,893, incorporated herein by reference in its entirety for all purposes. Additional amplification plus invasive cleavage detection configurations, termed the QuARTS method, are described in, e.g., in U.S. Pat. Nos. 8,361,720; 8,715,937; 8,916,344; 9,212,392, and U.S. patent application Ser. No. 15/841,006 each of which is incorporated herein by reference for all purposes. The term “invasive cleavage structure” as used herein refers to a cleavage structure comprising i) a target nucleic acid, ii) an upstream nucleic acid (e.g., an invasive or “INVADER” oligonucleotide), and iii) a downstream nucleic acid (e.g., a probe), where the upstream and downstream nucleic acids anneal to contiguous regions of the target nucleic acid, and where an overlap forms between the a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, the 3′ portion of the upstream nucleic acid that overlaps with the downstream duplex is a non-base chemical moiety such as an aromatic ring structure, e.g., as disclosed, for example, in U.S. Pat. No. 6,090,543, incorporated herein by reference in its entirety. In some embodiments, one or more of the nucleic acids may be attached to each other, e.g., through a covalent linkage such as nucleic acid stem-loop, or through a non-nucleic acid chemical linkage (e.g., a multi-carbon chain). As used herein, the term “flap endonuclease assay” includes “INVADER” invasive cleavage assays and QuARTS assays, as described above.

The term “probe oligonucleotide” or “flap oligonucleotide” when used in reference to flap assay, refers to an oligonucleotide that interacts with a target nucleic acid to form a cleavage structure in the presence of an invasive oligonucleotide.

The term “invasive oligonucleotide” refers to an oligonucleotide that hybridizes to a target nucleic acid at a location adjacent to the region of hybridization between a probe and the target nucleic acid, wherein the 3′ end of the invasive oligonucleotide comprises a portion (e.g., a chemical moiety, or one or more nucleotides) that overlaps with the region of hybridization between the probe and target. The 3′ terminal nucleotide of the invasive oligonucleotide may or may not base pair a nucleotide in the target. In some embodiments, the invasive oligonucleotide contains sequences at its 3′ end that are substantially the same as sequences located at the 5′ end of a portion of the probe oligonucleotide that anneals to the target strand.

The term “flap endonuclease” or “FEN,” as used herein, refers to a class of nucleolytic enzymes, typically 5′ nucleases, that act as structure-specific endonucleases on DNA structures with a duplex containing a single stranded 5′ overhang, or flap, on one of the strands that is displaced by another strand of nucleic acid (e.g., such that there are overlapping nucleotides at the junction between the single and double-stranded DNA). FENs catalyze hydrolytic cleavage of the phosphodiester bond at the junction of single and double stranded DNA, releasing the overhang, or the flap. Flap endonucleases are reviewed by Ceska and Savers (Trends Biochem. Sci. 1998 23:331-336) and Liu et al (Annu. Rev. Biochem. 2004 73: 589-615; herein incorporated by reference in its entirety). FENs may be individual enzymes, multi-subunit enzymes, or may exist as an activity of another enzyme or protein complex (e.g., a DNA polymerase).

A flap endonuclease may be thermostable. For example, FEN-1 flap endonuclease from archival thermophiles organisms are typical thermostable. As used herein, the term “FEN-1” refers to a non-polymerase flap endonuclease from a eukaryote or archaeal organism. See, e.g., WO 02/070755, and Kaiser M. W., et al. (1999) J. Biol. Chem., 274:21387, which are incorporated by reference herein in their entireties for all purposes.

As used herein, the term “cleaved flap” refers to a single-stranded oligonucleotide that is a cleavage product of a flap assay.

The term “cassette,” when used in reference to a flap cleavage reaction, refers to an oligonucleotide or combination of oligonucleotides configured to generate a detectable signal in response to cleavage of a flap or probe oligonucleotide, e.g., in a primary or first cleavage structure formed in a flap cleavage assay. In preferred embodiments, the cassette hybridizes to a non-target cleavage product produced by cleavage of a flap oligonucleotide to form a second overlapping cleavage structure, such that the cassette can then be cleaved by the same enzyme, e.g., a FEN-1 endonuclease.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label, e.g., a fluorophore. In particularly preferred embodiments, a cassette comprises labeled moieties that produce a FRET effect.

As used herein, the term “FRET” refers to fluorescence resonance energy transfer, a process in which moieties (e.g., fluorophores) transfer energy e.g., among themselves, or, from a fluorophore to a non-fluorophore (e.g., a quencher molecule). In some circumstances, FRET involves an excited donor fluorophore transferring energy to a lower-energy acceptor fluorophore via a short-range (e.g., about 10 nm or less) dipole-dipole interaction. In other circumstances, FRET involves a loss of fluorescence energy from a donor and an increase in fluorescence in an acceptor fluorophore. In still other forms of FRET, energy can be exchanged from an excited donor fluorophore to a non-fluorescing molecule (e.g., a “dark” quenching molecule). FRET is known to those of skill in the art and has been described (See, e.g., Stryer et al., 1978, Ann. Rev. Biochem., 47:819; Selvin, 1995, Methods Enzymol., 246:300; Orpana, 2004 Biomol Eng 21, 45-50; Olivier, 2005 Mutant Res 573, 103-110, each of which is incorporated herein by reference in its entirety).

In an exemplary flap detection assay, an invasive oligonucleotide and flap oligonucleotide are hybridized to a target nucleic acid to produce a first complex having an overlap as described above. An unpaired “flap” is included on the 5′ end of the flap oligonucleotide. The first complex is a substrate for a flap endonuclease, e.g., a FEN-1 endonuclease, which cleaves the flap oligonucleotide to release the 5′ flap portion. In a secondary reaction, the released 5′ flap product serves as an invasive oligonucleotide on a FRET cassette to again create the structure recognized by the flap endonuclease, such that the FRET cassette is cleaved. When the fluorophore and the quencher are separated by cleavage of the FRET cassette, a detectable fluorescent signal above background fluorescence is produced.

The term “real time” as used herein in reference to detection of nucleic acid amplification or signal amplification refers to the detection or measurement of the accumulation of products or signal in the reaction while the reaction is in progress, e.g., during incubation or thermal cycling. Such detection or measurement may occur continuously, or it may occur at a plurality of discrete points during the progress of the amplification reaction, or it may be a combination. For example, in a polymerase chain reaction, detection (e.g., of fluorescence) may occur continuously during all or part of thermal cycling, or it may occur transiently, at one or more points during one or more cycles. In some embodiments, real time detection of PCR or QuARTS reactions is accomplished by determining a level of fluorescence at the same point (e.g., a time point in the cycle, or temperature step in the cycle) in each of a plurality of cycles, or in every cycle. Real time detection of amplification may also be referred to as detection “during” the amplification reaction.

As used herein, the term “quantitative amplification data set” refers to the data obtained during quantitative amplification of the target sample, e.g., target DNA. In the case of quantitative PCR or QuARTS assays, the quantitative amplification data set is a collection of fluorescence values obtained at during amplification, e.g., during a plurality of, or all of the thermal cycles. Data for quantitative amplification is not limited to data collected at any particular point in a reaction, and fluorescence may be measured at a discrete point in each cycle or continuously throughout each cycle.

The abbreviations “Ct” and “Cp” as used herein in reference to data collected during real time PCR and PCR+INVADER assays refer to the cycle at which signal (e.g., fluorescent signal) crosses a predetermined threshold value indicative of positive signal. Various methods have been used to calculate the threshold that is used as a determinant of signal verses concentration, and the value is generally expressed as either the “crossing threshold” (Ct) or the “crossing point” (Cp). Either Cp values or Ct values may be used in embodiments of the methods presented herein for analysis of real-time signal for the determination of the percentage of variant and/or non-variant constituents in an assay or sample.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides.

The term “system” as used herein refers to a collection of articles for use for a particular purpose. In some embodiments, the articles comprise instructions for use, as information supplied on e.g., an article, on paper, or on recordable media (e.g., DVD, CD, flash drive, etc.). In some embodiments, instructions direct a user to an online location, e.g., a website.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### DESCRIPTION OF THE DRAWINGS

FIG. 1 shows schematic diagrams of marker target regions in unconverted form and bisulfite-converted form. Flap assay primers and probes for detection of bisulfite-converted target DNA are shown.

FIGS. 2-5 provide tables comparing Reduced Representation Bisulfite Sequencing (RRBS) results for selecting markers associated with lung carcinomas as described in Example 2, with each row showing the mean values for the indicated marker region (identified by chromosome and start and stop positions). The ratio of mean methylation for each tissue type (normal (Norm), adenocarcinoma (Ad), large cell carcinoma (LC), small cell carcinoma (SC), squamous cell carcinoma (SQ) and undefined cancer (UND)) is compared to the mean methylation of buffy coat samples from normal subjects (WBC or BC)) is shown for each region, and genes and transcripts identified with each region are indicated.

FIG. 2 provides a table comparing RRBS results for selecting markers associated with lung adenocarcinoma.

FIG. 3 provides a table comparing RRBS results for selecting markers associated with lung large cell carcinoma.

FIG. 4 provides a table comparing RRBS results for selecting markers associated with lung small cell carcinoma.

FIG. 5 provides a table comparing RRBS results for selecting markers associated with lung squamous cell carcinoma.

FIG. 6 provides a table of nucleic acid sequences of assay targets and detection oligonucleotides, with corresponding SEQ ID NOS. Target nucleic acids, in particular target DNAs (including bisulfite-converted DNAs) are shown for convenience as single strands but it is understood that embodiments of the technology encompass the complementary strands of the depicted sequences. For example, primers and flap oligonucleotides may be selected to hybridize to the targets as shown, or to strands that are complementary to the targets as shown.

FIG. 7 provides a graph showing a 6-marker logistic fit of data from Example 3, using markers SHOX2, SOBP, ZNF781, BTACT, CYP26C1, and DLX4. The ROC curve analysis shows an area under the curve (AUC) of 0.973.

FIG. 8 provides a graph showing a 6-marker logistic fit of data from Example 3, using markers SHOX2, SOBP, ZNF781, CYP26C1, SUCLG2, and SKI. The ROC curve analysis shows an area under the curve (AUC) of 0.97982.

FIG. 9A-9I show graphs showing individual marker logistic fit of data from Example 6.

FIG. 10 provides a graph showing a 6-marker logistic fit of data from Example 6, using markers BARX1, FLJ45983, SOBP, HOPX, IFFO1, and ZNF781.

### DETAILED DESCRIPTION OF THE INVENTION

Provided herein is technology relating to selection of nucleic acid markers for use in assays for detection and quantification of DNA, e.g., methylated DNA, and use of the markers in nucleic acid detection assays. In particular, the technology relates to use of methylation assays to detect lung cancer.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

In some embodiments, a marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

The technology is not limited by sample type. For example, in some embodiments the sample is a stool sample, a tissue sample, sputum, a blood sample (e.g., plasma, serum, whole blood), an excretion, or a urine sample.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc.

The technology provides reagents for detecting a differentially methylated region (DMR). In some embodiments, an oligonucleotide is provided, the oligonucleotide comprising a sequence complementary to a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably to a marker selected from the subset SLC12A8, KLHDC7B, PARP15, OPLAH, BCL2L11, MAX.chr12.526, HOXB2, EMX1, CYP26C1, SOBP, SUCLG2, SHOX2, ZDHHC1, NFIX, FLJ45983, HOXA9, B3GALT6, ZNF781, SP9, BARX1, and SKI; or a marker selected from any of the subsets of markers defining the group consisting of ZNF781, BARX1, and EMX1; the group consisting of SHOX2, SOBP, ZNF781, CYP26C1, SUCLG2, and SKI; the group consisting of SLC12A8, KLHDC7B, PARP15, OPLAH, BCL2L11, MAX.chr12.526, HOXB2, and EMX1; the group consisting of SHOX2, SOBP, ZNF781, BTACT, CYP26C1, and DLX4; or the group consisting of SHOX2, SOBP, ZNF781, CYP26C1, SUCLG2, and SKI.

Kit embodiments are provided, e.g., a kit comprising a bisulfite reagent; and a control nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB 28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, and having a methylation state associated with a subject who does not have a cancer (e.g., lung cancer). In some embodiments, kits comprise a bisulfite reagent and an oligonucleotide as described herein. In some embodiments, kits comprise a bisulfite reagent; and a control nucleic acid comprising a sequence from such a chromosomal region and having a methylation state associated with a subject who has lung cancer.

The technology is related to embodiments of compositions (e.g., reaction mixtures). In some embodiments are provided a composition comprising a nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, and a bisulfite reagent. Some embodiments provide a composition comprising a nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, and an oligonucleotide as described herein. Some embodiments provide a composition comprising a nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, and a methylation-specific restriction enzyme. Some embodiments provide a composition comprising a nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372. HOXA9. TRH. SP9, DMRTA2. ARHGEF4. CYP26C1, ZNF781. PTGDR. GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERAMT3, NFIX, SIPR4, SKI, SUCLG2, TBX15, ZDHHC1, ZNF329, IFFO1, and HOPX, preferably from any of the subsets of markers as recited above, and a polymerase.

Additional related method embodiments are provided for screening for a neoplasm (e.g., lung carcinoma) in a sample obtained from a subject, e.g., a method comprising determining a methylation state of a marker in the sample comprising a base in a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above; comparing the methylation state of the marker from the subject sample to a methylation state of the marker from a normal control sample from a subject who does not have lung cancer; and determining a confidence interval and/or a p value of the difference in the methylation state of the subject sample and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001. Some embodiments of methods provide steps of reacting a nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the chromosomal region from a subject who does not have lung cancer to identify differences in the two sequences; and identifying the subject as having a neoplasm when a difference is present.

Systems for screening for lung cancer in a sample obtained from a subject are provided by the technology. Exemplary embodiments of systems include, e.g., a system for screening for lung cancer in a sample obtained from a subject, the system comprising an analysis component configured to determine the methylation state of a sample, a software component configured to compare the methylation state of the sample with a control sample or a reference sample methylation state recorded in a database, and an alert component configured to alert a user of a cancer-associated methylation state. An alert is determined in some embodiments by a software component that receives the results from multiple assays (e.g., determining the methylation states of multiple markers, e.g., a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, and calculating a value or result to report based on the multiple results. Some embodiments provide a database of weighted parameters associated with each a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above, provided herein for use in calculating a value or result and/or an alert to report to a user (e.g., such as a physician, nurse, clinician, etc.). In some embodiments all results from multiple assays are reported and in some embodiments one or more results are used to provide a score, value, or result based on a composite of one or more results from multiple assays that is indicative of a lung cancer risk in a subject.

In some embodiments of systems, a sample comprises a nucleic acid comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above. In some embodiments the system further comprises a component for isolating a nucleic acid, a component for collecting a sample such as a component for collecting a stool sample. In some embodiments, the system comprises nucleic acid sequences comprising a chromosomal region having an annotation selected from BARX1, LOC100129726, SPOCK2, TSC22D4·MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above. In some embodiments the database comprises nucleic acid sequences from subjects who do not have lung cancer. Also provided are nucleic acids, e.g., a set of nucleic acids, each nucleic acid having a sequence comprising a chromosomal region having an annotation selected from BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF329, IFFO1, and HOPX, preferably from any of the subsets of markers as recited above.

Related system embodiments comprise a set of nucleic acids as described, and a database of nucleic acid sequences associated with the set of nucleic acids. Some embodiments further comprise a bisulfite reagent. And, some embodiments further comprise a nucleic acid sequencer.

In certain embodiments, methods for characterizing a sample obtained from a human subject are provided, comprising a) obtaining a sample from a human subject; b) assaying a methylation state of one or more markers in the sample, wherein the marker comprises a base in a chromosomal region having an annotation selected from the following groups of markers: BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1. MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERAMT3, NFIX, SIPR4, SKI, SUCLG2, TBX15, ZDHHC1, ZNF329, IFFO1, and HOPX, preferably from any of the subsets of markers as recited above; and c) comparing the methylation state of the assayed marker to the methylation state of the marker assayed in a subject that does not have a neoplasm.

In some embodiments, the technology is related to assessing the presence of and methylation state of one or more of the markers identified herein in a biological sample. These markers comprise one or more differentially methylated regions (DMR) as discussed herein. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14: 7421-7427) and another example involves methylation-specific arbitrarily primed PCR (Gonzalgo et al. (1997) Cancer Res. 57: 594-599). In some embodiments, changes in methylation patterns at specific CpG sites are monitored by digestion of genomic DNA with methylation-specific restriction enzymes, particularly methylation-sensitive enzymes, followed by Southern analysis of the regions of interest (digestion-Southern method). In some embodiments, analyzing changes in methylation patterns involves a process comprising digestion of genomic DNA with one or more methylation-specific restriction enzymes, and analyzing regions for cleavage or non-cleavage indicating the methylation status of analyzed regions. In some embodiments, analysis of the treated DNA comprises PCR amplification, with the amplification result indicating whether the DNA was or was not cleaved by the restriction enzyme. In some embodiments, one or more of the presence, absence, amount, size, and sequence of an amplification product produced is assessed to analyze the methylation status of a DNA of interest. See, e.g., Melnikov, et al. (2005) Nucl. Acids Res, 33(10):e93; Hua, et al. (2011) Exp. Mol. Pathol. 91(1):455-60; and Singer-Sam et al. (1990) Nucl. Acids Res. 18: 687. In addition, other techniques have been reported that utilize bisulfite treatment of DNA as a starting point for methylation analysis. These include methylation-specific PCR (MSP) (Herman et al. (1992) Proc. Natl. Acad. Sci. USA 93: 9821-9826) and restriction enzyme digestion of PCR products amplified from bisulfite-converted DNA (Sadri and Hornsby (1996) Nucl. Acids Res. 24: 5058-5059; and Xiong and Laird (1997) Nucl. Acids Res. 25: 2532-2534). PCR techniques have been developed for detection of gene mutations (Kuppuswamy et al. (1991) Proc. Natl. Acad. Sci. USA 88: 1143-1147) and quantification of allelic-specific expression (Szabo and Mann (1995) Genes Dev. 9: 3097-3108; and Singer-Sam et al. (1992) PCR Methods Appl. 1: 160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated and the resulting methylation-specific sequence is determined using either a real-time PCR or other exponential amplification, e.g., a QuARTS assay (e.g., as provided by U.S. Pat. Nos. 8,361,720; 8,715,937; 8,916,344; and 9,212,392, and U.S. patent application Ser. No. 15/841,006).

For example, in some embodiments, methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amounts of methylated DNAs relative to the total amount of DNAs in the population, e.g., (number of methylated DNAs)/(the number of methylated DNAs+number of unmethylated DNAs)×100.

Also provided herein are compositions and kits for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, or other assays). In some embodiments, the kits containing one or more reagent necessary, sufficient, or useful for conducting a method are provided. Also provided are reactions mixtures containing the reagents. Further provided are master mix reagent sets containing a plurality of reagents that may be added to each other and/or to a test sample to complete a reaction mixture.

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

Genomic DNA may be isolated by any means, including the use of commercially available kits. Briefly, wherein the DNA of interest is encapsulated by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The technology relates to the analysis of any sample that may be associated with lung cancer, or that may be examined to establish the absence of lung cancer. For example, in some embodiments the sample comprises a tissue and/or biological fluid obtained from a patient. In some embodiments, the sample comprises a secretion. In some embodiments, the sample comprises sputum, blood, serum, plasma, gastric secretions, lung tissue samples, lung cells or lung DNA recovered from stool. In some embodiments, the subject is human. Such samples can be obtained by any number of means known in the art, such as will be apparent to the skilled person.

### I. Methylation Assays to Detect Lung Cancer

Candidate methylated DNA markers were identified by unbiased whole methylome sequencing of selected lung cancer case and lung control tissues. The top marker candidates were further evaluated in 255 independent patients with 119 controls, of which 37 were from benign nodules, and 136 cases inclusive of all lung cancer subtypes. DNA extracted from patient tissue samples was bisulfite treated and then candidate markers and β-actin (ACTB) as a normalizing gene were assayed by Quantitative Allele-Specific Real-time Target and Signal amplification (QuARTS amplification). QuARTS assay chemistry yields high discrimination for methylation marker selection and screening.

On receiver operator characteristics analyses of individual marker candidates, areas under the curve (AUCs) ranged from 0.512 to 0.941. At 100% specificity, a combined panel of 8 methylation markers (SLC12A8, KLHDC7B, PARP15, OPLAH, BCL2L11, MAX.12.526, HOXB2, and EMX1) yielded a sensitivity of 98.5% across all subtypes of lung cancer. Furthermore, using the 8 markers panel, benign lung nodules yielded no false positives.

### II. Methylation Detection Assays and Kits

The markers described herein find use in a variety of methylation detection assays. The most frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-specific restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophore per flap, providing exponential signal amplification. QUARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

In some embodiments, the bisulfite-treated DNA is purified prior to the quantification. This may be conducted by any means known in the art, such as but not limited to ultrafiltration, e.g., by means of Microcon™ columns (manufactured by Millipore™). The purification is carried out according to a modified manufacturer's protocol (see, e.g., PCT/EP2004/011715, which is incorporated by reference in its entirety). In some embodiments, the bisulfite treated DNA is bound to a solid support, e.g., a magnetic bead, and desulfonation and washing occurs while the DNA is bound to the support. Examples of such embodiments are provided, e.g., in WO 2013/116375 and U.S. Pat. No. 9,315,853. In certain preferred embodiments, support-bound DNA is ready for a methylation assay immediately after desulfonation and washing on the support. In some embodiments, the desulfonated DNA is eluted from the support prior to assay.

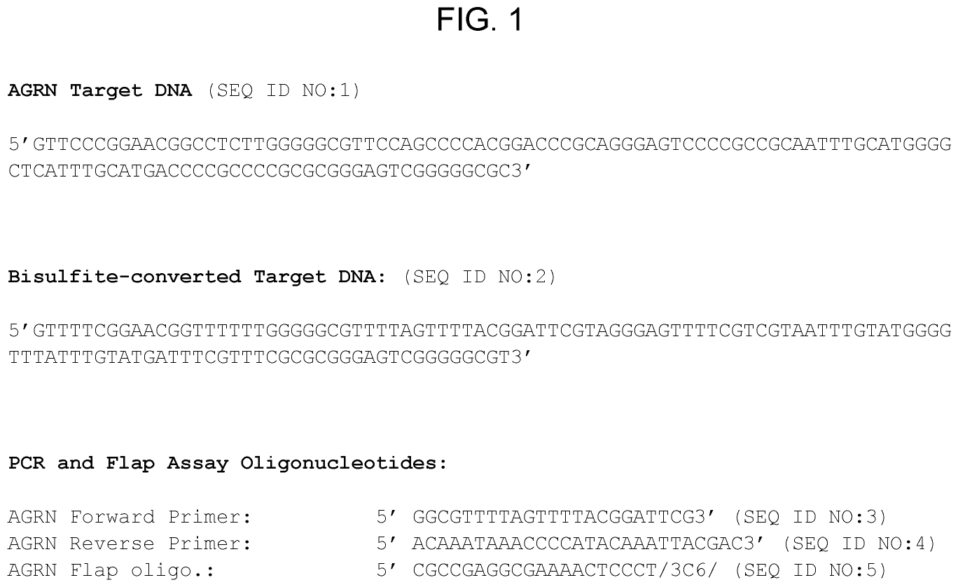

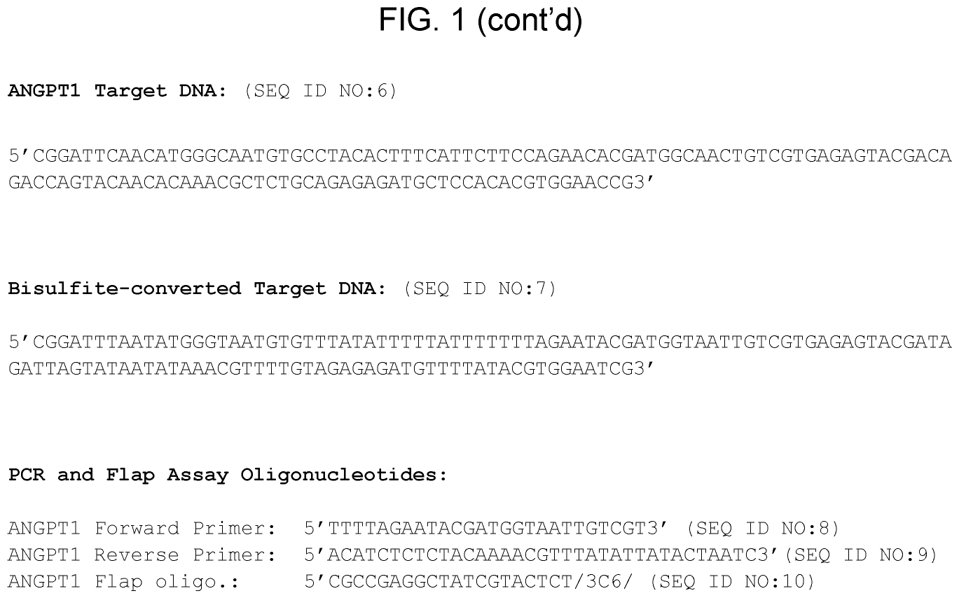

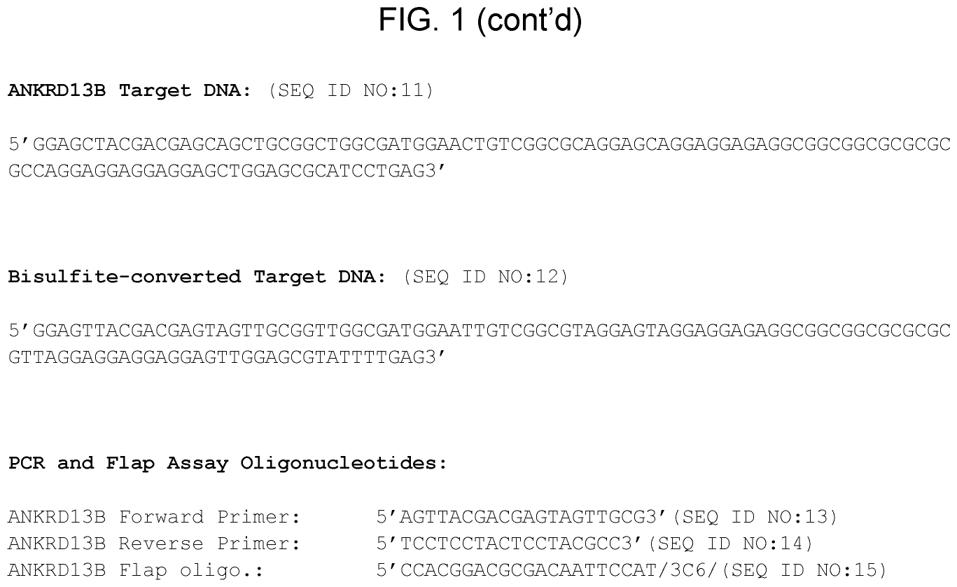

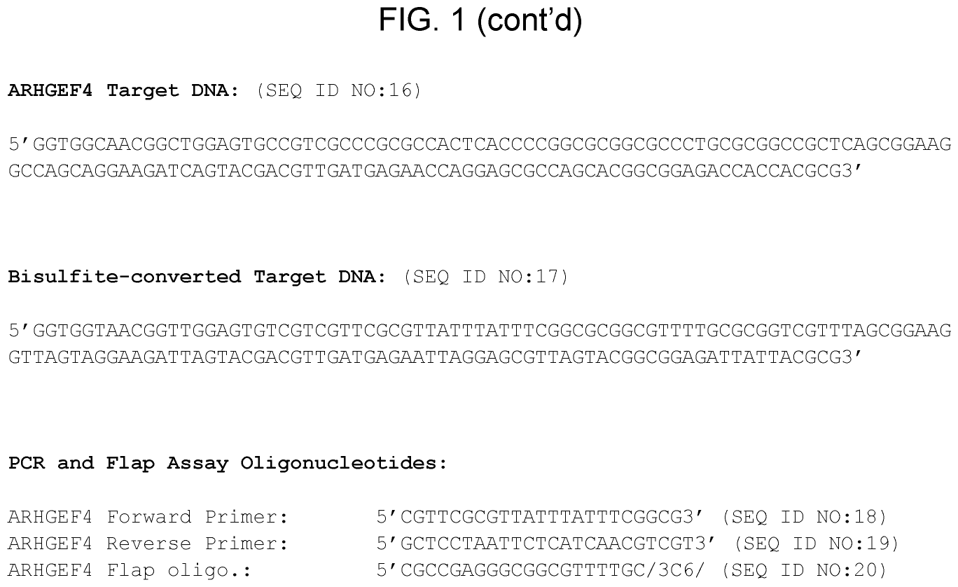

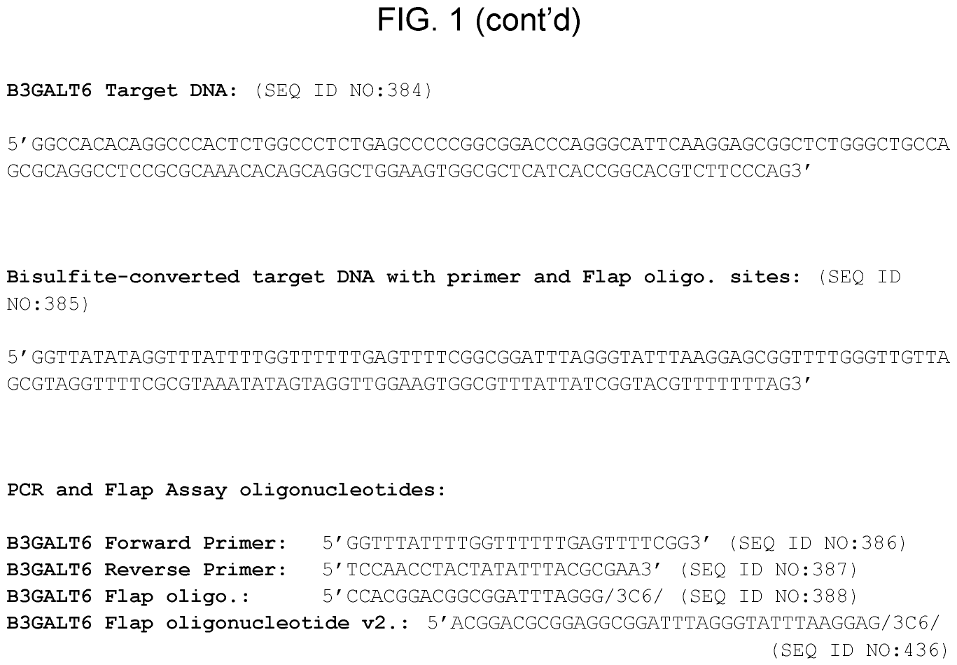

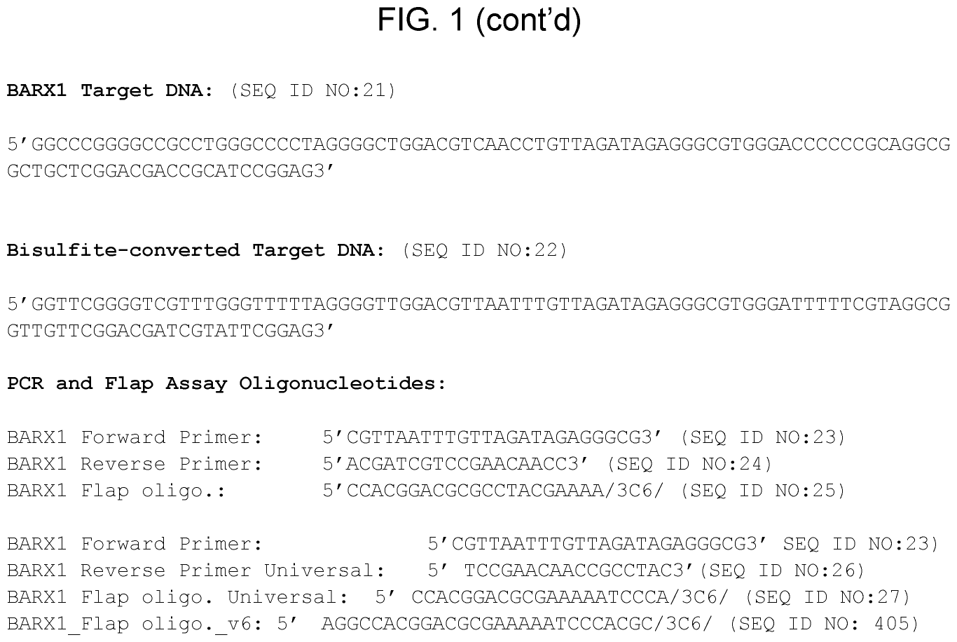

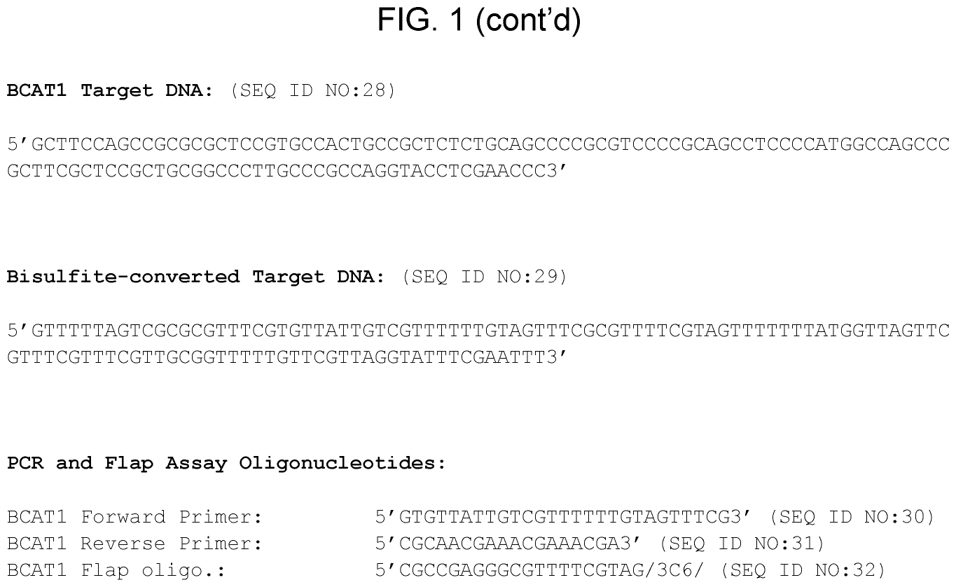

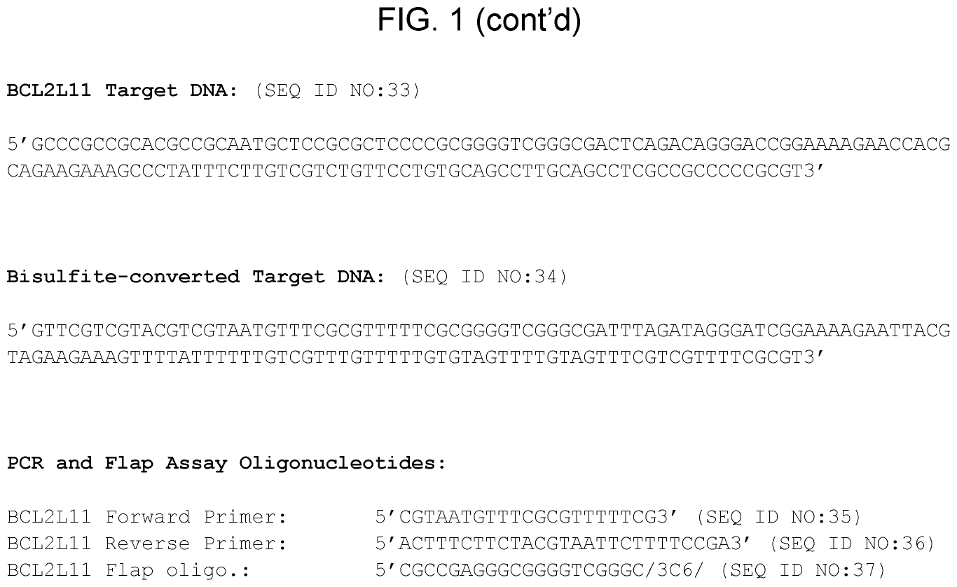

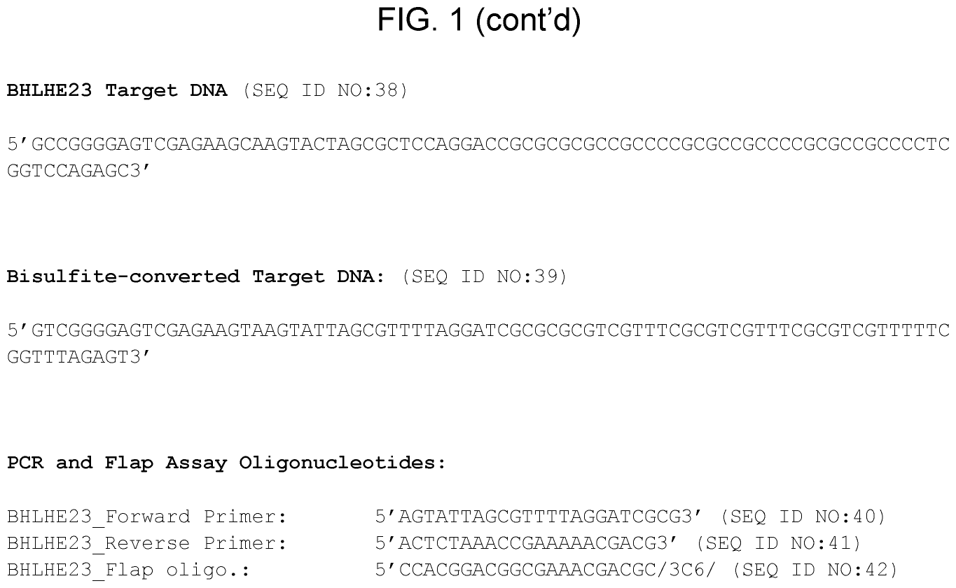

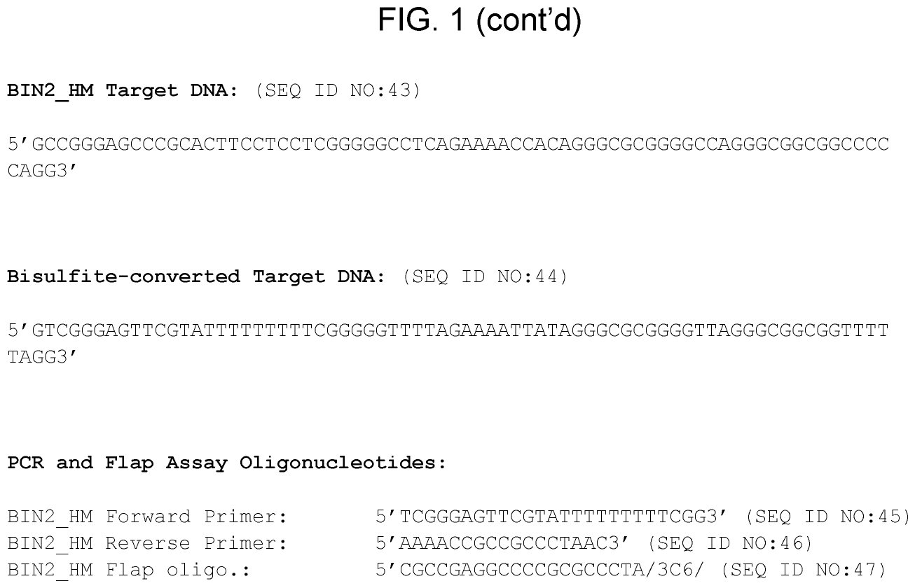

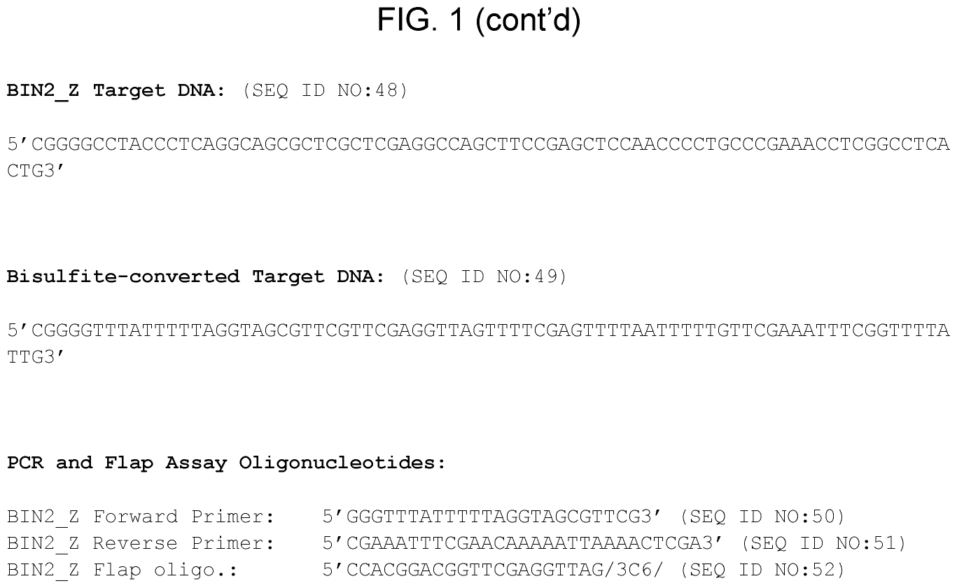

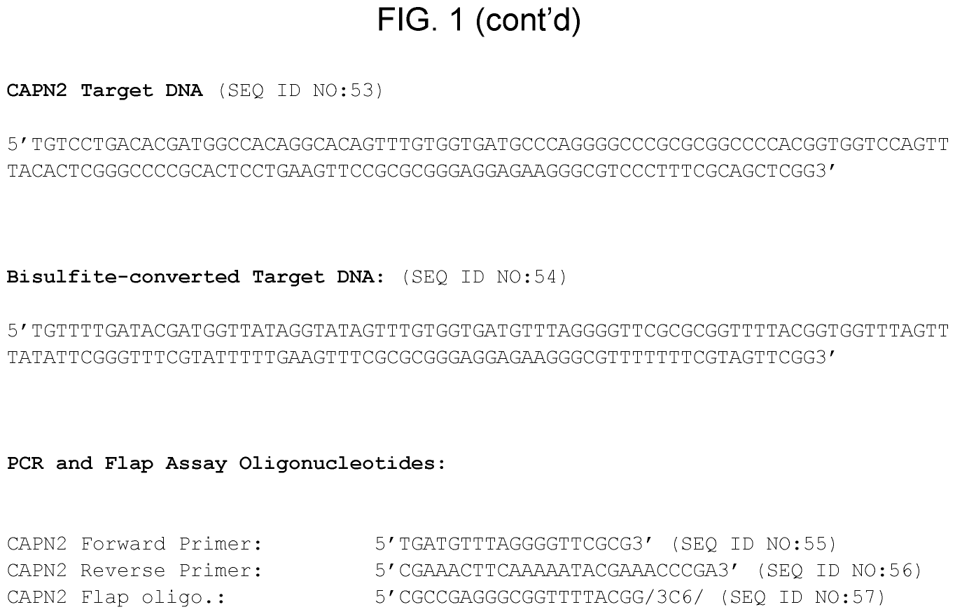

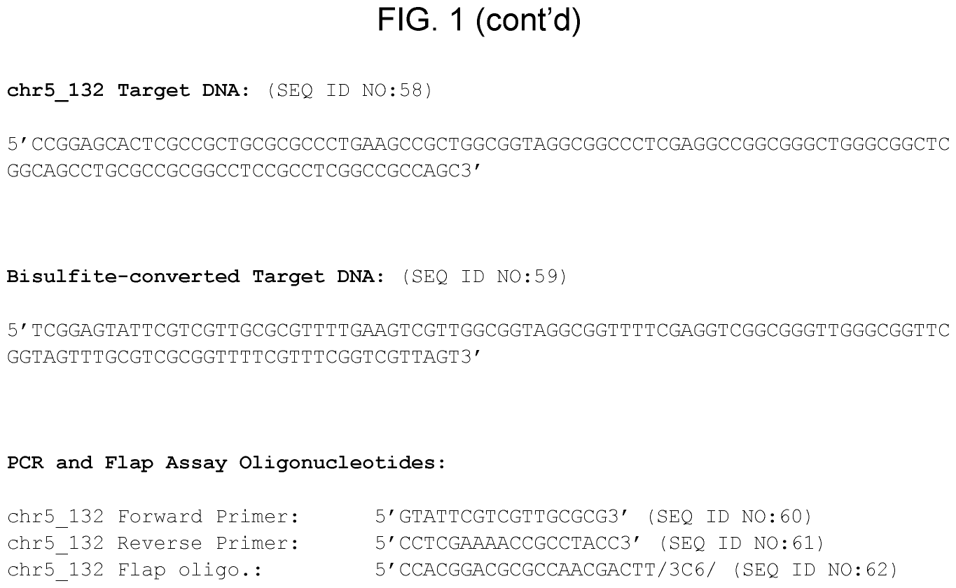

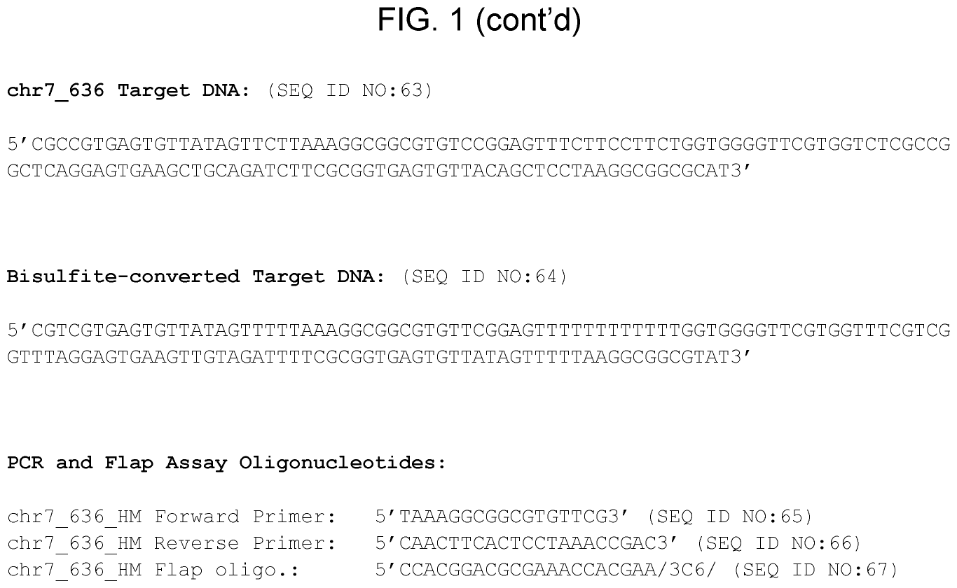

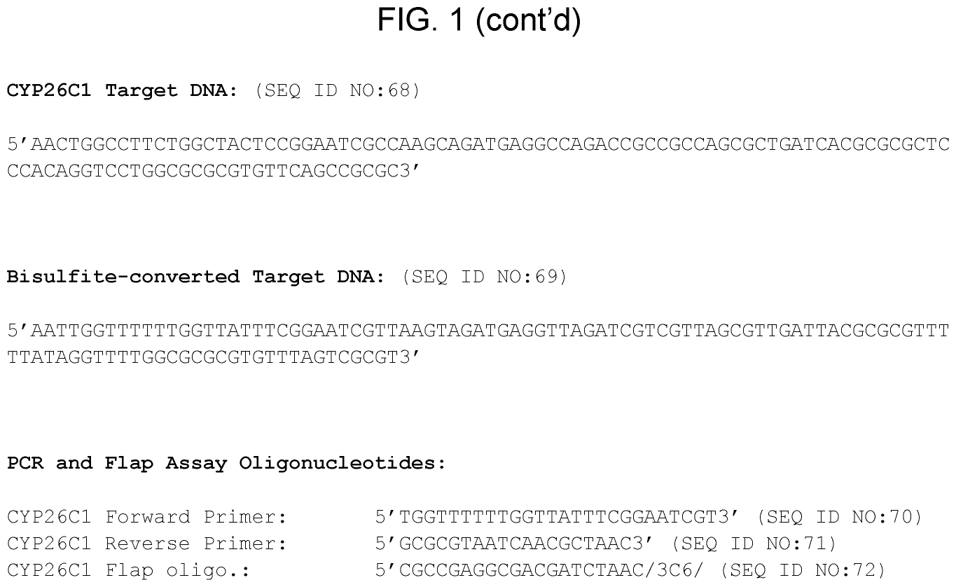

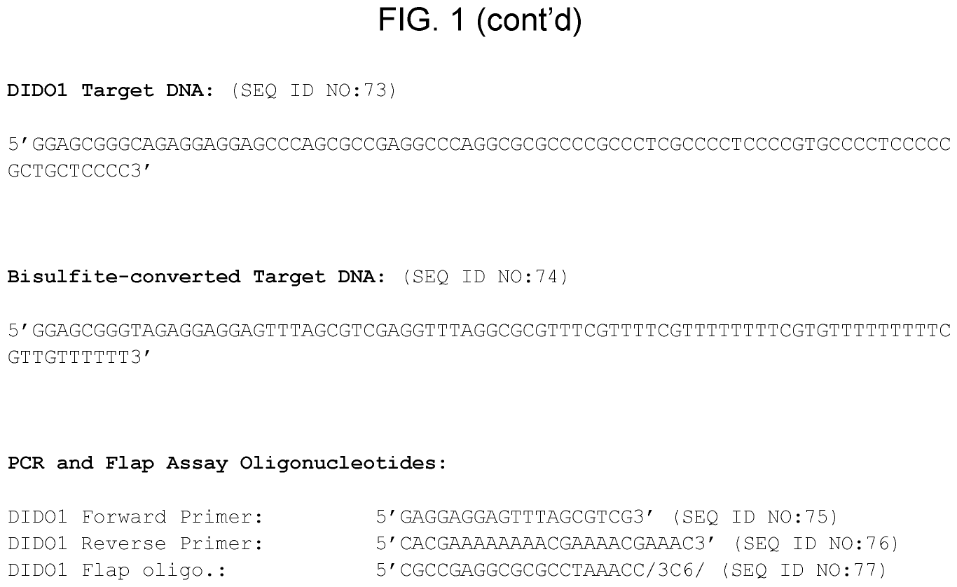

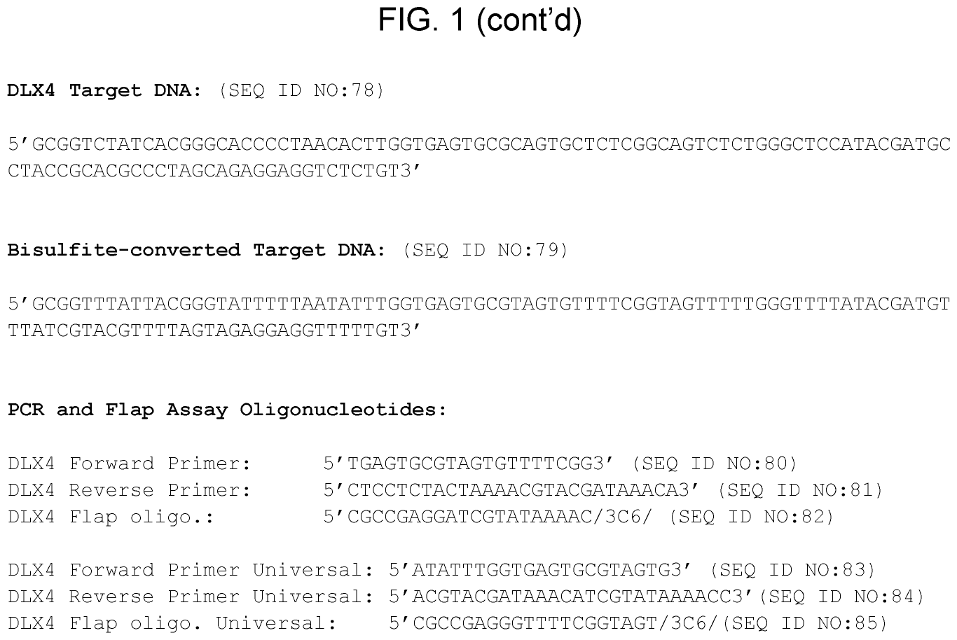

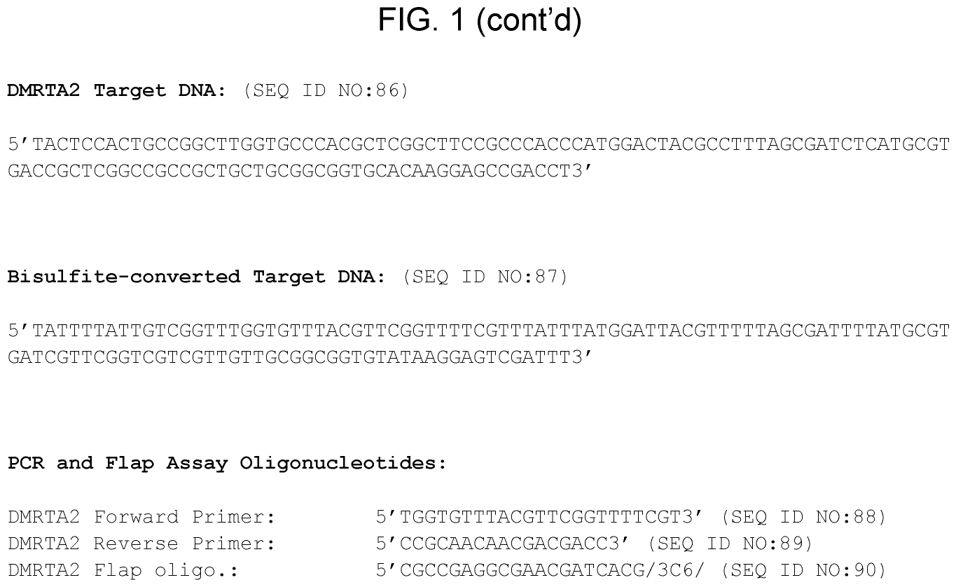

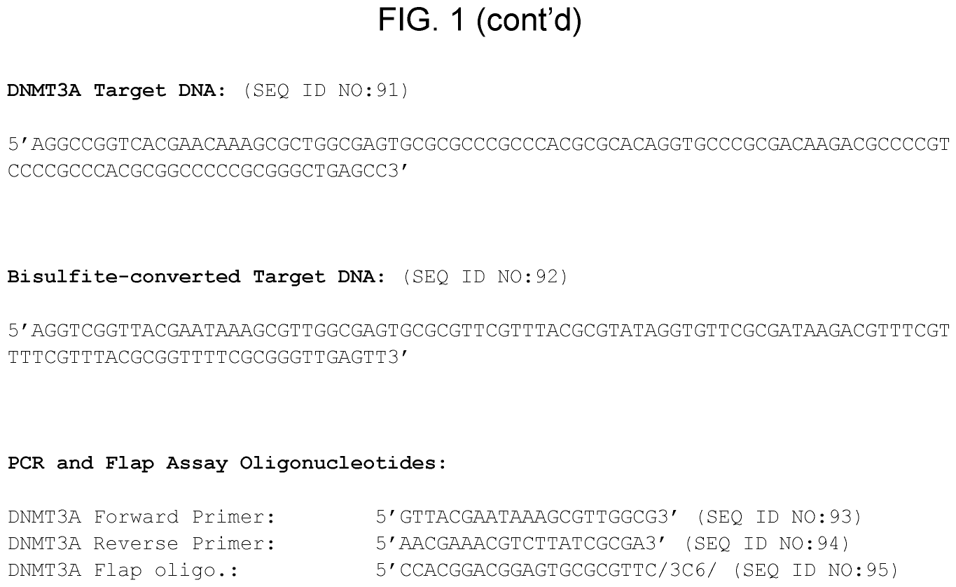

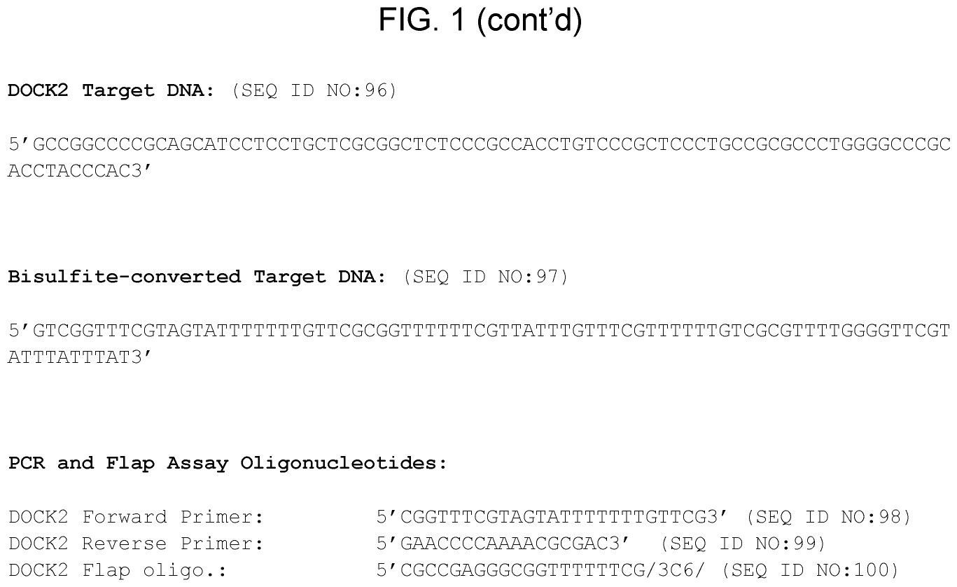

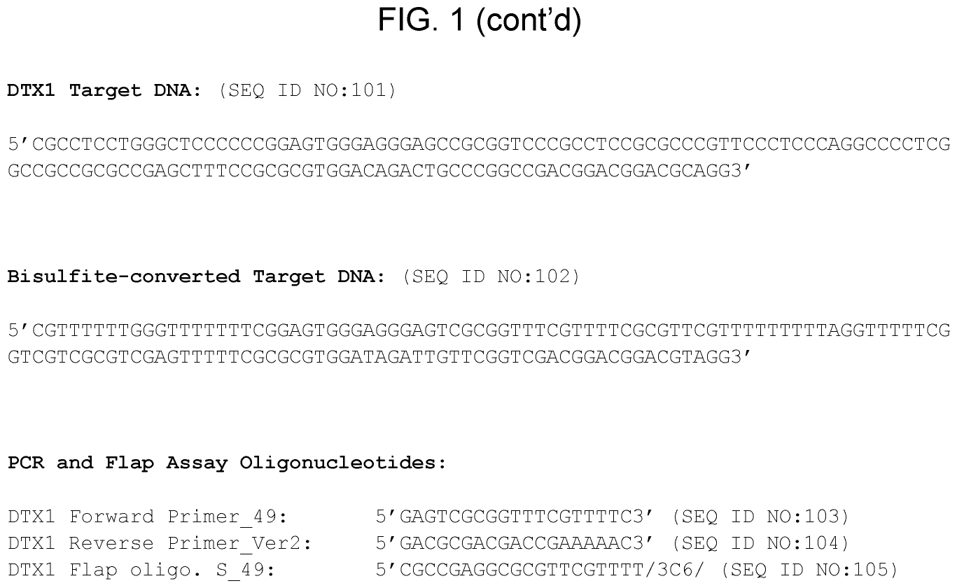

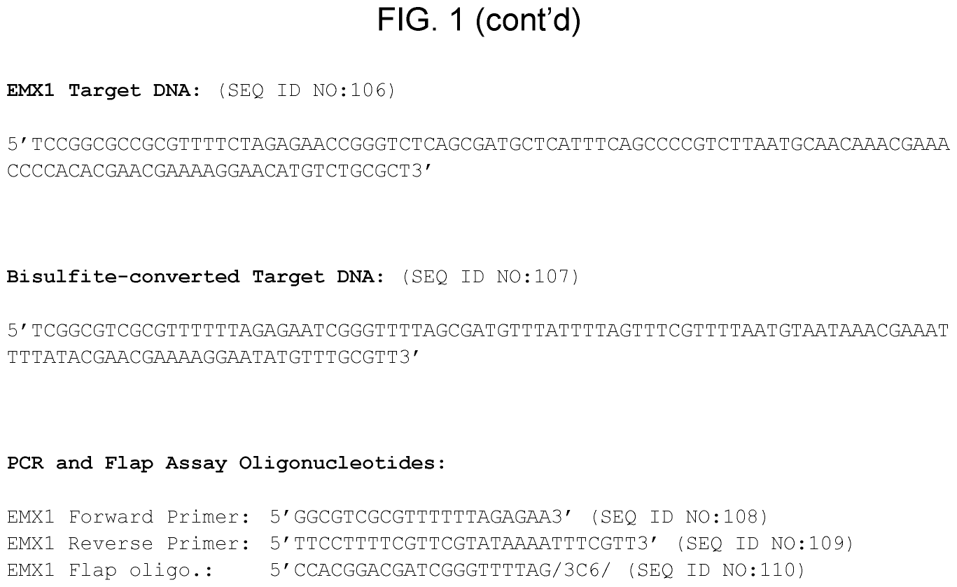

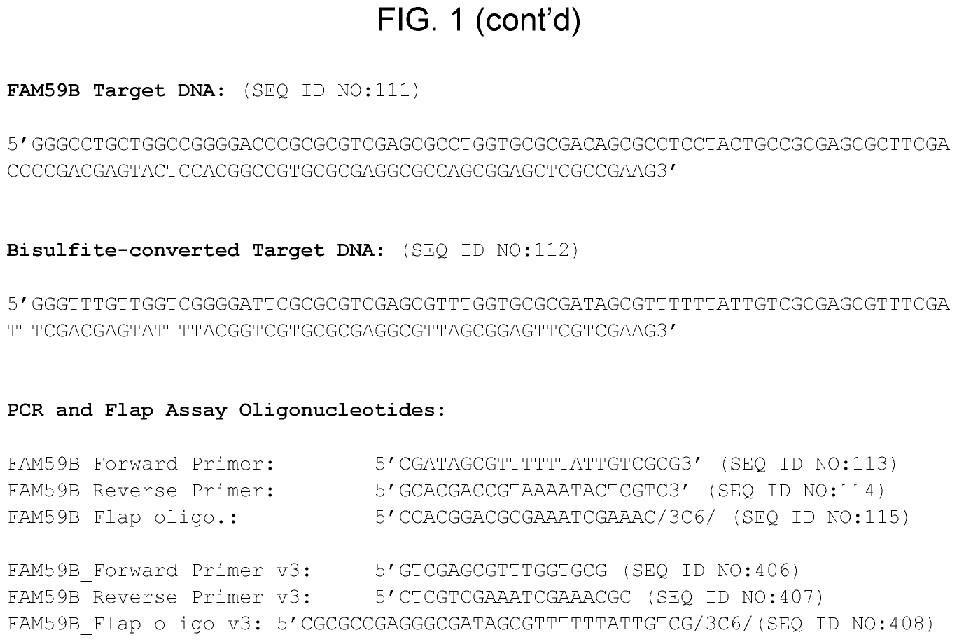

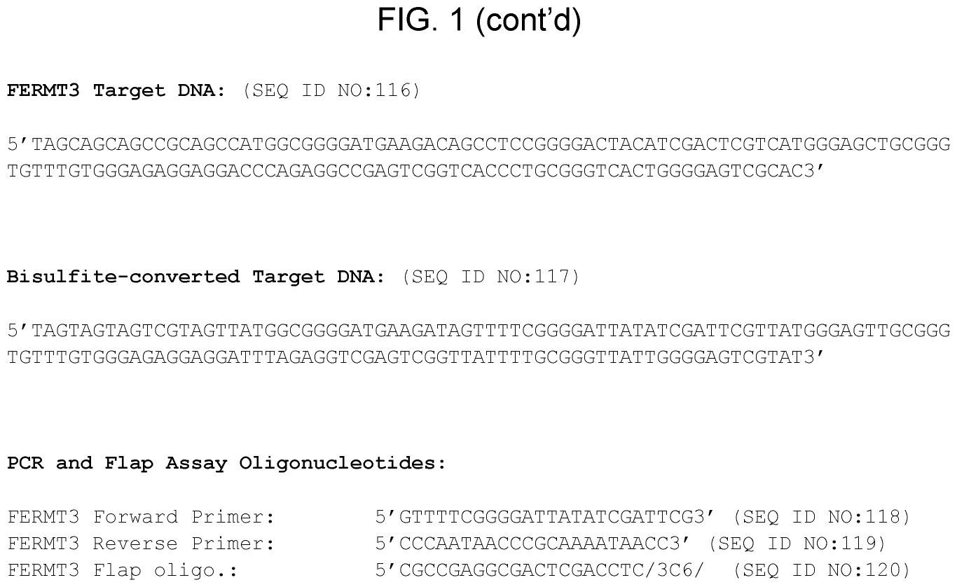

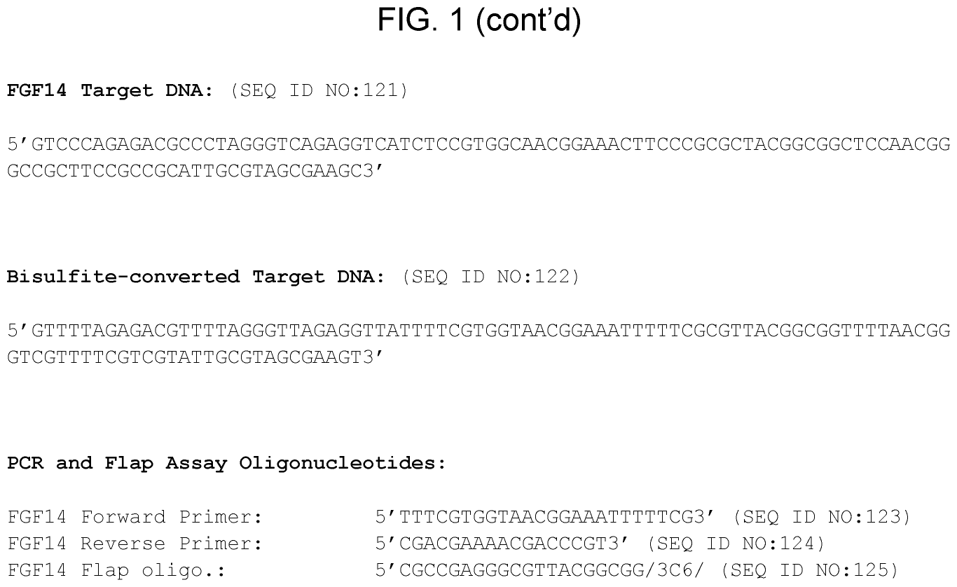

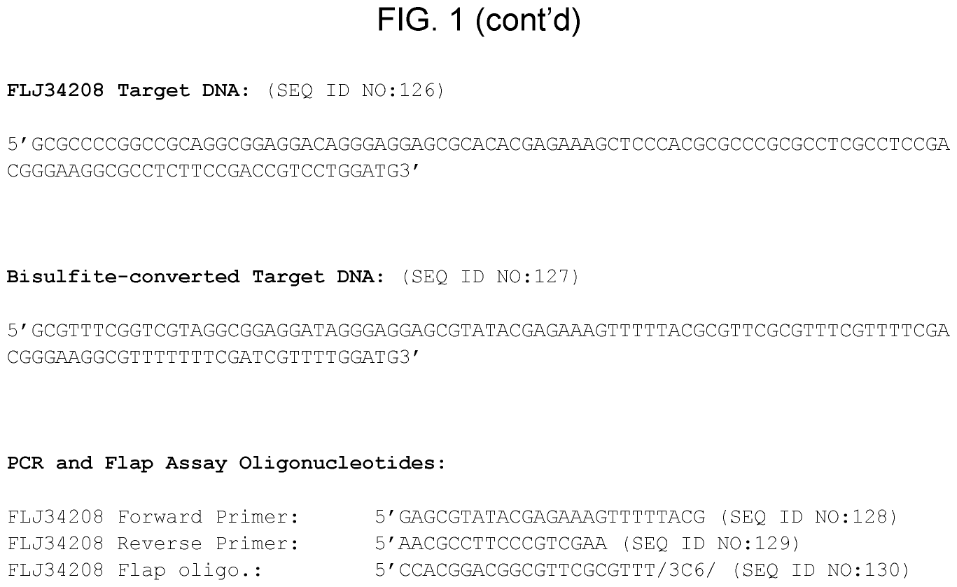

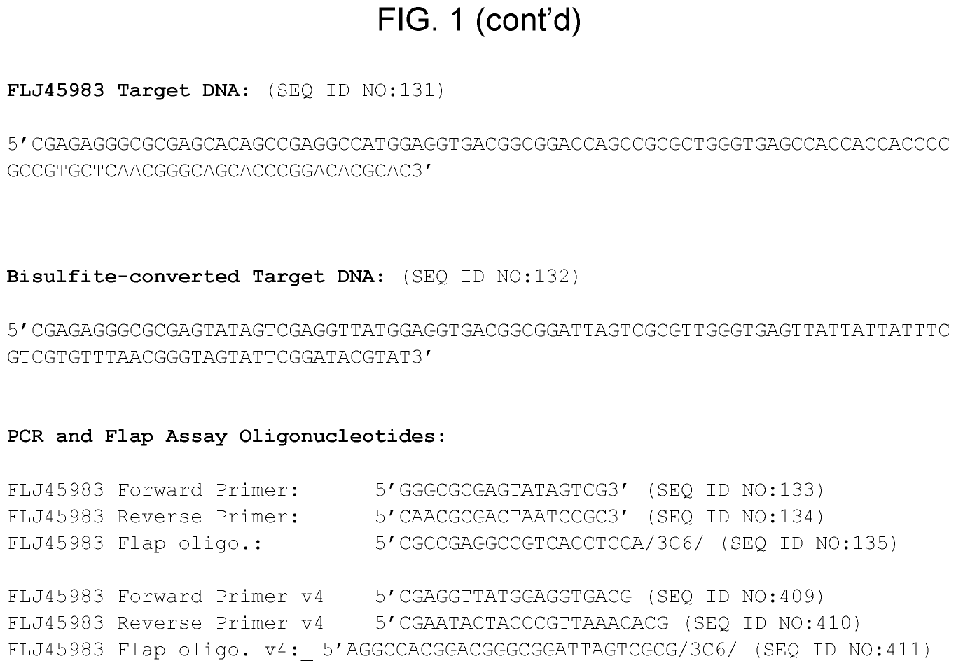

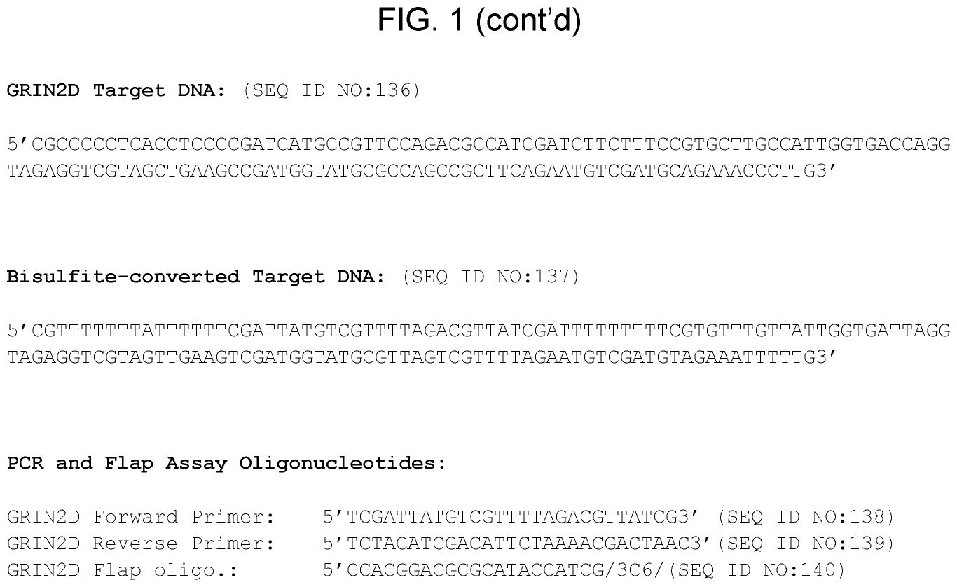

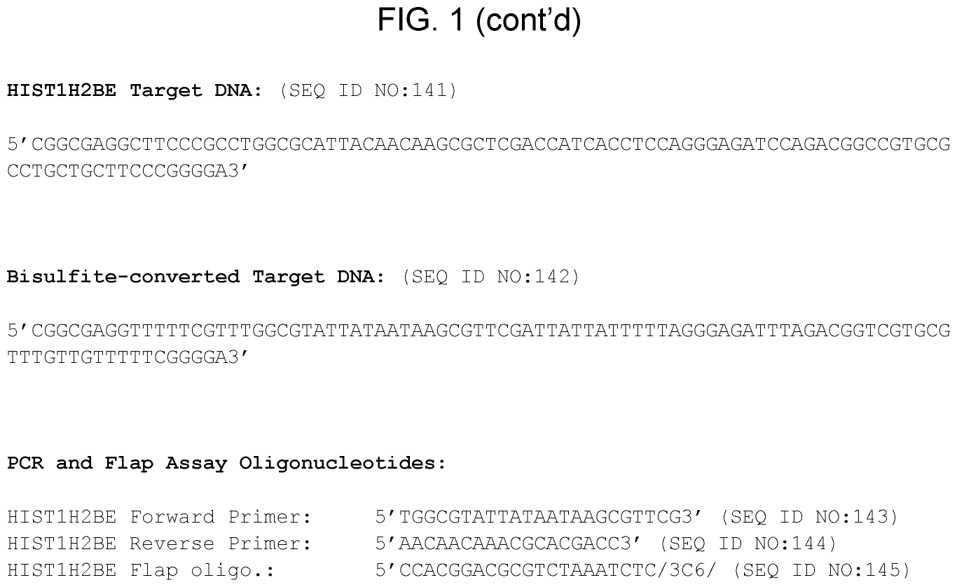

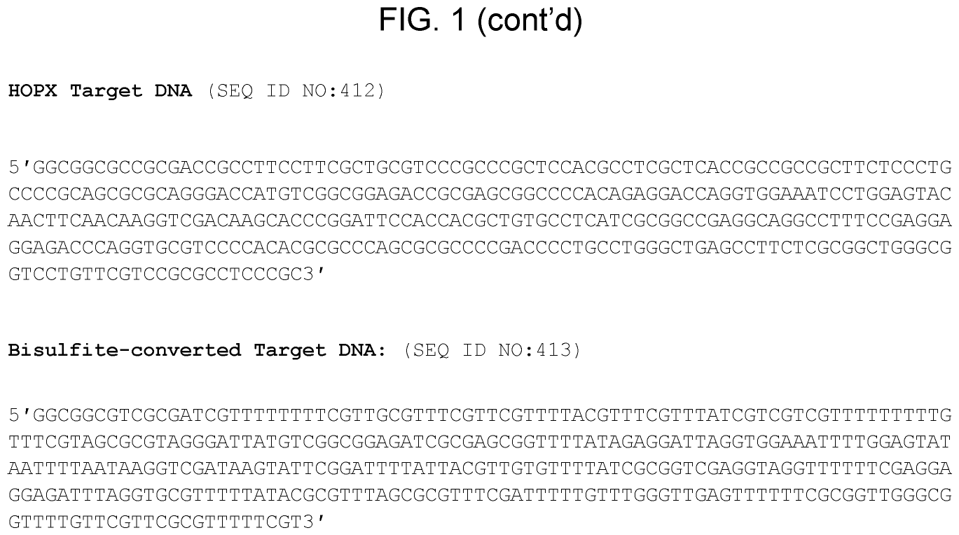

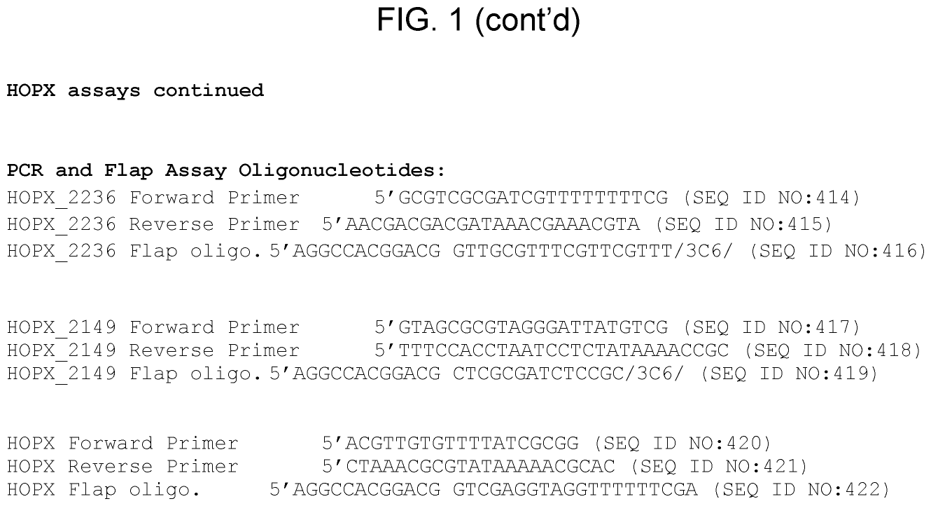

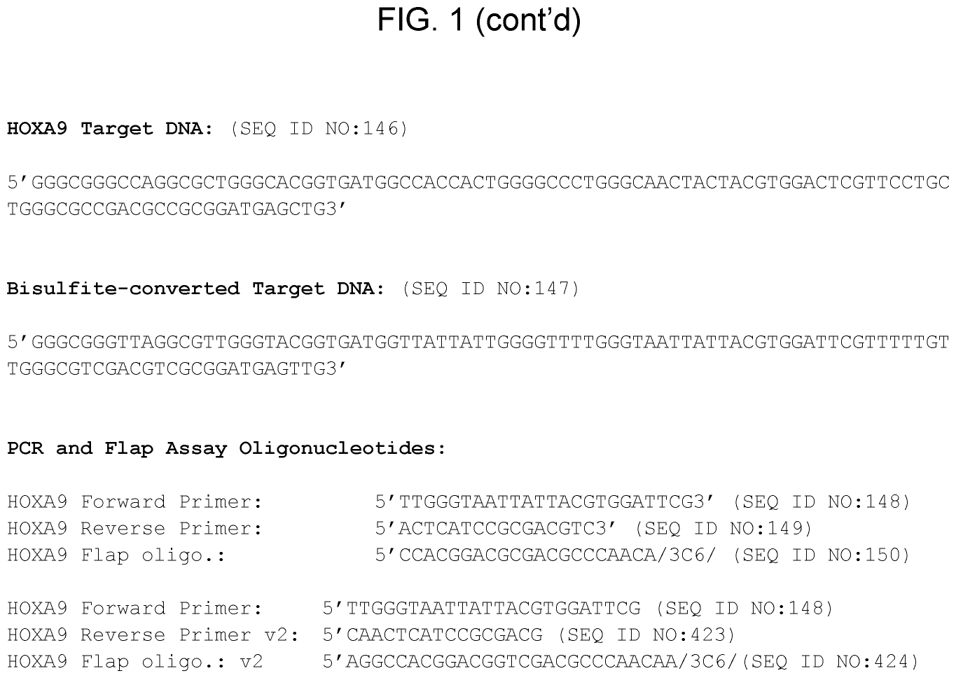

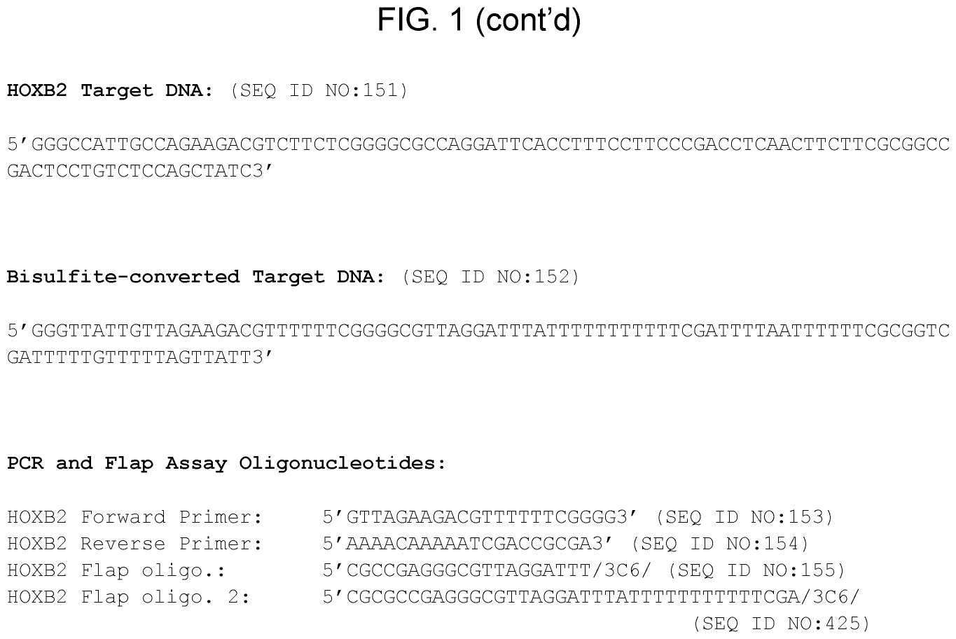

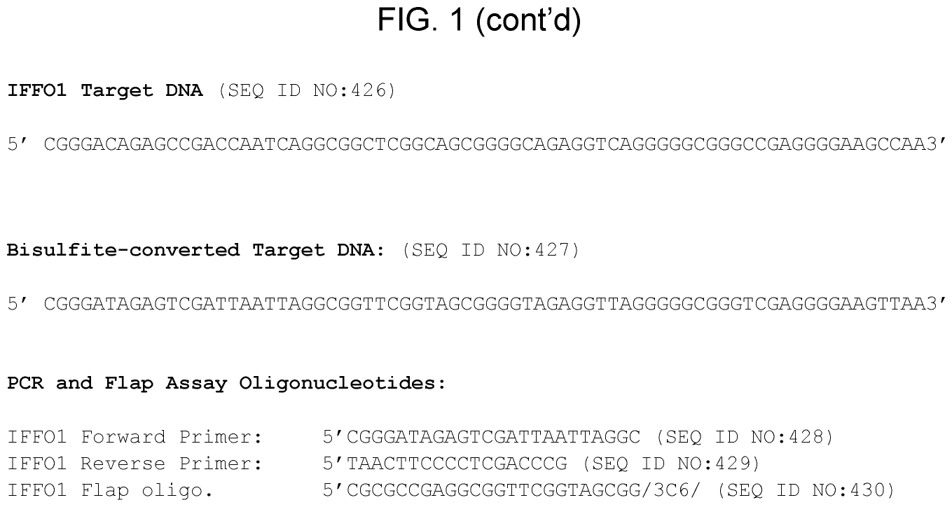

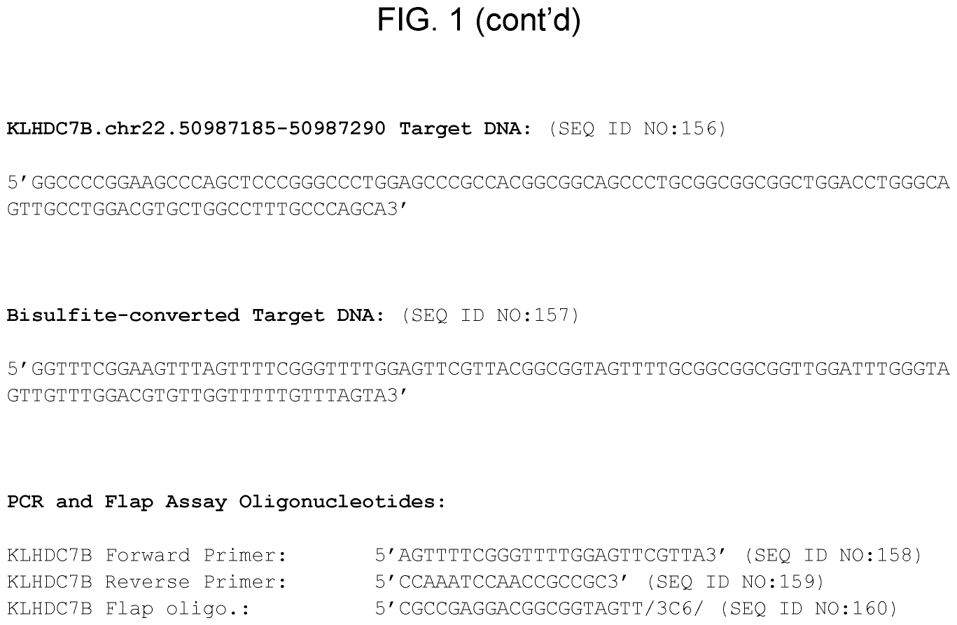

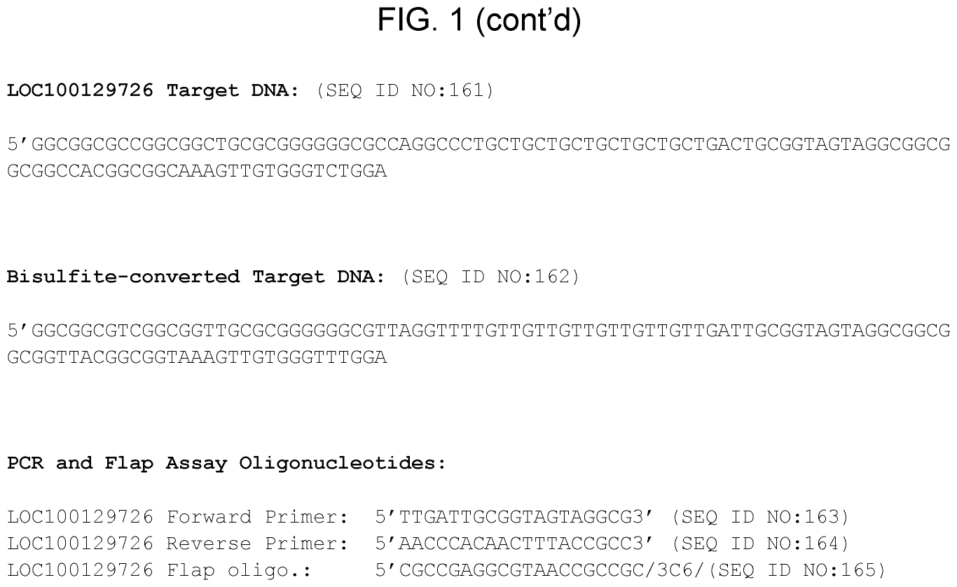

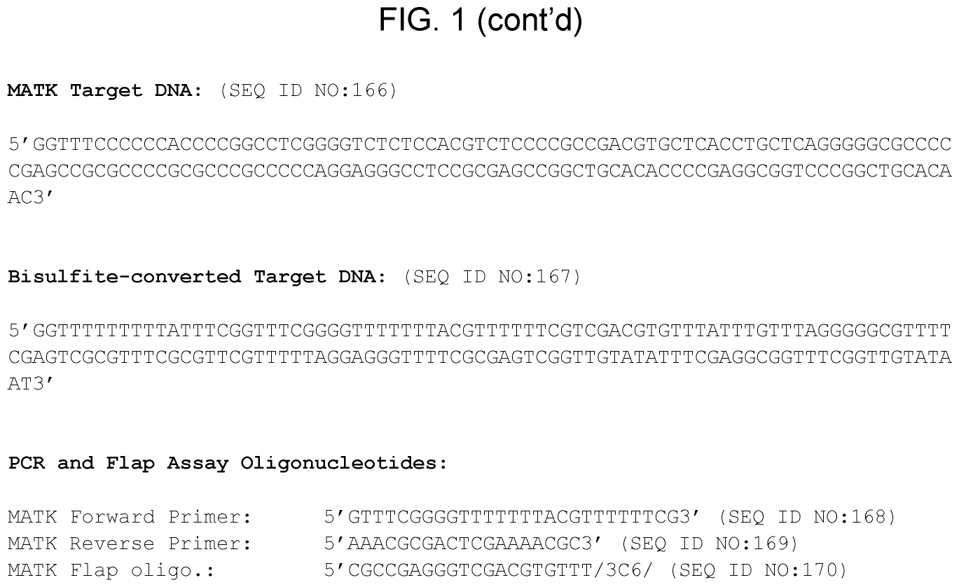

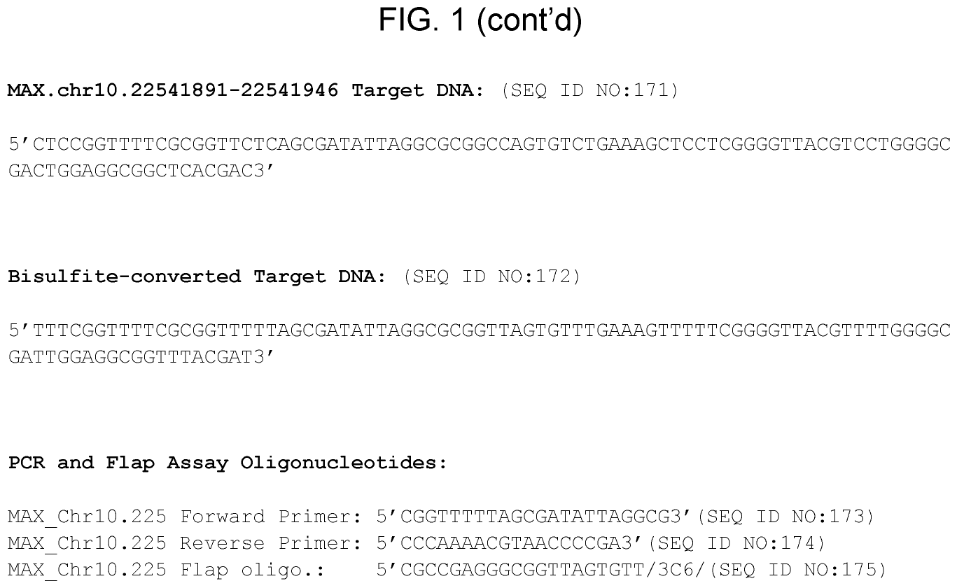

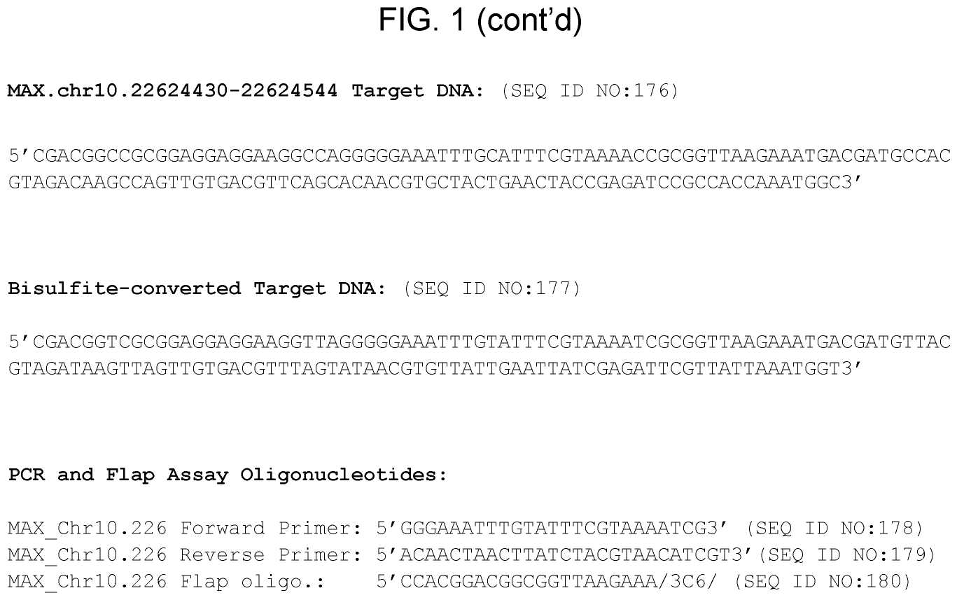

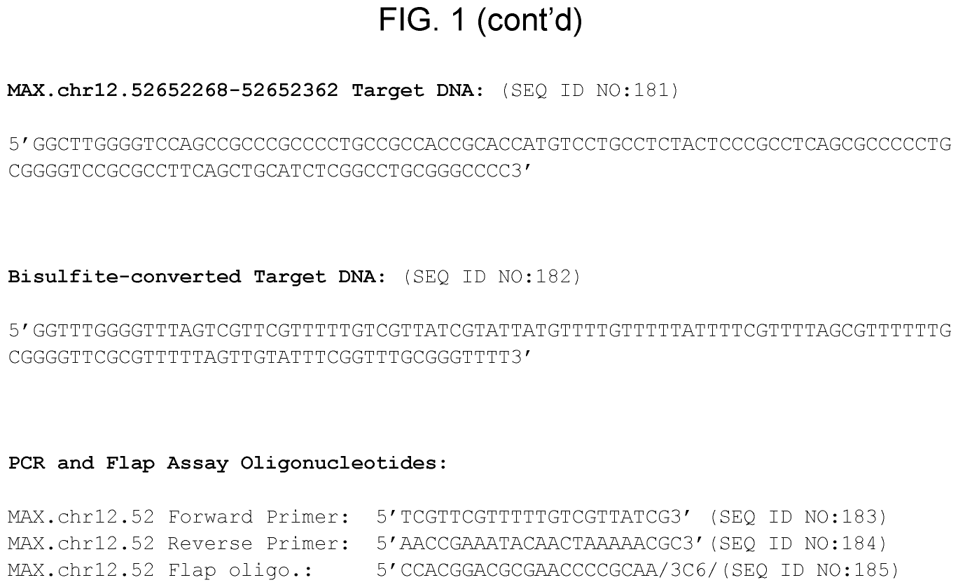

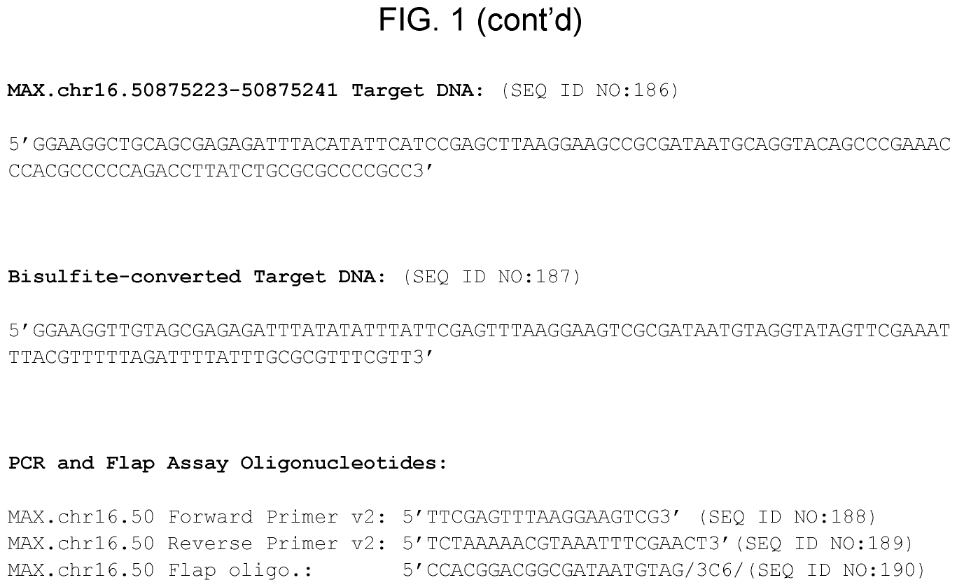

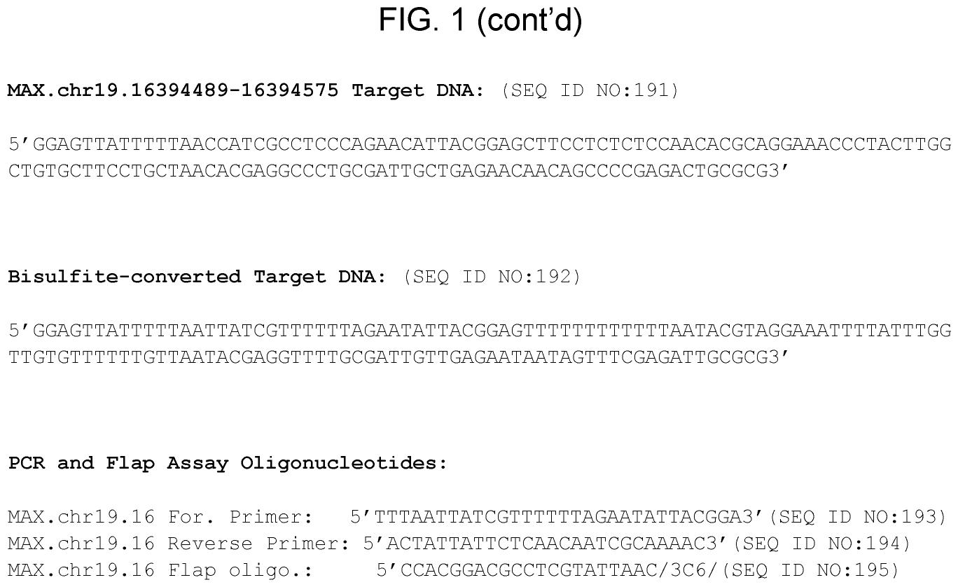

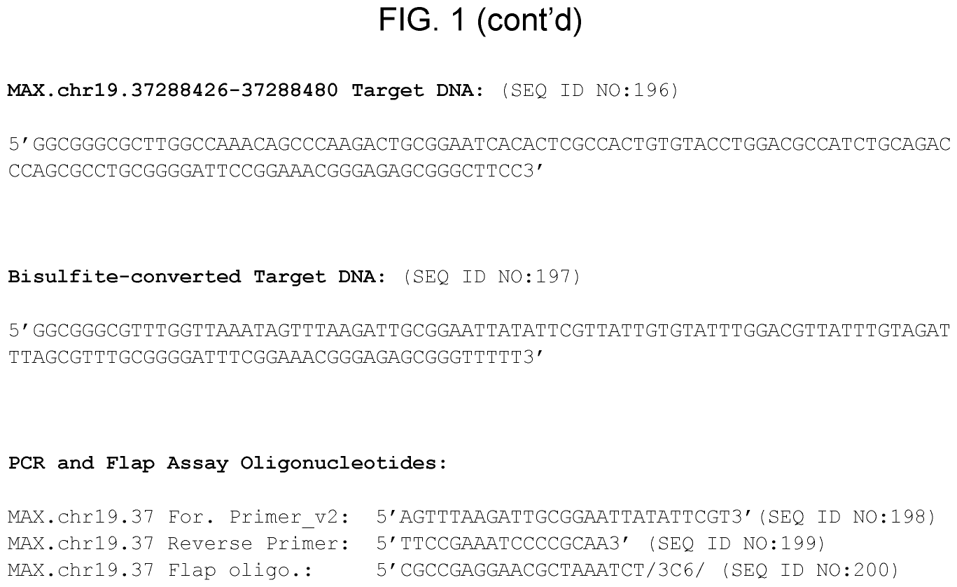

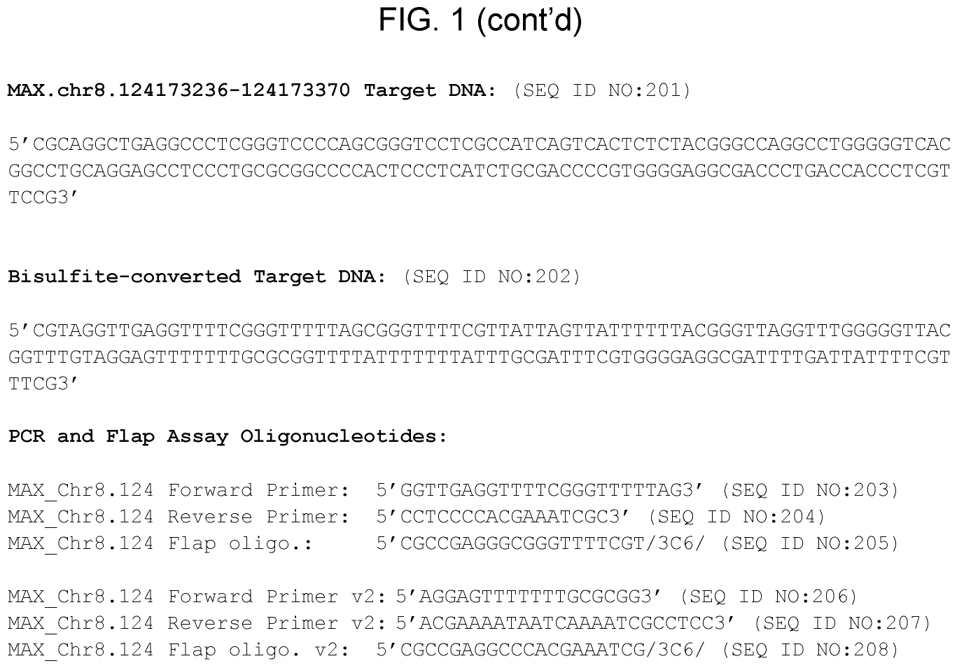

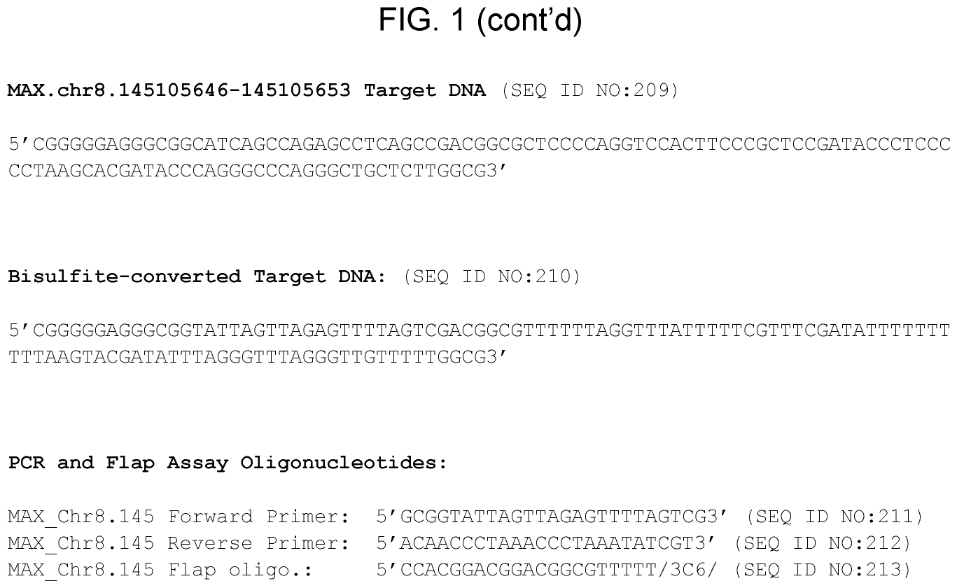

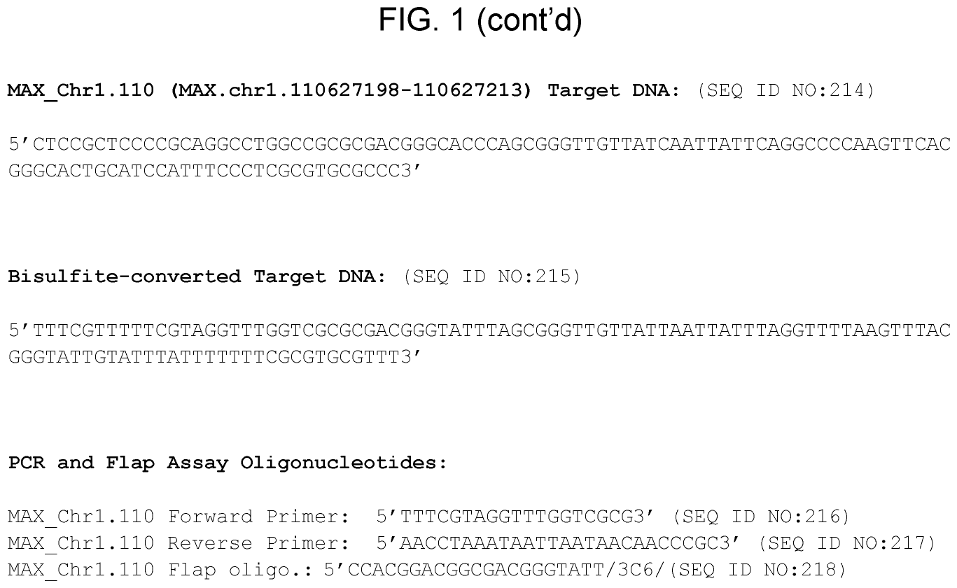

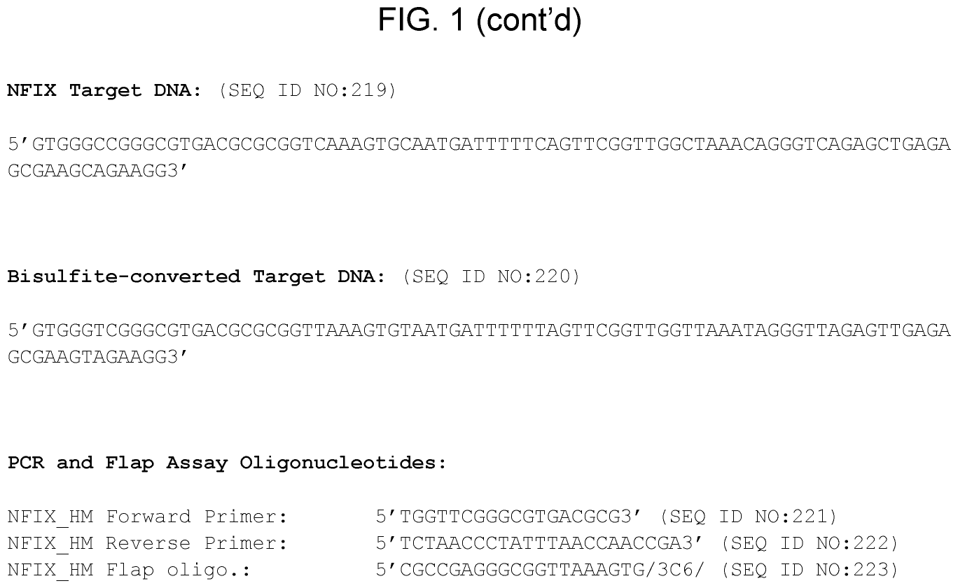

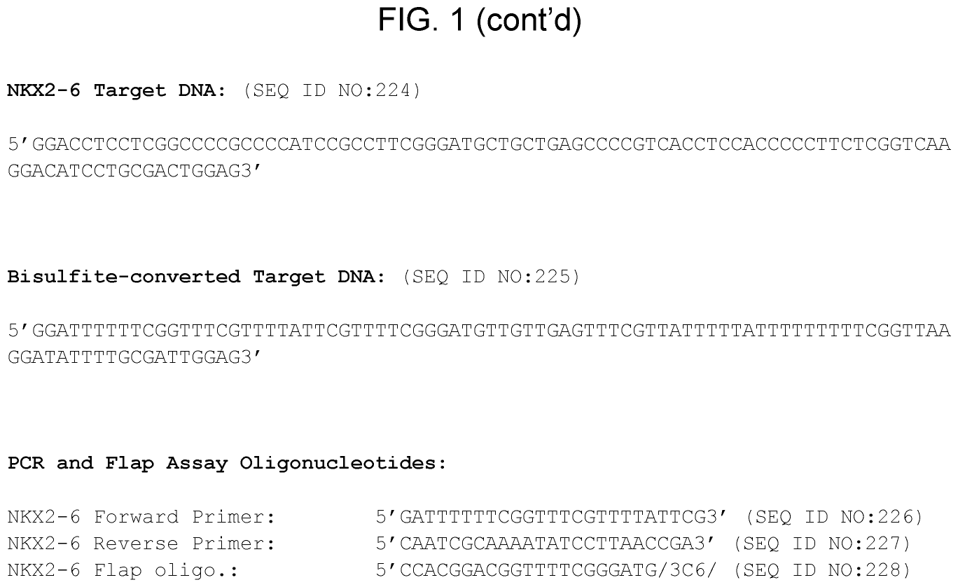

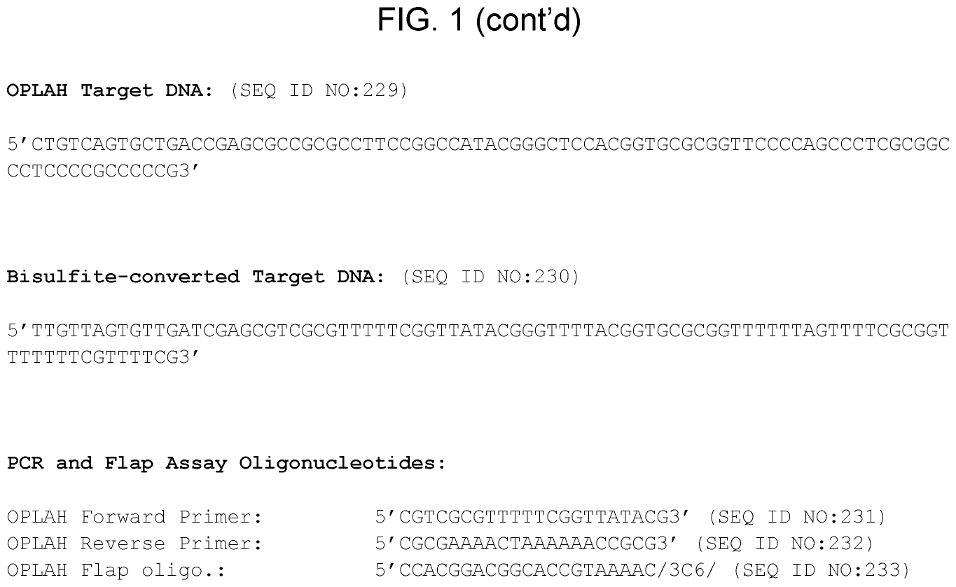

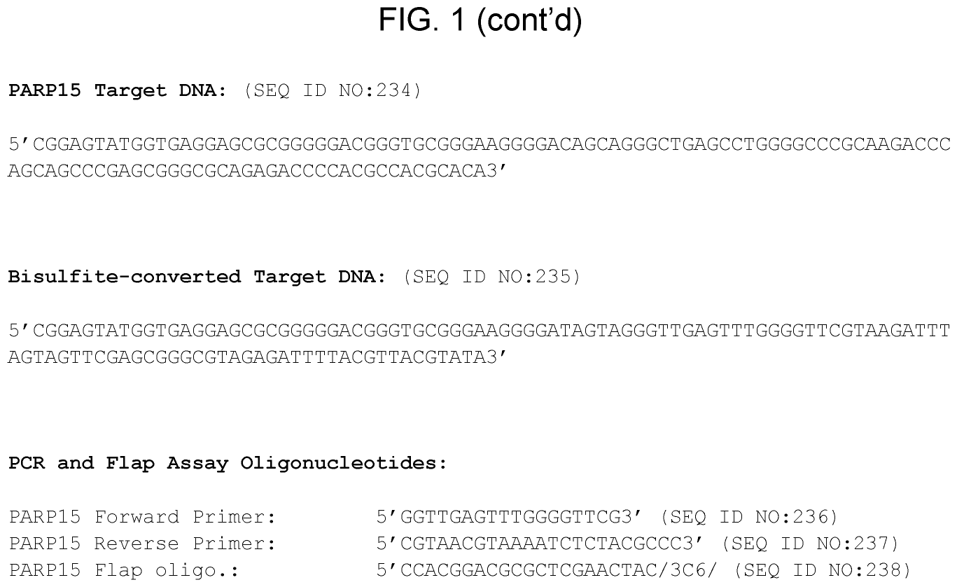

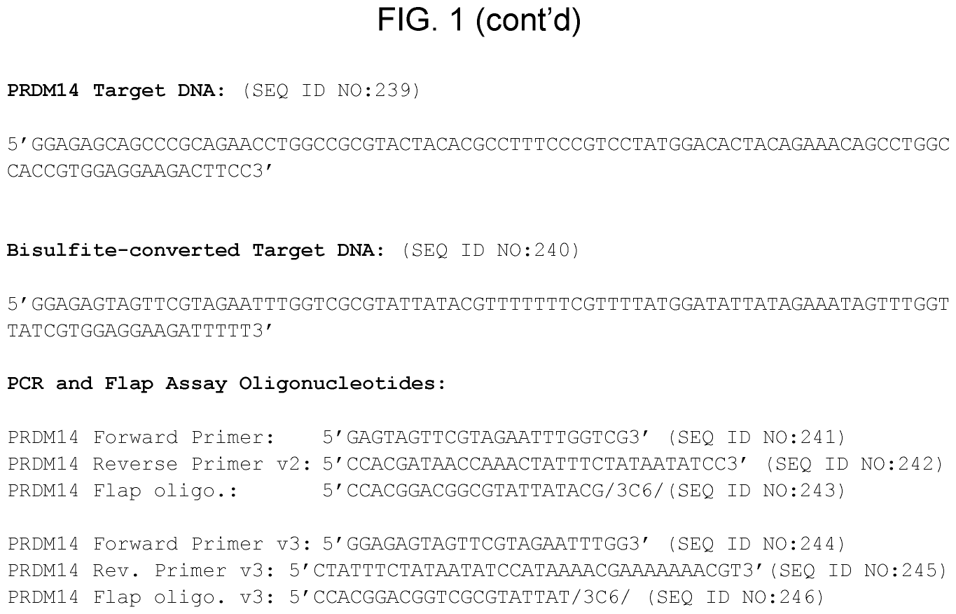

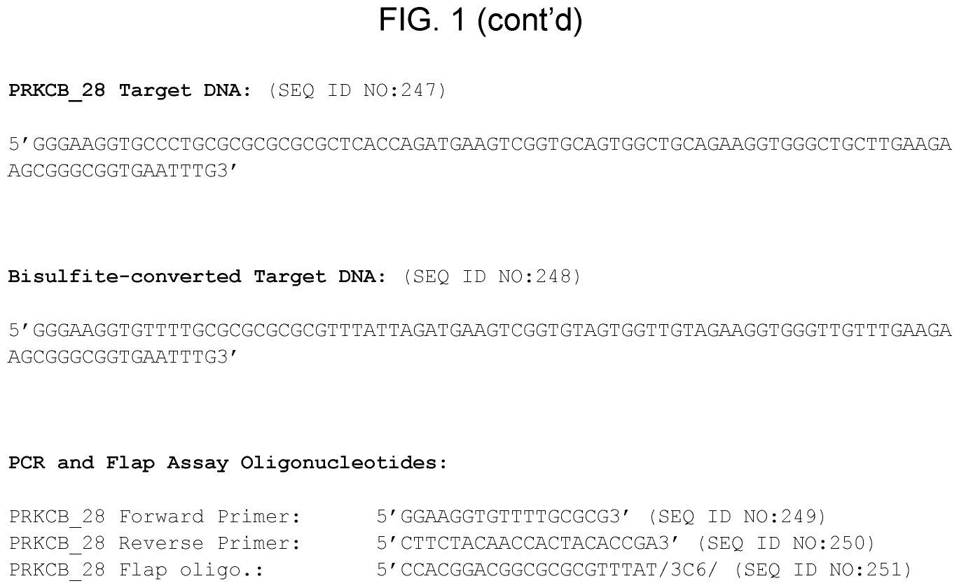

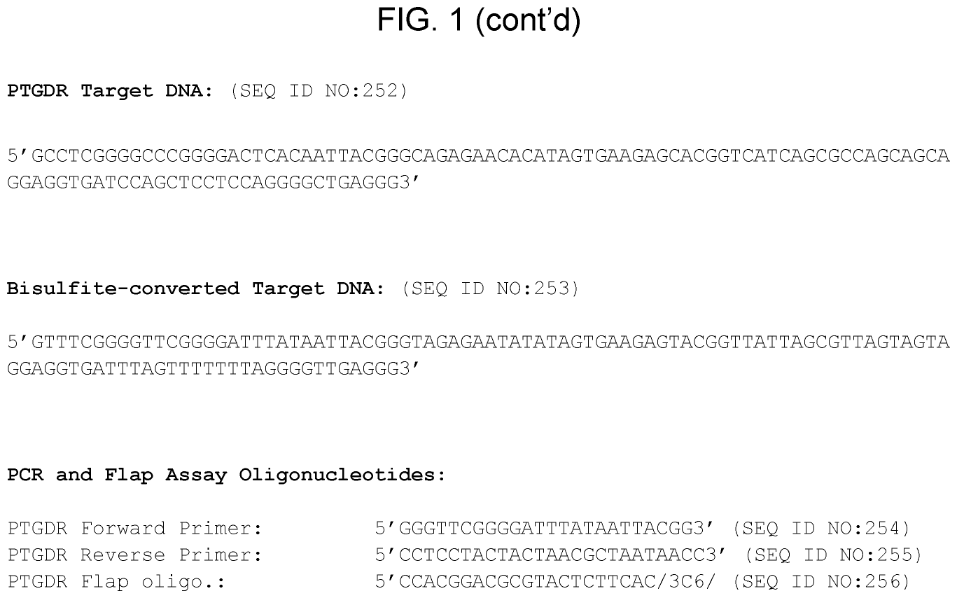

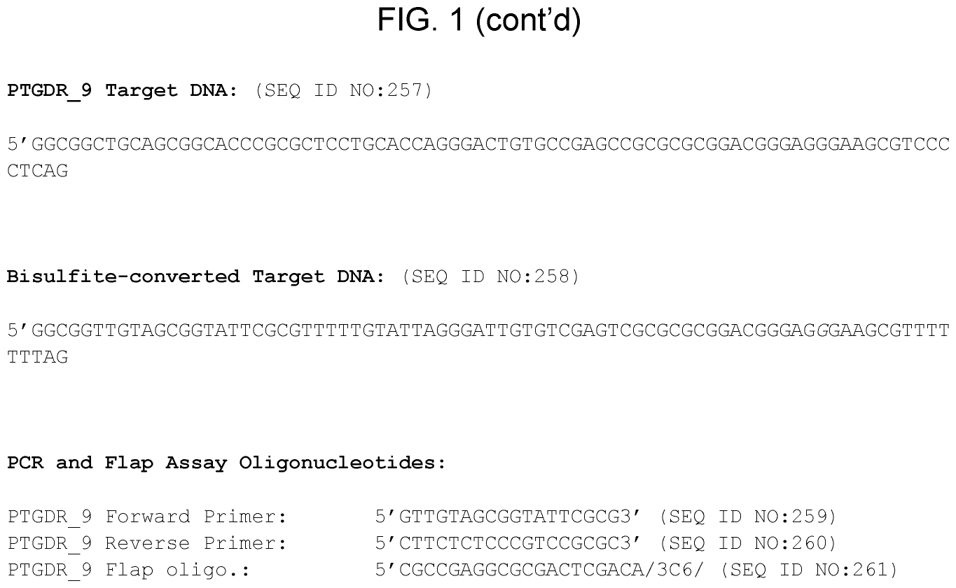

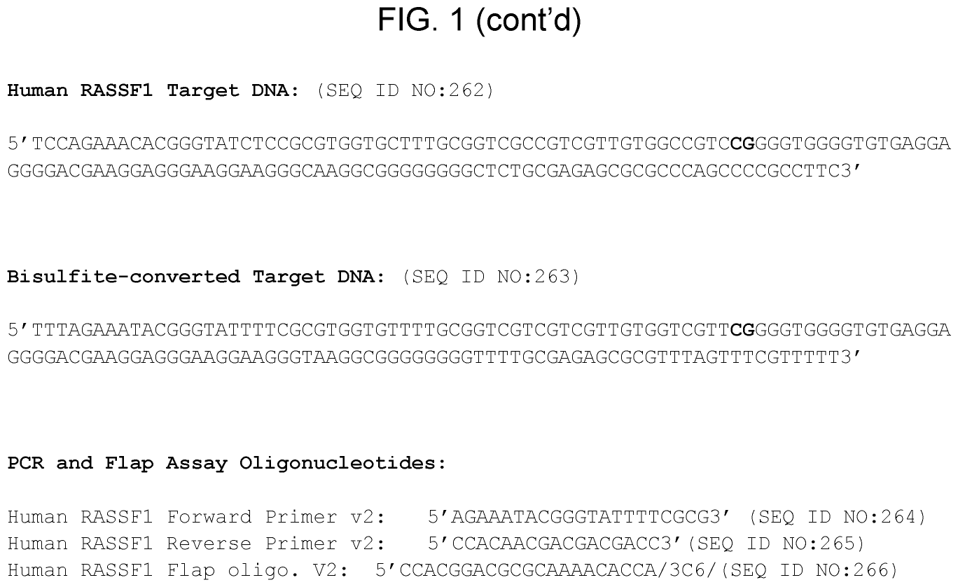

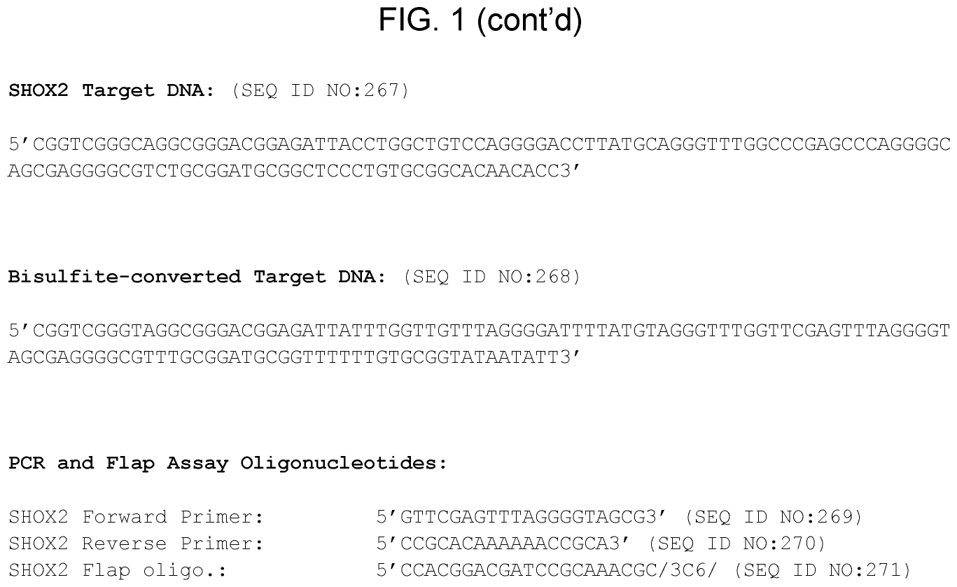

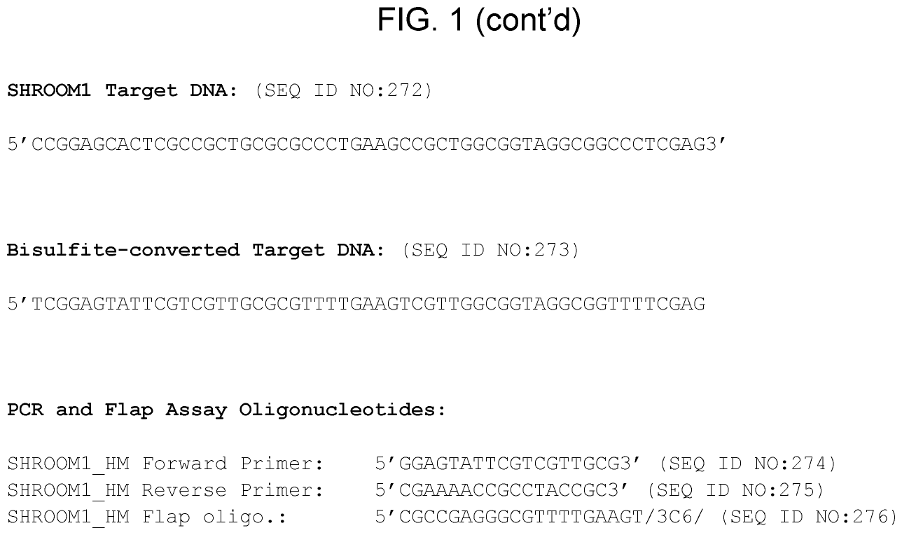

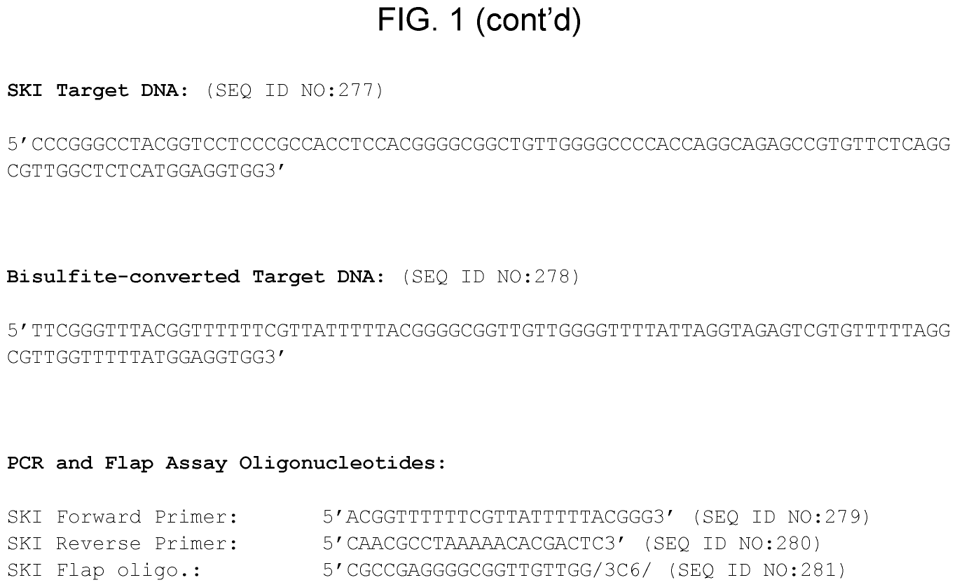

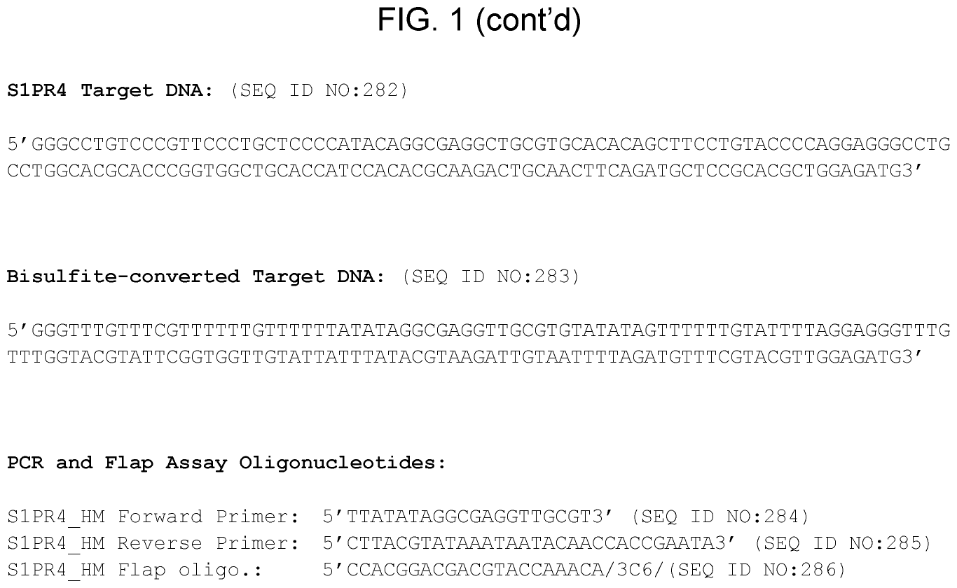

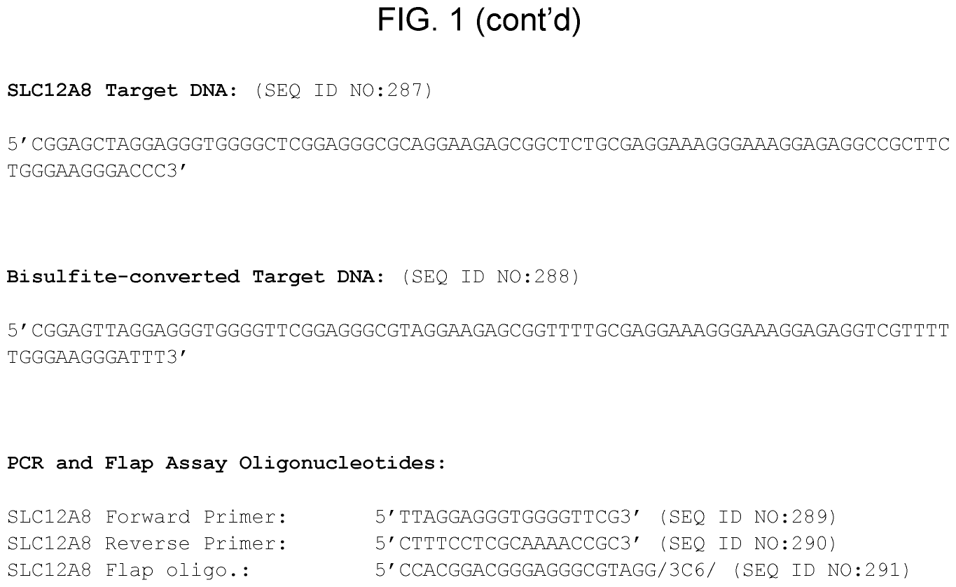

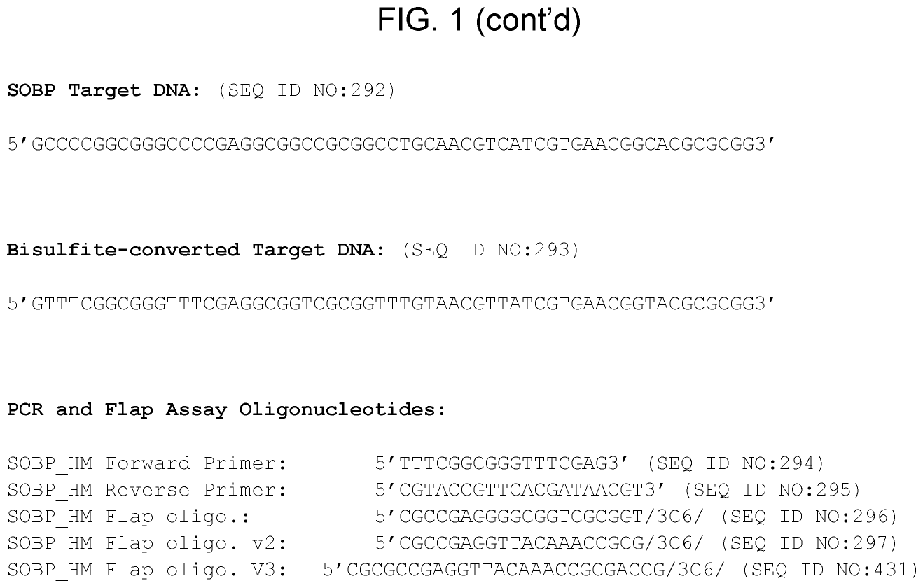

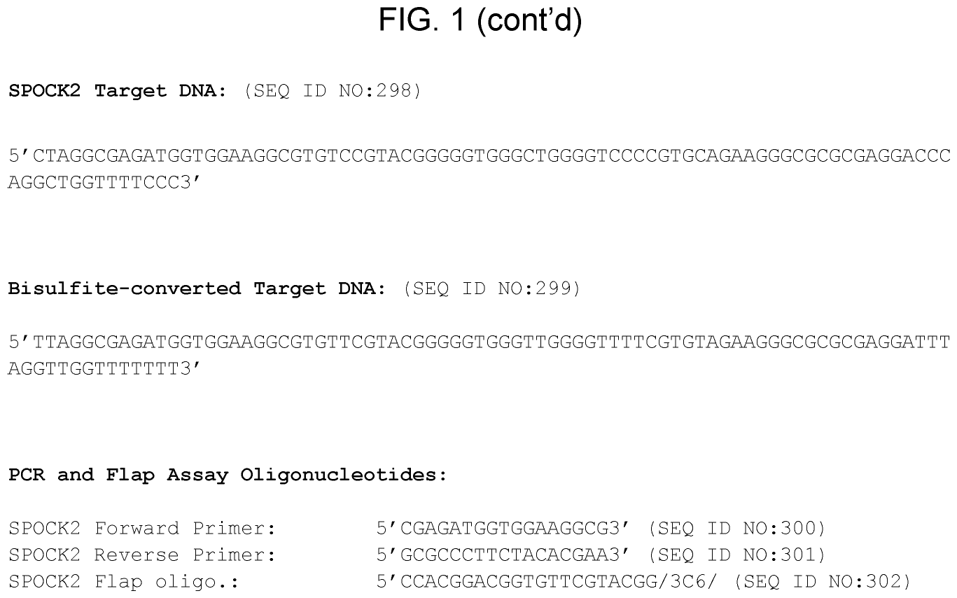

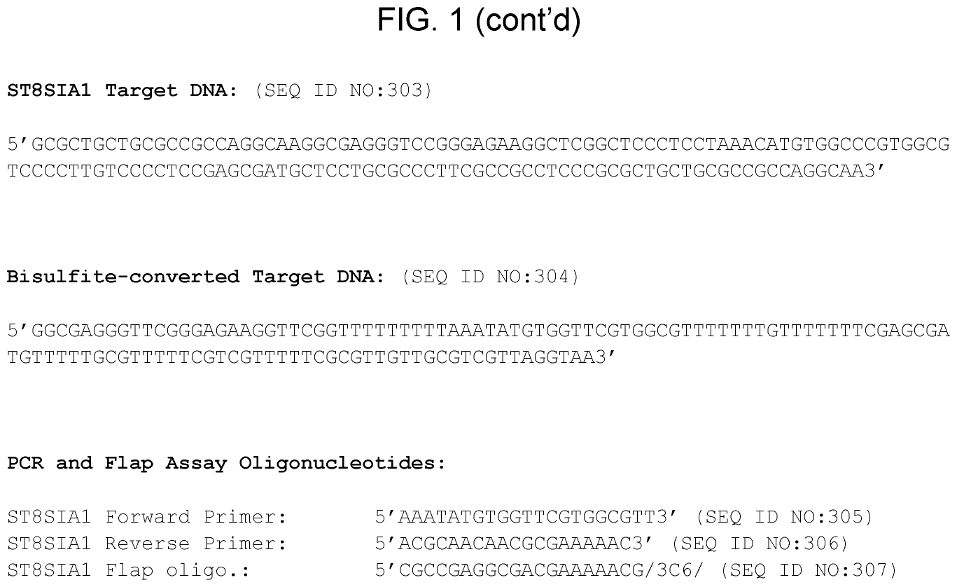

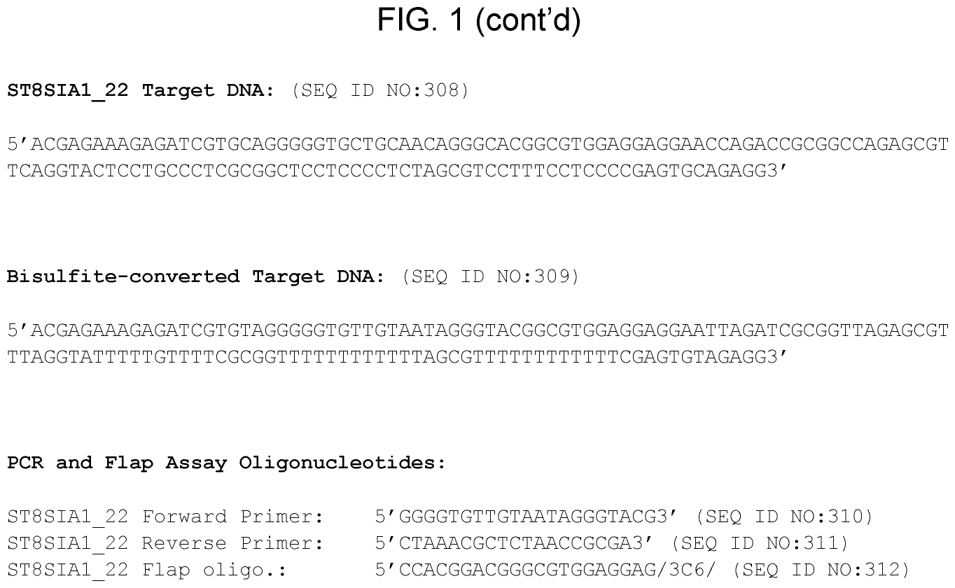

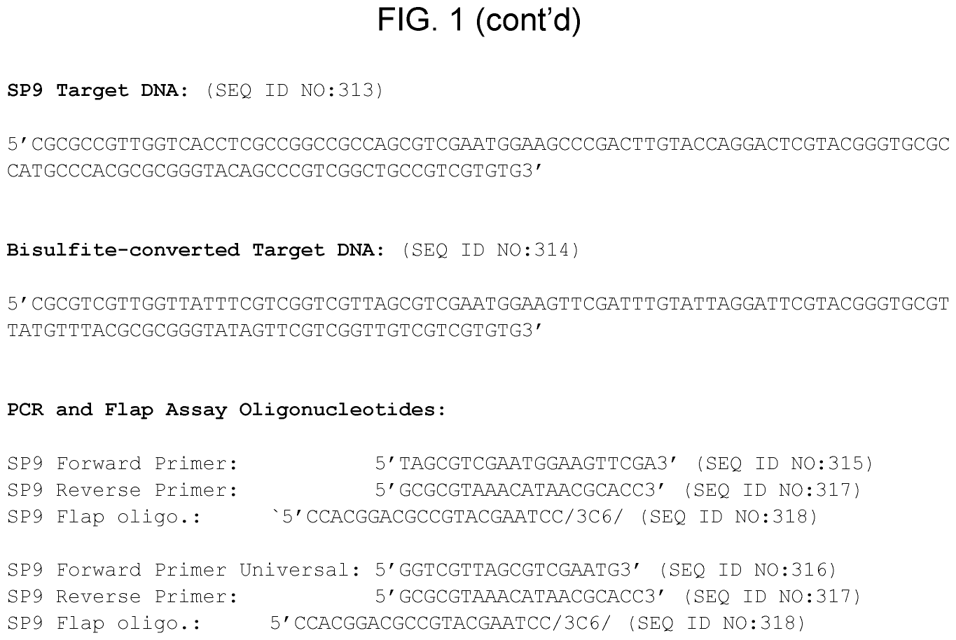

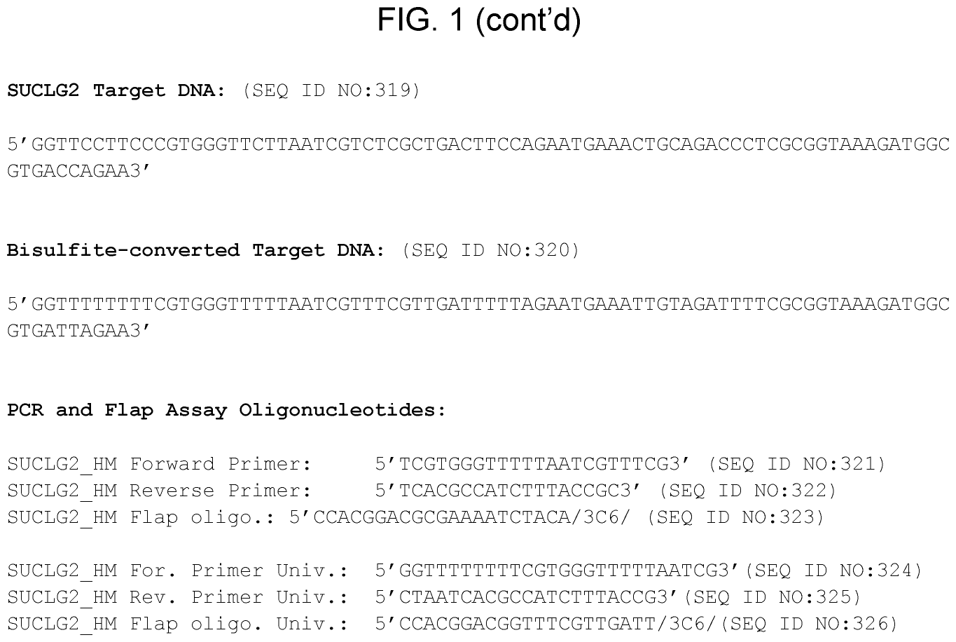

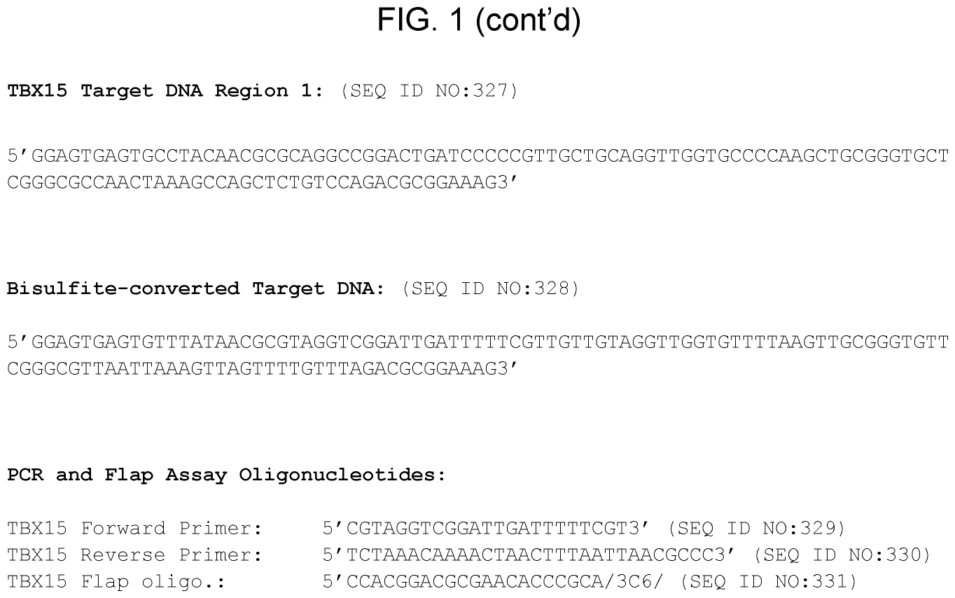

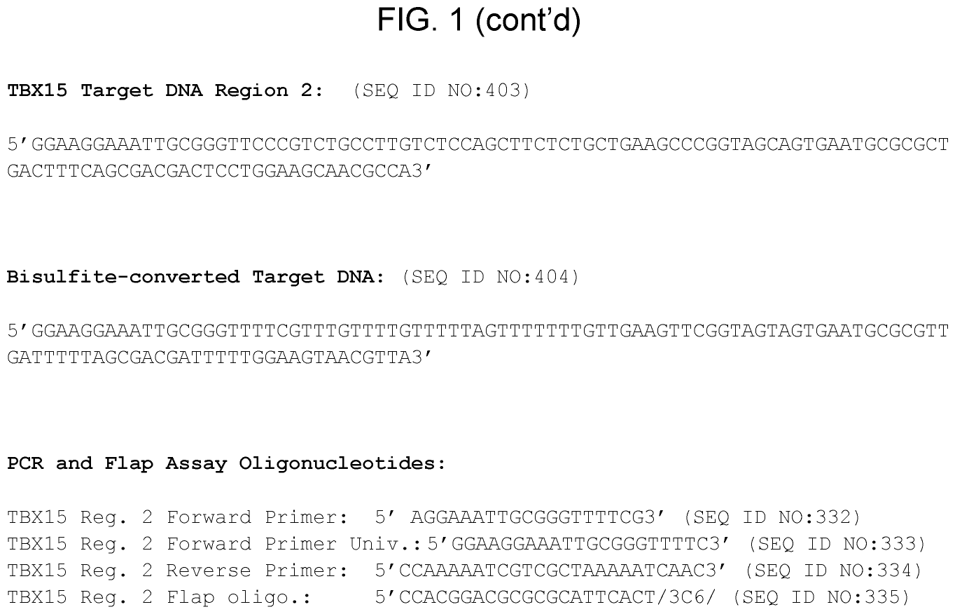

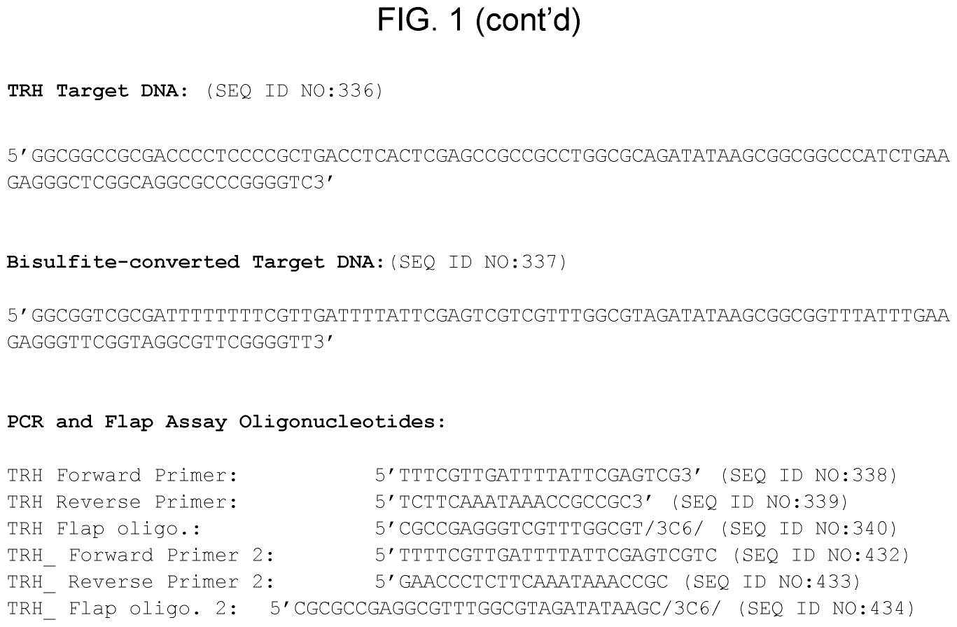

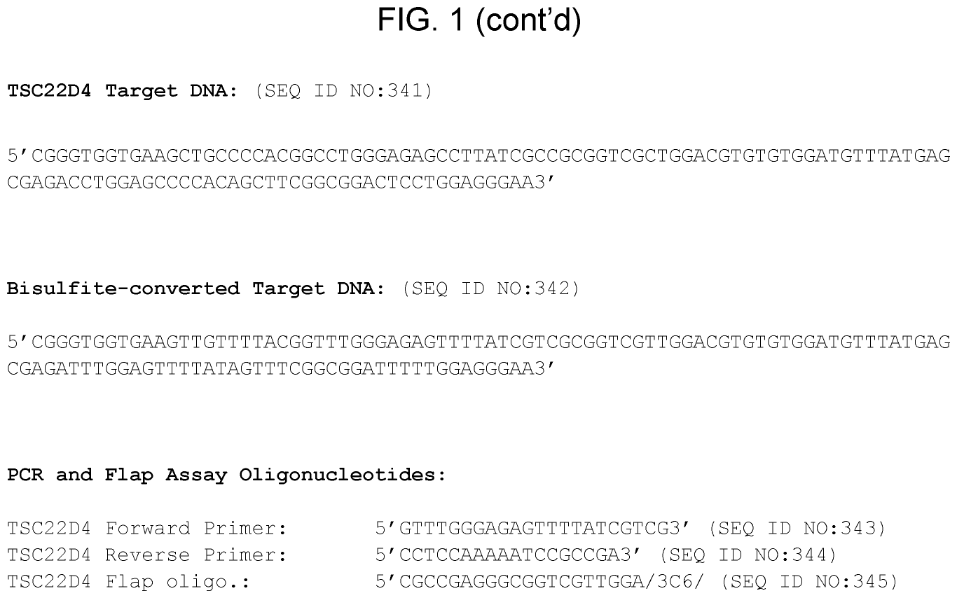

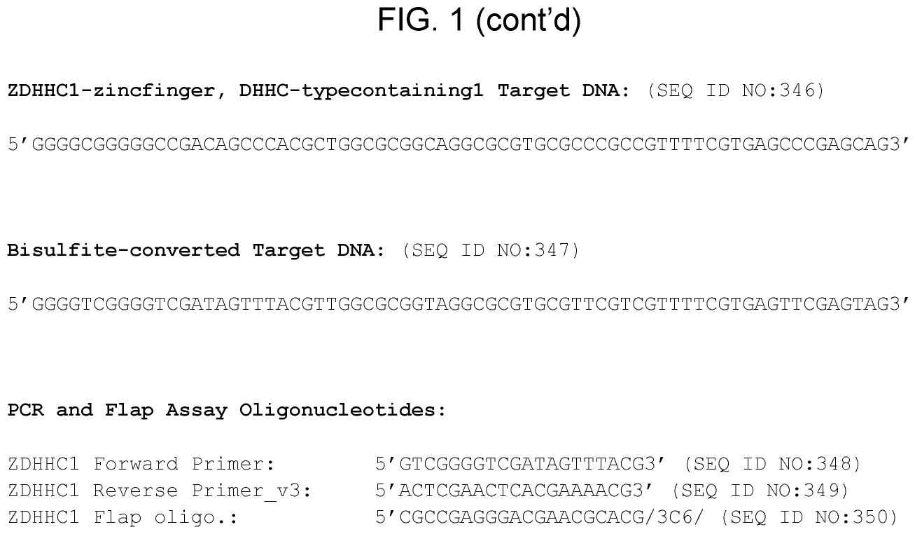

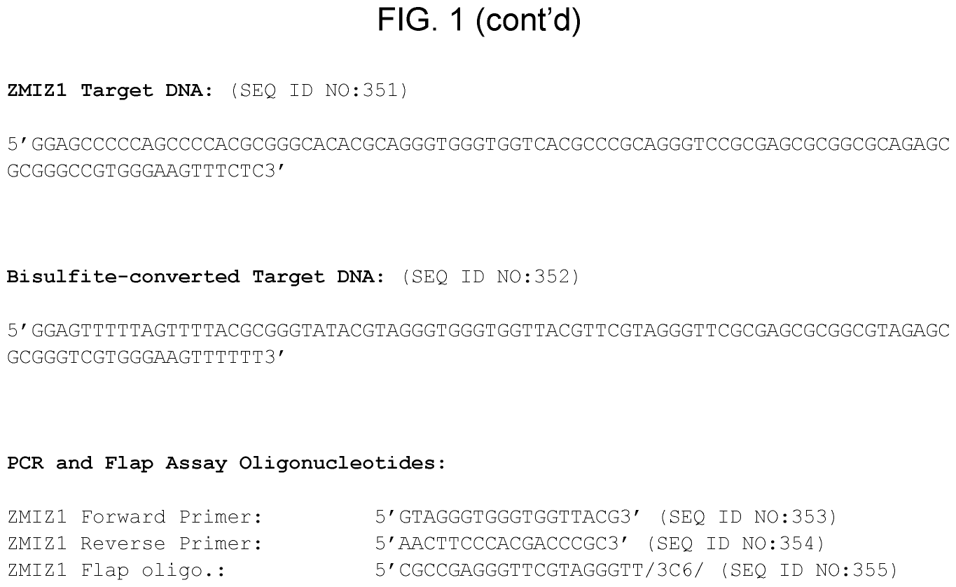

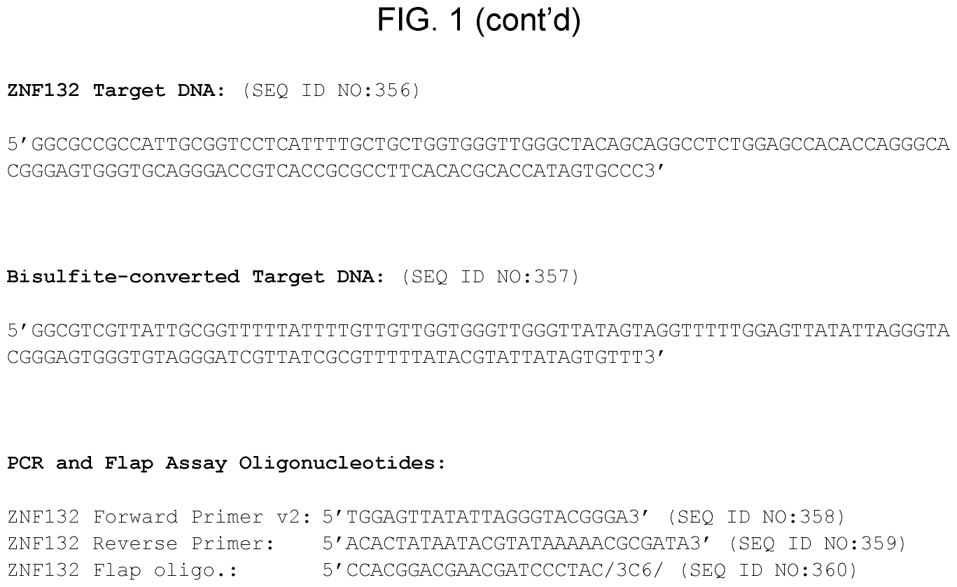

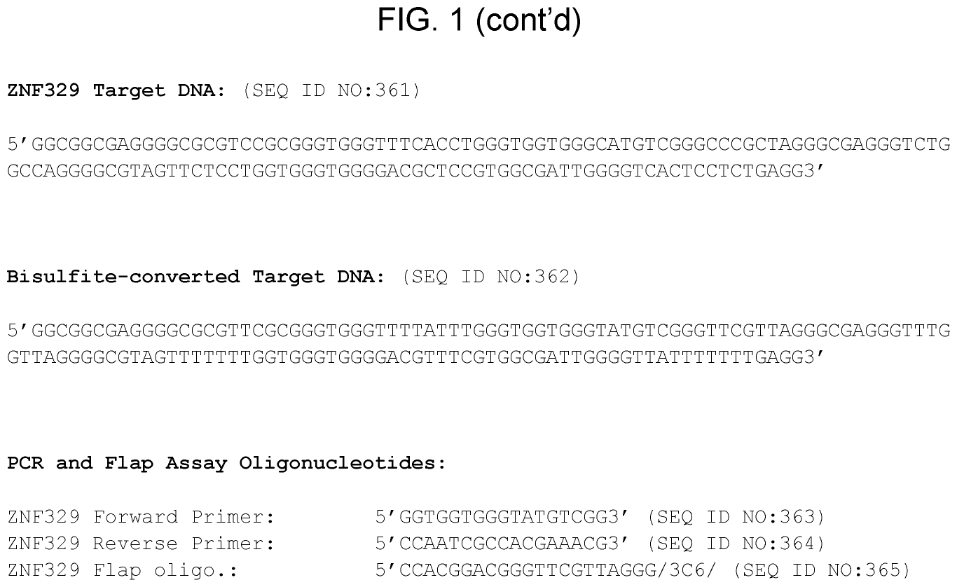

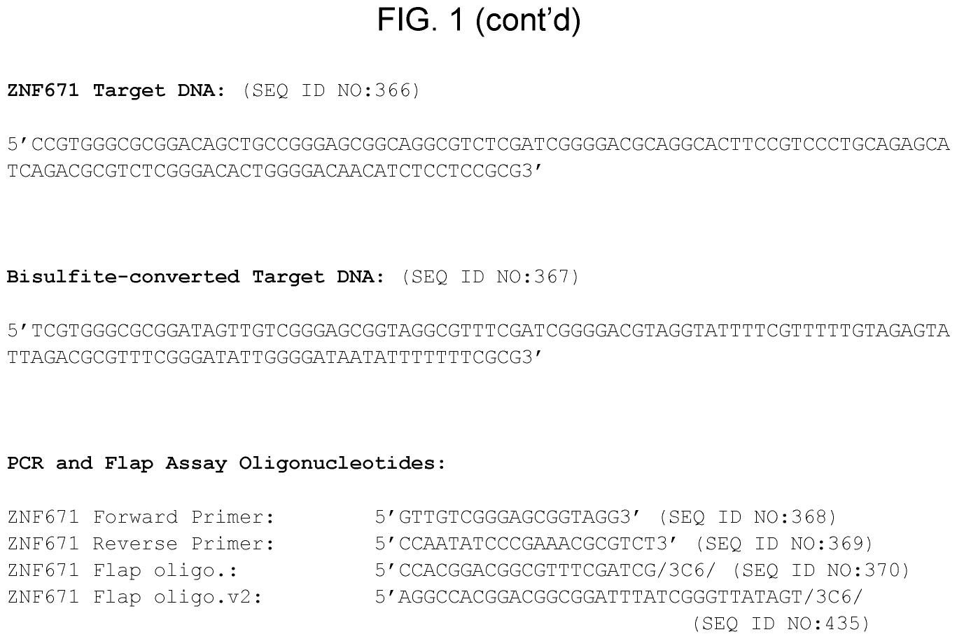

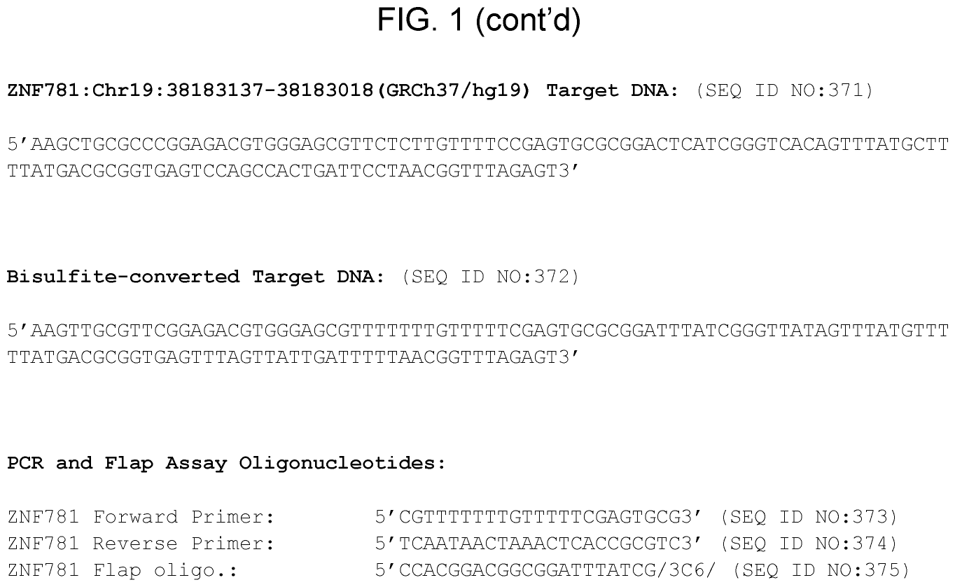

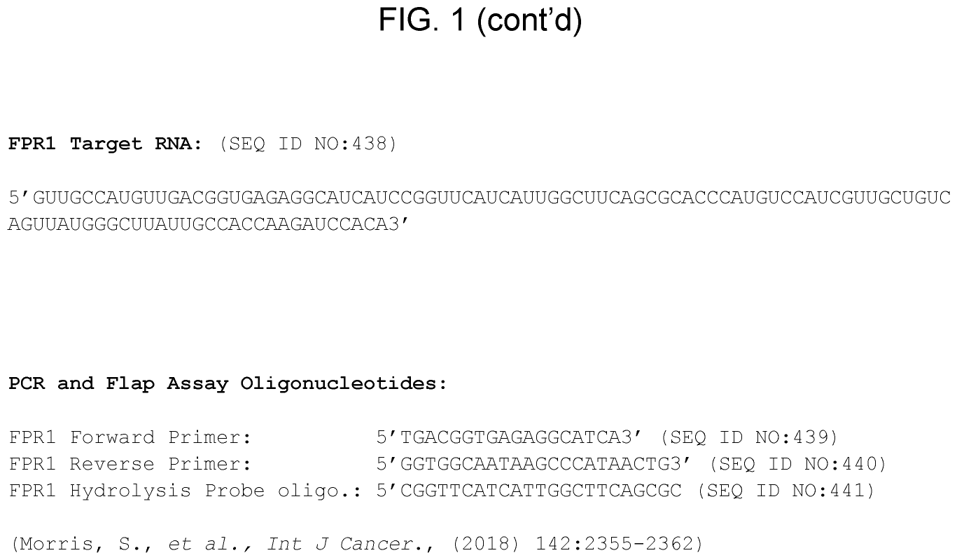

In some embodiments, fragments of the treated DNA are amplified using sets of primer oligonucleotides according to the present invention (e.g., see FIG. 1) and an amplification enzyme. The amplification of several DNA segments can be carried out simultaneously in one and the same reaction vessel. Typically, the amplification is carried out using a polymerase chain reaction (PCR).

Methods for isolating DNA suitable for these assay technologies are known in the art. In particular, some embodiments comprise isolation of nucleic acids as described in U.S. Pat. Nos. 9,000,146 and 9,163,278, each incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QUARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances compromise the accuracy and precision of these assays directed to detecting such low amounts of a target. Accordingly, provided herein are methods providing the requisite management of volume and concentration to produce such DNA samples.

In some embodiments, the sample comprises blood, serum, plasma, or saliva. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

It is contemplated that embodiments of the technology are provided in the form of a kit. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

### III. Applications

In some embodiments, diagnostic assays identify the presence of a disease or condition in an individual. In some embodiments, the disease is cancer (e.g., lung cancer). In some embodiments, markers whose aberrant methylation is associated with a lung cancer (e.g., one or more markers selected from the markers listed in Table 1, or preferably one or more of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH. PARP15, KLHDC7B, SLC12A8, BHLHE23, CAPN2, FGF14, FLJ34208, B3GALT6, BIN2_Z, DNMT3A, FERAMT3, NFIX, SIPR4, SKI, SUCLG2, TBX15, ZDHHC1. ZNF329, IFFO1, and HOPX) are used. In some embodiments, an assay further comprises detection of a reference gene (e.g., β-actin, ZDHHC1, B3GALT6. See, e.g., U.S. patent application Ser. No. 14/966,617, filed Dec. 11, 2015, and U.S. Pat. Appl. No. 62/364,082, filed Jul. 19, 2016, each of which is incorporated herein by reference for all purposes).

In some embodiments, the technology finds application in treating a patient (e.g., a patient with lung cancer, with early stage lung cancer, or who may develop lung cancer), the method comprising determining the methylation state of one or more markers as provided herein and administering a treatment to the patient based on the results of determining the methylation state. The treatment may be administration of a pharmaceutical compound, a vaccine, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, the technology finds application in methods for diagnosing lung cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a biomarker, the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of making determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers disclosed herein.

Further, in some embodiments of the technology, multiple determinations of the biomarkers over time can be made to facilitate diagnosis and/or prognosis. A temporal change in the biomarker can be used to predict a clinical outcome, monitor the progression of lung cancer, and/or monitor the efficacy of appropriate therapies directed against the cancer. In such an embodiment for example, one might expect to see a change in the methylation state of one or more biomarkers disclosed herein (and potentially one or more additional biomarker(s), if monitored) in a biological sample over time during the course of an effective therapy.

The technology further finds application in methods for determining whether to initiate or continue prophylaxis or treatment of a cancer in a subject. In some embodiments, the method comprises providing a series of biological samples over a time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with risk for developing lung, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted above, in some embodiments multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition, the chance of a given outcome (e.g., suffering from lung cancer) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in tissue taken from donors with lung cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having lung cancer if, when compared to a control methylation state, there is a measurable difference in the methylation state of at least one biomarker in the sample. Conversely, when no change in methylation state is identified in the biological sample, the subject can be identified as not having lung cancer, not being at risk for the cancer, or as having a low risk of the cancer. In this regard, subjects having lung cancer or risk thereof can be differentiated from subjects having low to substantially no cancer or risk thereof. Those subjects having a risk of developing lung cancer can be placed on a more intensive and/or regular screening schedule. On the other hand, those subjects having low to substantially no risk may avoid being subjected to screening procedures, until such time as a future screening, for example, a screening conducted in accordance with the present technology, indicates that a risk of lung cancer has appeared in those subjects.

As mentioned above, depending on the embodiment of the method of the present technology, detecting a change in methylation state of the one or more biomarkers can be a qualitative determination or it can be a quantitative determination. As such, the step of diagnosing a subject as having, or at risk of developing, lung cancer indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

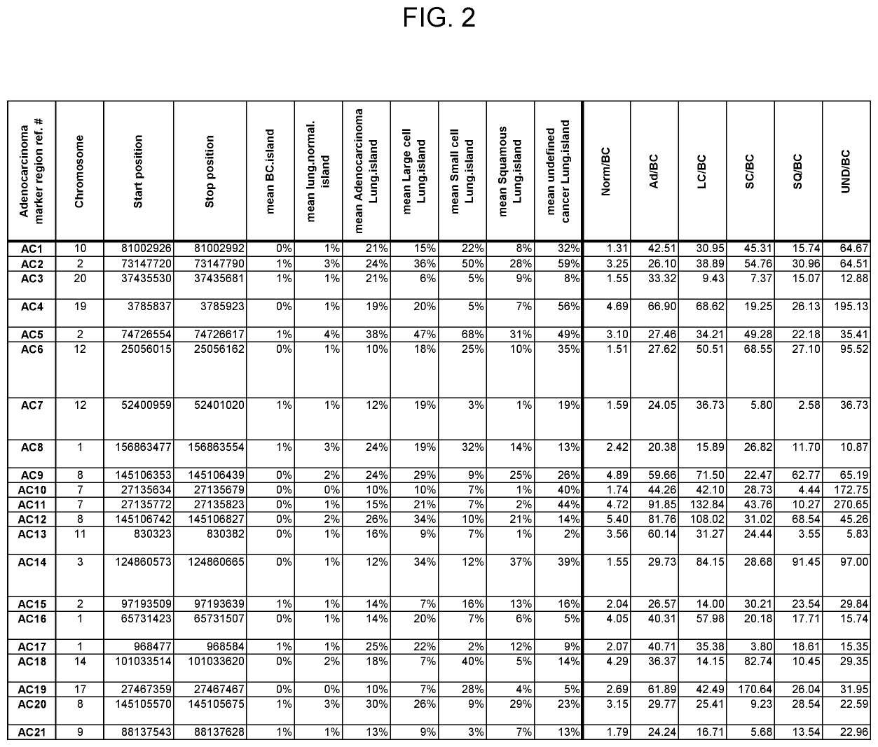

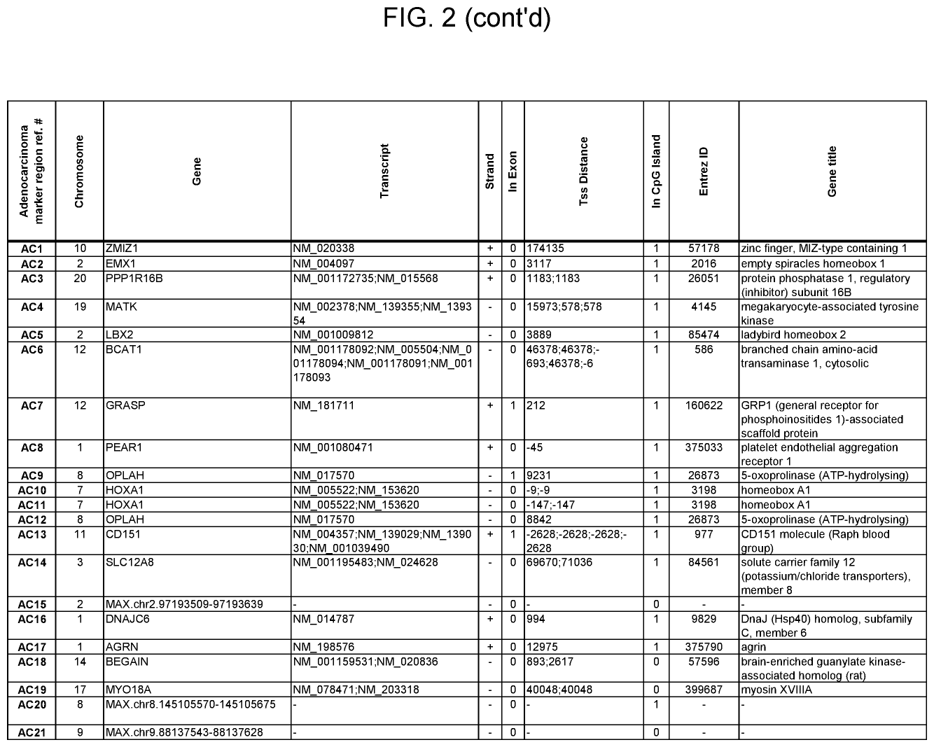

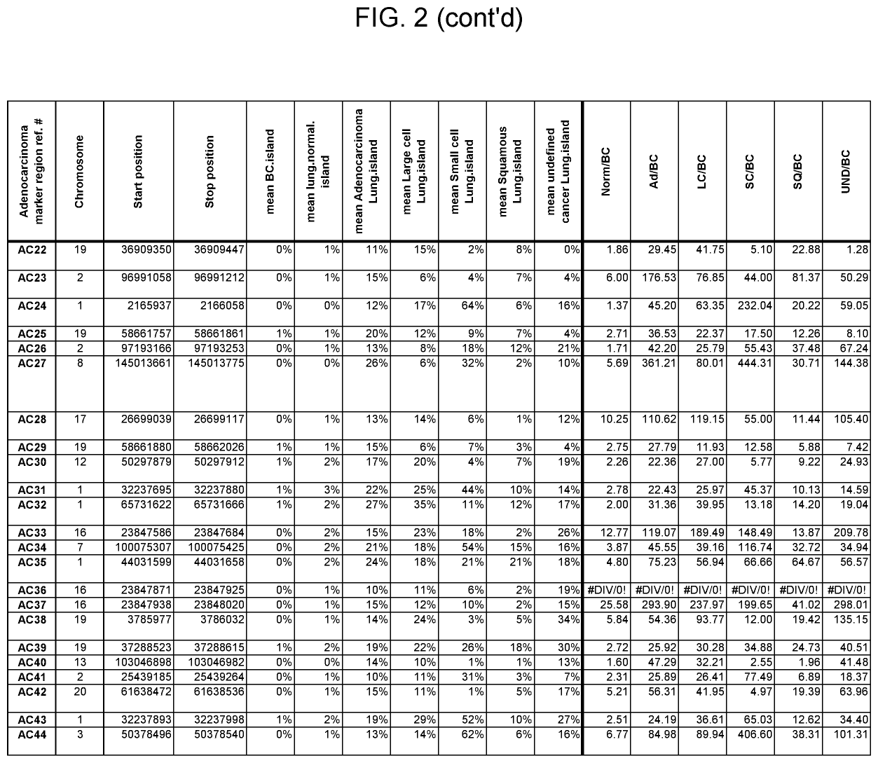

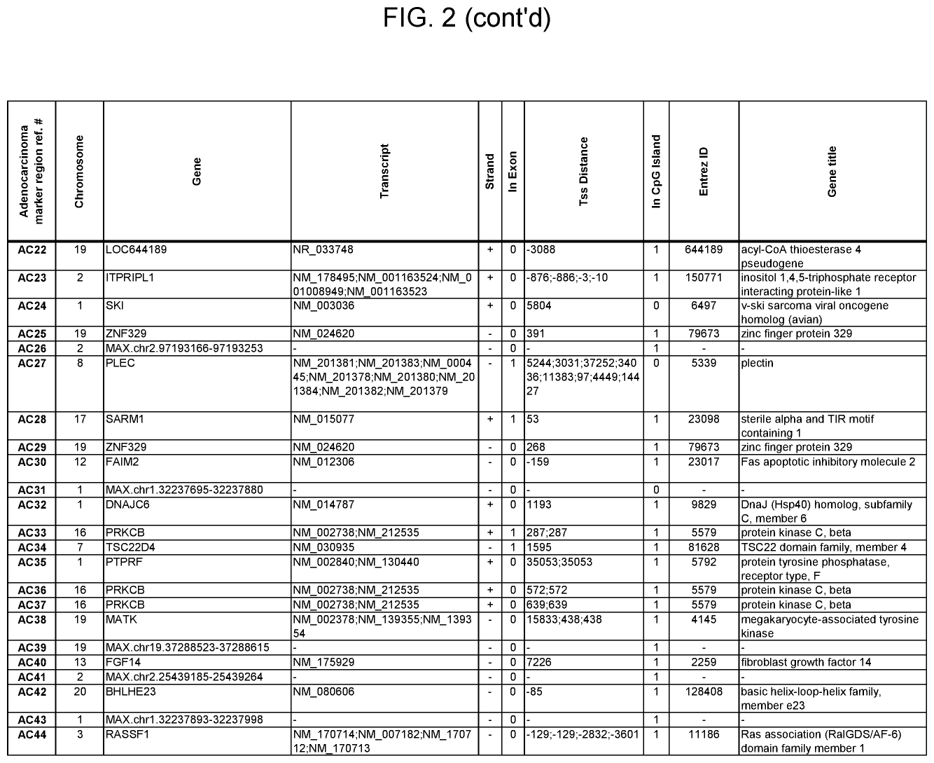

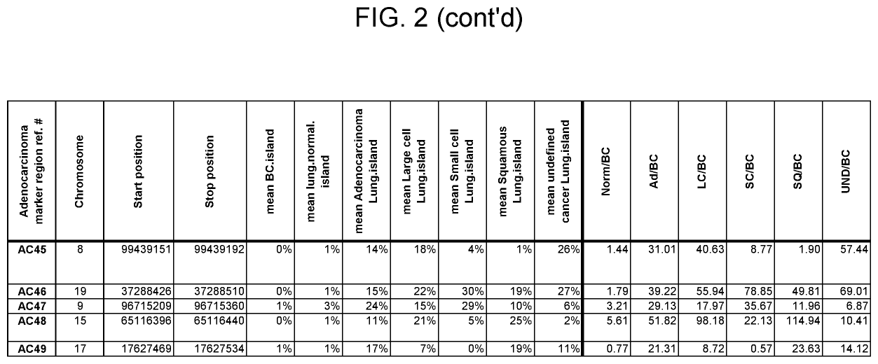

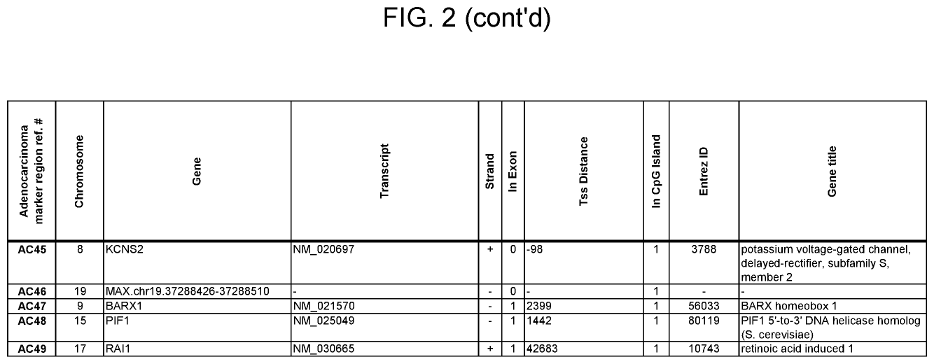

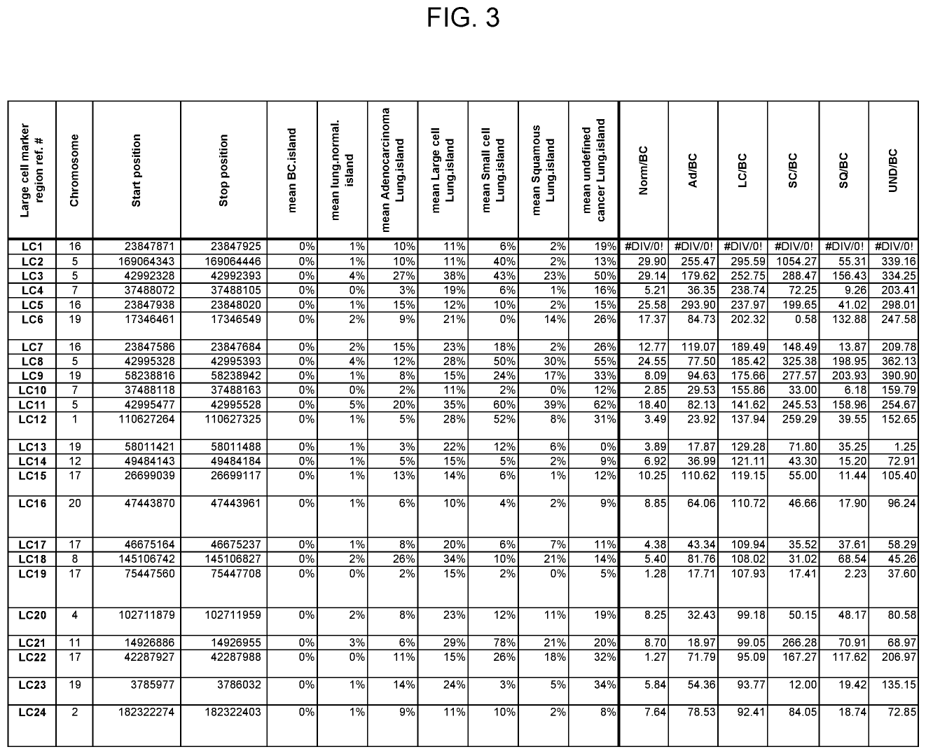

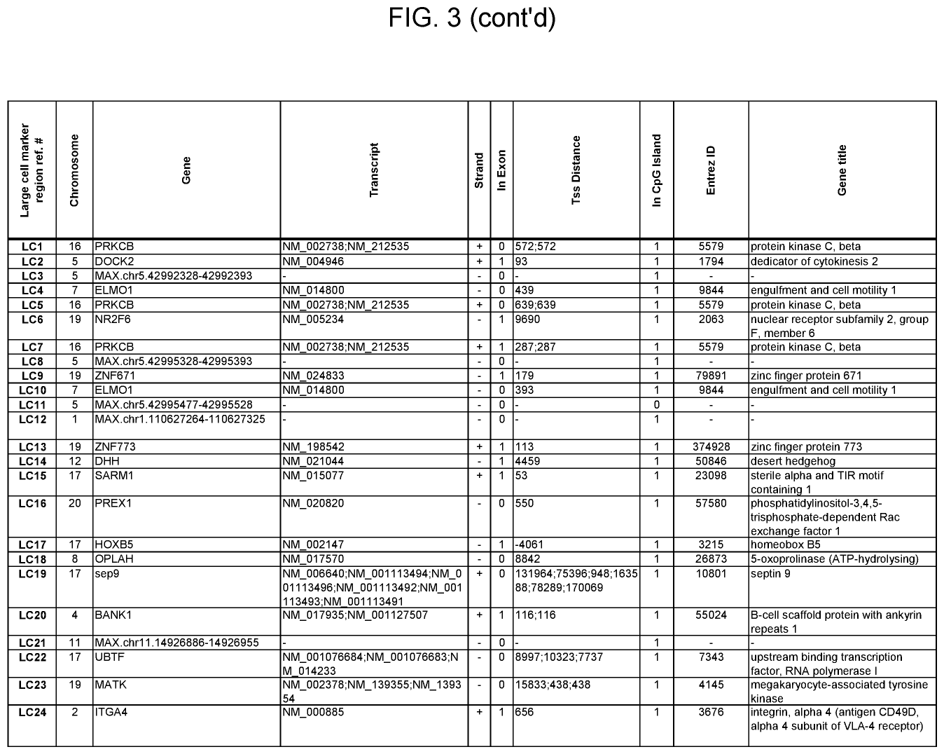

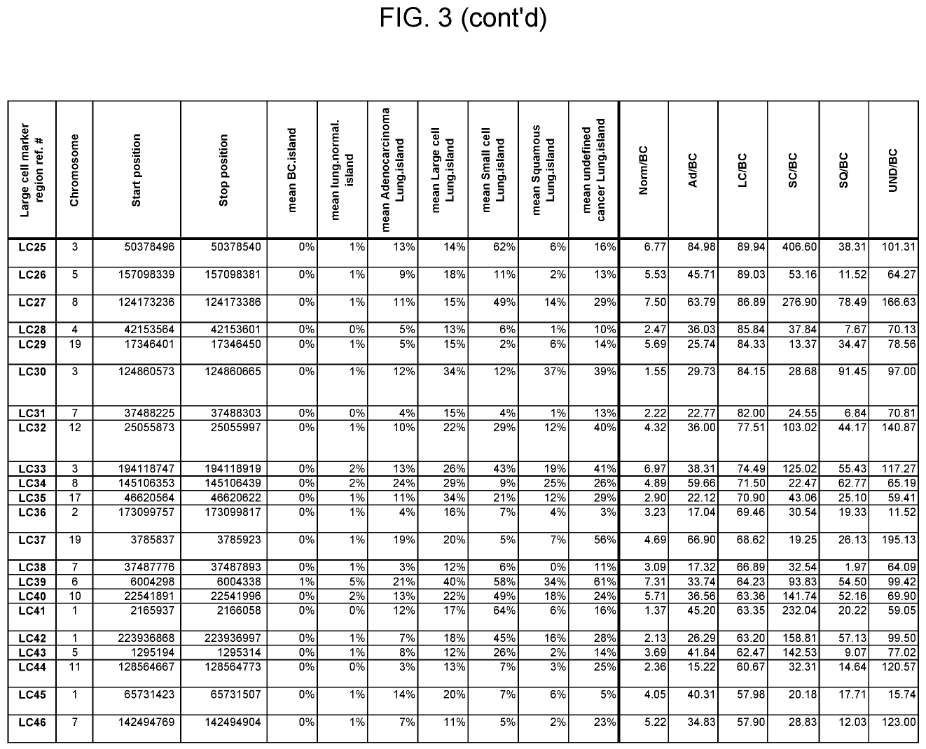

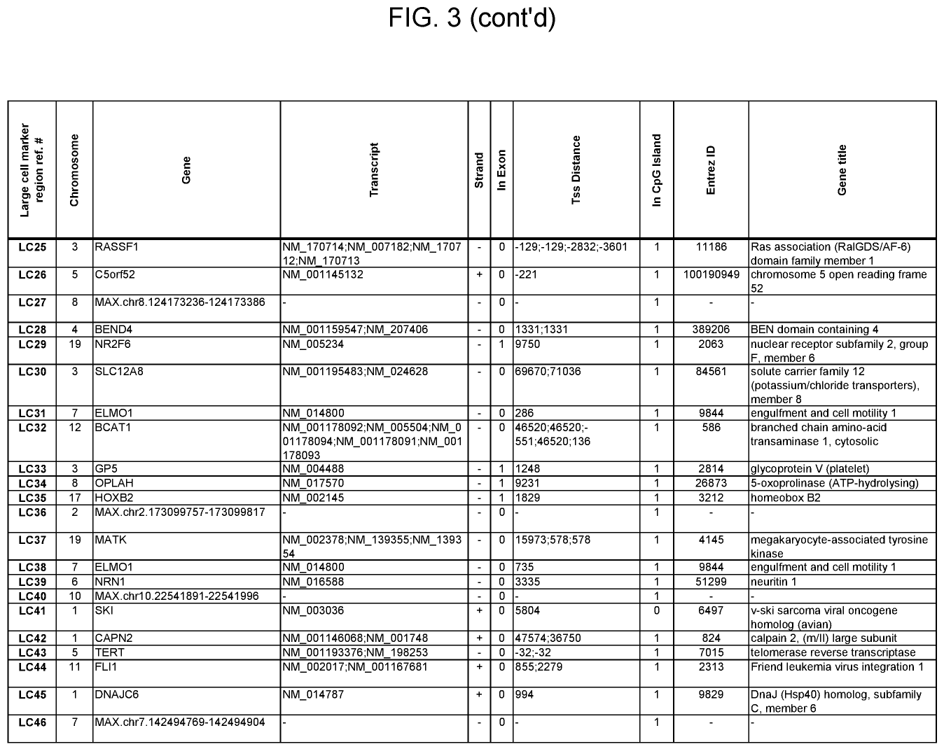

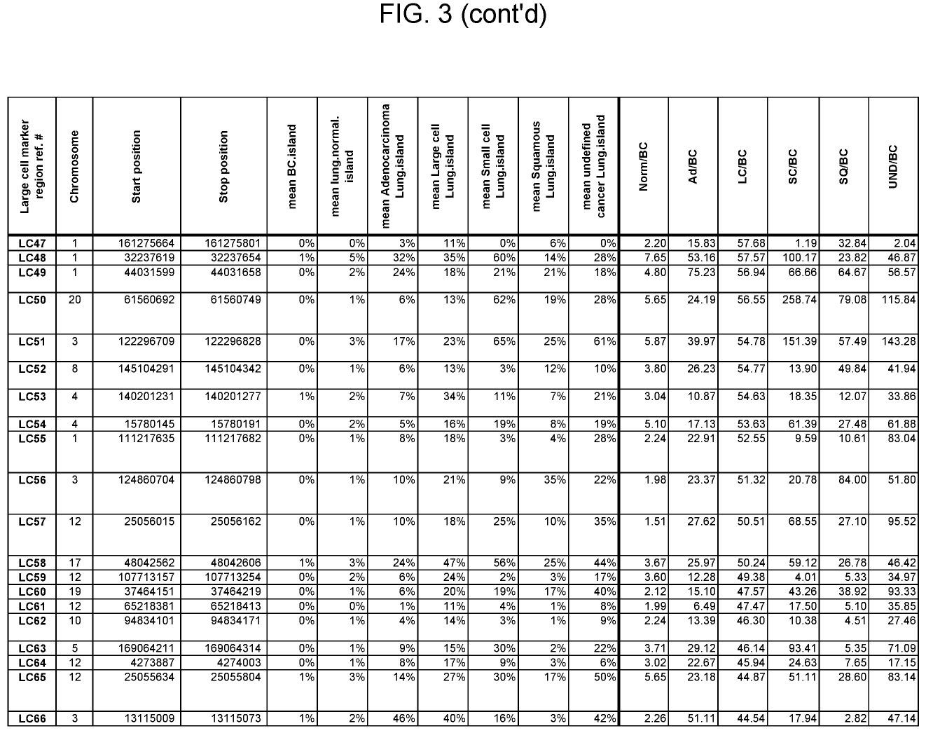

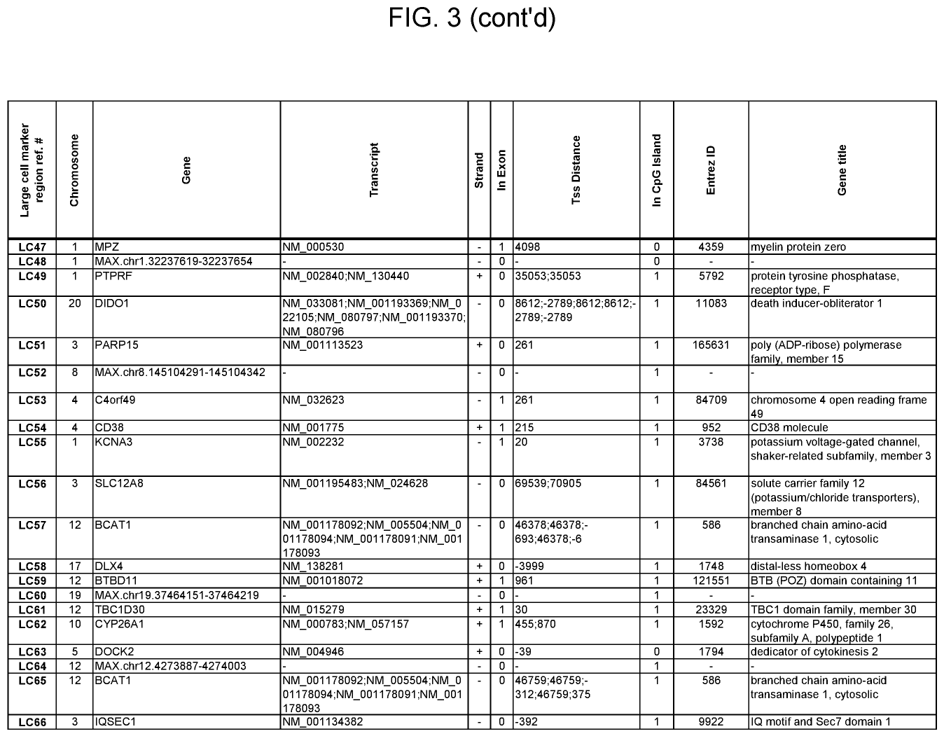

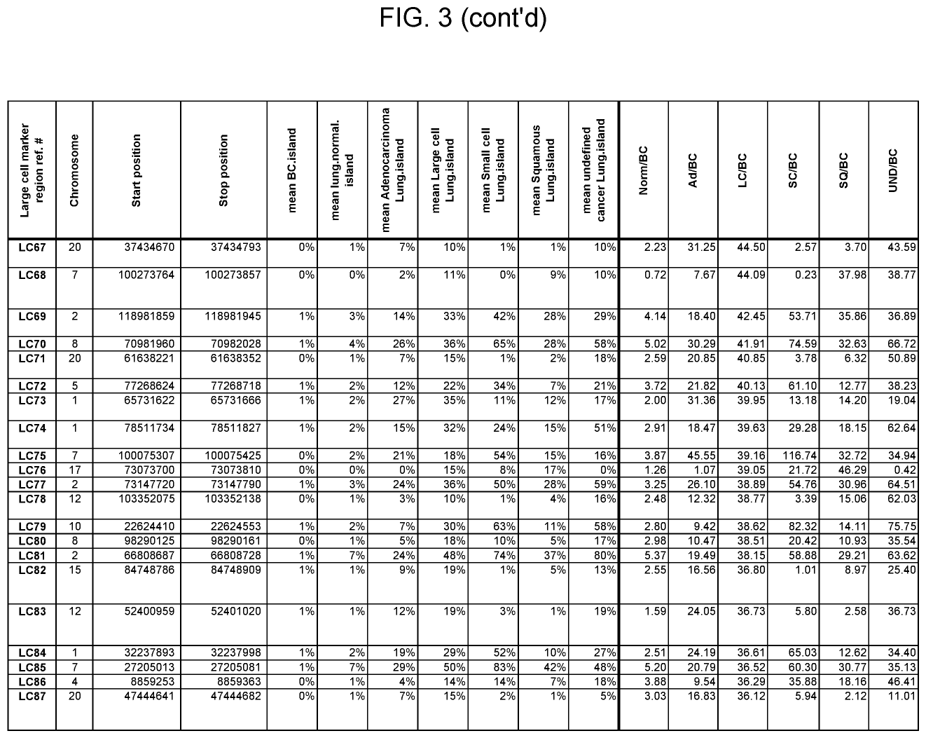

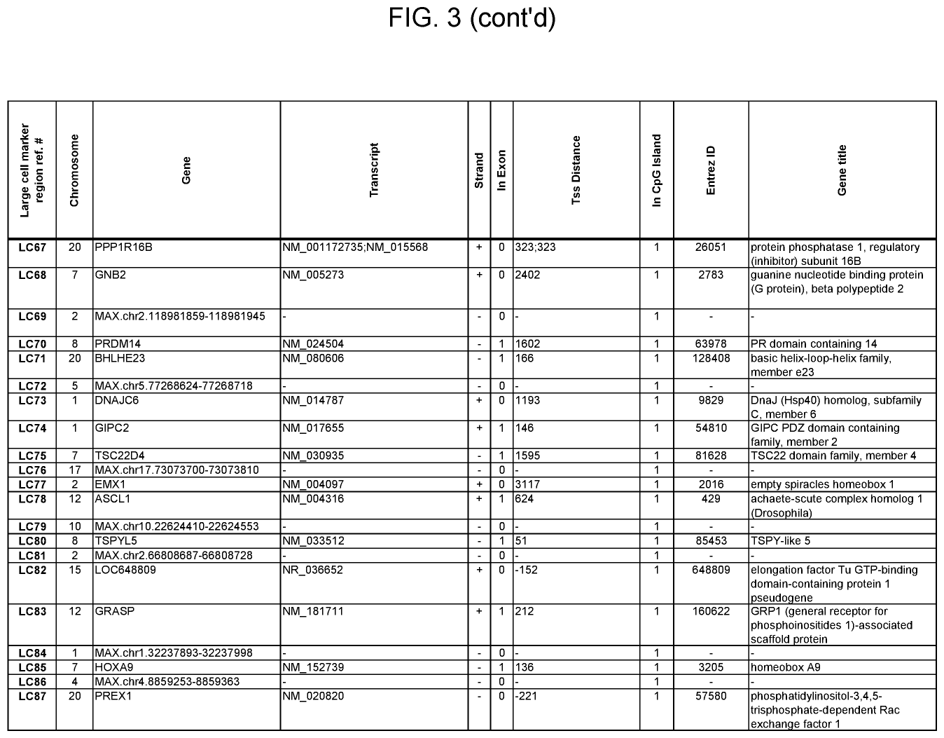

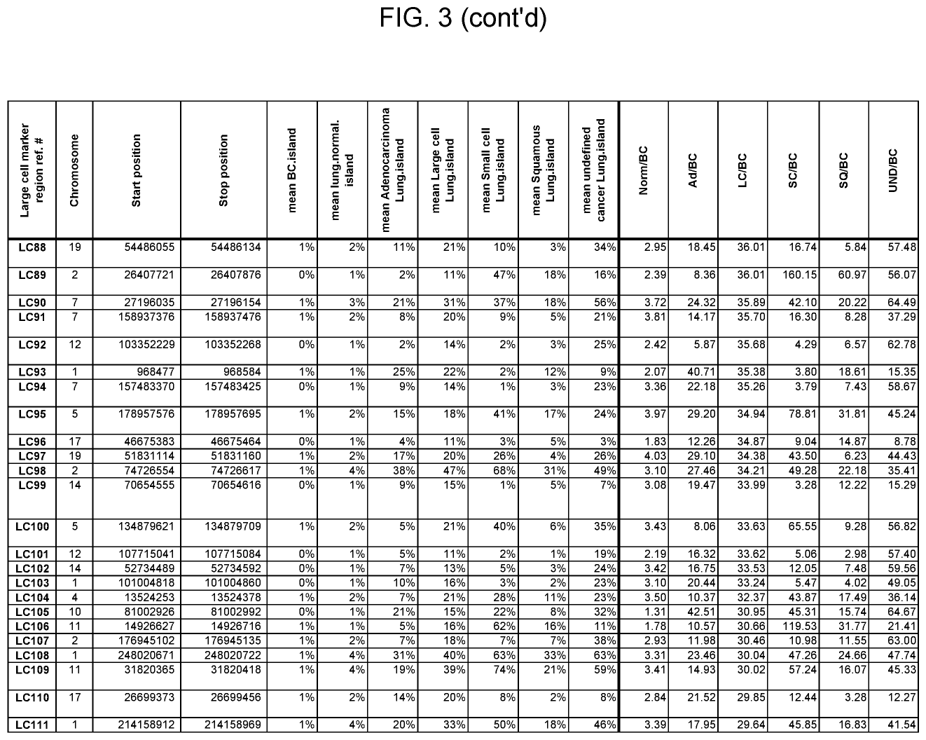

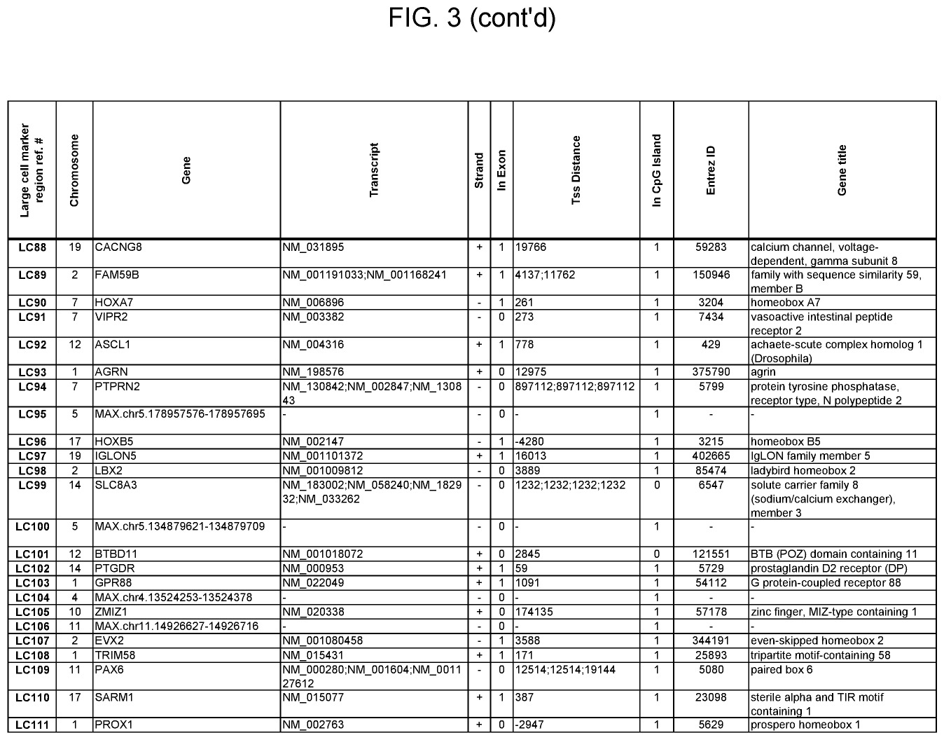

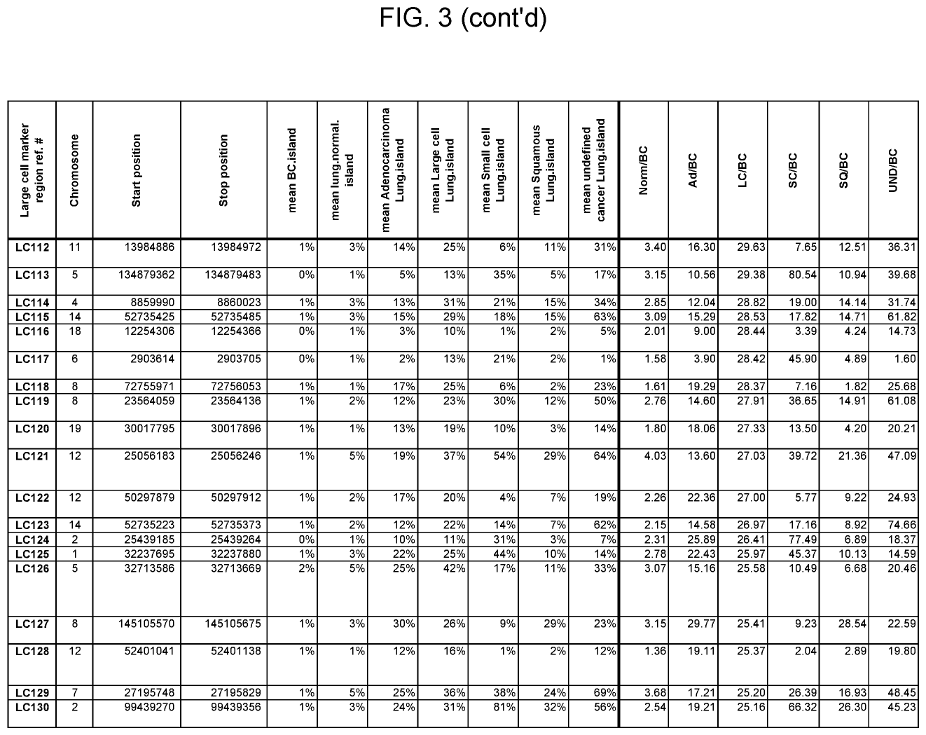

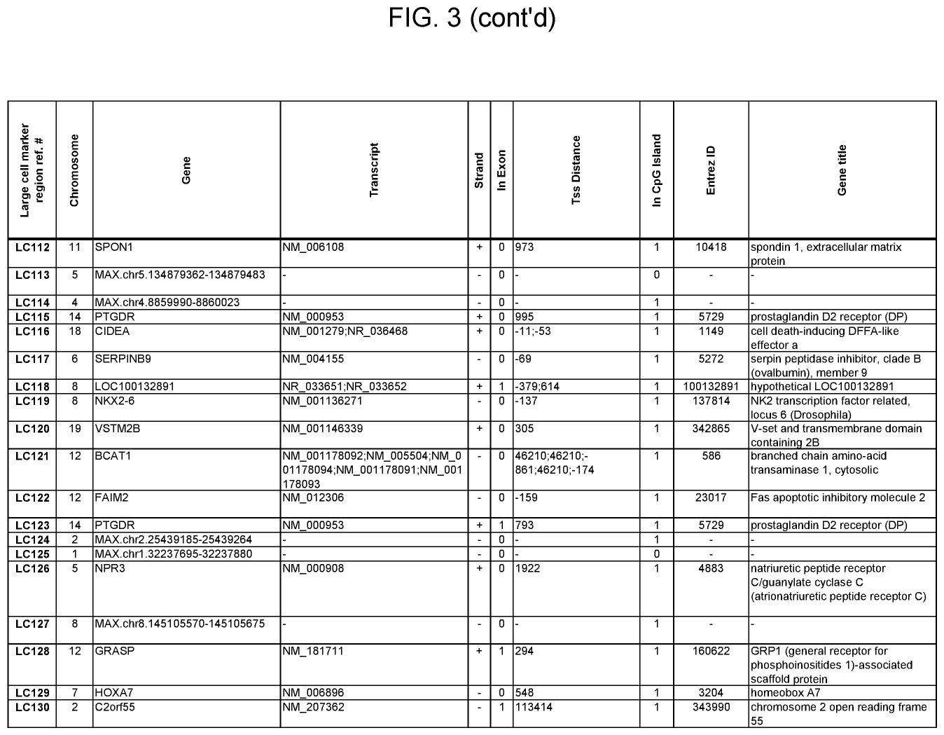

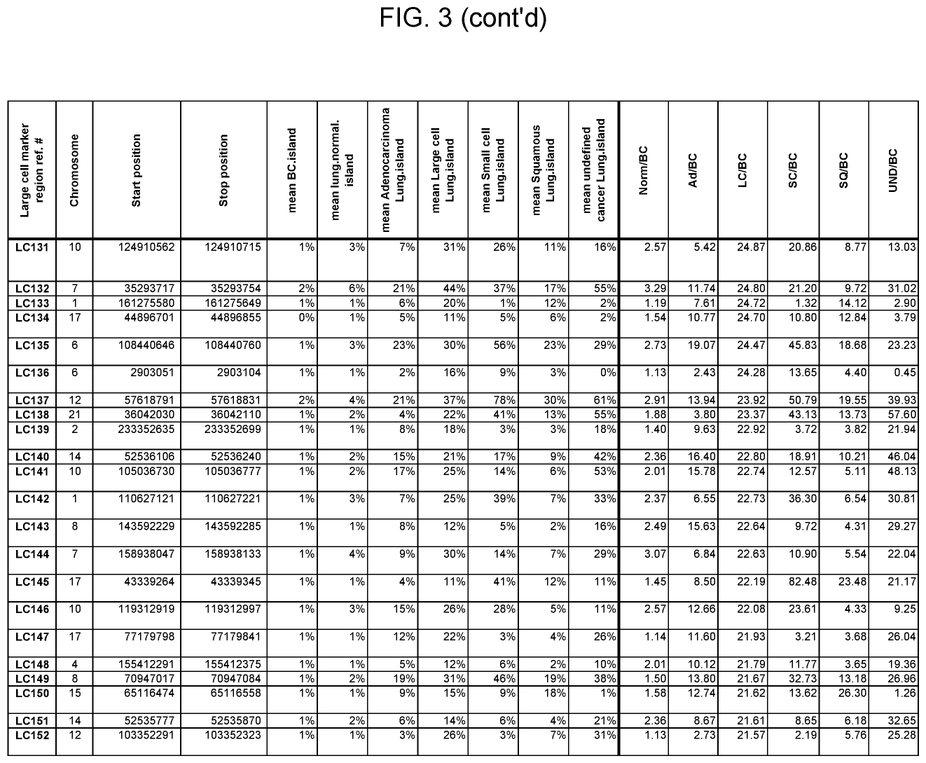

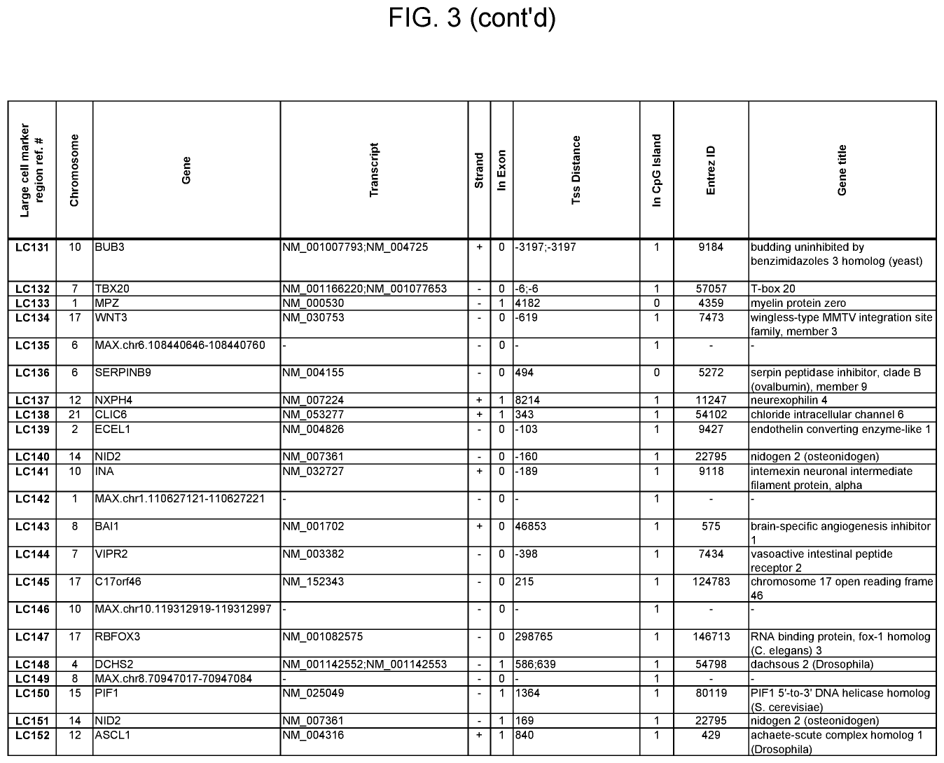

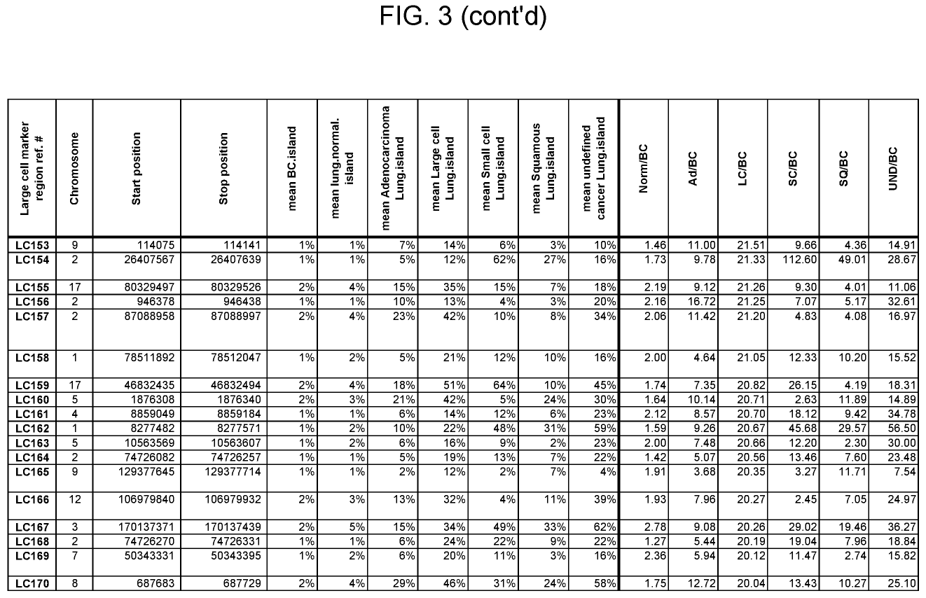

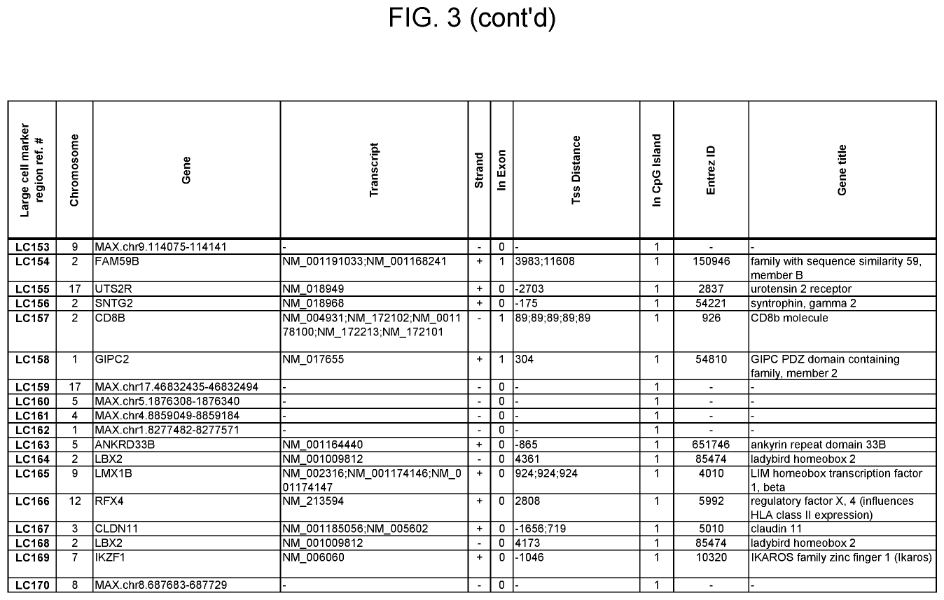

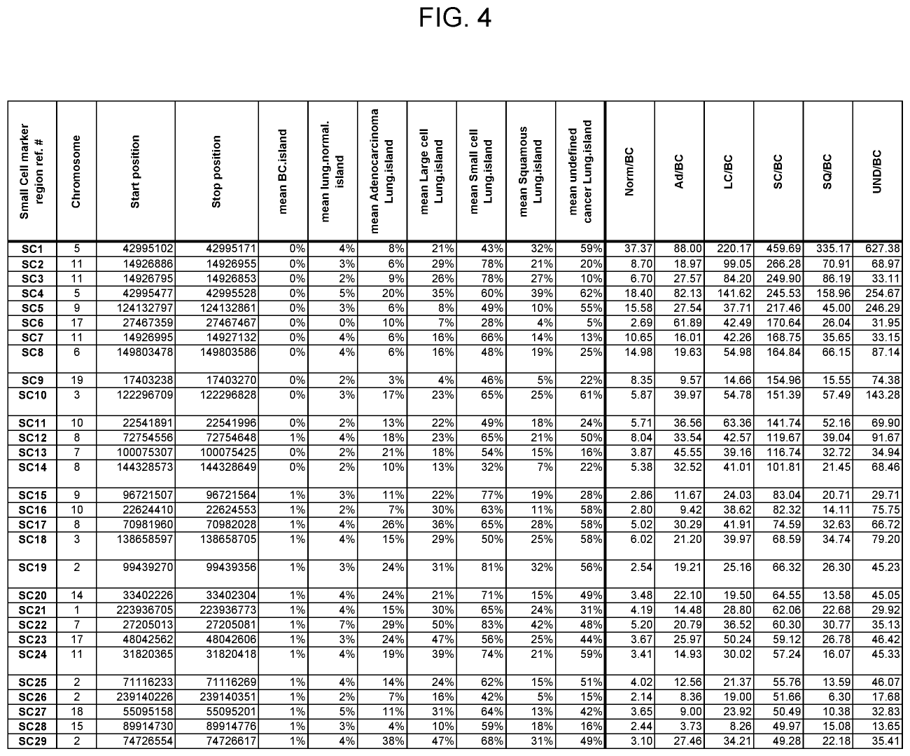

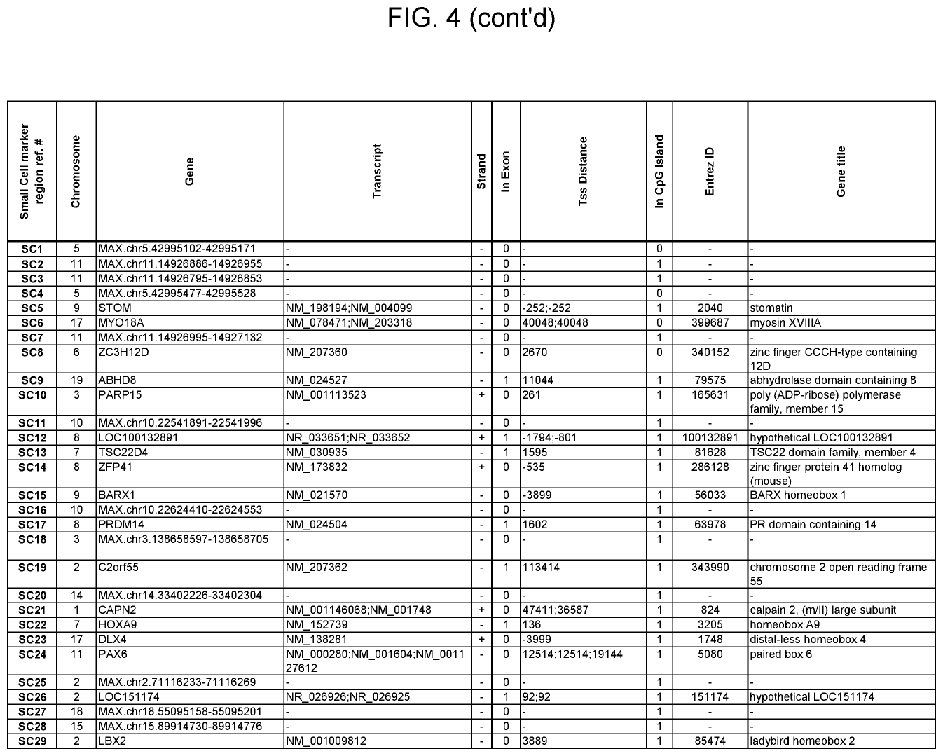

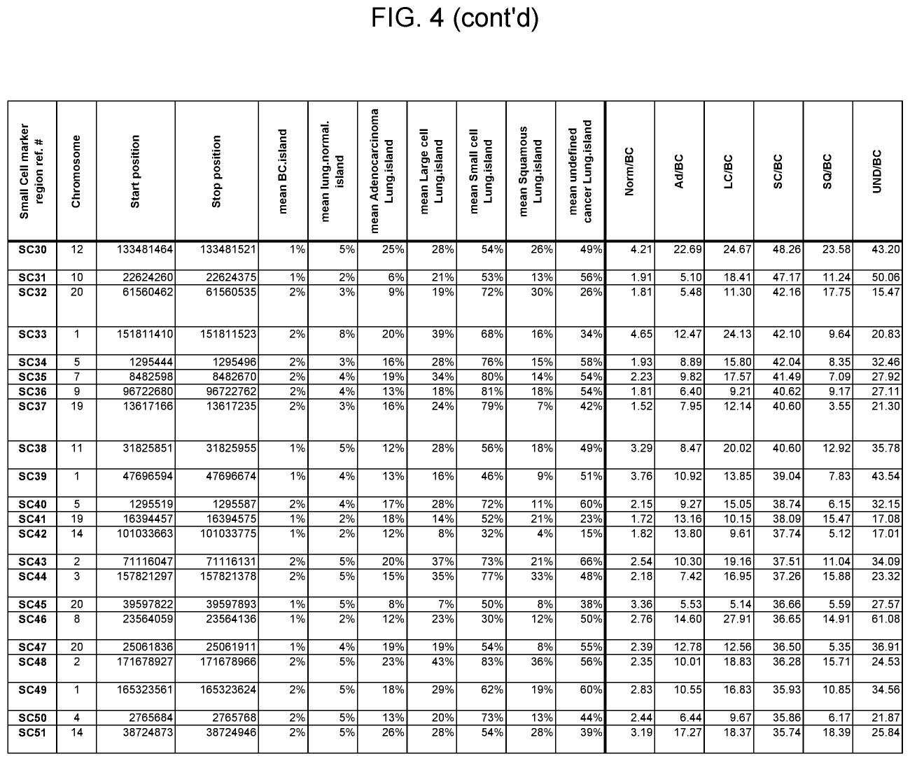

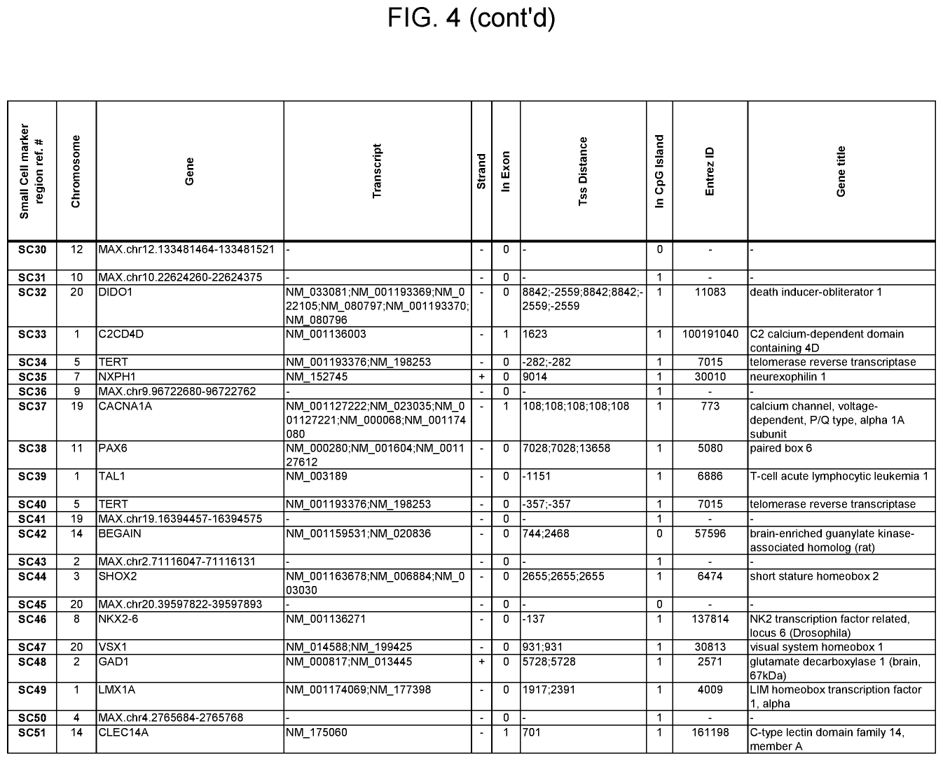

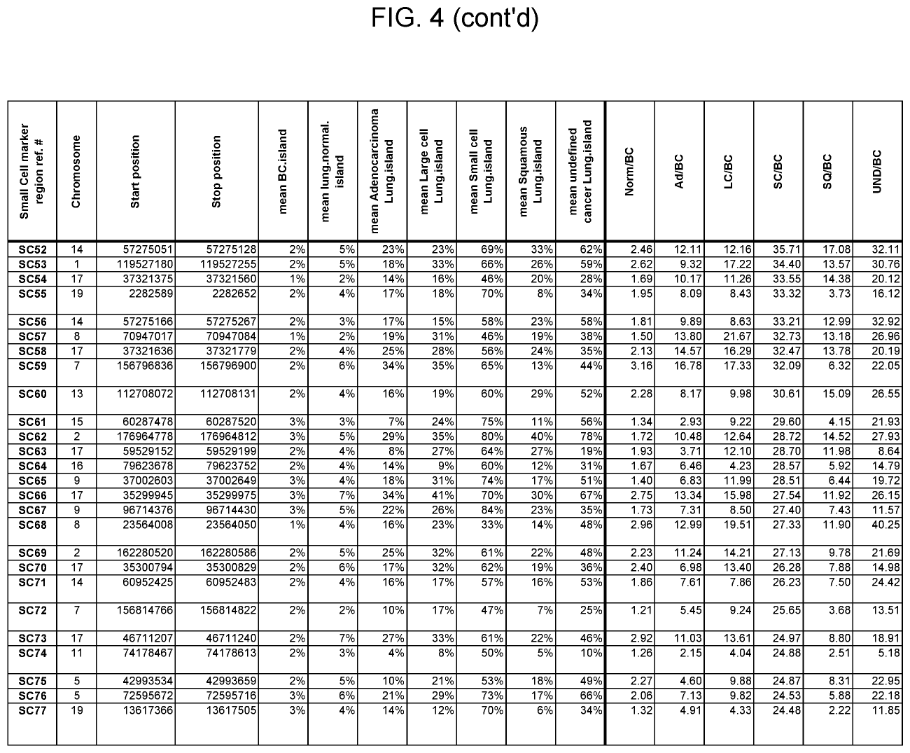

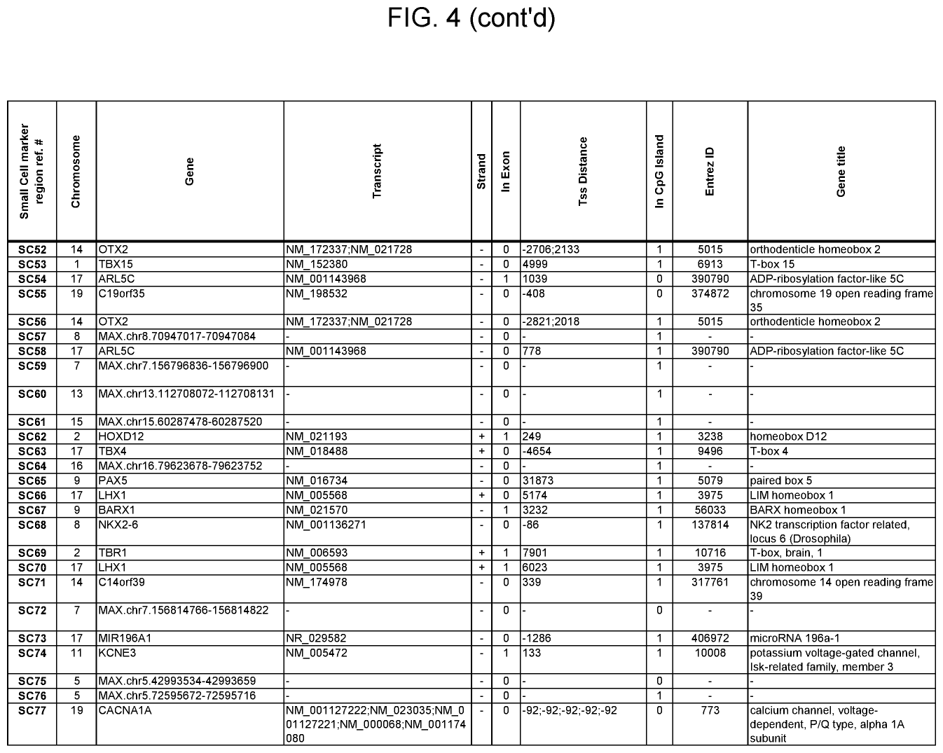

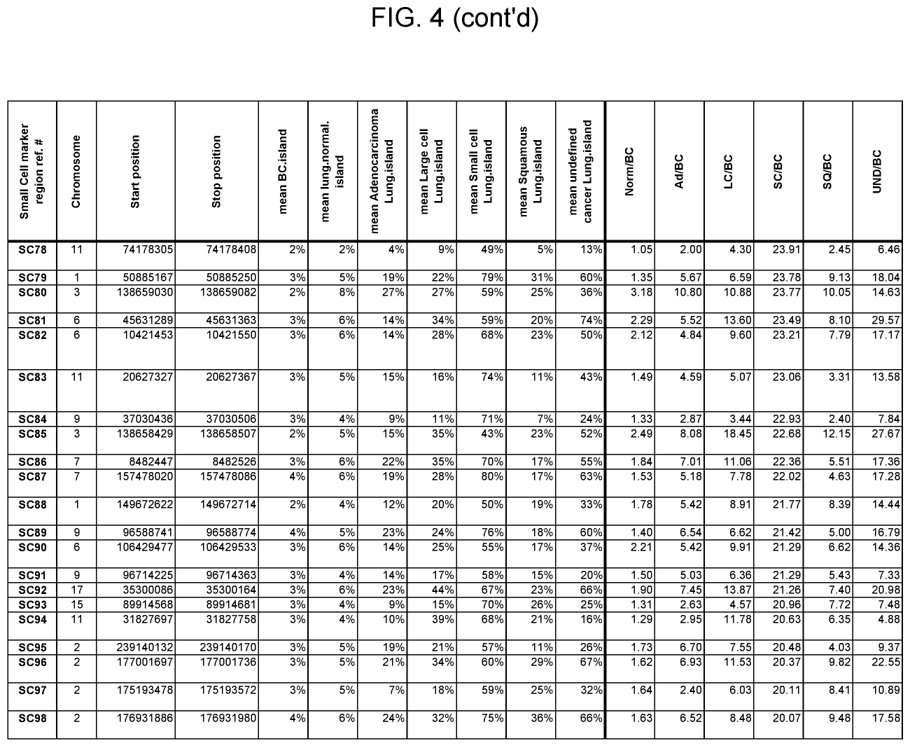

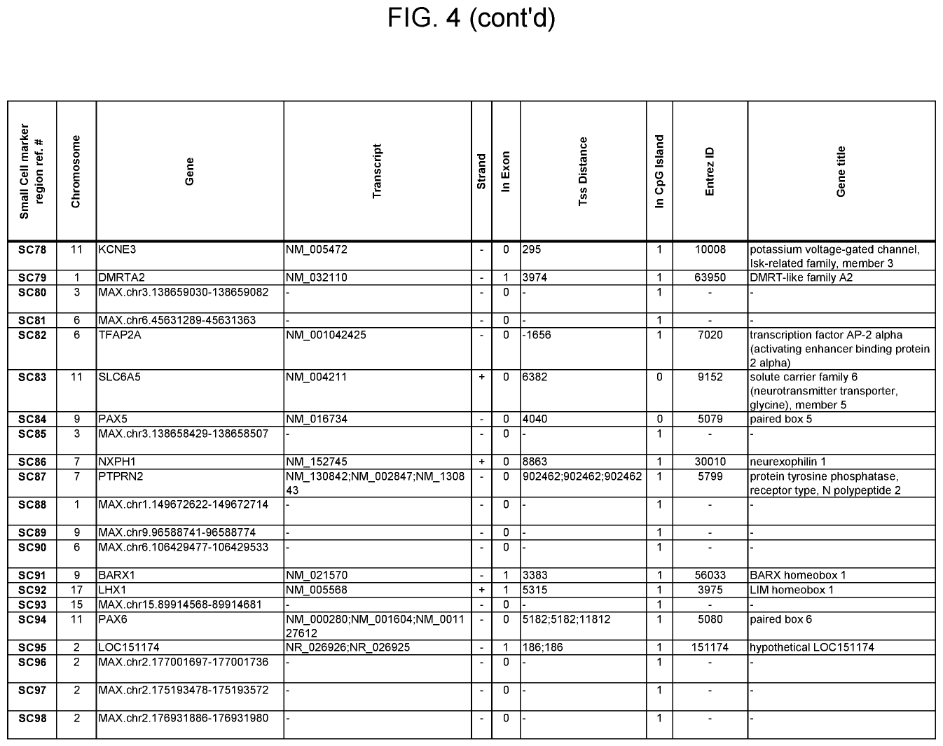

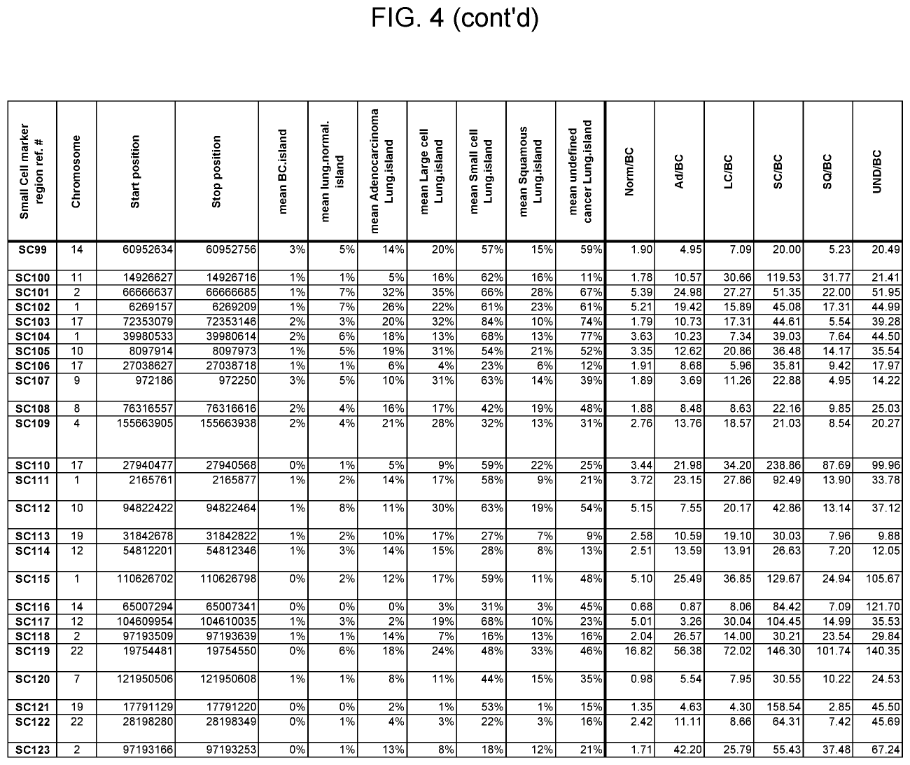

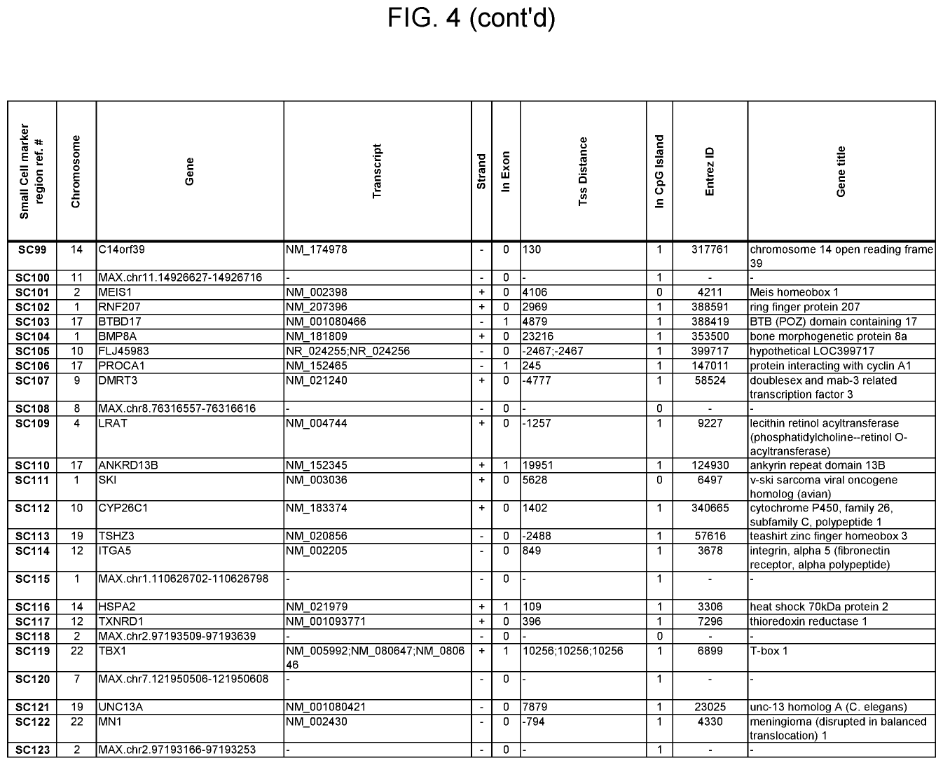

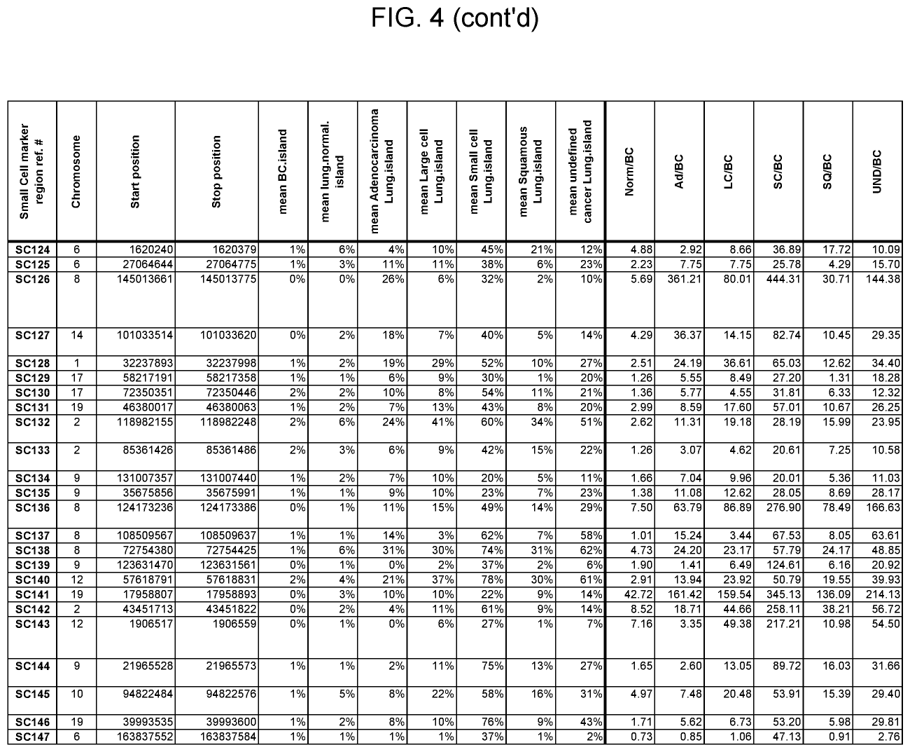

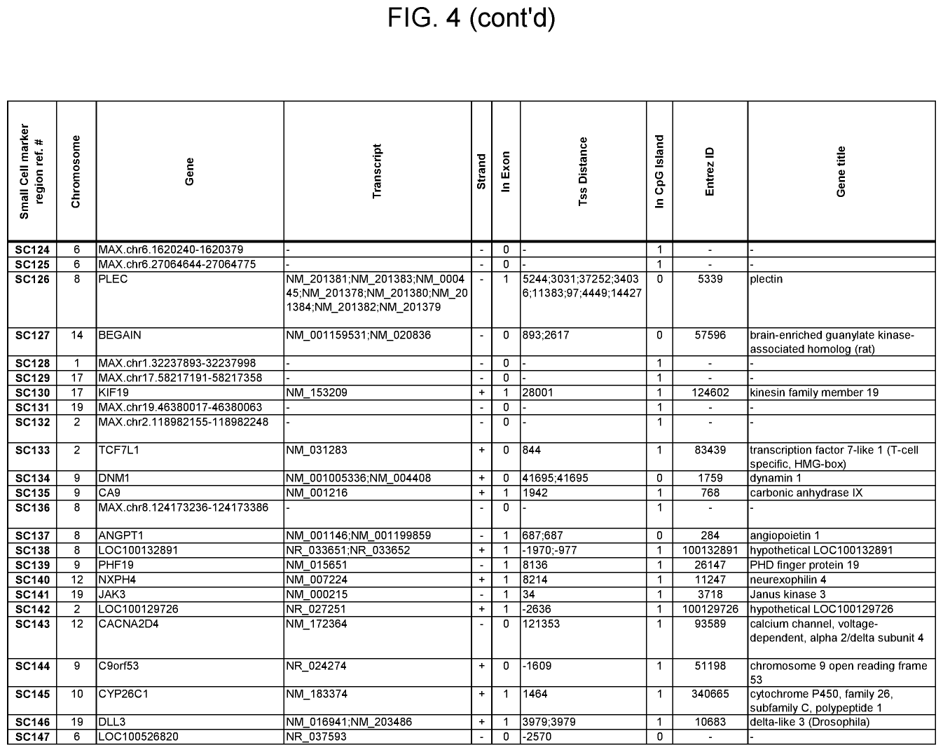

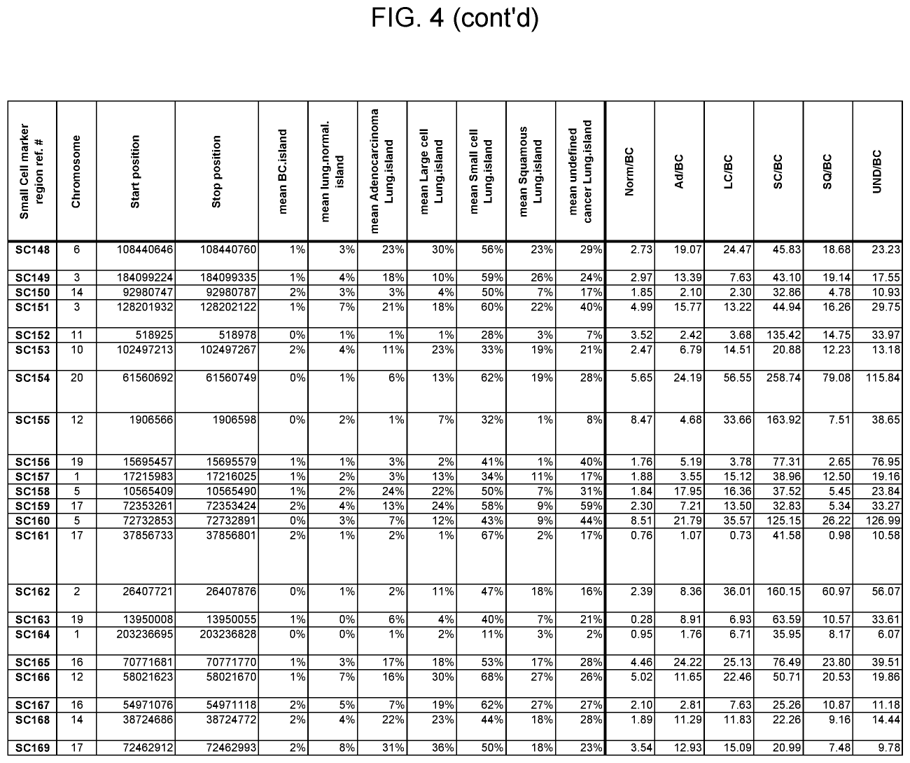

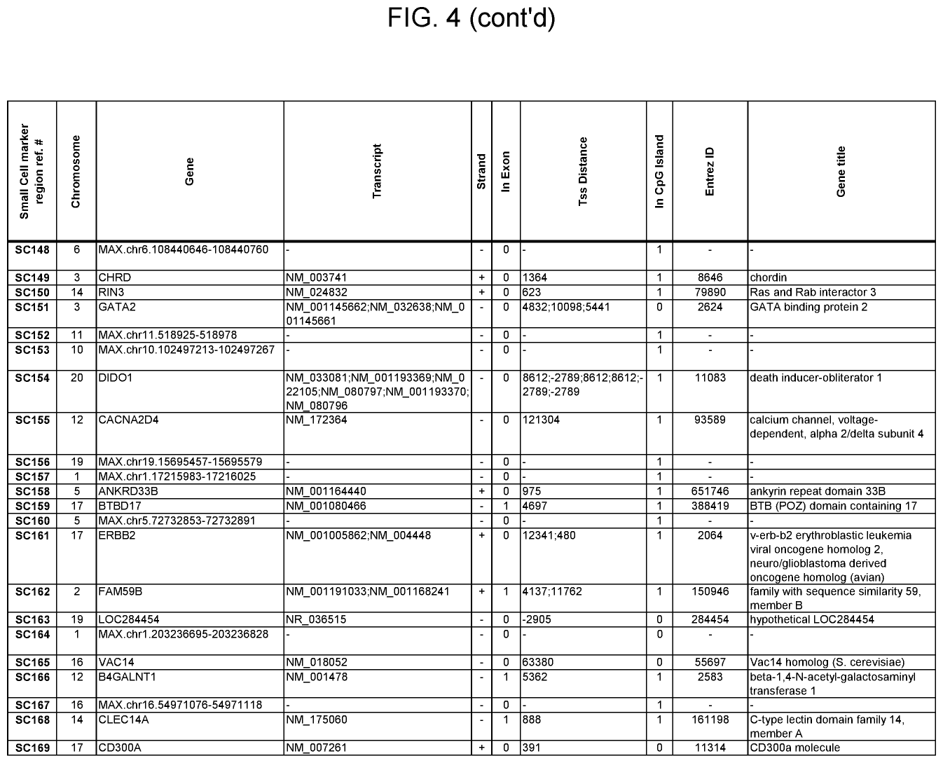

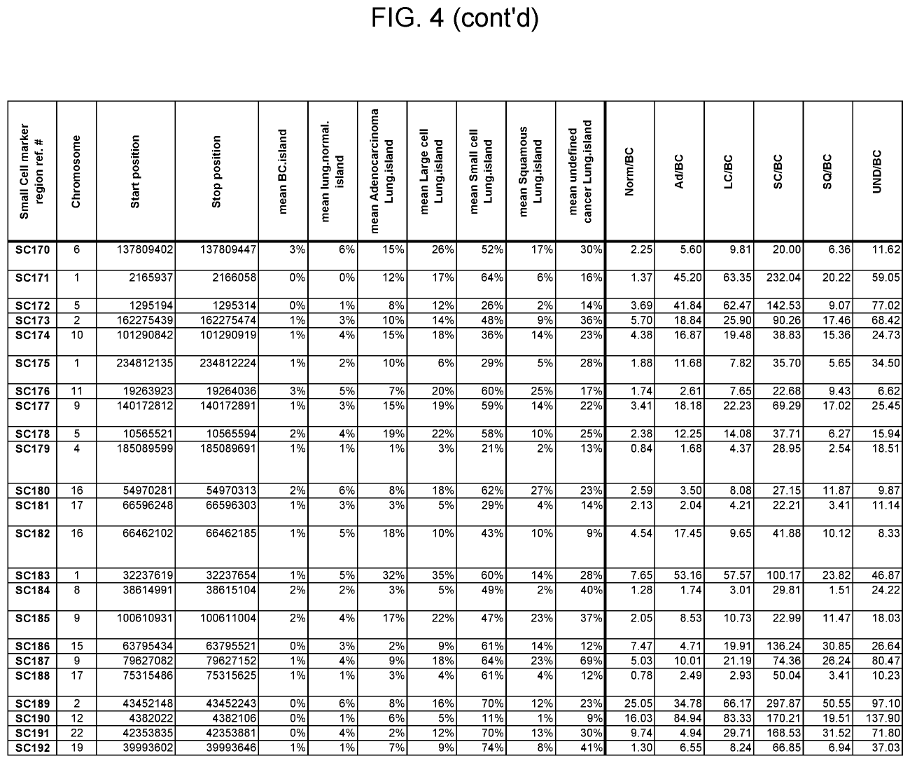

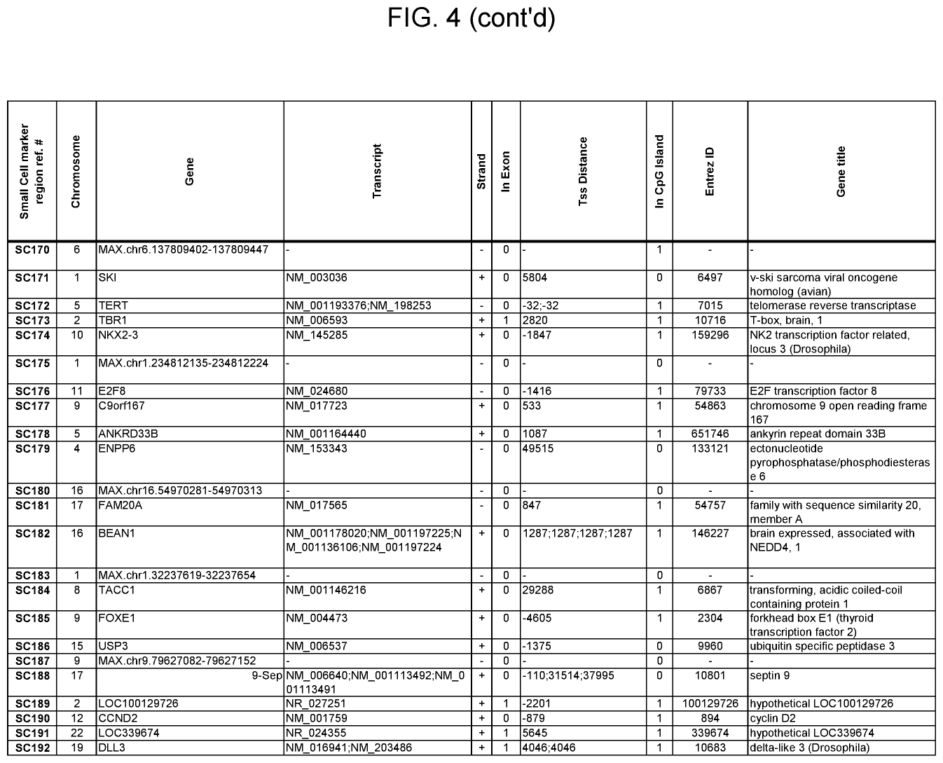

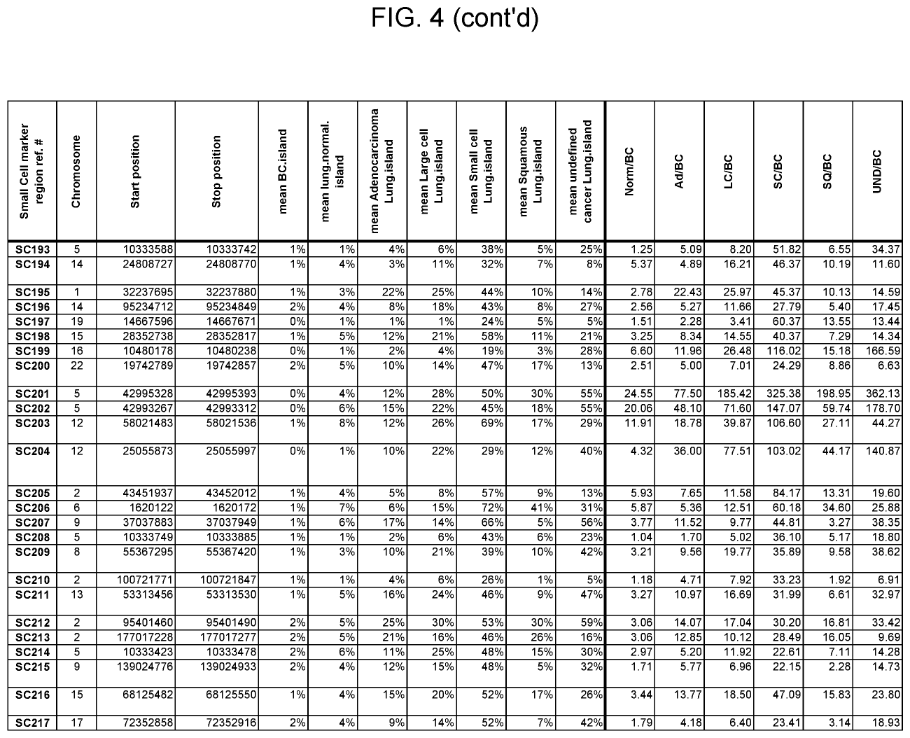

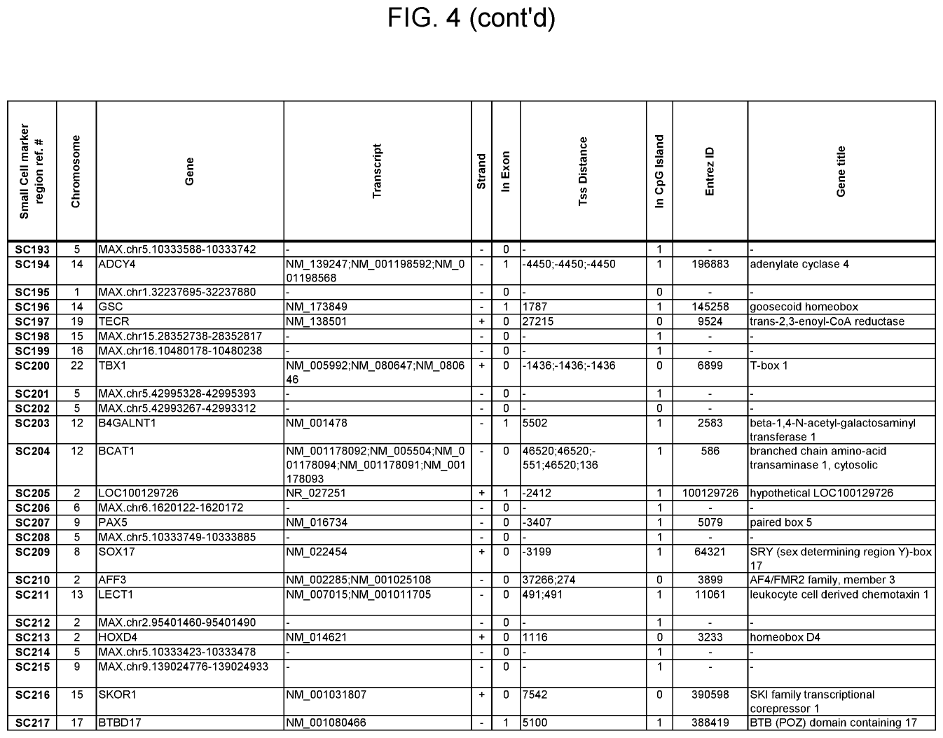

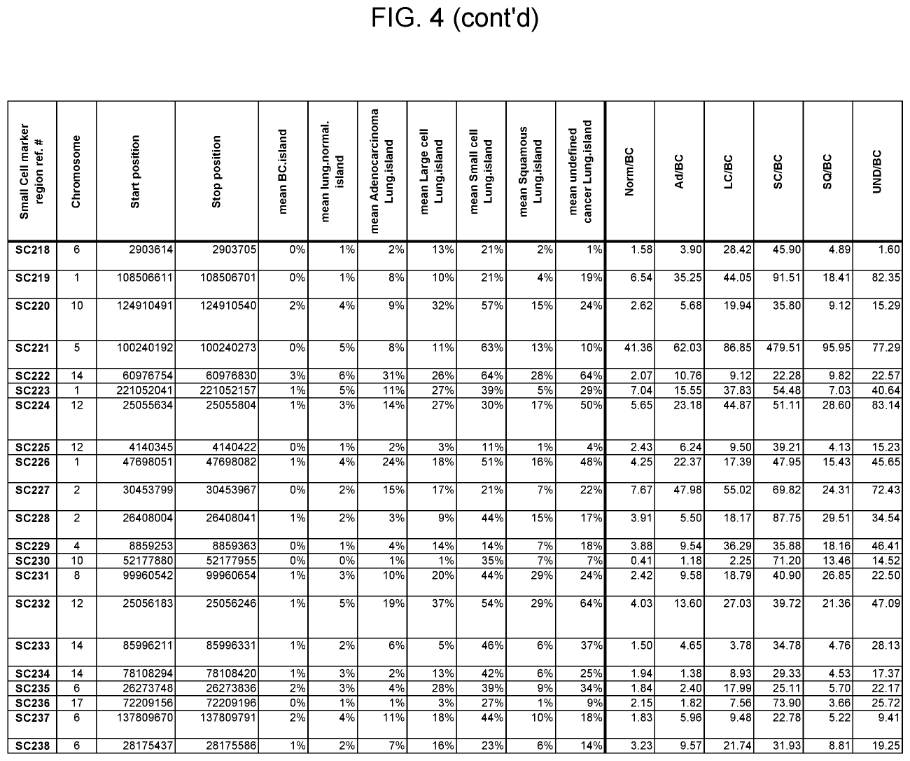

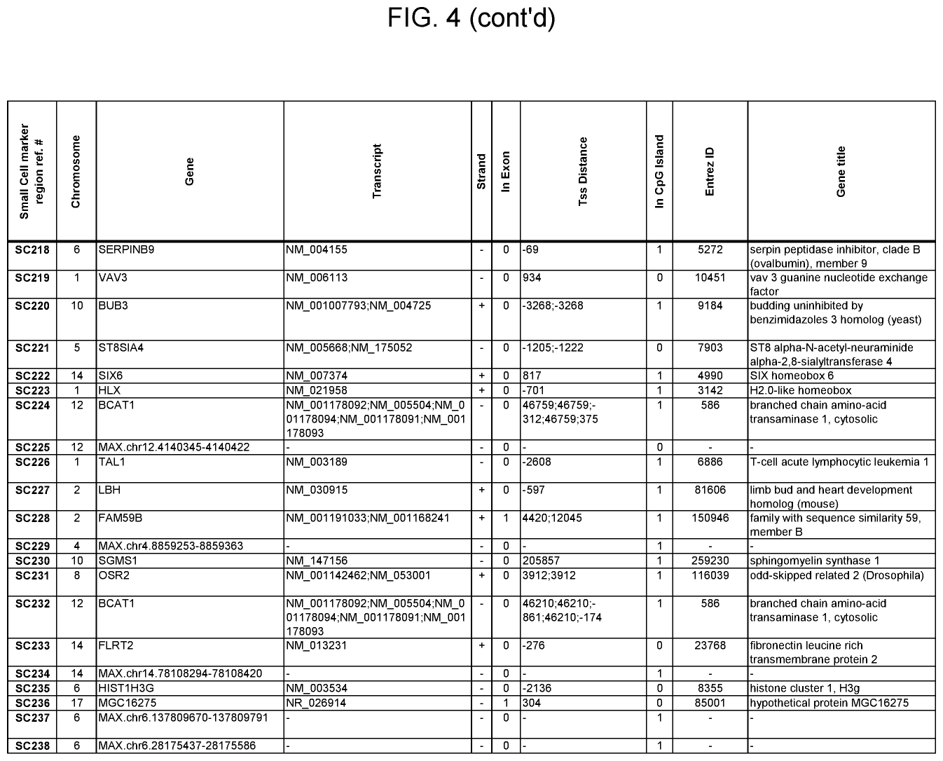

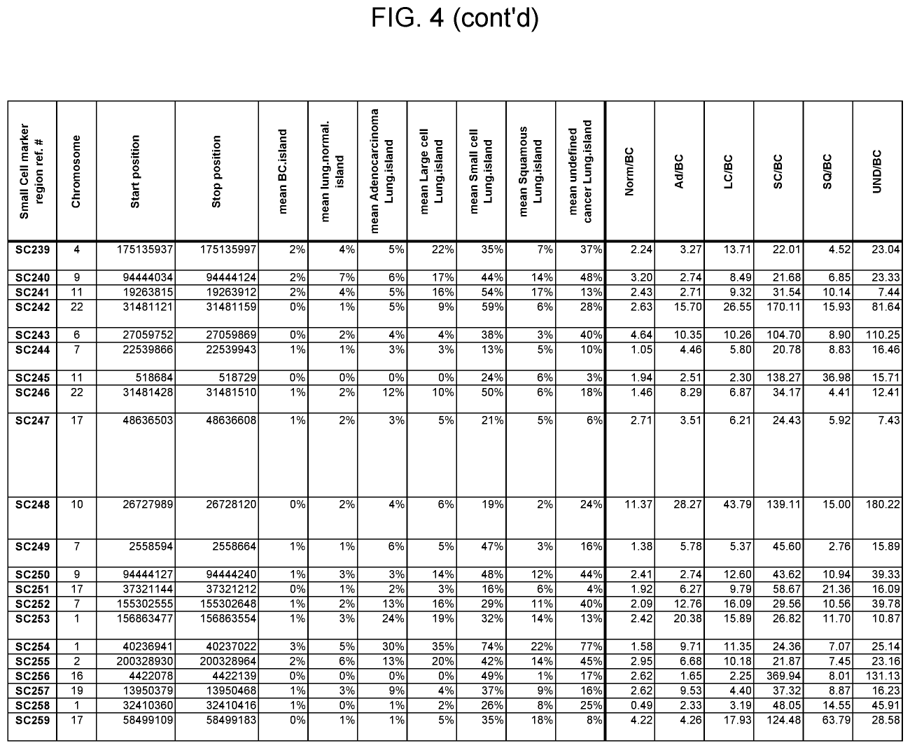

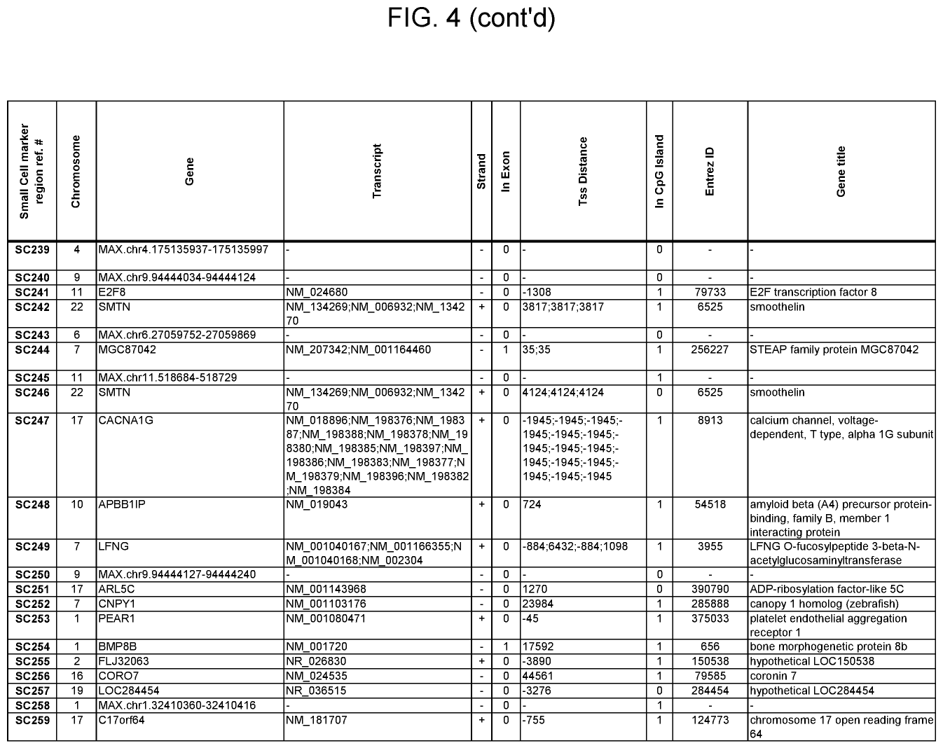

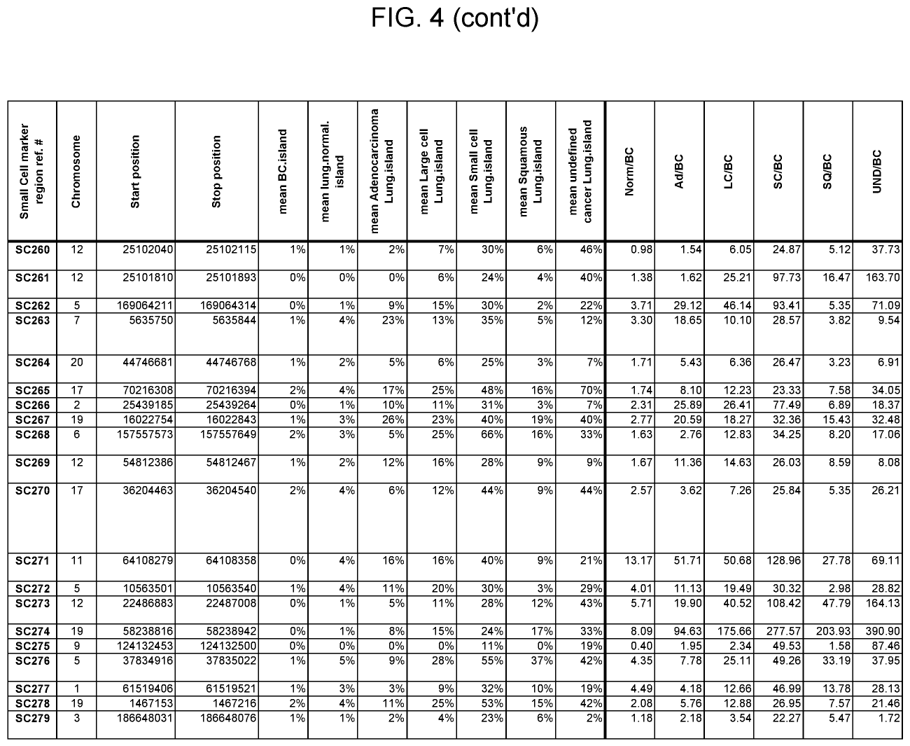

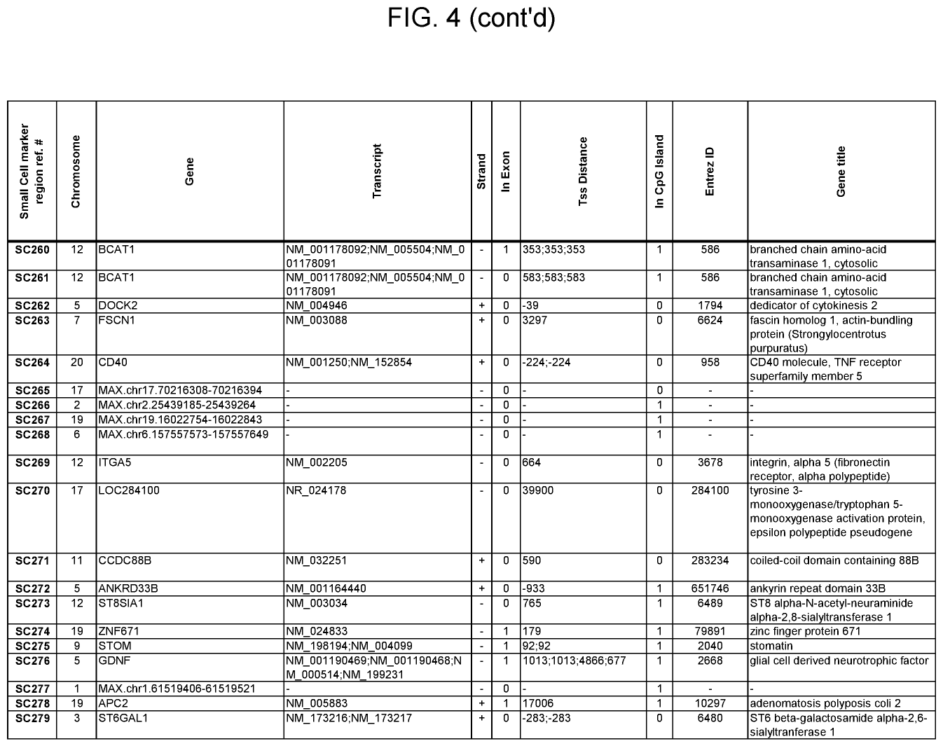

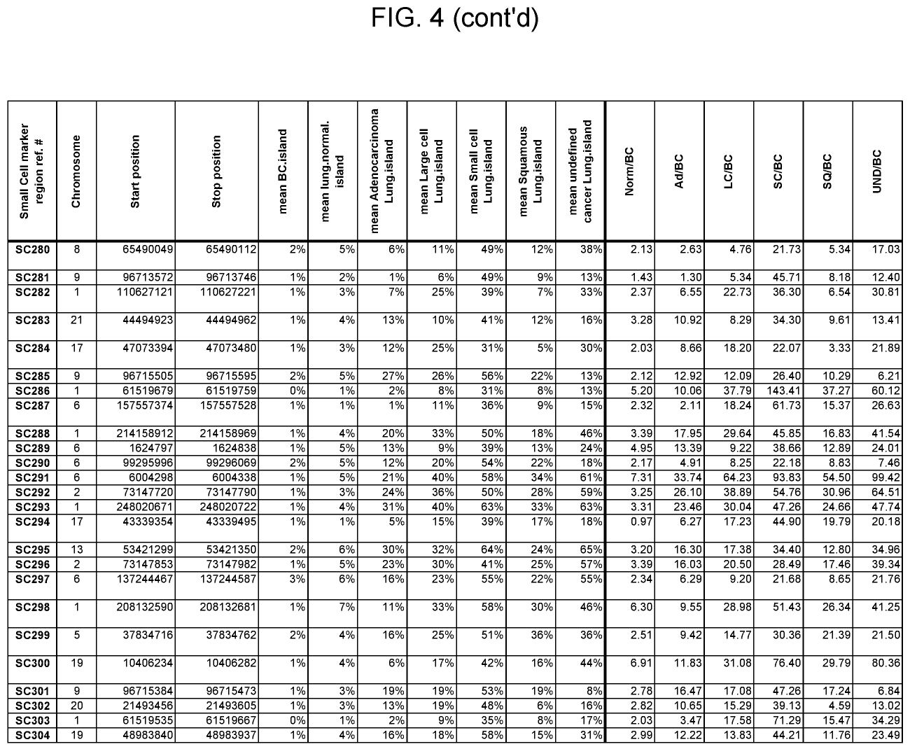

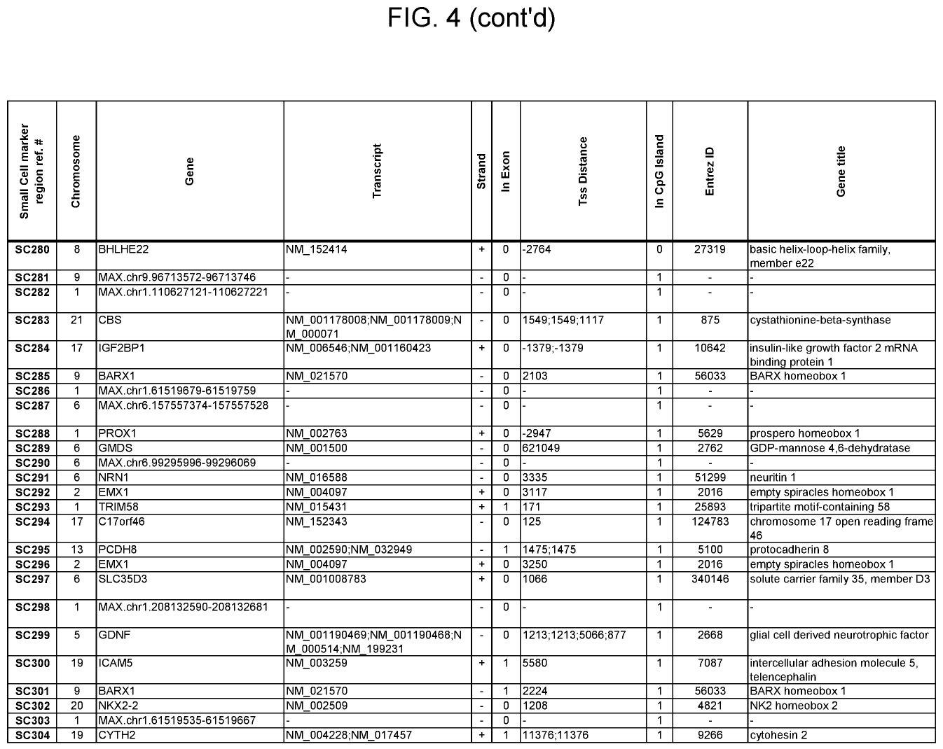

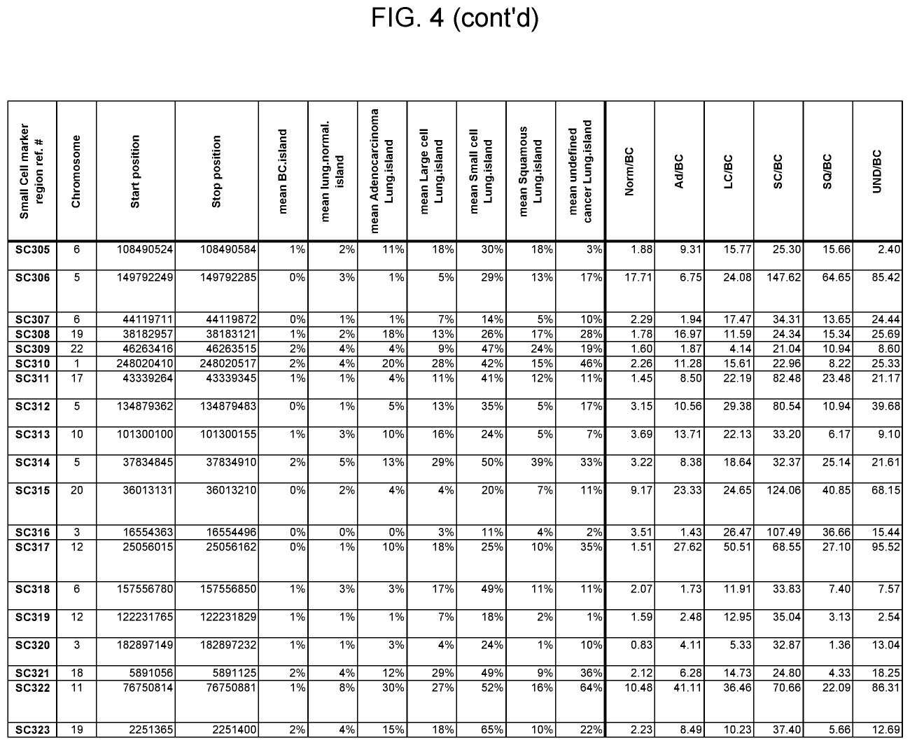

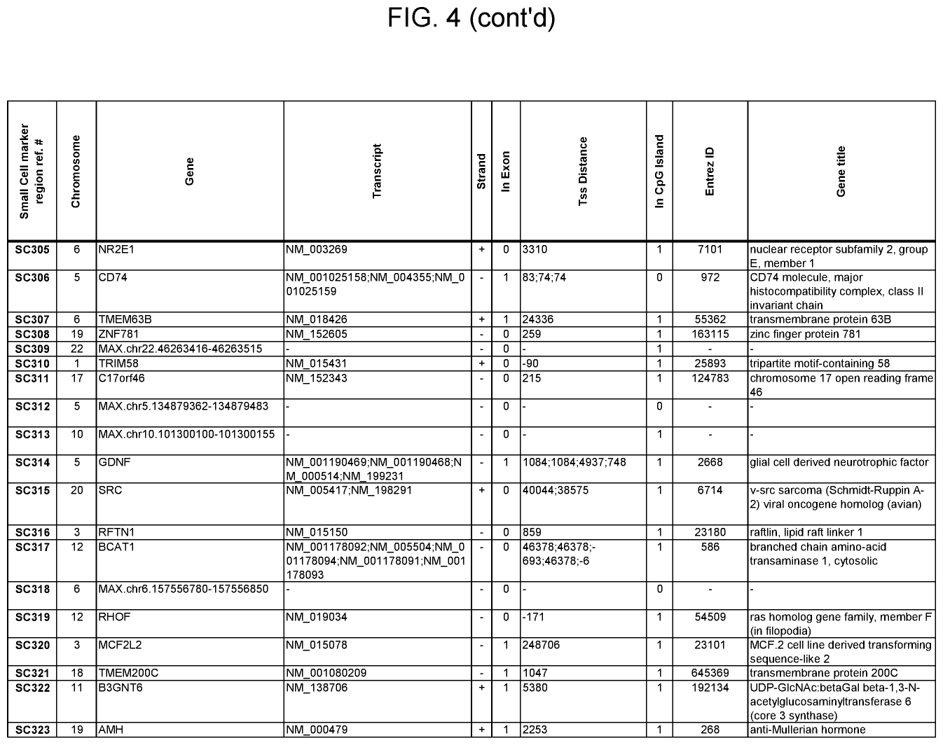

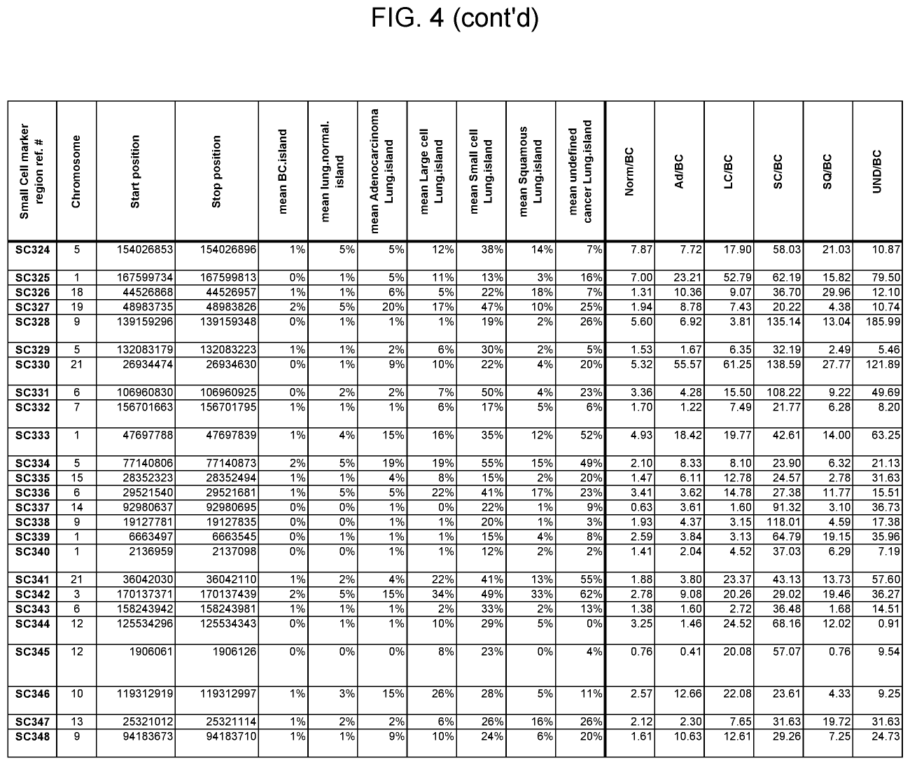

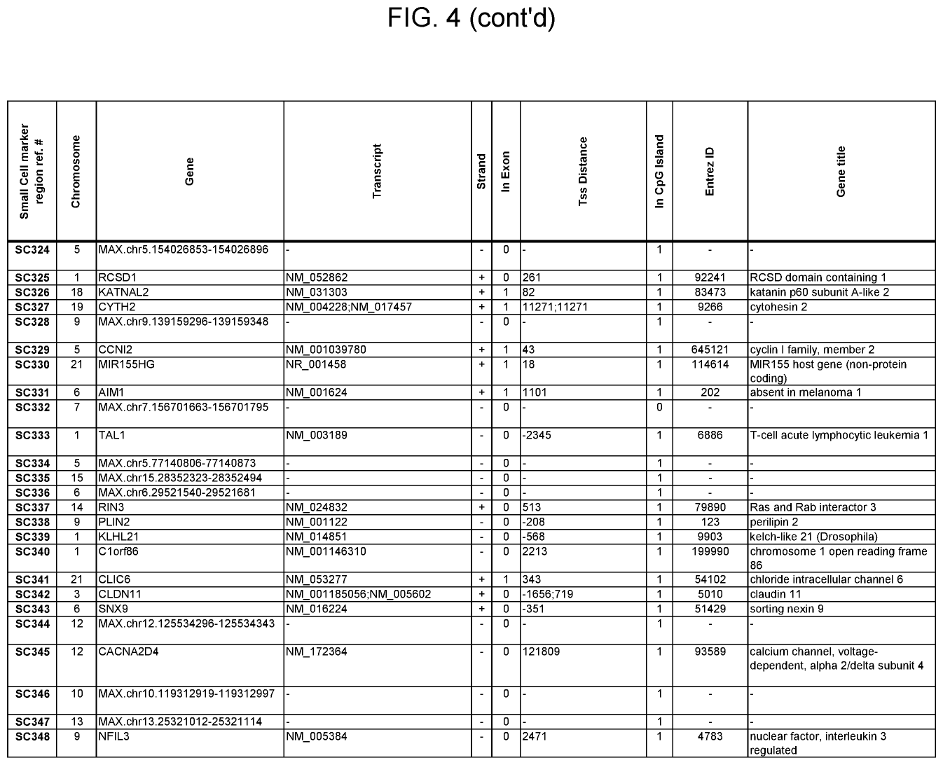

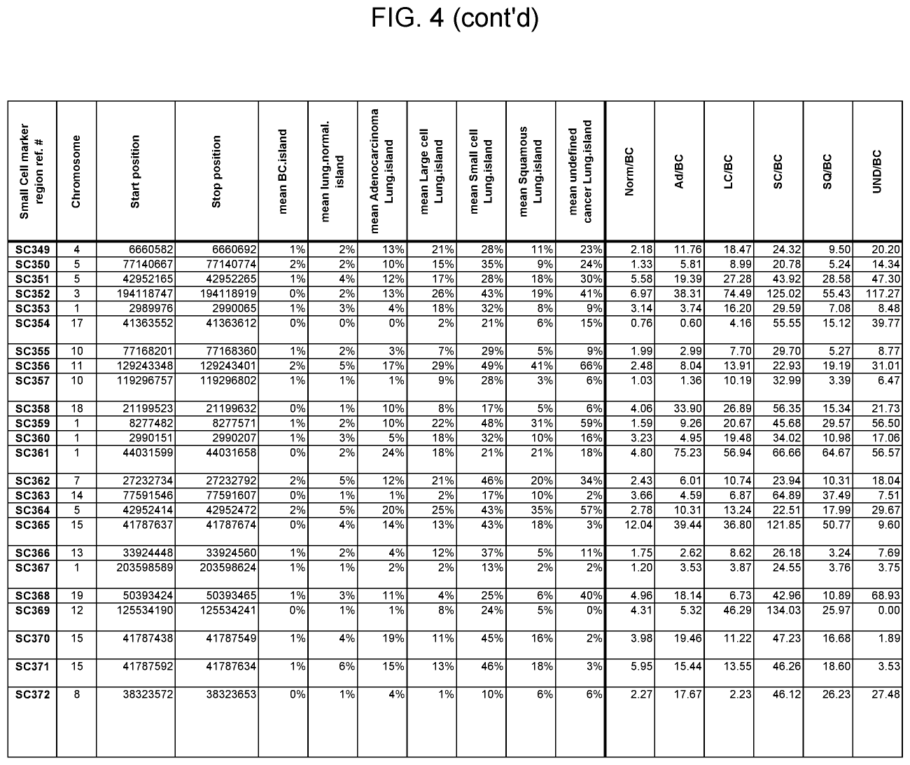

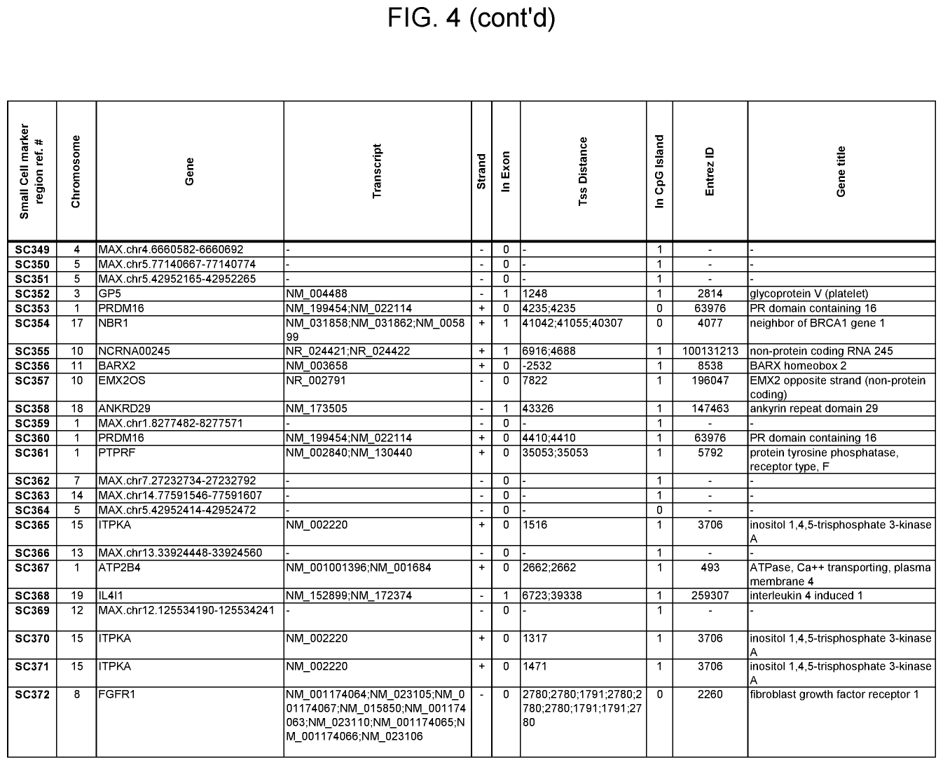

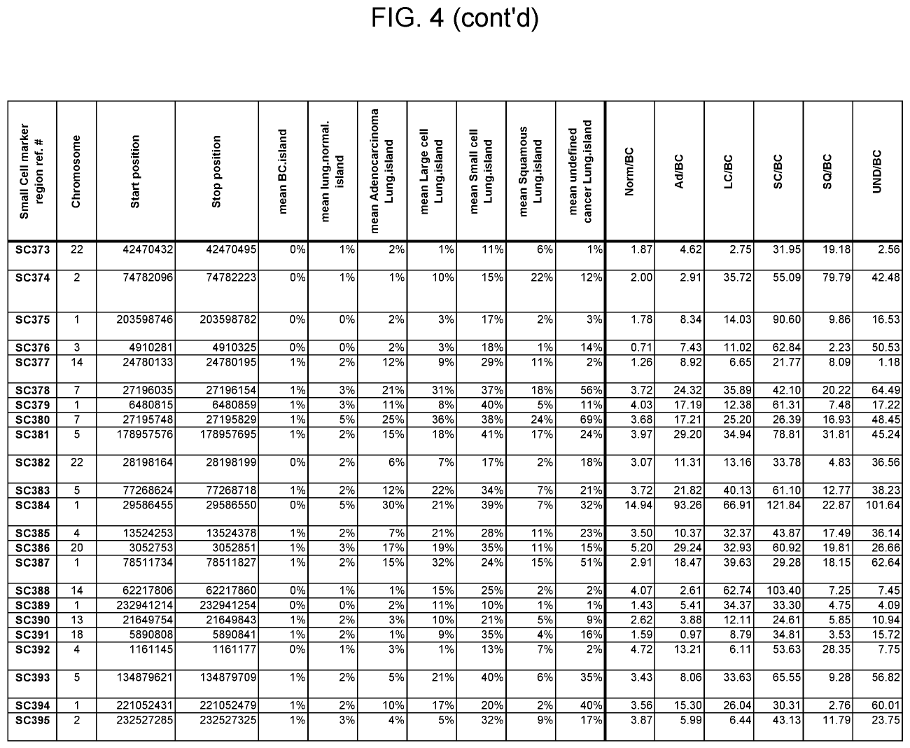

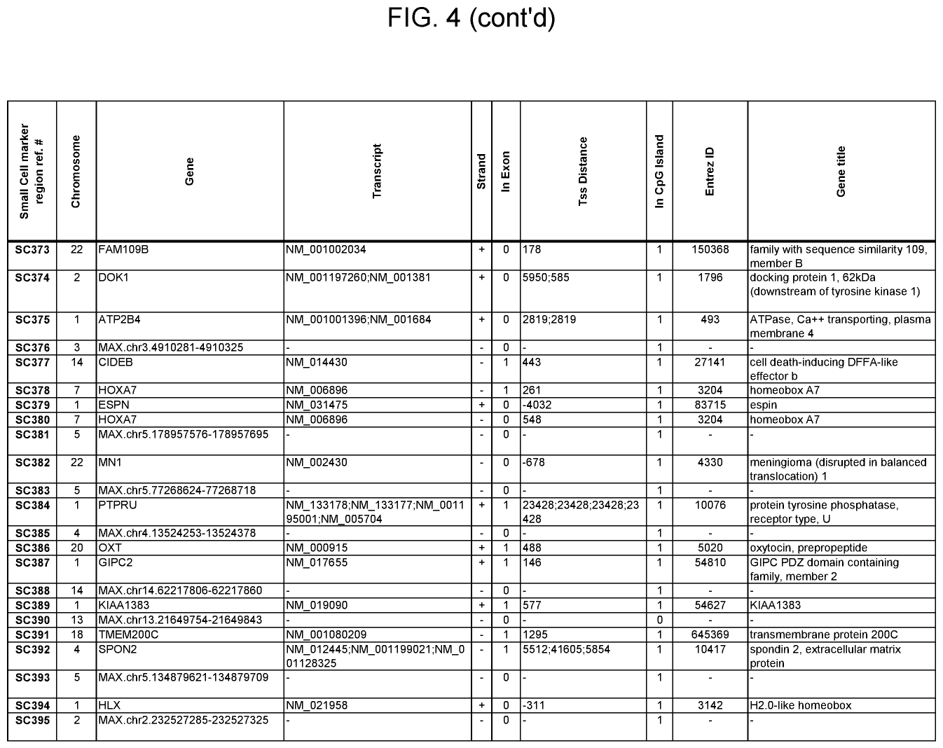

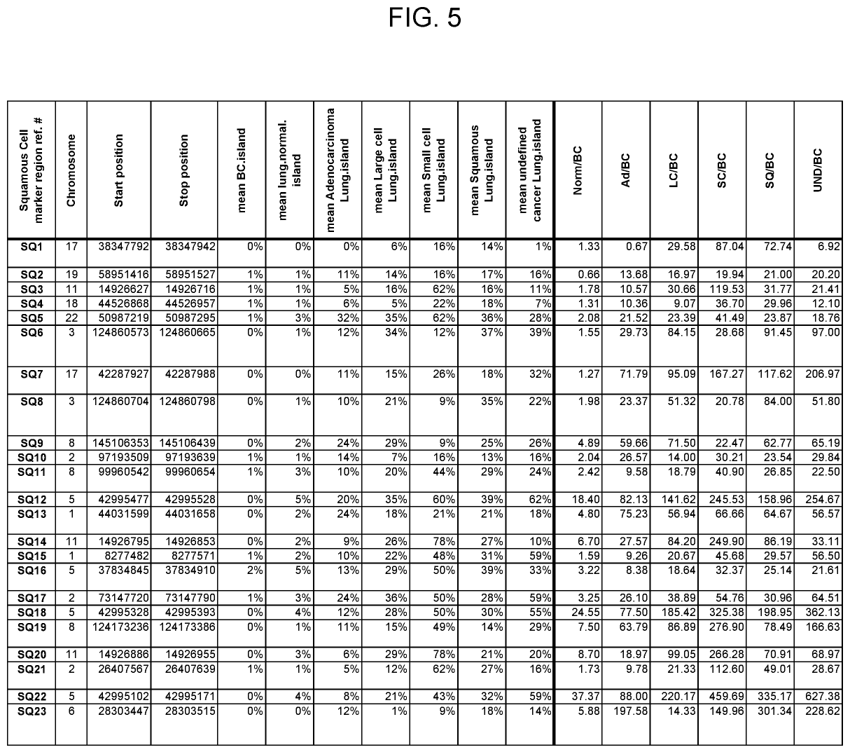

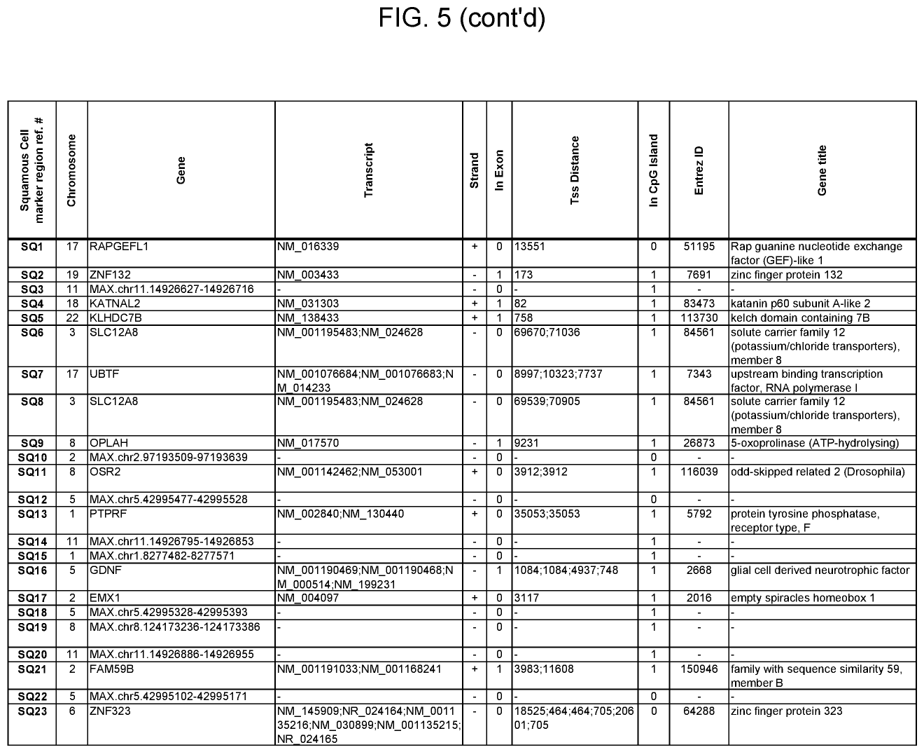

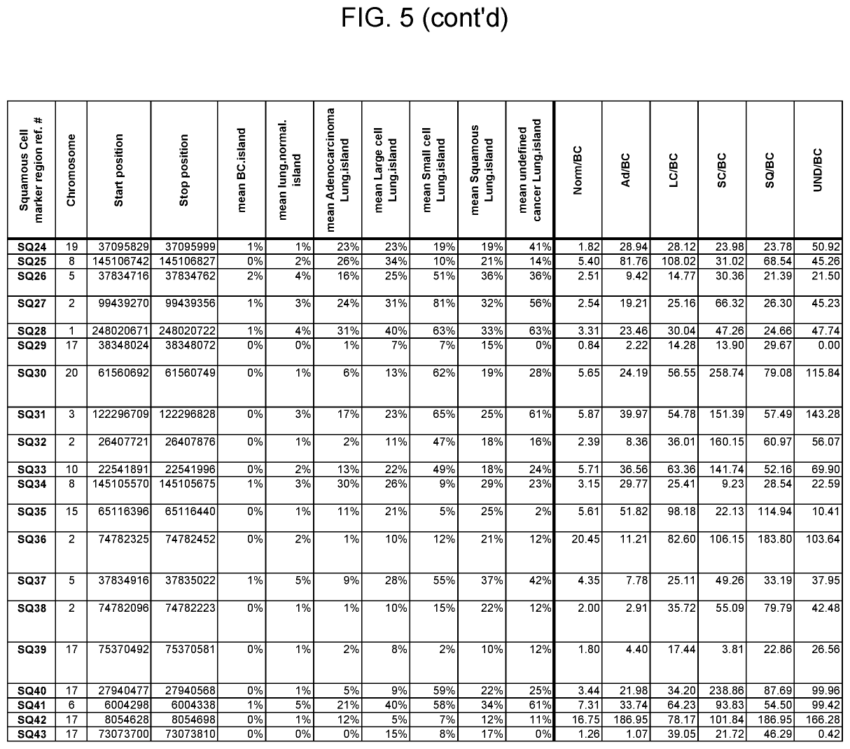

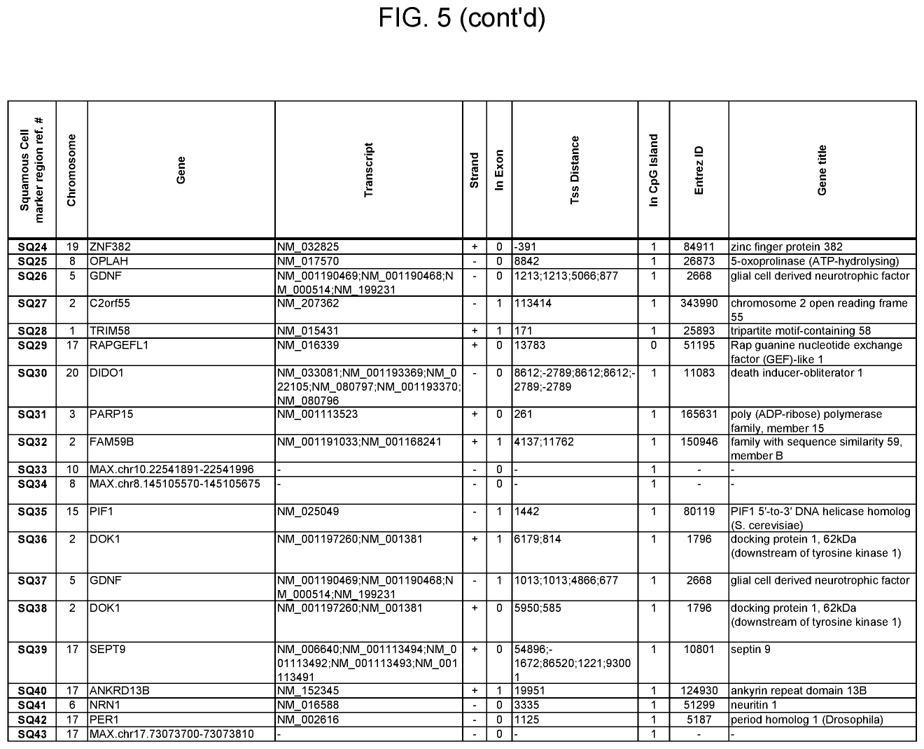

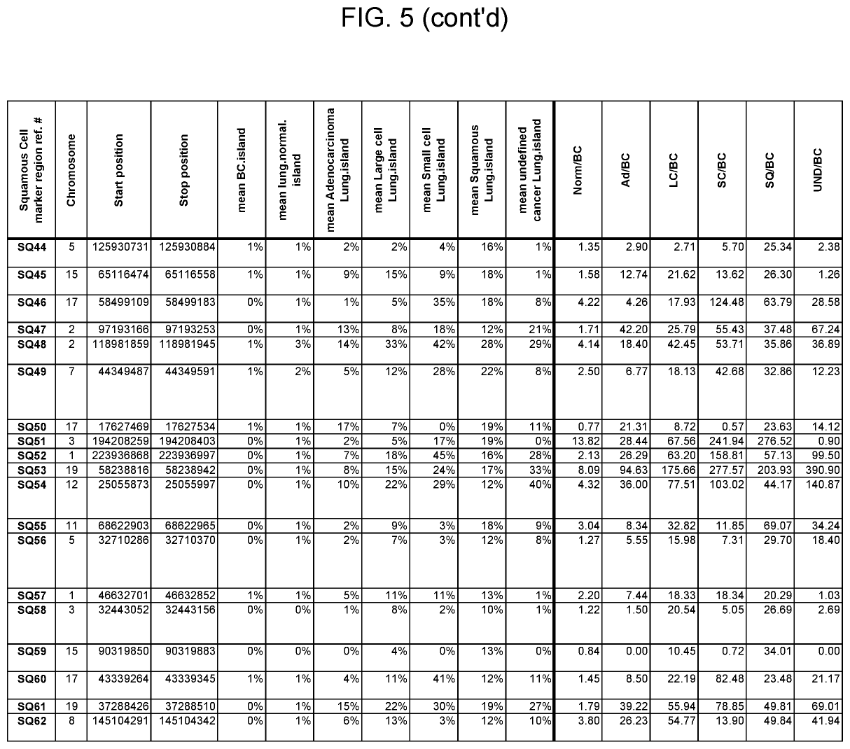

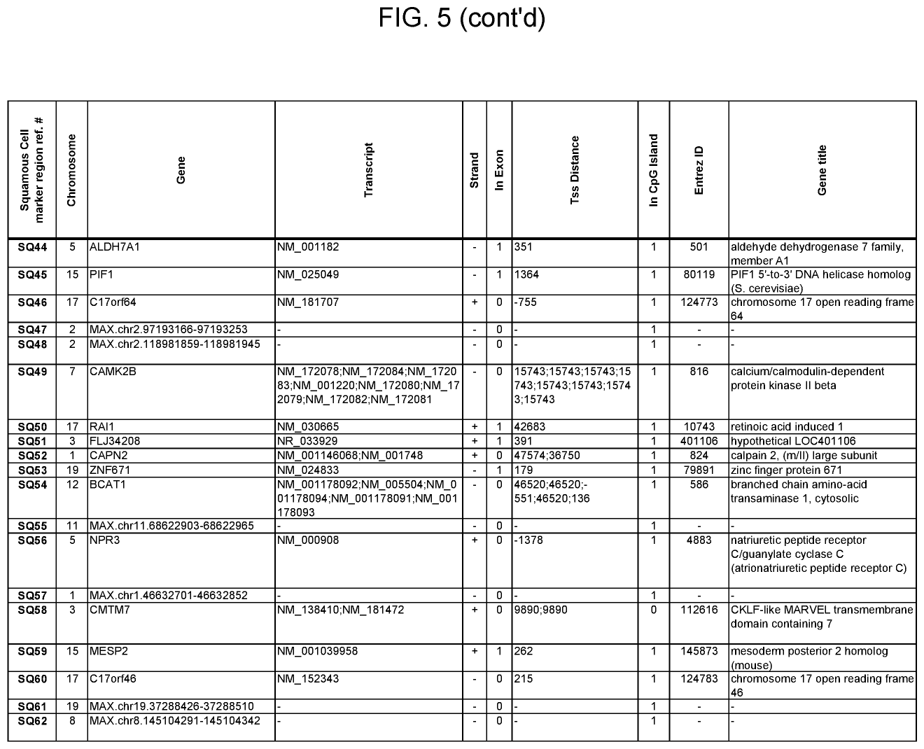

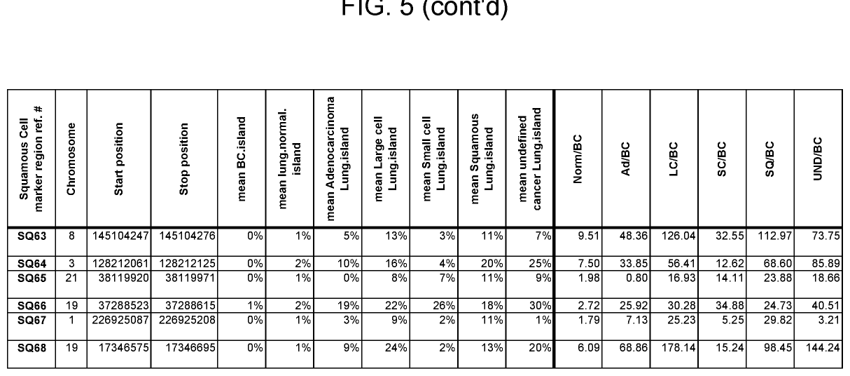

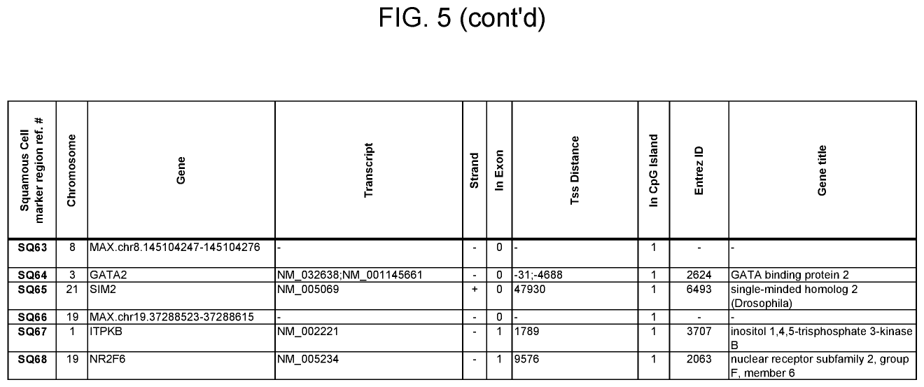

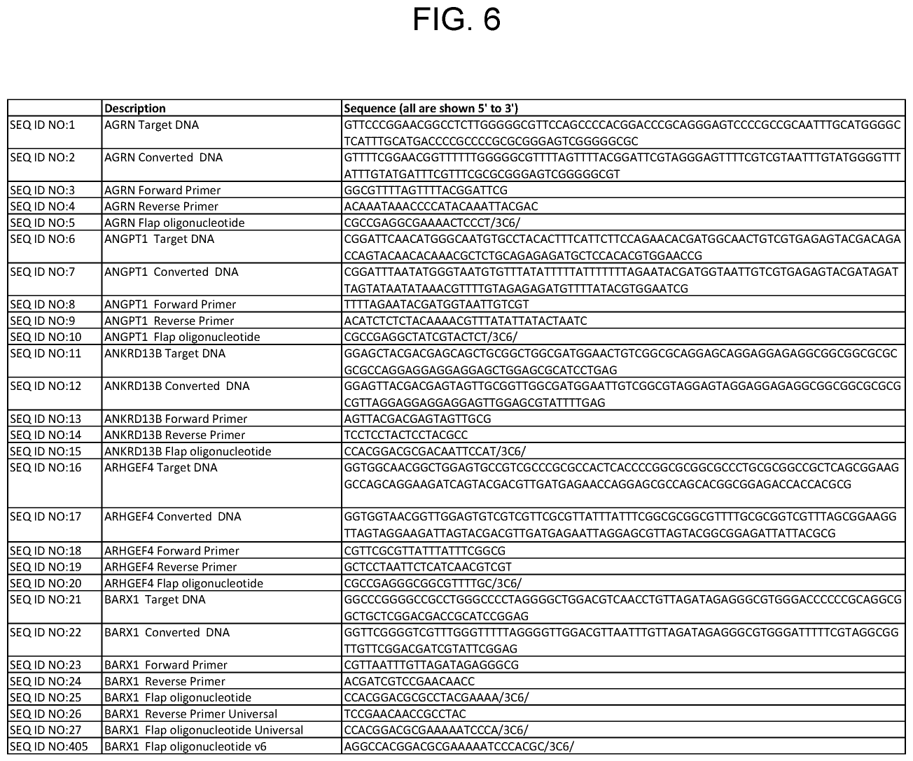

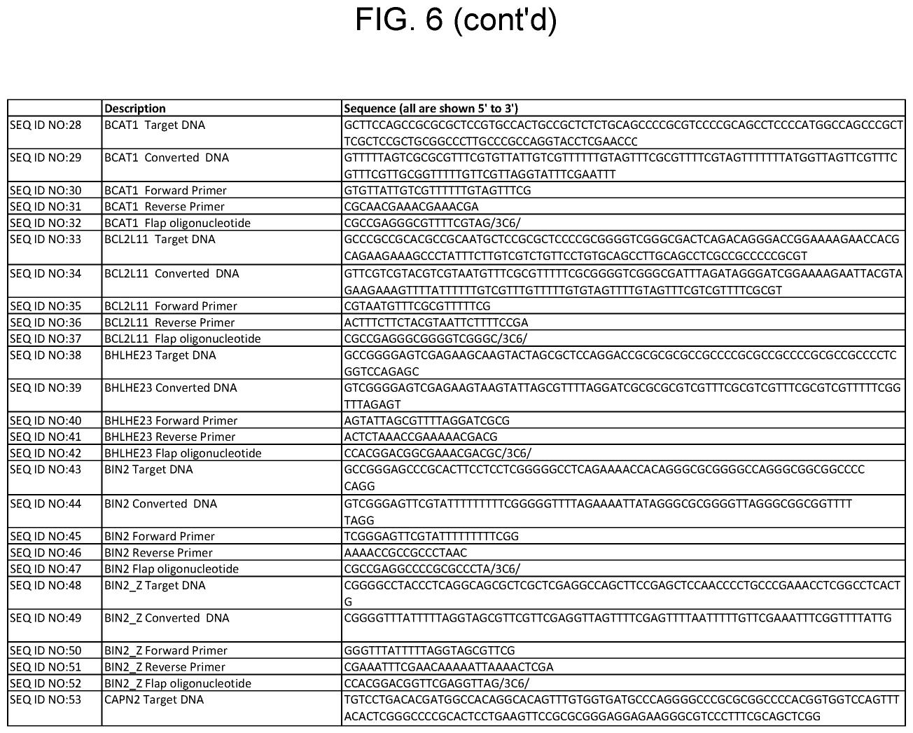

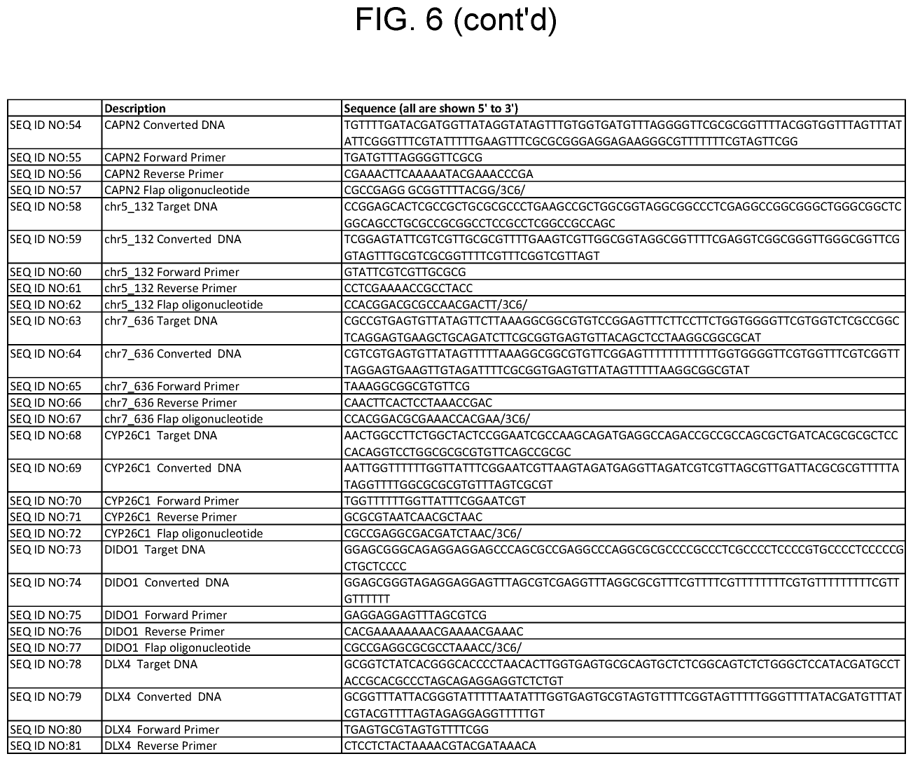

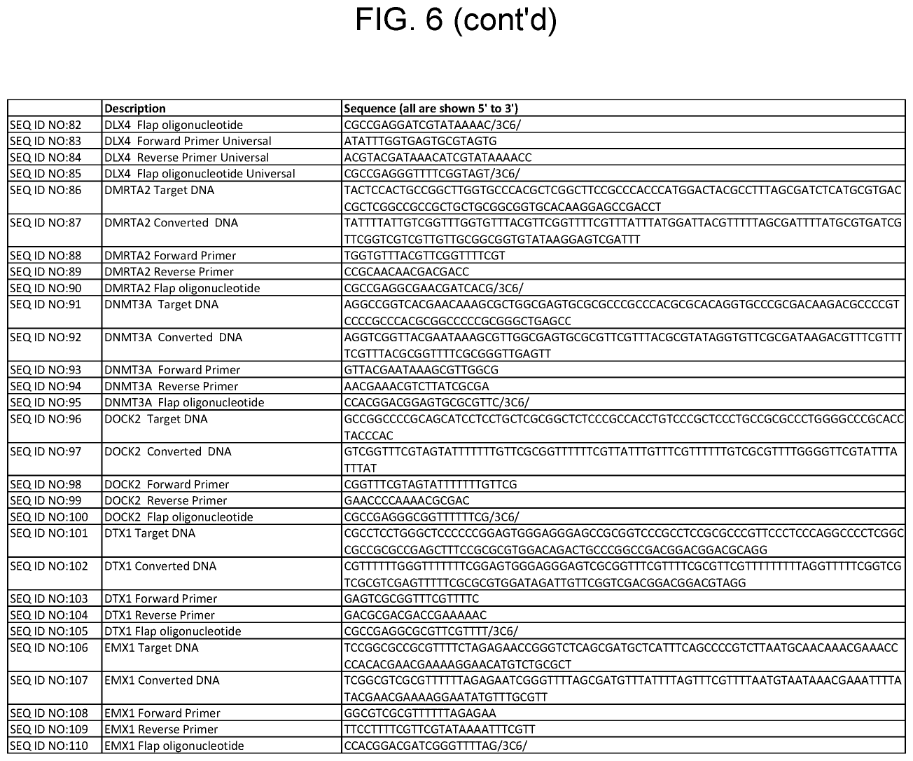

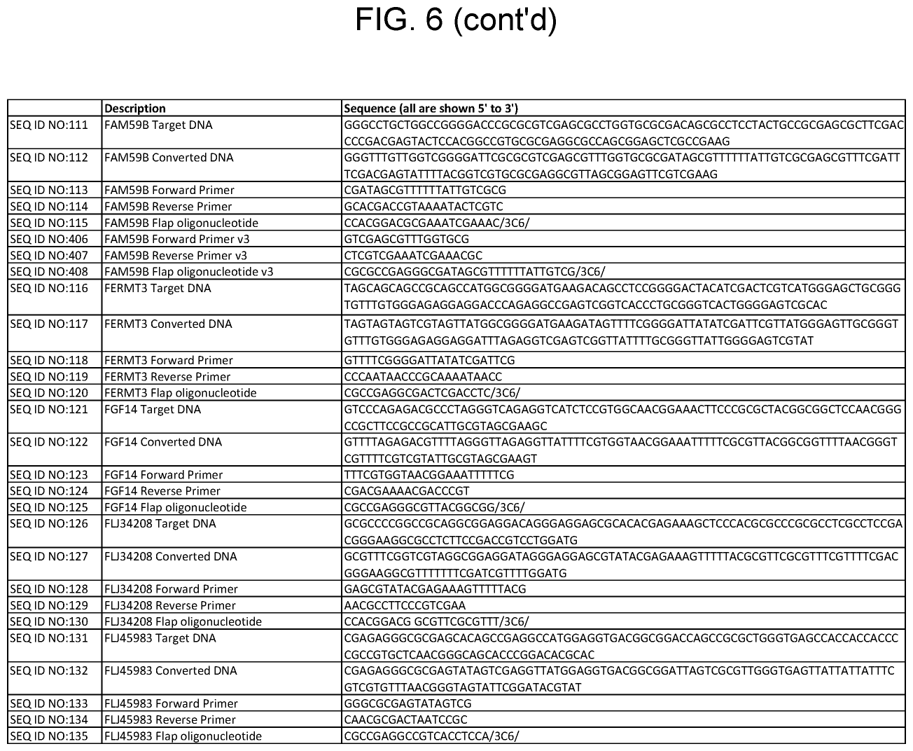

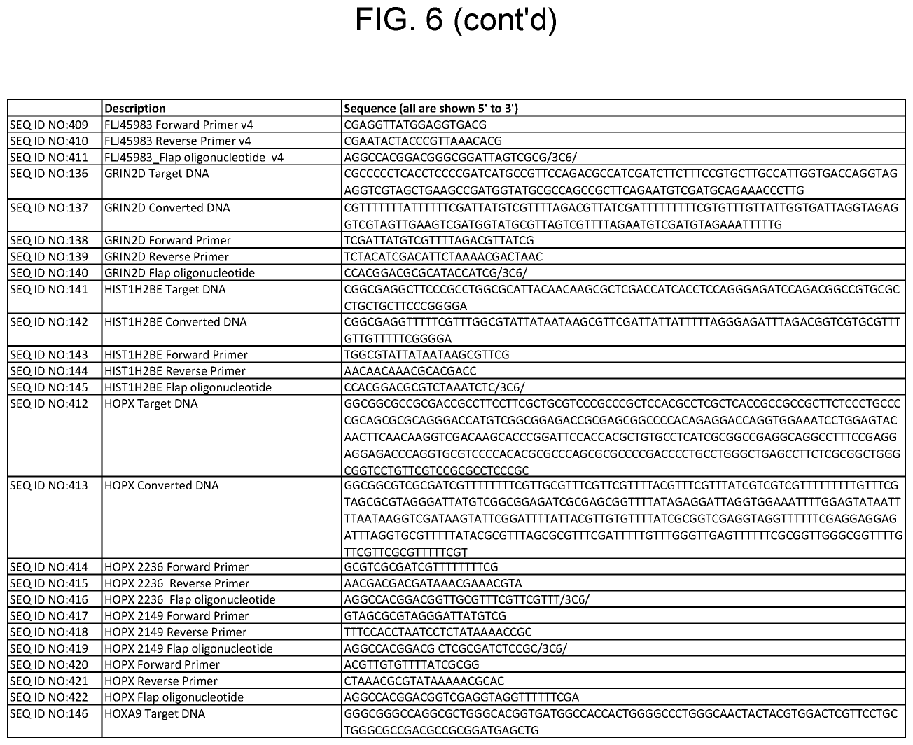

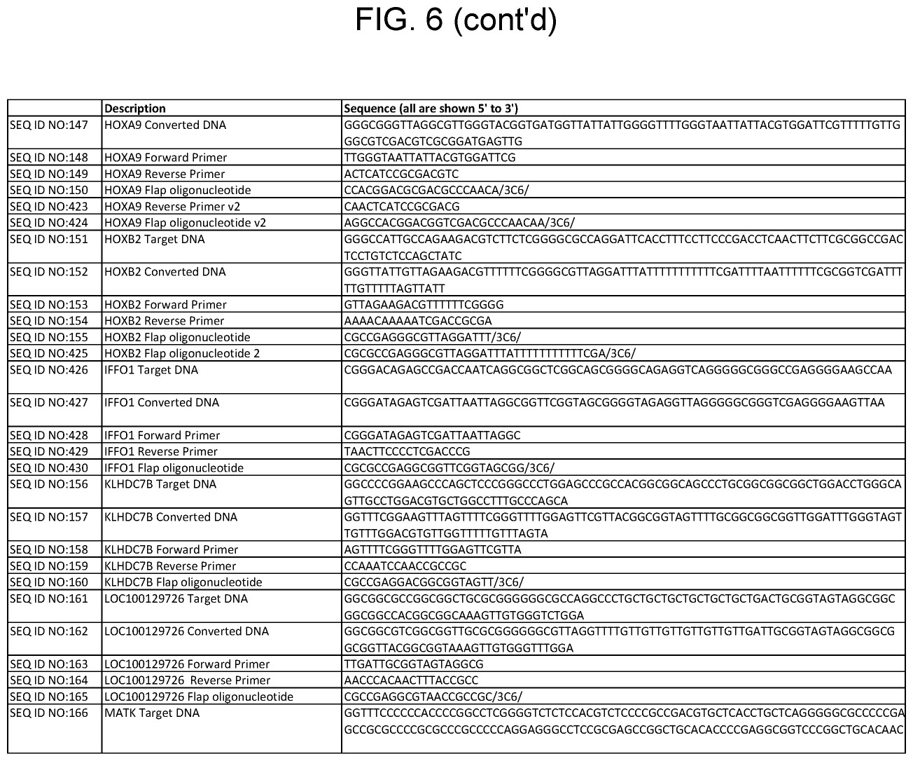

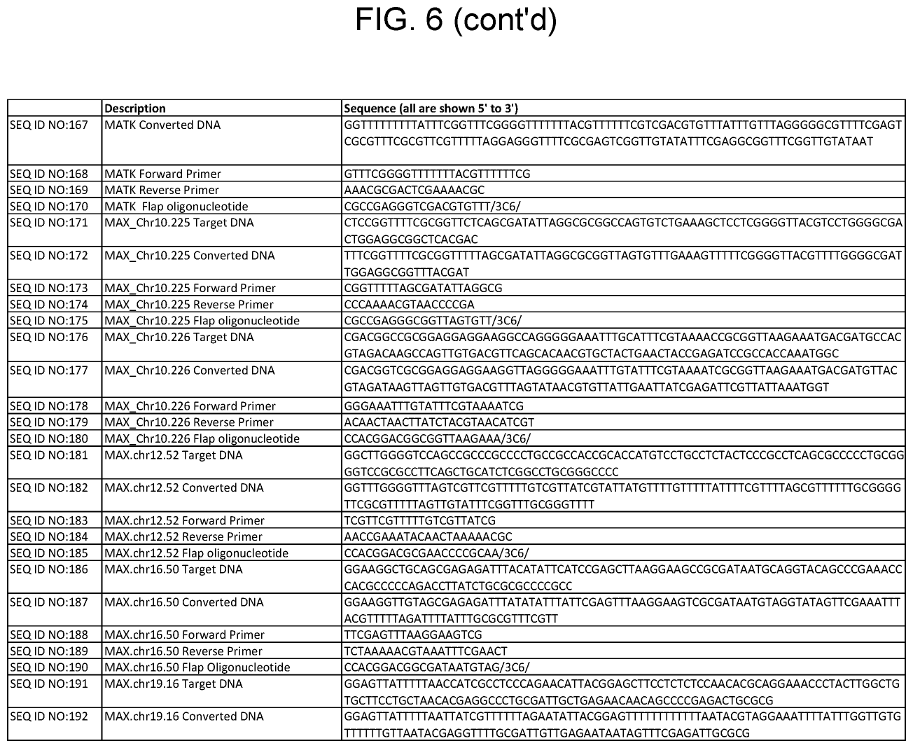

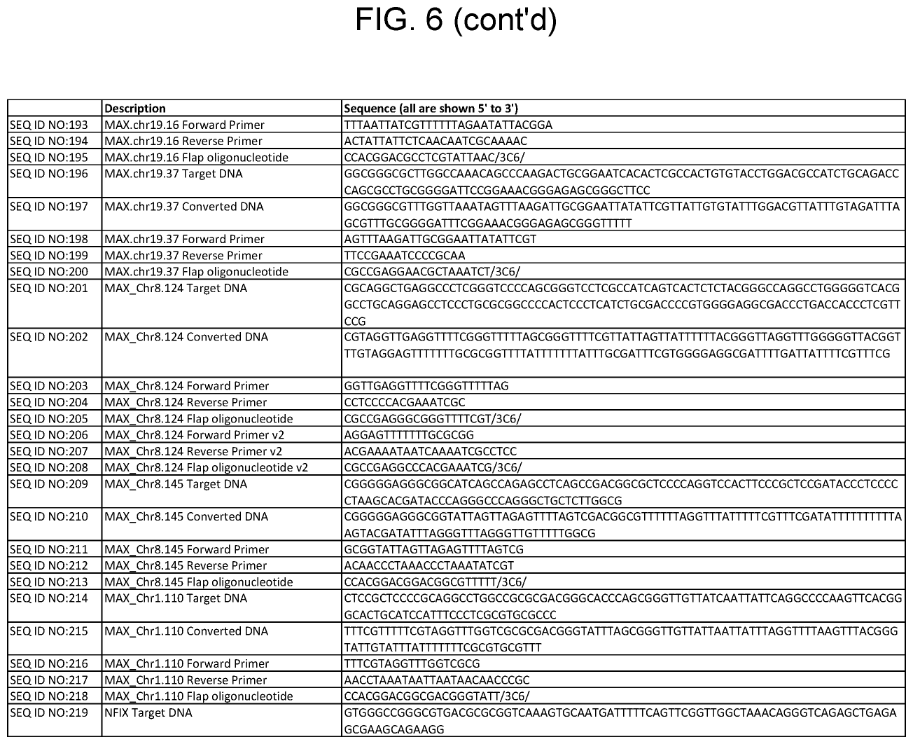

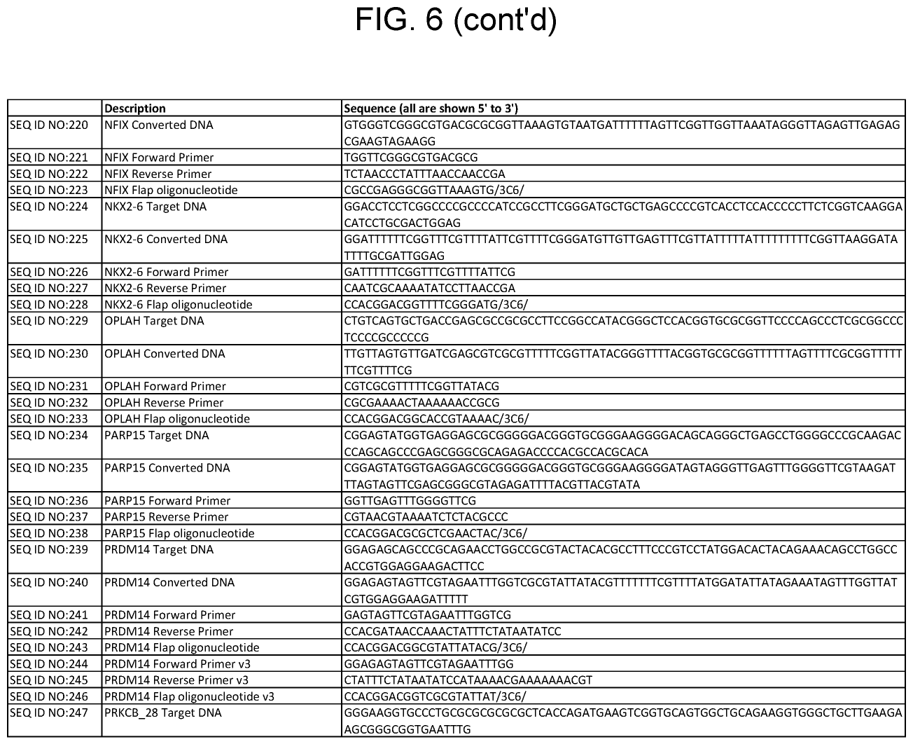

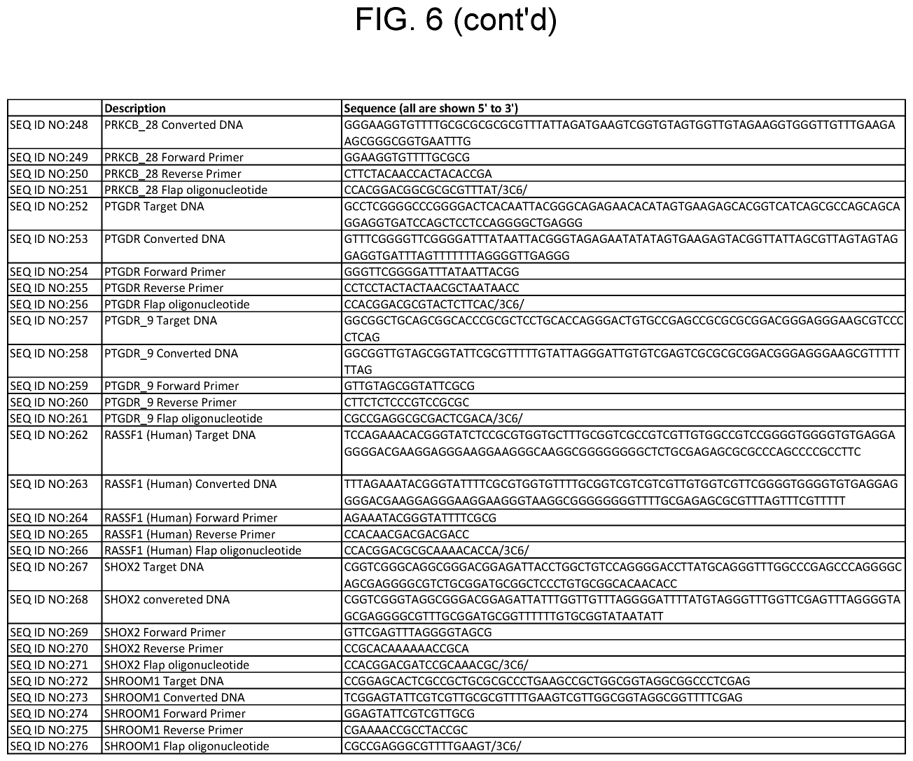

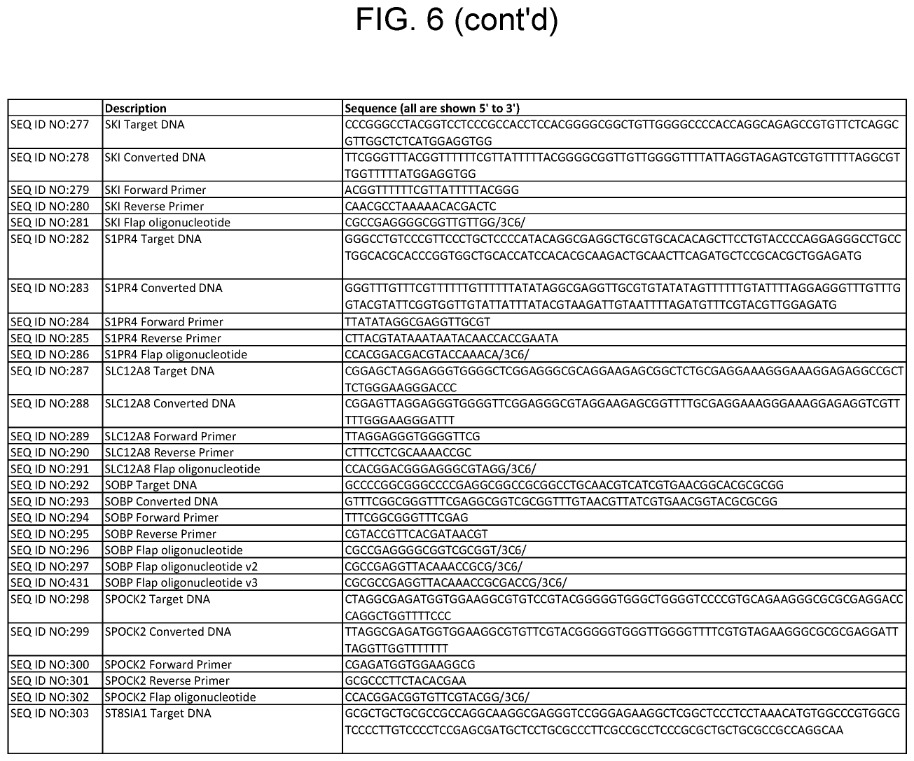

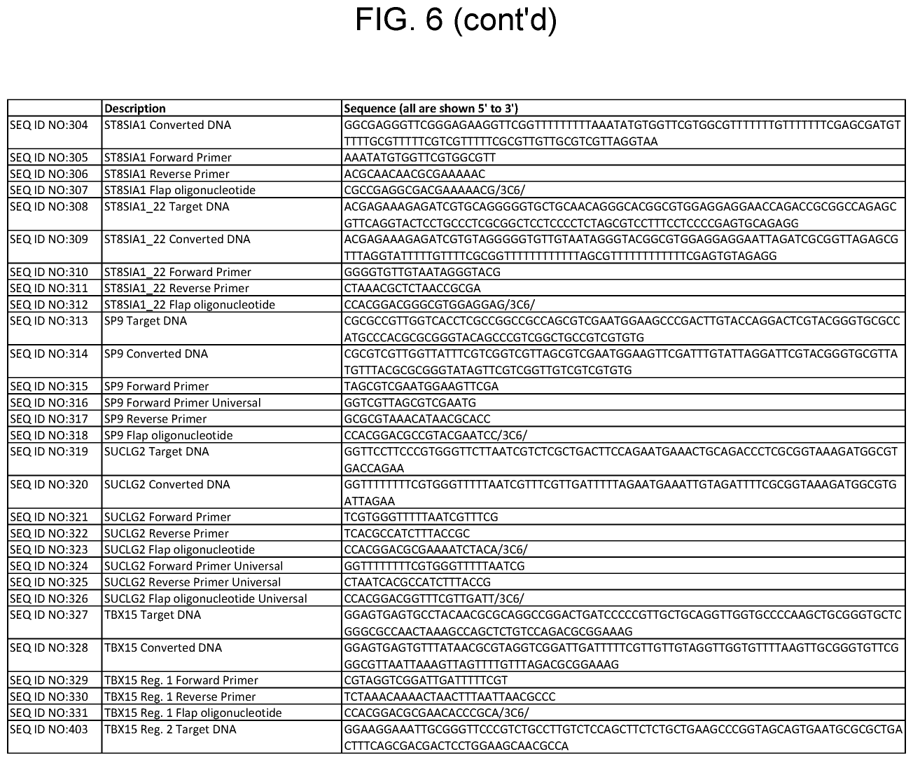

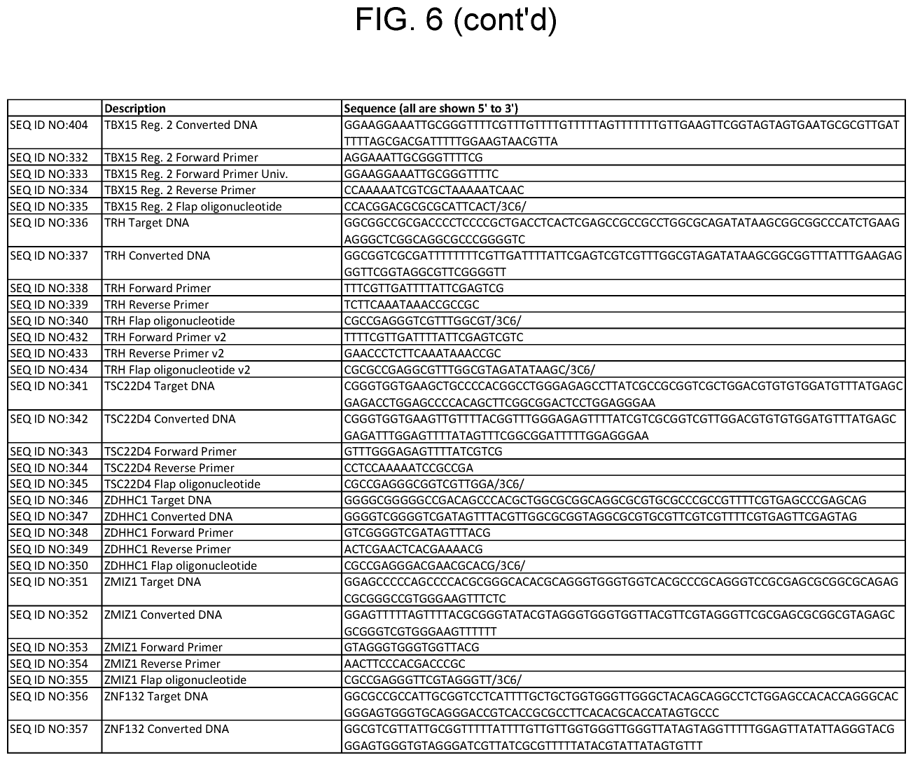

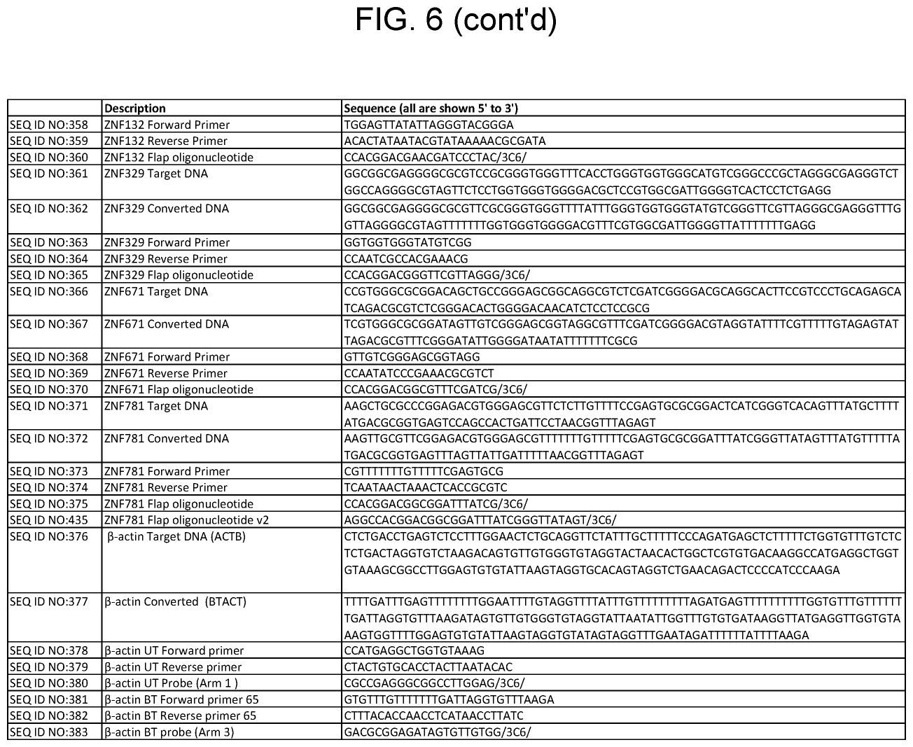

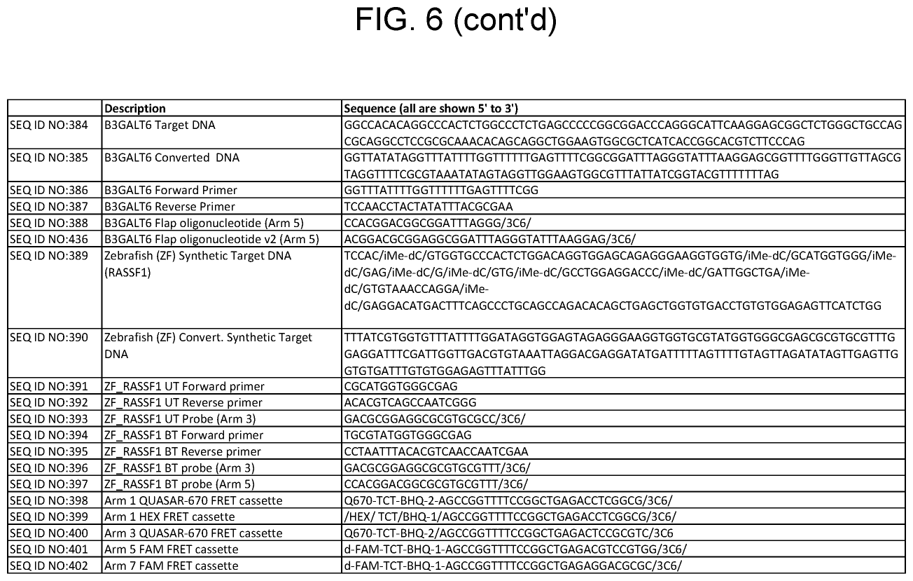

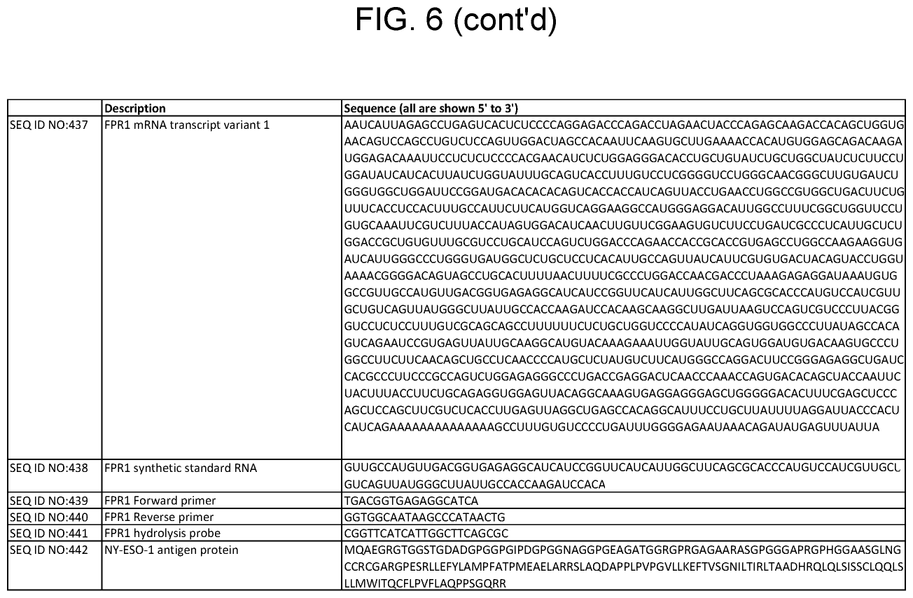

In some embodiments, a sample from a subject having or suspected of having lung cancer is screened using one or more methylation markers and suitable assay methods that provide data that differentiate between different types of lung cancer, e.g., non-small cell (adenocarcinoma, large cell carcinoma, squamous cell carcinoma) and small cell carcinomas. See, e.g., marker ref. #AC27 (FIG. 2; PLEC), which is highly methylated (shown as mean methylation compared to mean methylation at that locus in normal buffy coat) in adenocarcinoma and small cell carcinomas, but not in large cell or squamous cell carcinoma; marker ref. #AC23 (FIG. 2; ITPRIPL1), which is more highly methylated in adenocarcinoma than in any other sample type; marker ref. #LC2 (FIG. 3; DOCK2)), which is more highly methylated in large cell carcinomas than in any other sample type; marker ref #SC221 (FIG. 4; ST8SIA4), which is more highly methylated in small cell carcinomas than in any other sample type; and marker ref. #SQ36 (FIG. 5, DOK1), which is more highly methylated in squamous cell carcinoma than in than in any other sample type.

Methylation markers selected as described herein may be used alone or in combination (e.g., in panels) such that analysis of a sample from a subject reveals the presence of a lung neoplasm and also provides sufficient information to distinguish between lung cancer type, e.g., small cell carcinoma vs. non-small cell carcinoma. In preferred embodiments, a marker or combination of markers further provide data sufficient to distinguish between adenomcarcinomas, large cell carcinomas, and squamous cell carcinomas; and/or to characterize carcinomas of undetermined or mixed pathologies. In other embodiments, methylation markers or combinations thereof are selected to provide a positive result (i.e., a result indicating the presence of lung neoplasm) regardless of the type of lung carcinoma present, without differentiating data.

Over recent years, it has become apparent that circulating epithelial cells, representing metastatic tumor cells, can be detected in the blood of many patients with cancer. Molecular profiling of rare cells is important in biological and clinical studies. Applications range from characterization of circulating epithelial cells (CEpCs) in the peripheral blood of cancer patients for disease prognosis and personalized treatment (See e.g., Cristofanilli M, et al. (2004) N Engl J Med 351:781-791; Hayes D F, et al. (2006) Clin Cancer Res 12:4218-4224; Budd G T, et al., (2006) Clin Cancer Res 12:6403-6409; Moreno J G, et al. (2005) Urology 65:713-718; Pantel et al., (2008) Nat Rev 8:329-340; and Cohen S J, et al. (2008) J Clin Oncol 26:3213-3221). Accordingly, embodiments of the present disclosure provide compositions and methods for detecting the presence of metastatic cancer in a subject by identifying the presence of methylation markers in plasma or whole blood.

### EXPERIMENTAL EXAMPLES

### Example 1

### Sample Preparation Methods

### Methods for DNA Isolation and QUARTS Assay

The following provides exemplary method for DNA isolation prior to analysis, and an exemplary QUARTS assay, such as may be used in accordance with embodiments of the technology. Application of QuARTS technology to DNA from blood and various tissue samples is described in this example, but the technology is readily applied to other nucleic acid samples, as shown in other examples.

DNA Isolation from Cells and Plasma

For cell lines, genomic DNA may be isolated from cell conditioned media using, for example, the “Maxwell® RSC ccfDNA Plasma Kit (Promega Corp., Madison, Wis.). Following the kit protocol, 1 mL of cell conditioned media (CCM) is used in place of plasma, and processed according to the kit procedure. The elution volume is 100 μL, of which 70 μL are generally used for bisulfite conversion.

An exemplary procedure for isolating DNA from a 4 mL sample of plasma is as follows:
To a 4 mL sample of plasma, 300 μL of Proteinase K (20 mg/mL) is added and mixed.
Add 3 μL of 1 μg/μL of Fish DNA to the plasma-proteinase K mixture.
Add 2 mL of plasma lysis buffer to plasma.
   Plasma lysis buffer is:
      4.3M guanidine thiocyanate
      10% IGEPAL CA-630 (Octylphenoxy poly(ethyleneoxy)ethanol, branched)
      (5.3 g of IGEPAL CA-630 combined with 45 mL of 4.8 M guanidine thiocyanate)
Incubate mixtures at 55° C. for 1 hour with shaking at 500 rpm.
Add and mix:
   3 mL of plasma lysis buffer
   200 μL magnetic silica binding beads (16 μg of beads/μL)
   Add 2 mL of 100% isopropanol
(optionally mix after each addition and/or optionally pre-mix the lysis buffer and isopropanol before adding to the mixture)
Incubate at 30° C. for 30 minutes with shaking at 500 rpm.
Place tube(s) on magnet and let the beads collect. Aspirate and discard the supernatant.
Add 750 μL GuHCl-EtOH to vessel containing the binding beads and mix.
   GuHCl-EtOH wash buffer is:
      3M GuHCl (guanidine hydrochloride)
      57% EtOH (ethyl alcohol)
Shake at 400 rpm for 1 minute.
Transfer samples to a deep well plate or 2 mL microcentrifuge tubes.
Place tubes on magnet and let the beads collect for 10 minutes. Aspirate and discard the supernatant.
Add 1000 μL wash buffer (10 mM Tris HCl, 80% EtOH) to the beads, and incubate at 30° C. for 3 minutes with shaking.
Place tubes on magnet and let the beads collect. Aspirate and discard the supernatant.
Add 500 μL wash buffer to the beads and incubate at 30° C. for 3 minutes with shaking.
Place tubes on magnet and let the beads collect. Aspirate and discard the supernatant.
Add 250 μL wash buffer and incubate at 30° C. for 3 minutes with shaking.
Place tubes on magnet and let the beads collect. Aspirate and discard the remaining buffer.
Add 250 μL wash buffer and incubate at 30° C. for 3 minutes with shaking.
Place tubes on magnet and let the beads collect. Aspirate and discard the remaining buffer.
Dry the beads at 70° C. for 15 minutes, with shaking.
Add 125 μL elution buffer (10 mM Tris HCl, pH 8.0, 0.1 mM EDTA) to the beads and incubate at 65° C. for 25 minutes with shaking.
Place tubes on magnet and let the beads collect for 10 minutes.
Aspirate and transfer the supernatant containing the DNA to a new vessel or tube.

### Bisulfite Conversion

### I. Sulfonation of DNA Using Ammonium Hydrogen Sulfite

1. In each tube, combine 64 μL DNA, 7 μL 1 N NaOH, and 9 μL of carrier solution containing 0.2 mg/mL BSA and 0.25 mg/mL of fish DNA.
2. Incubate at 42° C. for 20 minutes.
3. Add 120 μL of 45% ammonium hydrogen sulfite and incubate at 66° for 75 minutes.
4. Incubate at 4° C. for 10 minutes.

### II. Desulfonation Using Magnetic Beads

### Materials

Magnetic beads (Promega MagneSil Paramagnetic Particles, Promega catalogue number AS1050, 16 μg/μL).
Binding buffer: 6.5-7 M guanidine hydrochoride.
Post-conversion Wash buffer: 80% ethanol with 10 mM Tris HCl (pH 8.0).
Desulfonation buffer: 70% isopropyl alcohol, 0.1 N NaOH was selected for the desulfonation buffer.

Samples are mixed using any appropriate device or technology to mix or incubate samples at the temperatures and mixing speeds essentially as described below. For example, a Thermomixer (Eppendorf) can be used for the mixing or incubation of samples. An exemplary desulfonation is as follows:
1. Mix bead stock thoroughly by vortexing bottle for 1 minute.
2. Aliquot 50 μL of beads into a 2.0 mL tube (e.g., from USA Scientific).
3. Add 750 μL of binding buffer to the beads.
4. Add 150 μL of sulfonated DNA from step I.
5. Mix (e.g., 1000 RPM at 30° C. for 30 minutes).
6. Place tube on the magnet stand and leave in place for 5 minutes. With the tubes on the stand, remove and discard the supernatant.
7. Add 1,000 μL of wash buffer. Mix (e.g., 1000 RPM at 30° C. for 3 minutes).
8. Place tube on the magnet stand and leave in place for 5 minutes. With the tubes on the stand, remove and discard the supernatant.
9. Add 250 μL of wash buffer. Mix (e.g., 1000 RPM at 30° C. for 3 minutes).
10. Place tube on magnetic rack; remove and discard supernatant after 1 minute.
11. Add 200 μL of desulfonation buffer. Mix (e.g., 1000 RPM at 30° C. for 5 minutes).
12. Place tube on magnetic rack; remove and discard supernatant after 1 minute.
13. Add 250 μL of wash buffer. Mix (e.g., 1000 RPM at 30° C. for 3 minutes).
14. Place tube on magnetic rack; remove and discard supernatant after 1 minute.
15. Add 250 μL of wash buffer to the tube. Mix (e.g., 1000 RPM at 30° C. for 3 minutes).
16. Place tube on magnetic rack; remove and discard supernatant after 1 minute.
17. Incubate all tubes at 30° C. with the lid open for 15 minutes.
18. Remove tube from magnetic rack and add 70 μL of elution buffer directly to the beads.
19. Incubate the beads with elution-buffer (e.g., 1000 RPM at 40° C. for 45 minutes).
20. Place tubes on magnetic rack for about one minute; remove and save the supernatant.

The converted DNA is then used in a detection assay, e.g., a pre-amplification and/or flap endonuclease assays, as described below.

See also U.S. Patent Appl. Ser. Nos. 62/249,097, filed Oct. 30, 2015; Ser. Nos. 15/335,111 and 15/335,096, both filed Oct. 26, 2016; and International Appl. Ser. No. PCT/US16/58875, filed Oct. 26, 2016, each of which is incorporated herein by reference in its entirety, for all purposes.

### QuARTS Assay

The QuARTS technology combines a polymerase-based target DNA amplification process with an invasive cleavage-based signal amplification process. The technology is described, e.g., in U.S. Pat. Nos. 8,361,720; 8,715,937; 8,916,344; and 9,212,392, and U.S. patent application Ser. No. 15/841,006, each of which is incorporated herein by reference. Fluorescence signal generated by the QuARTS reaction is monitored in a fashion similar to real-time PCR and permits quantitation of the amount of a target nucleic acid in a sample.

An exemplary QuARTS reaction typically comprises approximately 400-600 nmol/L (e.g., 500 nmol/L) of each primer and detection probe, approximately 100 nmol/L of the invasive oligonucleotide, approximately 600-700 nmol/L of each FRET cassette (FAM, e.g., as supplied commercially by Hologic, Inc.; HEX, e.g., as supplied commercially by BioSearch Technologies; and Quasar 670, e.g., as supplied commercially by BioSearch Technologies), 6.675 ng/μL FEN-1 endonuclease (e.g., Cleavase® 2.0, Hologic, Inc.), 1 unit Taq DNA polymerase in a 30 μL reaction volume (e.g., GoTaq® DNA polymerase, Promega Corp., Madison, Wis.), 10 mmol/L 3-(n-morpholino) propanesulfonic acid (MOPS), 7.5 mmol/L MgCl₂, and 250 μmol/L of each dNTP. Exemplary QuARTS cycling conditions are as shown in the table below. In some applications, analysis of the quantification cycle (C_{q}) provides a measure of the initial number of target DNA strands (e.g., copy number) in the sample.

| Stage | Temp/Time | # of Cycles |
|---|---|---|
| Denaturation | 95° C./3′ | 1 |
| Amplification 1 | 95° C./20″ | 10 |
| | 67° C./30″ | |
| | 70° C./30″ | |
| Amplification 2 | 95° C./20″ | 37 |
| | 53° C./1′ | |
| | 70° C./30″ | |
| Cooling | 40° C./30″ | 1 |

### Multiplex Targeted Pre-Amplification of Large-Volume Bisulfite-Converted DNA

To pre-amplify most or all of the bisulfite-treated DNA from an input sample, a large volume of the treated DNA may be used in a single, large-volume multiplex amplification reaction. For example, DNA is extracted from a cell lines (e.g., DFCI032 cell line (adenocarcinoma); H1755 cell line (neuroendocrine), using, for example, the Maxwell Promega blood kit #AS1400, as described above. The DNA is bisulfite converted, e.g., as described above.

A pre-amplification is conducted, for example, in a reaction mixture containing 7.5 mM MgCl₂, 10 mM MOPS, 0.3 mM Tris-HCl, pH 8.0, 0.8 mM KCl, 0.1 μg/μL BSA, 0.0001% Tween-20, 0.0001% IGEPAL CA-630, 250 μM each dNTP, oligonucleotide primers, (e.g., for 12 targets, 12 primer pairs/24 primers, in equimolar amounts (including but not limited to the ranges of, e.g., 200-500 nM each primer), or with individual primer concentrations adjusted to balance amplification efficiencies of the different target regions), 0.025 units/μL HotStart GoTaq concentration, and 20 to 50% by volume of bisulfite-treated target DNA (e.g., 10 μL of target DNA into a 50 μL reaction mixture, or 50 μL of target DNA into a 125 μL reaction mixture). Thermal cycling times and temperatures are selected to be appropriate for the volume of the reaction and the amplification vessel. For example, the reactions may be cycled as follows

| Stage | Temp/Time | #of Cycles |
|---|---|---|
| Pre-incubation | 95° C./5′ | 1 |
| Amplification 1 | 95° C./30″ | 10-12 |
| | 64° C./30″ | |
| | 72° C./30″ | |
| Cooling | 4° C./Hold | 1 |

After thermal cycling, aliquots of the pre-amplification reaction (e.g., 10 L) are diluted to 500 μL in 10 mM Tris, 0.1 mM EDTA, with or without fish DNA. Aliquots of the diluted pre-amplified DNA (e.g., 10 μL) are used in a QuARTS PCR-flap assay, e.g., as described above. See also U.S. Patent Appl. Ser. No. 62/249,097, filed Oct. 30, 2015; application Ser. No. 15/335,096, filed Oct. 26, 2016, and PCT/US16/58875, filed Oct. 26, 2016, each of which is incorporated herein by reference in its entirety for all purposes.

### Example 2

### Selection and Testing of Methylation Markers

### Marker Selection Process:

Reduced Representation Bisulfite Sequencing (RRBS) data was obtained on tissues from 16 adenocarcinoma lung cancer, 11 large cell lung cancer, 14 small cell lung cancer, 24 squamous cell lung cancer, and 18 non-cancer lung as well as RRBS results of buffy coat samples obtained from 26 healthy patients.

After alignment to a bisulfite-converted form of the human genome sequence, average methylation at each CpG island was computed for each sample type (i.e., tissue or buffy coat) and marker regions were selected based on the following criteria:
Regions were selected to be 50 base pairs or longer.
For QuARTS flap assay designs, regions were selected to have a minimum of 1 methylated CpG under each of: a) the probe region, b) the forward primer binding region, and c) the reverse primer binding region. For the forward and reverse primers, it is preferred that the methylated CpGs are close to the 3′-ends of the primers, but not at the 3′terminal nucleotide. Exemplary flap endonuclease assay oligonucleotides are shown in FIG. 1.
Preferably, buffy coat methylation at any CpG in a region of interest is no more than >0.5%.
Preferably, cancer tissue methylation in a region of interest is >10%.
For assays designed for tissue analysis, normal tissue methylation in a region of interest is preferably <0.5%.

RRBS data for different lung cancer tissue types is shown in FIGS. 2-5. Based on the criteria above, the markers shown in the table below were selected and QuARTS flap assays were designed for them, as shown in FIG. 1.

TABLE 1

| Marker Name | Genomic coordinates |
|---|---|
| AGRN | chr1:968467-968582, strand=+ |
| ANGPT1 | chr8:108509559-108509684, strand=− |
| ANKRD13B | chr17:27940470-27940578, strand=+ |
| ARHGEF4 | chr2:131792758-131792900, strand=− |
| B3GALT6 | chr1: 1163595-1163733, strand=+ |
| BARX1 | chr9:96721498-96721597, strand=− |
| BCAT1 | chr12:25055868-25055986, strand=− |
| BCL2L11 | chr2: 111876620-111876759, strand=− |
| BHLHE23 | chr20:61638462-61638546, strand=− |
| BIN2 | chr12:51717898-51717971, strand=− |
| BIN2_Z | chr12:51718088-51718165, strand=+ |
| CAPN2 | chr1:223936858-223936998, strand=+ |
| chr17_737 | chr17:73749814-73749919, strand=− |
| chr5_132 | chr5:132161371-132161482,Strand=+ |
| chr7_636 | chr7:104581684-104581817, Strand=− |
| CYP26C1 | chr10: 94822396-94822502, strand=+ |
| DIDO1 | chr20:61560669-61560753, strand=− |
| DLX4 | chr17:48042426-48042820, strand=− |
| DMRTA2 | chr1:50884390-50884519, strand=− |
| DNMT3A | chr2:25499967-25500072, strand=− |
| DOCK2 | chr5:169064370- 169064454, strand=− |
| EMX1 | chr2: 73147685-73147792, strand=+ |
| FAM59B | chr2:26407701-26407828, strand=+ |
| FERMT3 | chr11:63974820-63974959, strand=+ |
| FGF14 | chr13:103046888-103046991, strand=+ |
| FLJ34208 | chr3:194208249-194208355, strand=+ |
| FLJ45983 | chr10:8097592-8097699, strand=+ |
| GRIN2D | chr19:48918160-48918300, strand=− |
| HIST1H2BE | chr6:26184248-26184340, strand=+ |
| HOPX | chr4:57521932-57522261 5′pad=0 |
| | 3′pad=0 strand=− |
| IFFO1 | chr12:6665277-6665348 strand=+ |
| HOXA9 | chr7:27205002-27205102, strand=− |
| HOXB2 | chr17:46620545-46620639, strand=− |
| KLHDC7B | chr22: 50987199-50987256, strand=+ |
| LOC100129726 | chr2:43451705-43451810, strand=+ |
| MATK | chr19:3786127-3786197, strand=+ |
| MAX.chr10.22541891- | chr10:22541881-22541975, strand=+ |
| 22541946 | |
| MAX.chr10.22624430- | chr10:22624411-22624553, strand=− |
| 22624544 | |
| MAX.chr12.52652268- | chr12:52652262-52652377, strand=− |
| 52652362 | |
| MAX.chr16.50875223- | chr16:50875167-50875274, strand=− |
| 50875241 | |
| MAX.chr19.16394489- | chr19:16394457-16394593, strand=− |
| 16394575 | |
| MAX.chr19.37288426- | range=chr19:37288396-37288512, |
| 37288480 | strand=− |
| MAX.chr8.124173236- | chr8:124173231-124173386, strand=− |
| 124173370 | |
| MAX.chr8.145105646- | chr8:145105572-145105685, strand=− |
| 145105653 | |
| MAX_Chr1.110 | chr1:110627118-110627224 strand=− |
| NFIX | chr19:13207426-13207513, strand=+ |
| NKX2-6 | chr8:23564052-23564145, strand=− |
| OPLAH | chr8:145106777-145106865, strand=− |
| PARP15 | chr3:122296692-122296805, strand=+ |
| PRDM14 | chr8:70981945-70982039, strand=− |
| PRKAR1B | chr7:644172-644237, strand=+ |
| PRKCB_28 | chr16:23847607-23847698, strand=− |
| PTGDR | chr14:52735270-52735400, strand=− |
| PTGDR_9 | chr14:52735221-52735300, strand=+ |
| RASSF1 | chr3:50378408-50378550, strand=− |
| SHOX2 | chr3:157821263-157821382, strand=− |
| SHROOM1 | chr5:132161371-132161425, strand=+ |
| SIPR4 | chr19:3179921-3180068 strand=− |
| SKI | chr1:2232328-2232423, strand=+ |
| SLC12A8 | chr3:124860704-124860791, strand=+ |
| SOBP | chr6: 107956176-107956234, strand=+ |
| SP9 | chr2:175201210-175201341, strand=− |
| SPOCK2 | chr10:73847236-73847324, strand=− |
| ST8SIA1 | chr12:22487518-22487630, strand=+ |
| ST8SIA1_22 | chr12:22486873-22487009, strand=− |
| SUCLG2 | chr3:67706477-677065610, strand=− |
| TBX15 Region 1 | chr1:119527066-119527655, strand=+ |
| TBX15 Region 2 | chr1:119532813-119532920 strand=− |
| TRH | chr3:129693481-129693580, strand=+ |
| TSC22D4 | chr7:100075328-100075445, strand=− |
| ZDHHC1 | chr16:67428559-67428628, strand=− |
| ZMIZ1 | chr10:81002910-81003005, strand=+ |
| ZNF132 | chr19:58951403-58951529, strand=− |
| ZNF329 | chr19: 58661889- 58662028, strand=− |
| ZNF671 | chr19:58238790-58238906, strand=+ |
| ZNF781 | ch19 : 38183018-38183137, strand=− |

Analyzing Selected Markers for Cross-Reactivity with Buffy Coat.

### 1) Buffy Coat Screening

Markers from the list above were screened on DNA extracted from buffy coat obtained from 10 mL blood of a healthy patient DNA was extracted using Promega Maxwell RSC system (Promega Corp., Fitchburg, Wis.) and converted using Zymo EZ DNA Methylation™ Kit (Zymo Research, Irvine, Calif.). Using biplexed reaction with bisulfite-converted β-actin DNA (“BTACT”), and using approximately 40,000 strands of target genomic DNA, the samples were tested using a QuARTS flap endonuclease assay as described above, to test for cross reactivity. Doing so, the assays for 3 markers showed significant cross reactivity:

| | % Cross |
|---|---|
| Marker | reactivity |
| HIST1H2B | 72.93% |
| chr7_636 | 3495.47% |
| chr5_132 | 0.20% |

### 2) Tissue Screening

264 tissue samples were obtained from various commercial and non-commercial sources (Asuragen, BioServe, ConversantBio, Cureline, Mayo Clinic, M D Anderson, and PrecisionMed), as shown below in Table 2.

| No. of cases | Pathology | Subtype | Details |
|---|---|---|---|
| 82 | Normal | NA | 68 smokers, 34 |
| 37 | Normal | benign nodule | never smokers, 17 |
| | | | smoking unknown |
| 7 | NSCLC | bronchioalveolar | |
| 13 | NSCLC | large cell | |
| 2 | NSCLC | neuroendocrine | |
| 42 | NSCLC | squamous cell | |
| 68 | NSCLC | adenocarcinomas | |
| 4 | SCLC | small cell | |
| 9 | NSCLC | carcinoid | |

Tissue sections were examined by a pathologist, who circled histologically distinct lesions to direct the micro-dissection. Total nucleic acid extraction was performed using the Promega Maxwell RSC system. Formalin-fixed, paraffin-embedded (FFPE) slides were scraped and the DNA was extracted using the Maxwell® RSC DNA FFPE Kit (#AS1450) using the manufacturer's procedure but skipping the RNase treatment step. The same procedure was used for FFPE curls. For frozen punch biopsy samples, a modified procedure using the lysis buffer from the RSC DNA FFPE kit with the Maxwell® RSC Blood DNA kit (#AS1400) was utilized omitting the RNase step. Samples were eluted in 10 mM Tris, 0.1 mM EDTA, pH 8.5 and 10 uL were used to setup 6 multiplex PCR reactions.

The following multiplex PCR primer mixes were made at 10× concentration (10×=2 μM each primer):
Multiplex PCR reaction 1 consisted of each of the following markers: BARX1, LOC100129726, SPOCK2, TSC22D4, PARP15, MAX.chr8.145105646-145105653, ST8SIA1_22, ZDHHC1, BIN2_Z, SKI, DNMT3A, BCL2L11, RASSF1, FERMT3, and BTACT.
Multiplex PCR reaction 2 consisted of each of the following markers: ZNF671, ST8SIA1, NKX6-2, SLC12A8, FAM59B, DIDO1, MAX_Chr1.110, AGRN, PRKCB_28, SOBP, and BTACT.
Multiplex PCR reaction 3 consisted of each of the following markers: MAX.chr10.22624430-22624544, ZMIZ1, MAX.chr8.145105646-145105653, MAX.chr10.22541891-22541946, PRDM14, ANGPT1, MAX.chr16.50875223-50875241, PTGDR 9, ANKRD13B, DOCK2, and BTACT.
Multiplex PCR reaction 4 consisted of each of the following markers: MAX.chr19.16394489-16394575, HOXB2, ZNF132, MAX.chr19.37288426-37288480, MAX.chr12.52652268-52652362, FLJ45983, HOXA9, TRH, SP9, DMRTA2, and BTACT.
Multiplex PCR reaction 5 consisted of each of the following markers: EMX1, ARHGEF4, OPLAH, CYP26C1, ZNF781, DLX4, PTGDR, KLHDC7B, GRIN2D, chr17_737, and BTACT.
Multiplex PCR reaction 6 consisted of each of the following markers: TBX15, MATK, SHOX2, BCAT1, SUCLG2, BIN2, PRKAR1B, SHROOM1, S1PR4, NFIX, and BTACT.

Each multiplex PCR reaction was setup to a final concentration of 0.2 μM reaction buffer, 0.2 μM each primer, 0.05 μM Hotstart Go Taq (5 U/μL), resulting in 40 μL of master mix that was combined with 10 μL of DNA template for a final reaction volume of 50 μL.

The thermal profile for the multiplex PCR entailed a pre-incubation stage of 95° for 5 minutes, 10 cycles of amplification at 95° for 30 seconds, 64° for 30 seconds, 72° for 30 seconds, and a cooling stage of 4° that was held until further processing. Once the multiplex PCR was complete, the PCR product was diluted 1:10 using a diluent of 20 ng/μL of fish DNA (e.g., in water or buffer, see U.S. Pat. No. 9,212,392, incorporated herein by reference) and 10 μL of diluted amplified sample were used for each QuARTS assay reaction.

Each QuARTS assay was configured in triplex form, consisting of 2 methylation markers and BTACT as the reference gene.
From multiplex PCR product 1, the following 7 triplex QuARTS assays were run: (1) BARX1, LOC100129726, BTACT; (2) SPOCK2, TSC22D4, BTACT; (3) PARP15, MAXchr8145105646-145105653, BTACT; (4) ST8SIA1_22, ZDHHC1, BTACT; (5) BIN2_Z, SKI, BTACT; (6) DNMT3A, BCL2L11, BTACT; (7) RASSF1, FERMT3, and BTACT.
From multiplex PCR product 2, the following 5 triplex QuARTS assays were run: (1) ZNF671, ST8SIA1, BTACT; (2) NKX6-2, SLC12A8, BTACT; (3) FAM59B, DIDO1, BTACT; (4) MAX_Chr1110, AGRN, BTACT; (5) PRKCB_28, SOBP, and BTACT.
From multiplex PCR product 3, the following 5 triplex QuARTS assays were run: (1) MAXchr1022624430-22624544, ZMIZ1, BTACT; (2) MAXchr8145105646-145105653, MAXchr1022541891-22541946, BTACT; (3) PRDM14, ANGPT1, BTACT; (4) MAXchr1650875223-50875241, PTGDR_9, BTACT; (5) ANKRD13B, DOCK2, and BTACT.
From multiplex PCR product 4, the following 5 triplex QuARTS assays were run: (1) MAXchr1916394489-16394575, HOXB2, BTACT; (2) ZNF132, MAXchr1937288426-37288480, BTACT; (3) MAXchr1252652268-52652362, FLJ45983, BTACT; (4) HOXA9, TRH, BTACT; (5) SP9, DMRTA2, and BTACT.
From multiplex PCR product 5, the following 5 triplex QuARTS assays were run: (1) EMX1, ARHGEF4, BTACT; (2) OPLAH, CYP26C1, BTACT; (3) ZNF781, DLX4, BTACT; (4) PTGDR, KLHDC7B, BTACT; (5) GRIN2D, chr17_737, and BTACT.
From multiplex PCR product 6, the following 5 triplex QuARTS assays were run: (1) TBX15, MATK, BTACT; (2) SHOX2, BCAT1, BTACT; (3) SUCLG2, BIN2, BTACT; (4) PRKAR1B, SHROOM1, BTACT; (5) SIPR4, NFIX, and BTACT.

### 3) Data Analysis:

For tissue data analysis, markers that were selected based on RRBS criteria with <0.5% methylation in normal tissue and >10% methylation in cancer tissue were included. This resulted in 51 markers for further analysis.

To determine marker sensitivities, the following was performed:
1. % methylation for each marker was computed by dividing strand values obtained for that specific marker by the strand values of ACTB (β-actin).
2. The maximum % methylation for each marker was determined on normal tissue. This is defined as 100% specificity.
3. The cancer tissue positivity for each marker was determined as the number of cancer tissues that had greater than the maximum normal tissue % methylation for that marker.

The sensitivities for the 51 markers are shown below.

TABLE 2

| | Maximum % | | | |
|---|---|---|---|---|
| | methylation | Cancer (N = 136) | | |
| Marker | for normal | # Negative | # Positive | sensitivity |
| BARX1 | 1.665 | 66 | 70 | 51% |
| LOC100129726 | 1.847 | 109 | 27 | 20% |
| SPOCK2 | 0.261 | 86 | 50 | 37% |
| TSC22D4 | 0.618 | 70 | 66 | 49% |
| MAX.chr8.124 | 0.293 | 45 | 91 | 67% |
| RASSF1 | 1.605 | 79 | 57 | 42% |
| ZNF671 | 0.441 | 73 | 63 | 46% |
| ST8SIA1 | 1.56 | 119 | 17 | 13% |
| NKX6_2 | 15.58 | 102 | 34 | 25% |
| FAM59B | 0.433 | 85 | 51 | 38% |
| DIDO1 | 2.29 | 93 | 43 | 32% |
| MAX_Chr1.110 | 0.076 | 85 | 51 | 38% |
| AGRN | 2.16 | 66 | 70 | 51% |
| SOBP | 38.5 | 110 | 26 | 19% |
| MAX_chr10.226 | 0.7 | 52 | 84 | 62% |
| ZMIZ1 | 0.025 | 72 | 64 | 47% |
| MAX_chr8.145 | 5.56 | 57 | 79 | 58% |
| MAX_chr10.225 | 0.77 | 72 | 64 | 47% |
| PRDM14 | 0.22 | 35 | 101 | 74% |
| ANGPT1 | 1.6 | 99 | 37 | 27% |
| MAX.chr16.50 | 0.27 | 92 | 44 | 32% |
| PTGDR_9 | 4.62 | 82 | 54 | 40% |
| ANKRD13B | 7.03 | 93 | 43 | 32% |
| DOCK2 | 0.001 | 71 | 65 | 48% |
| MAX_chr19.163 | 0.61 | 56 | 80 | 59% |
| ZNF132 | 1.3 | 83 | 53 | 39% |
| MAX chr19.372 | 0.676 | 79 | 57 | 42% |
| HOXA9 | 16.7 | 53 | 83 | 61% |
| TRH | 2.64 | 61 | 75 | 55% |
| SP9 | 14.99 | 75 | 61 | 45% |
| DMRTA2 | 7.9 | 55 | 81 | 60% |
| ARHGEF4 | 7.41 | 113 | 23 | 17% |
| CYP26C1 | 39.2 | 101 | 35 | 26% |
| ZNF781 | 5.28 | 44 | 92 | 68% |
| PTGDR | 6.13 | 76 | 60 | 44% |
| GRIN2D | 16.1 | 113 | 23 | 17% |
| MATK | 0.04 | 93 | 43 | 32% |
| BCAT1 | 0.64 | 75 | 61 | 45% |
| PRKCB_28 | 1.68 | 57 | 79 | 58% |
| ST8SIA_22 | 1.934 | 55 | 81 | 60% |
| FLJ45983 | 8.34 | 39 | 97 | 71% |
| DLX4 | 15.1 | 41 | 95 | 70% |
| SHOX2 | 7.48 | 32 | 104 | 76% |
| EMX1 | 11.34 | 34 | 102 | 75% |
| HOXB2 | 0.114 | 61 | 75 | 55% |
| MAX.chr12.526 | 5.58 | 34 | 102 | 75% |
| BCL2L11 | 10.7 | 44 | 92 | 68% |
| OPLAH | 5.11 | 29 | 107 | 79% |
| PARP15 | 3.077 | 42 | 94 | 69% |
| KLHDC7B | 8.86 | 38 | 98 | 72% |
| SLC12A8 | 0.883 | 34 | 102 | 75% |

Combinations of markers may be used to increase specificity and sensitivity. For example, a combination of the 8 markers SLC12A8, KLHDC7B, PARP15, OPLAH, BCL2L11, MAX.chr12.26, HOXB2, and EMX1 resulted in 98.5% sensitivity (134/136 cancers) for all of the cancer tissues tested, with 100% specificity.

In some embodiments, markers are selected for sensitive and specific detection associated with a particular type of lung cancer tissue, e.g., adenocarcinoma, large cell carcinoma, squamous cell carcinoma, or small cell carcinoma, e.g., by use of markers that show sensitivity and specificity for particular cancer types or combinations of types.

This panel of methylated DNA markers assayed on tissue achieves extremely high discrimination for all types of lung cancer while remaining negative in normal lung tissue and benign nodules. Assays for this panel of markers can be also be applied to blood or bodily fluid-based testing, and finds applications in, e.g., lung cancer screening and discrimination of malignant from benign nodules.

### Example 3

### Testing a 30-Marker Set on Plasma Samples

From the list of markers in Example 2, 30 markers were selected for use in testing DNA from plasma samples from 295 subjects (64 with lung cancer, 231 normal controls. DNA was extracted from 2 mL of plasma from each subject and treated with bisulfite as described in Example 1. Aliquots of the bisulfite-converted DNA were used in two multiplex QuARTS assays, as described in Example 1. The markers selected for analysis are:
1. BARX1
2. BCL2L11
3. BIN2_Z
4. CYP26C1
5. DLX4
6. DMRTA2
7. DNMT3A
8. EMX1
9. FERMT3
10. FLJ45983
11. HOXA9
12. KLHDC7B
13. MAX.chr10.22624430-22624544
14. MAX.chr12.52652268-52652362
15. MAX.chr8.124173236-124173370
16. MAX.chr8.145105646-145105653
17. NFIX
18. OPLAH
19. PARP15
20. PRKCB_28
21. S1PR4
22. SHOX2
23. SKI
24. SLC12A8
25. SOBP
26. SP9
27. SUCLG2
28. TBX15
29. ZDHHC1
30. ZNF781

The target sequences, bisulfite converted target sequences, and the assay oligonucleotides for these markers were as shown in FIG. 1. The primers and flap oligonucleotides (probes) used for each converted target were as follows:

TABLE 3

| | Oligonucleotide | | | SEQ ID |
|---|---|---|---|---|
| Marker | Name | Component | Sequence (5′-3′) | NO: |
| BARX1 | BARX1_FP | Forward | CGTTAATTTGTTAGATAGAGGGCG | 23 |
| | | Primer | | |
| | BARX1_RP | Reverse | ACGATCGTCCGAACAACC | 24 |
| | | Primer | | |
| | BARX1_PB_A5 | Flap Oligo. | CCACGGACGCGCCTACGAAAA/3C6/ | 25 |
| SLC12A8 | SLC12A8_FP | Forward | TTAGGAGGGTGGGGTTCG | 289 |
| | | Primer | | |
| | SLC12A8_RP | Reverse | CTTTCCTCGCAAAACCGC | 290 |
| | | Primer | | |
| | SLC12A8_Pb_A1 | Flap Oligo. | CCACGGACGGGAGGGCGTAGG/3C6/ | 291 |
| PARP15 | PARP15_FP | Forward | GGTTGAGTTTGGGGTTCG | 236 |
| | | Primer | | |
| | PARP15_RP | Reverse | CGTAACGTAAAATCTCTACGCCC | 237 |
| | | Primer | | |
| | PARP15_Pb_A5 | Flap Oligo. | CCACGGACGCGCTCGAACTAC/3C6/ | 238 |
| MAX.Chr8. | MAX.Chr8.124_ | Forward | GGTTGAGGTTTTCGGGTTTTTAG | 203 |
| 124 | FP | Primer | | |
| | MAX.Chr8.124_ | Reverse | CCTCCCCACGAAATCGC | 204 |
| | RP | Primer | | |
| | MAX.Chr8.124_ | Flap Oligo. | CGCCGAGGGCGGGTTTTCGT/3C6/ | 205 |
| | Pb_A1 | | | |
| SHOX2 | SHOX2_FP | Forward | GTTCGAGTTTAGGGGTAGCG | 269 |
| | | Primer | | |
| | SHOX2_RP | Reverse | CCGCACAAAAAACCGCA | 270 |
| | | Primer | | |
| | SHOX2_Pb_A5 | Flap Oligo. | CCACGGACGATCCGCAAACGC/3C6/ | 271 |
| ZDHHC1 | ZDHHC1FP | Forward | GTCGGGGTCGATAGTTTACG | 348 |
| | | Primer | | |
| | ZDHHC1RP_V3 | Reverse | ACTCGAACTCACGAAAACG | 349 |
| | | Primer | | |
| | ZDHHC1Probe_v3_ | Flap Oligo. | CGCCGAGGGACGAACGCACG/3C6/ | 350 |
| | A1 | | | |
| BIN2_Z | BIN2_FP_Z | Forward | GGGTTTATTTTTAGGTAGCGTTCG | 50 |
| | | Primer | | |
| | BIN2_RP_Z | Reverse | CGAAATTTCGAACAAAAATTAAAACTCGA | 51 |
| | | Primer | | |
| | BIN2_Pb_A5_Z | Flap Oligo. | CCACGGACGGTTCGAGGTTAG/3C6/ | 52 |
| SKI | SKI_FP | Forward | ACGGTTTTTTCGTTATTTTTACGGG | 279 |
| | | Primer | | |
| | SKI_RP | Reverse | CAACGCCTAAAAACACGACTC | 280 |
| | | Primer | | |
| | SKI_Pb_A1 | Flap Oligo. | CGCCGAGGGGCGGTTGTTGG/3C6/ | 281 |
| DNMT3A | DNMT3A_FP | Forward | GTTACGAATAAAGCGTTGGCG | 93 |
| | | Primer | | |
| | DNMT3A_RP | Reverse | AACGAAACGTCTTATCGCGA | 94 |
| | | Primer | | |
| | DNMT3A_Pb_A5 | Flap Oligo. | CCACGGACGGAGTGCGCGTTC/3C6/ | 95 |
| BC2L11 | BCL2L11_FP | Forward | CGTAATGTTTCGCGTTTTTCG | 35 |
| | | Primer | | |
| | BCL2L11_RP | Reverse | ACTTTCTTCTACGTAATTCTTTTCCGA | 36 |
| | | Primer | | |
| | BCL2L11_Pb_A1 | Flap Oligo. | CGCCGAGGGCGGGGTCGGGC/3C6/ | 37 |
| TBX15 | TBX15_Reg2_FP | Forward | AGGAAATTGCGGGTTTTCG | 332 |
| | | Primer | | |
| | TBX15_Reg2_RP | Reverse | CCAAAAATCGTCGCTAAAAATCAAC | 334 |
| | | Primer | | |
| | TBX15_Reg2_Pb_ | Flap Oligo. | CCACGGACGCGCGCATTCACT/3C6/ | 335 |
| | A5 | | | |
| FERMT3 | FERMT3_FP | Forward | GTTTTCGGGGATTATATCGATTCG | 118 |
| | | Primer | | |
| | FERMT3_RP | Reverse | CCCAATAACCCGCAAAATAACC | 119 |
| | | Primer | | |
| | FERMT3_Pb_A1 | Flap Oligo. | CGCCGAGGCGACTCGACCTC/3C6/ | 120 |
| PRKCB_28 | PRKCB_28_FP | Forward | GGAAGGTGTTTTGCGCG | 249 |
| | | Primer | | |
| | PRKCB_28_RP | Reverse | CTTCTACAACCACTACACCGA | 250 |
| | | Primer | | |
| | PRKCB_28_Pb_ | Flap Oligo. | CCACGGACGGCGCGCGTTTAT/3C6/ | 251 |
| | A5 | | | |
| SOBP_HM | SOBP_HM_FP | Forward | TTTCGGCGGGTTTCGAG | 294 |
| | | Primer | | |
| | SOBP_HM_RP | Reverse | CGTACCGTTCACGATAACGT | 295 |
| | | Primer | | |
| | SOBP_HM_Pb_ | Flap Oligo. | CGCCGAGGGGCGGTCGCGGT/3C6/ | 296 |
| | A1 | | | |
| MAX.chr8. | MAX.Chr8.145_ | Forward | GCGGTATTAGTTAGAGTTTTAGTCG | 211 |
| 145 | FP | Primer | | |
| | MAX.Chr8.145_ | Reverse | ACAACCCTAAACCCTAAATATCGT | 212 |
| | RP | Primer | | |
| | MAX.Chr8.145_ | Flap Oligo. | CCACGGACGGACGGCGTTTTT/3C6/ | 213 |
| | Pb_A5 | | | |
| MAX.chr10. | MAX.Chr10.226_ | Forward | GGGAAATTTGTATTTCGTAAAATCG | 178 |
| 226 | FP | Primer | | |
| | MAX.Chr10.226_ | Reverse | ACAACTAACTTATCTACGTAACATCGT | 179 |
| | RP | Primer | | |
| | MAX_Chr10.226_ | Flap Oligo. | CGCCGAGGGCGGTTAAGAAA/3C6/ | 180 |
| | Pb_A1 | | | |
| MAX.chr12. | MAX.Chr12.52_ | Forward | TCGTTCGTTTTTGTCGTTATCG | 183 |
| 52 | FP | Primer | | |
| | MAX.Chr12.52_ | Reverse | AACCGAAATACAACTAAAAACGC | 184 |
| | RP | Primer | | |
| | MAX.Chr12.52Pb | Flap Oligo. | CCACGGACGCGAACCCCGCAA/3C6/ | 185 |
| | A1 | | | |
| FU45983 | FU45983_FP | Forward | GGGCGCGAGTATAGTCG | 133 |
| | | Primer | | |
| | FU45983_RP | Reverse | CAACGCGACTAATCCGC | 134 |
| | | Primer | | |
| | FU45983_Pb_A1 | Flap Oligo. | CGCCGAGGCCGTCACCTCCA/3C6/ | 135 |
| HOXA9 | HOXA9_FP | Forward | TTGGGTAATTATTACGTGGATTCG | 148 |
| | | Primer | | |
| | HOXA9_RP | Reverse | ACTCATCCGCGACGTC | 149 |
| | | Primer | | |
| | HOXA9_Pb_A5 | Flap Oligo. | CCACGGACGCGACGCCCAACA/3C6/ | 150 |
| EMX1 | EMX1_FP | Forward | GGCGTCGCGTTTTTTAGAGAA | 108 |
| | | Primer | | |
| | EMX1_RP | Reverse | TTCCTTTTCGTTCGTATAAAATTTCGTT | 109 |
| | | Primer | | |
| | EMX1PbA1 | Flap Oligo. | CGCCGAGGATCGGGTTTTAG/3C6/ | 110 |
| SP9 | SP9_FP | Forward | TAGCGTCGAATGGAAGTTCGA | 315 |
| | | Primer | | |
| | SP9_RP | Reverse | GCGCGTAAACATAACGCACC | 317 |
| | | Primer | | |
| | SP9_Pb_A5 | Flap Oligo. | CCACGGACGCCGTACGAATCC/3C6/ | 318 |
| DMRTA2 | DMRTA2_FP | Forward | TGGTGTTTACGTTCGGTTTTCGT | 88 |
| | | Primer | | |
| | DMRTA2_RP | Reverse | CCGCAACAACGACGACC | 89 |
| | | Primer | | |
| | DMRTA2_Pb_A1 | Flap Oligo. | CGCCGAGGCGAACGATCACG/3C6/ | 90 |
| OPLAH | FPrimerOPLAH | Forward | cGTcGcGTTTTTcGGTTATACG | 231 |
| | | Primer | | |
| | RPrimerOPLAH | Reverse | CGCGAAAACTAAAAAACCGCG | 232 |
| | | Primer | | |
| | ProbeA5OPLAH | Flap Oligo. | CCACGGACG-GCACCGTAAAAC/3C6/ | 233 |
| CYP26C1 | CYP26C1_FP | Forward | TGGTTTTTTGGTTATTTCGGAATCGT | 70 |
| | | Primer | | |
| | CYP26C1_RP | Reverse | GCGCGTAATCAACGCTAAC | 71 |
| | | Primer | | |
| | CYP26C1_Pb_A1 | Flap Oligo. | CGCCGAGGCGACGATCTAAC/3C6/ | 72 |
| ZNF781 | ZNF781F.primer | Forward | | |
| | | Primer | CGTTTTTTTGTTTTTCGAGTGCG | 373 |
| | ZNF781R.primer | Reverse | | |
| | | Primer | TCAATAACTAAACTCACCGCGTC | 374 |
| | ZNF781probe.A5 | Flap Oligo. | CCACGGACGGCGGATTTATCG/3C6/ | 375 |
| DLX4 | DLX4_FP | Forward | TGAGTGCGTAGTGTTTTCGG | 80 |
| | | Primer | | |
| | DLX4_RP | Reverse | CTCCTCTACTAAAACGTACGATAAACA | 81 |
| | | Primer | | |
| | DLX4_Pb_A1 | Flap Oligo. | CGCCGAGGATCGTATAAAAC/3C6/ | 82 |
| SUCLG2 | SUCLG2_HM_FP | Forward | TCGTGGGTTTTTAATCGTTTCG | 321 |
| | | Primer | | |
| | SUCLG2_HM_RP | Reverse | TCACGCCATCTTTACCGC | 322 |
| | | Primer | | |
| | SUCLG2_HM_Pb_ | Flap Oligo. | CCACGGACGCGAAAATCTACA/3C6/ | 323 |
| | A5 | | | |
| KLHDC7B | KLHDC7B_FP | Forward | AGTTTTCGGGTTTTGGAGTTCGTTA | 158 |
| | | Primer | | |
| | KLHDC7B_RP | Reverse | CCAAATCCAACCGCCGC | 159 |
| | | Primer | | |
| | KLHDC7B_Pb_A1 | Flap Oligo. | CGCCGAGGACGGCGGTAGTT/3C6/ | 160 |
| S1PR4_HM | S1PR4_HM_FP | Forward | TTATATAGGCGAGGTTGCGT | 284 |
| | | Primer | | |
| | S1PR4_HM_RP | Reverse | CTTACGTATAAATAATACAACCACCGAATA | 285 |
| | | Primer | | |
| | S1PR4_HM_Pb_ | Flap Oligo. | CCACGGACGACGTACCAAACA/3C6/ | 286 |
| | A5 | | | |
| NFIX_HM | NFIX_HM_FP | Forward | TGGTTCGGGCGTGACGCG | 221 |
| | | Primer | | |
| | NFIX_HM_RP | Reverse | TCTAACCCTATTTAACCAACCGA | 222 |
| | | Primer | | |
| | NFIX_HM_Pb_A1 | Flap Oligo. | CGCCGAGGGCGGTTAAAGTG/3C6/ | 223 |
| Reference | Oligonucleotide | | | |
| DNAs | Name | Component | Sequence (5′-3′) | |
| Zebrafish | ZF_RASSF1_FP | BT Forward | TGCGTATGGTGGGCGAG | 394 |
| Synthetic | | Primer | | |
| (RASSF1) | ZF_RASSF1_RP | BT Reverse | CCTAATTTACACGTCAACCAATCGAA | 395 |
| BT | | Primer | | |
| converted) | ZF_RASSF1_Pb_ | BT Flap Oligo. | CCACGGACGGCGCGTGCGTTT/3C6/ | 397 |
| † | A5 | | | |
| B3GALT6* | B3GALT6_FP_V2 | Forward | GGTTTATTTTGGTTTTTTGAGTTTTCGG | 386 |
| | | Primer | | |
| | B3GALT6_RP | Reverse | TCCAACCTACTATATTTACGCGAA | 387 |
| | | Primer | | |
| | B3GALT6_Pb_A1 | Flap Oligo. | CCACGGACGGCGGATTTAGGG/3C6/ | 388 |
| BTACT | ACTB_BT_FP65 | Forward | GTGTTTGTTTTTTTGATTAGGTGTTTAAGA | 381 |
| | | Primer | | |
| | ACTB_BT_RP65 | Reverse | CTTTACACCAACCTCATAACCTTATC | 382 |
| | | Primer | | |
| | ACTBBTPbA3 | Flap Oligo. | GACGCGGAGATAGTGTTGTGG/3C6/ | 383 |

*The B3GALT6 marker is used as both a cancer methylation marker and as a reference target. See U.S. Pat. Appl. Ser. No. 62/364,082, filed Jul. 19, 2016, which is incorporated herein by reference in its entirety.

†For zebrafish reference DNA see U.S. Pat. Appl. Ser. No. 62/364,049, filed Jul. 19, 2016, which is incorporated herein by reference in its entirety.

The DNA prepared from plasma as described above was amplified in two multiplexed pre-amplification reactions, as described in Example 1. The multiplex pre-amplification reactions comprised reagents to amplify the following marker combinations.

TABLE 4

| Multiplex Mix 1 | Multiplex Mix 2 |
|---|---|
| B3GALT6 (reference) | B3GALT6 (reference) |
| ZF_RASSF1 (reference) | ZF_RASSF1 (reference) |
| BARX1 | CYP26C1 |
| BCL2L11 | DLX4 |
| BCL2L11 | DMRTA2 |
| BIN2_Z | EMX1 |
| DNMT3A | HOXA9 |
| FERMT3 | KLHDC7B |
| PARP15 | MAX.chr8.125 |
| PRKCB_28 | MAX_chr10.226 |
| SHOX2 | NFIX |
| SLC12A8 | OPLAH |
| SOBP | S1PR4 |
| TBX15_Reg2 | SP9 |
| ZDHHC1 | SUCLG2 |
| | ZNF781 |

Following pre-amplification, aliquots of the pre-amplified mixtures were diluted 1:10 in 10 mM Tris HCl, 0.1 mM EDTA, then were assayed in triplex QuARTS PCR-flap assays, as described in Example 1. The Group 1 triplex reactions used pre-amplified material from Multiplex Mix 1, and the Group 2 reactions used the pre-amplified material from Multiplex Mix 2. The triplex combinations were as follows:

| | |
|---|---|
| Group 1: | |
| ZF_RASSF1-B3GALT6-BTACT | (ZBA Triplex) |
| BARX1-SLC12A8-BTACT | (BSA2 Triplex) |
| PARP15-MAX.chr8.124-BTACT | (PMA Triplex) |
| SHOX2-ZDHHC1-BTACT | (SZA2 Triplex) |
| BIN2_Z-SKI-BTACT | (BSA Triplex) |
| DNMT3A-BCL2L11-BTACT | (DBA Triplex) |
| TBX15-FERMT3-BTACT | (TFA Triplex) |
| PRKCB_28-SOBP-BTACT | (PSA2 Triplex) |
| Group 2: | |
| ZF_RASSF1-B3GALT6-BTACT | (ZBA Triplex) |
| MAX.chr8.145-MAX_chr10.226-BTACT | (MMA2 Triplex) |
| MAX.chr12.526-FLJ45983-BTACT | (MFA Triplex) |
| HOXA9-EMX1-BTACT | (HEA Triplex) |
| SP9-DMRTA2-BTACT | (SDA Triplex) |
| OPLAH-CYP26C1-BTACT | (OCA Triplex) |
| ZNF781-DLX4-BTACT | (ZDA Triplex) |
| SUCLG2-KLHDC7B-BTACT | (SKA Triplex) |
| S1PR4-NFIX-BTACT | (SNA Triplex) |

Each triplex acronym uses the first letter of each gene name (for example, the combination of HOXA9-EMX1-BTACT=“HEA”). If an acronym is repeated for a different combination of markers or from another experiment, the second grouping having that acronym includes the number 2. The dye reporters used on the FRET cassettes for each member of the triplexes listed above is FAM-HEX-Quasar670, respectively.

Plasmids containing target DNA sequences were used to calibrate the quantitative reactions. For each calibrator plasmid, a series of 10× calibrator dilution stocks, having from 10 to 10⁶ copies of the target strand per μl in fish DNA diluent (20 ng/mL fish DNA in 10 mM Tris-HCl, 0.1 mM EDTA) were prepared. For triplex reactions, a combined stock having plasmids that contain each of the targets of the triplex were used. A mixture having each plasmid at 1×10⁵ copies per μL was prepared and used to create a 1:10 dilution series. Strands in unknown samples were back calculated using standard curves generated by plotting Cp vs Log (strands of plasmid).

Using receiver operating characteristic (ROC) curve analysis, the area under the curve (AUC) for each marker was calculated and is shown in the table below, sorted by Upper 95 Pct Coverage Interval.

TABLE 5

| | | Sensitivity at |
|---|---|---|
| Marker Name | AUC | 90% specificity |
| CYP26C1 | 0.940 | 80% |
| SOBP | 0.929 | 80% |
| SHOX2 | 0.905 | 73% |
| SUCLG2 | 0.905 | 64% |
| NFIX | 0.895 | 63% |
| ZDHHC1 | 0.890 | 69% |
| BIN2_Z | 0.872 | 59% |
| DLX4 | 0.856 | 56% |
| FLJ45983 | 0.834 | 67% |
| HOXA9 | 0.824 | 53% |
| TBX15 | 0.813 | 53% |
| ACTB | 0.803 | 50% |
| S1PR4 | 0.802 | 55% |
| SP9 | 0.782 | 38% |
| FERMT3 | 0.773 | 36% |
| ZNF781 | 0.769 | 55% |
| B3GALT6 | 0.746 | 39% |
| BTACT | 0.742 | 44% |
| BCL2L11 | 0.732 | 39% |
| PARP15 | 0.673 | 31% |
| DNMT3A | 0.689 | 20% |
| MAX.chr12.526 | 0.668 | 33% |
| MAX.chr10.226 | 0.671 | 30% |
| SLC12A8 | 0.655 | 19% |
| BARX1 | 0.663 | 25% |
| KLHDC7B | 0.604 | 10% |
| OPLAH | 0.571 | 14% |
| MAX.chr8.145 | 0.572 | 16% |
| SKI | 0.521 | 14% |

The markers worked very well in distinguishing samples from cancer patients from samples from normal subjects (see ROC table, above). Use of the markers in combination improved sensitivity. For example, using a logistic fit of the data and a six-marker fit, ROC curve analysis shows an AUC=0.973.

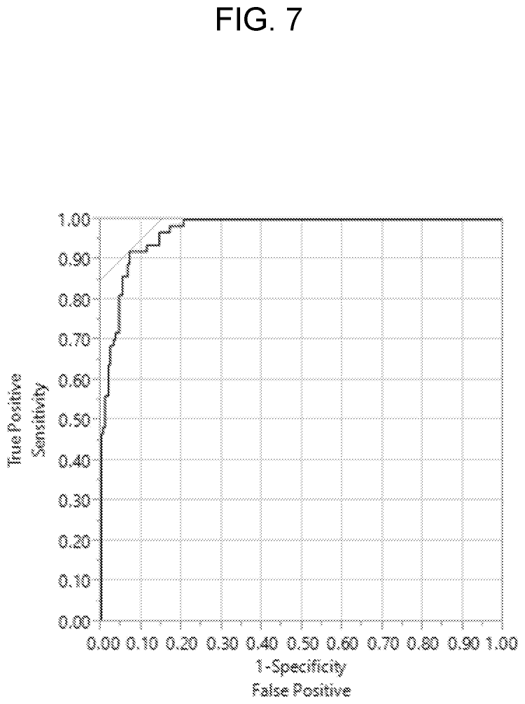

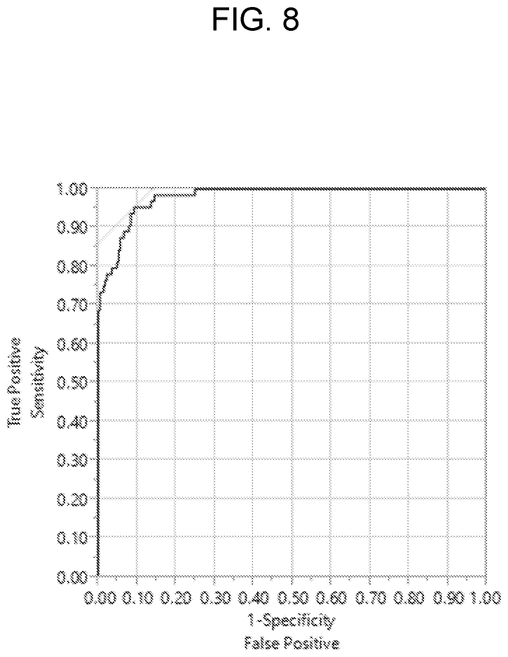

Using a 6-marker fit, sensitivity of 92.2% is obtained at 93% specificity. The group of 6 markers that together resulted in the best fit was SHOX2, SOBP, ZNF781, BTACT, CYP26C1, and DLX4 (see FIG. 7). Using SHOX2, SOBP, ZNF781, CYP26C1, SUCLG2, and SKI gave an ROC curve with AUC of 0.97982 (see FIG. 8).

### Example 4

Archival plasmas from a second independent study group were tested in blinded fashion. Lung cancer cases and controls (apparently healthy smokers) for each group were balanced on age and sex (23 cases, 80 controls). Using multiplex PCR followed by QuARTS (Quantitative Allele-Specific Real-time Target and Signal amplification) assay as described in Example 1, a post-bisulfite quantification of methylated DNA markers on DNA extracted from plasma was performed. Top individual methylation markers from Example 3 were tested in this experiment to identify optimal marker panels for lung cancer detection (2 ml/patient).

Results: 13 high performance methylated DNA markers were tested (CYP26C1, SOBP, SUCLG2, SHOX2, ZDHHC1, NFIX, FLJ45983, HOXA9, B3GALT6, ZNF781, SP9, BARX1, and EMX1). Data were analyzed using two methods: a logistic regression fit and a regression partition tree approach. The logistic fit model identified a 4-marker panel (ZNF781, BARX1, EMX1, and SOBP) with an AUC of 0.96 and an overall sensitivity of 91% and 90% specificity. Analysis of the data using a regression partition tree approach identified 4 markers (ZNF781, BARX1, EMX1, and HOXA9) with AUC of 0.96 and an overall sensitivity of 96% and specificity of 94%. For both approaches, B3GALT6 was used as a standardizing marker of total DNA input. These panels of methylated DNA markers assayed in plasma achieved high sensitivity and specificity for all types of lung cancer.

### Example 5

### Differentiating Lung Cancers

Using the methods described above, methylation markers are selected that exhibit high performance in detecting methylation associated with specific types of lung cancer.

For a subject suspected of having lung cancer, a sample is collected, e.g., a plasma sample, and DNA is isolated from the sample and treated with bisulfite reagent, e.g., as described in Example 1. The converted DNA is analyzed using a multiplex PCR followed by QuARTS flap endonuclease assay as described in Example 1, configured to provide different identifiable signals for different methylation markers or combinations of methylation markers, thereby providing data sets configured to specifically identify the presence of one or more different types of lung carcinoma in the subject (e.g., adenocarcinoma, large cell carcinoma, squamous cell carcinoma, and/or small cell carcinoma). In preferred embodiments, a report is generated indicating the presence or absence of an assay result indicative of the presence of lung carcinoma and, if present, further indicative of the presence of one or more identified types of lung carcinoma. In some embodiments, samples from a subject are collected over the course of a period of time or a course of treatment, and assay results are compared to monitor changes in the cancer pathology.

Marker and marker panels sensitive to different types of lung cancer find use, e.g., in classifying type(s) of cancer present, identifying mixed pathologies, and/or in monitoring cancer progression overtime and/or in response to treatment.

### Example 6

Using multiplex PCR followed by QuARTS (Quantitative Allele-Specific Real-time Target and Signal amplification) assay as described in Example 1, a post-bisulfite quantification of methylated DNA markers on DNA extracted from plasma was performed. The target sequences, bisulfite converted target sequences, and the assay oligonucleotides for these markers were as shown in FIG. 1. The primers and flap oligonucleotides (probes) used for each converted target were as follows:

TABLE 6

| | Oligonucleotide | | | SEQ ID | |
|---|---|---|---|---|---|
| Marker | Name | Component | Sequence (5′-3′) | NO: | Arm |
| BARX1 | BARX1_FP | Primer | CGTTAATTTGTTAGATAGAGGGC | 23 | 5-FAM |
| | | | G | | |
| | BARX1_RP_ | Primer | TCCGAACAACCGCCTAC | 26 | |
| | universal | | | | |
| | BARX1_Pb_A5_ | Flap Oligo. | AGGCCACGGACG | 405 | |
| | 63_v6 | | CGAAAAATCCCACGC/3C6/ | | |
| FLJ45983 | FLJ45983_FP_ | Primer | CGAGGTTATGGAGGTGACG | 409 | 5-FAM |
| | v4 | | | | |
| | FLJ45983_RP_ | Primer | CGAATACTACCCGTTAAACACG | 410 | |
| | v4 | | | | |
| | FLJ45983_Pb_ | Flap Oligo. | AGGCCACGGACG | 411 | |
| | A5_63_v4 | | GGCGGATTAGTCGCG/3C6/ | | |
| HOXA9 | HOXA9_FP | Primer | TTGGGTAATTATTACGTGGATTC | 148 | 5-FAM |
| | | | G | | |
| | HOXA9_RP_v2 | Primer | CAACTCATCCGCGACG | 423 | |
| | HOXA9_Pb_A5_ | Flap Oligo. | AGGCCACGGACG | 424 | |
| | 63 | | GTCGACGCCCAACAA/3C6/ | | |
| HOPX | HOPX_2149_FP | Primer | GTAGCGCGTAGGGATTATGTCG | 417 | 5-FAM |
| | HOPX_2149_RP | Primer | TTTCCACCTAATCCTCTATAAAAC | 418 | |
| | | | CGC | | |
| | HOPX_2149_Pb_ | Flap Oligo. | AGGCCACGGACG | 419 | |
| | A5 | | CTCGCGATCTCCGC/3C6/ | | |
| ZNF781 | ZNF781F. | Primer | CGTTTTTTTGTTTTTCGAGTGCG | 373 | 5-FAM |
| | primer | | | | |
| | ZNF781R. | Primer | TCAATAACTAAACTCACCGCGTC | 374 | |
| | primer | | | | |
| | ZNF781_Pb_A5_ | Flap Oligo | AGGCCACGGACG | 435 | |
| | 63_v2 | | GCGGATTTATCGGGTTATAGT/3C6/ | | |
| HOXB2 | HOXB2_FP | Primer | GTTAGAAGACGTTTTTTCGGGG | 153 | 1-HEX |
| | HOXB2_RP | Primer | AAAACAAAAATCGACCGCGA | 154 | |
| | HOXB2_Pb_ | Flap Oligo. | CGCGCCGAGG | 425 | |
| | A1_63 | | GCGTTAGGATTTATTTTTTTTTTT | | |
| | | | CGA/3C6/ | | |
| IFFO1 | IFFO1_FP_HQ_ | Primer | CGGGATAGAGTCGATTAATTAG | 428 | 1-HEX |
| | corrected | | GC | | |
| | IFFO1_RP | Primer | TAACTTCCCCTCGACCCG | 429 | |
| | IFFO1_Pb_A1_ | Flap Oligo. | CGCGCCGAGG | 430 | |
| | 63 | | CGGTTCGGTAGCGG/3C6/ | | |
| SOBP | SOBP HM FP | Primer | TTTCGGCGGGTTTCGAG | 294 | 1-HEX |
| | SOBP HM RP | Primer | CGTACCGTTCACGATAACGT | 295 | |
| | SOBP HM Pb | Flap Oligo. | CGCGCCGAGG | 431 | |
| | A1 63 | | TTACAAACCGCGACCG/3C6/ | | |
| TRH | TRH_FP | Primer | TTTTCGTTGATTTTATTCGAGTCG | 432 | 1-HEX |
| | | | TC | | |
| | TRH_RP | Primer | GAACCCTCTTCAAATAAACCGC | 433 | |
| | TRH_Pb_A1_ | Flap Oligo. | CGCGCCGAGG | 434 | |
| | 63 | | CGTTTGGCGTAGATATAAGC/3C6/ | | |
| FAM59B | FAM59B_FP_ | Primer | GTCGAGCGTTTGGTGCG | 406 | 1-HEX |
| | V3 | | | | |
| | FAM59B_RP_ | Primer | CTCGTCGAAATCGAAACGC | 407 | |
| | V3 | | | | |
| | FAM59B_Pb_ | Flap Oligo. | CGCGCCGAGG | 408 | |
| | A1_63_V3 | | GCGATAGCGTTTTTTATTGTCG/3C6/ | | |

*All methylation assays were triplexed with an assay for bisulfite-converted B3GALT6 marker, reporting to Quasar:

| | Oligo- | | | | |
|---|---|---|---|---|---|
| | nucleotide | | | SEQ ID | |
| Marker | Name | component | Sequence (5′-3′) | NO: | |
| B3GALT6 | B3GALT6_FP_ | Primer | GGTTTATTTTGGTTTTTTGAGTTTTCGG | 386 | 3-Quasar |
| (BST) | V2 | | | | |
| | B3GALT6_ | Primer | TCCAACCTACTATATTTACGCGAA | 387 | |
| | RP | | | | |
| | B3GALT6_Pb_ | Flap Oligo. | ACGGACGCGGAG | 436 | |
| | A3_63 | | GCGGATTTAGGGTATTTAAGGAG/3C6/ | | |

The DNA prepared from plasma as described above was amplified in a multiplexed pre-amplification reaction, as described in Example 1. Following pre-amplification, aliquots of the pre-amplified mixtures were diluted 1:10 in 10 mM Tris HCl, 0.1 mM EDTA, then were assayed in triplex QuARTS PCR-flap assays, as described in Example 1. The triplex combinations were as follows:

Triplex Assays
BARX1/HOXB2/B3GALT6 (BHB)
FLJ45983/IFFO1/B3GALT6 (FIB)
HOXA9/SOBP/B3GALT6 (HSB)
HOPX 2149/TRH/B3GALT6 (HTB)
ZNF781/FAM59B/B3GALT6 (ZFB)

Plasmids containing target DNA sequences were used to calibrate the quantitative reactions. For each calibrator plasmid, a series of 10× calibrator dilution stocks, having from 10 to 10⁶ copies of the target strand per μl in fish DNA diluent (20 ng/mL fish DNA in 10 mM Tris-HCl, 0.1 mM EDTA) were prepared. For triplex reactions, a combined stock having plasmids that contain each of the targets of the triplex were used. A mixture having each plasmid at 1×10⁵ copies per μL was prepared and used to create a 1:10 dilution series. Strands in unknown samples were back calculated using standard curves generated by plotting Cp vs Log (strands of plasmid).

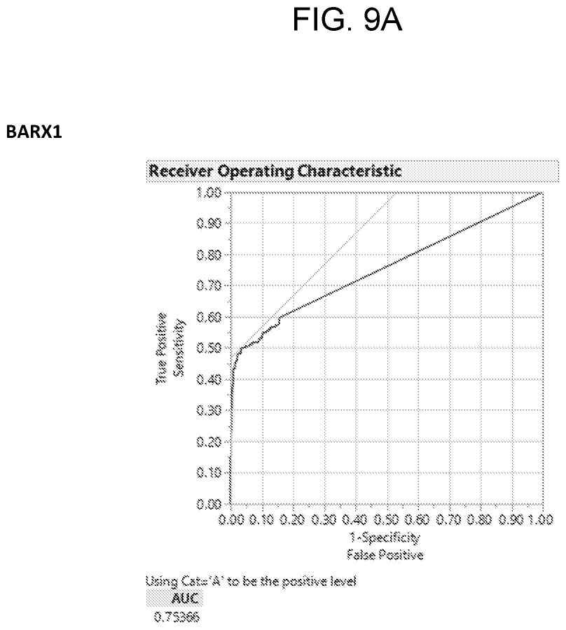

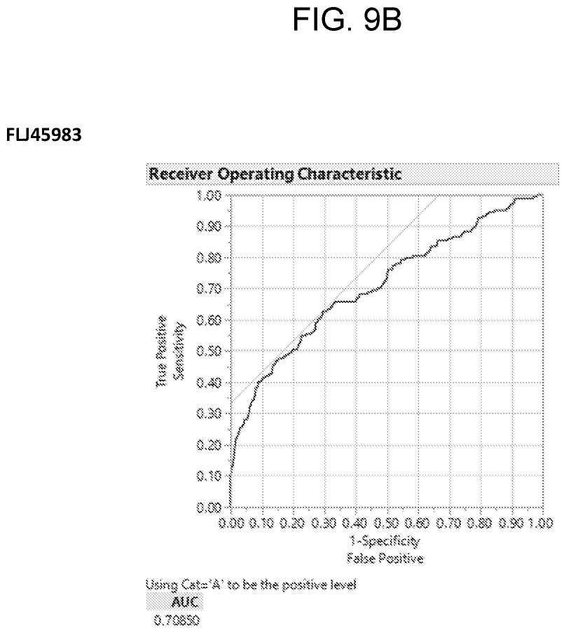

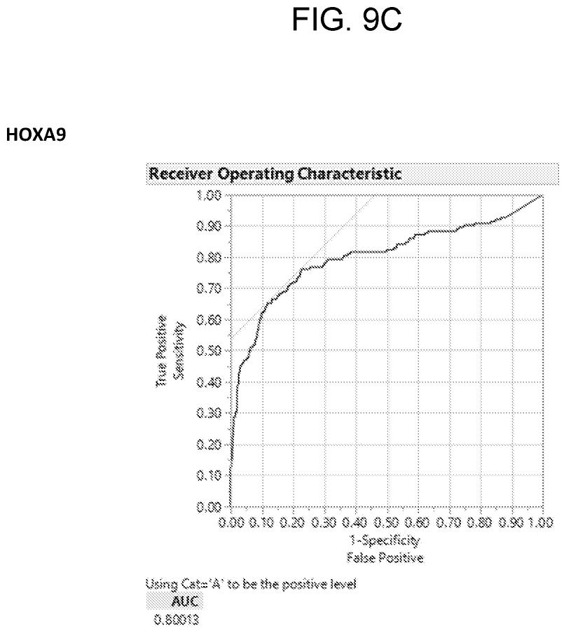

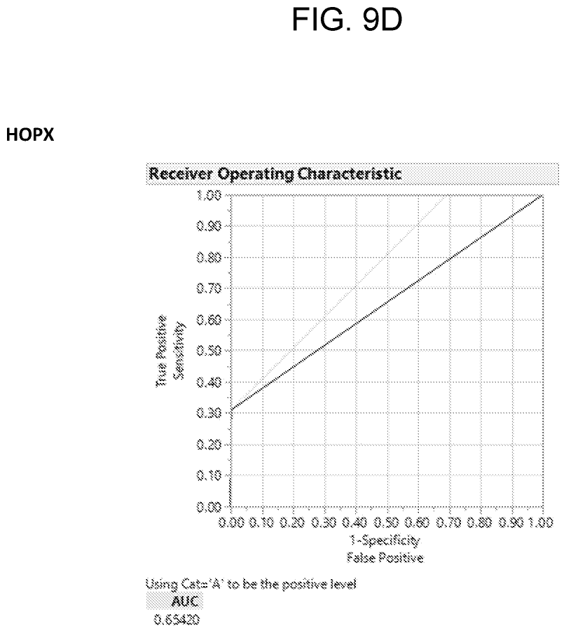

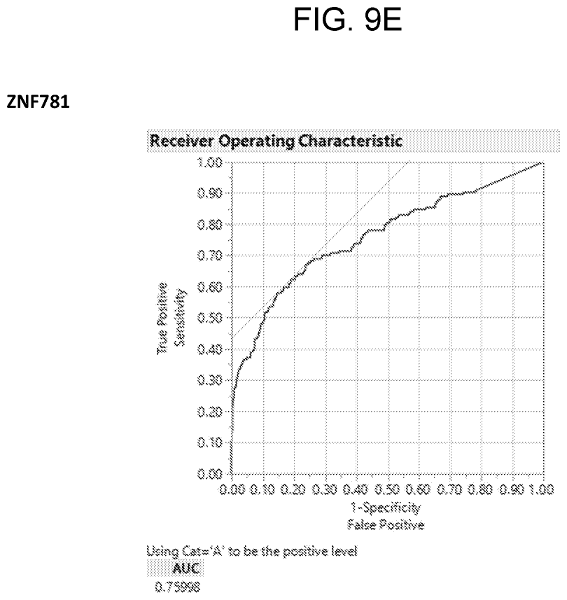

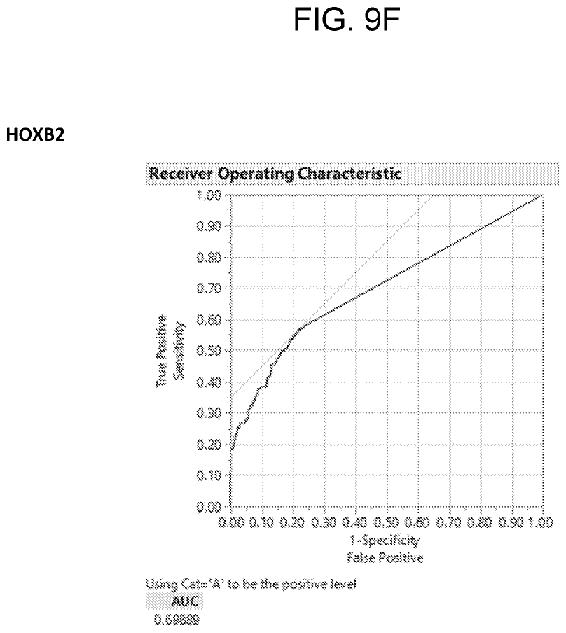

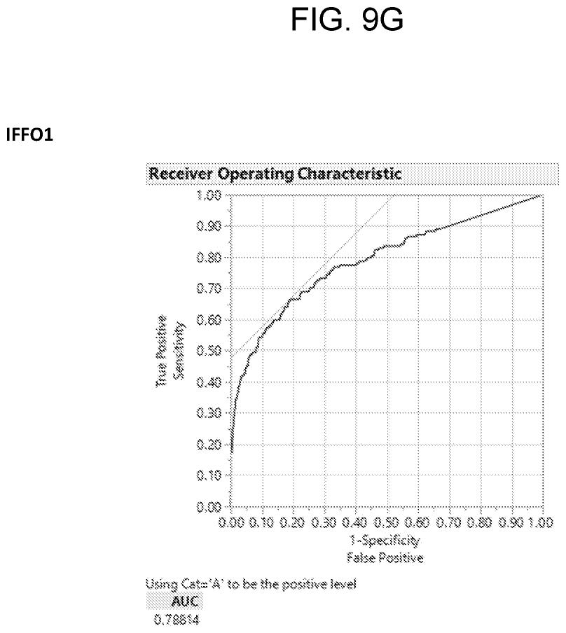

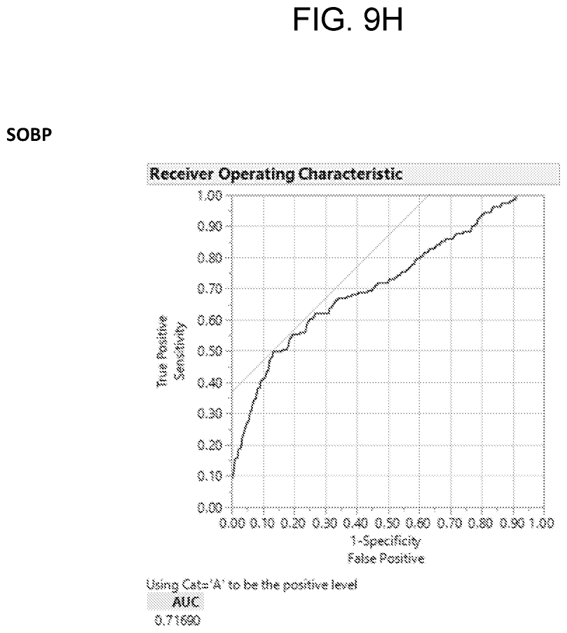

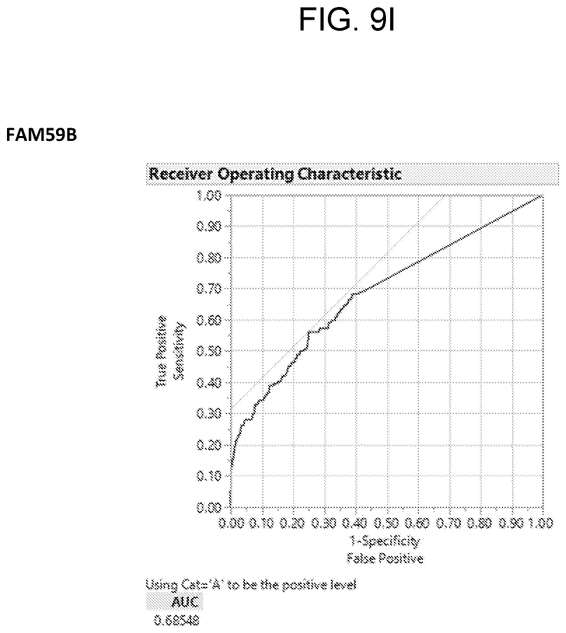

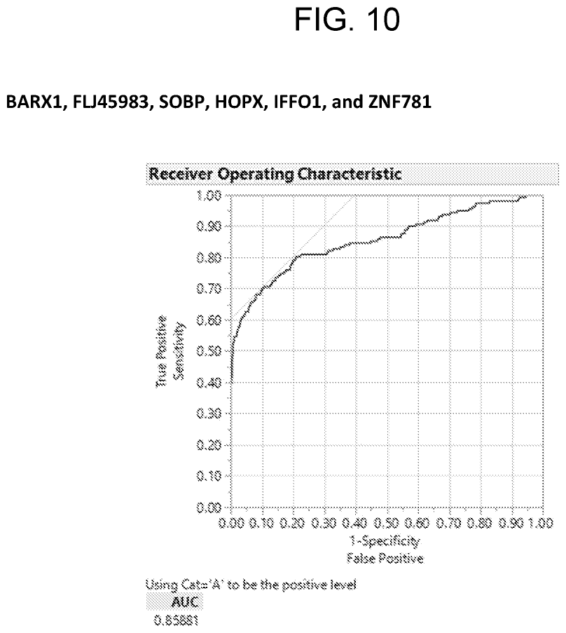

Individual Marker ROC using % methylation relative to B3GALT6 strands is shown in FIG. 9A to 9I. An ROC analysis for the combination of markers FIG. 10 provides a graph showing a 6-marker logistic fit using markers BARX1, FLJ45983, SOBP, HOPX, IFFO1, and ZNF781. The ROC curve analysis shows an area under the curve (AUC) of 0.85881. Use of the markers in combination improved sensitivity.

### Example 7

### Combination of mRNA and Methylation Markers to Improve Lung Cancer Detection Sensitivity

Expression level of FPR1 mRNA (Formyl Peptide Receptor 1) has been shown previously to be a lung cancer marker detectable in blood (Morris, S., et al., Int J Cancer., (2018) 142:2355-2362). In some embodiments, the methylation marker assays described above are used in combination with measurement of one or more expression markers. An exemplary combination assay comprises measurement of FPR1 mRNA levels and detection of methylation marker DNA(s) (e.g., as described in Examples 1-6) in a sample or samples from the same subject.

The FPR1 sequence (NM_001193306.1 Homo sapiens formyl peptide receptor 1 (FPR1), transcript variant 1, mRNA, is shown in SEQ ID NO:437. As described by Morris, et al. supra, blood samples are collected in a blood collection tube suitable for subsequent RNA detection (e.g., PAXgene Blood RNA Tube; Qiagen, Inc.) Samples may be assayed immediately or frozen until future analysis. RNA is extracted from a sample by standard methods, e.g., Qiasymphony PAXgene blood RNA kit. Levels of RNA, e.g., an mRNA marker, are determined using a suitable assay for measurement of specific RNAs present in a sample, e.g., RT-PCR. In some embodiments, a QuARTS flap endonuclease assay reaction comprising a reverse transcription step is used. See, e.g., U.S. patent application Ser. No. 15/587,806, which is incorporated herein by reference. In preferred embodiments, assay probes and/or primers for an RT-PCR or an RT-QuARTS assay are designed to span an exon junction(s) so that the assay will specifically detect mRNA targets rather than detecting the corresponding genomic loci.

An exemplary RT-QuARTS reaction contains 20 U of MMLV reverse transcriptase (MMLV-RT), 219 ng of Cleavase® 2.0, 1.5 U of GoTaq® DNA Polymerase, 200 nM of each primer, 500 nM each of probe and FRET oligonucleotides, 10 mM MOPS buffer, pH7.5, 7.5 mM MgCl₂, and 250 μM each dNTP. Reactions are typically run on a thermal cycler configured to collect fluorescence data in real time (e.g., continuously, or at the same point in some or all cycles). For example, a Roche LightCycler 480 system may be used under the following conditions: 42° C. for 30 minutes (RT reaction), 95° C. for 3 min, 10 cycles of 95° C. for 20 seconds, 63° C. for 30 sec, 70° C. for 30 sec, followed by 35 cycles of 95° C. for 20 sec, 53° C. for 1 min, 70° C. for 30 sec, and hold at 40° C. for 30 sec.

In some embodiments, RT-QuARTS assays may comprise a step of multiplex pre-amplification, e.g., to pre-amplify 2, 5, 10, 12, or more targets in a sample (or any number of targets greater than 1 target), as described above in Example 1. In preferred embodiments, an RT-pre-amplification is conducted in a reaction mixture containing, e.g., 20 U of MMLV reverse transcriptase, 1.5 U of GoTaq® DNA Polymerase, 10 mM MOPS buffer, pH7.5, 7.5 mM MgCl₂, 250 μM each dNTP, and oligonucleotide primers, (e.g., for 12 targets, 12 primer pairs/24 primers, in equimolar amounts (e.g., 200 nM each primer), or with individual primer concentrations adjusted to balance amplification efficiencies of the different targets).

Thermal cycling times and temperatures are selected to be appropriate for the volume of the reaction and the amplification vessel. For example, the reactions may be cycled as follows:

| | | #of |
|---|---|---|
| Stage | Temp/Time | Cycles |
| RT | 42° C./30′ | 1 |
| | 95° C./3′ | 1 |
| Amplification 1 | 95° C./20″ | 10 |
| | 63° C./30″ | |
| | 70° C./30″ | |
| Cooling | 4° C./Hold | 1 |

After thermal cycling, aliquots of the pre-amplification reaction (e.g., 10 μL) are diluted to 500 μL in 10 mM Tris, 0.1 mM EDTA, with or without fish DNA. Aliquots of the diluted pre-amplified DNA (e.g., 10 μL) are used in QuARTS PCR-flap assays, as described above.

In some embodiments, DNA targets, e.g., methylated DNA marker genes, mutation marker genes, and/or genes corresponding to the RNA marker, etc., may be amplified and detected along with the reverse-transcribed cDNAs in a QuARTS assay reaction, e.g., as described in Example 1, above. In some embodiments, DNA and cDNA are co-amplified and detected in a single-tube reaction, i.e., without the need to open the reaction vessel at any point between combining the reagents and collecting the output data. In other embodiments, marker DNA from the same sample or from a different sample may be separately isolated, with or without a bisulfite conversion step, and may be combined with sample RNA in an RT-QuARTS assay. In yet other embodiments, RNA and/or DNA samples may be pre-amplified as described above.

In Morris, ROC curve analysis of the FPR1 mRNA ratio relative to a housekeeping gene (HNRNPA1) resulted in a sensitivity of 68% at a specificity of 89%, and ROC curve analysis using methylation markers BARX1, FAM59B, HOXA9, SOBP, and IFFO1, as shown in FIG. 11B, results in a sensitivity of 77.2% at a specificity of 92.3%. Using these assays together results in a theoretical sensitivity of 92.7% at a specificity of 82%.

This analysis shows that a combination assay for levels of FPR1 mRNA along with detection of one or more methylation markers results in an assay having improved sensitivity compared to either method alone. A cancer detection assay that combines different classes of markers has the advantage of being able to detect the biological differences between early and late diseases stages as well as different biological responses or sources of cancer. It will be clear to one skilled in the art that other RNA targets, including mRNA targets other than or in addition to FPR1, such as LunX mRNA (Yu, et al., 2014, Chin J Cancer Res., 26:89-94), can be combined with methylation markers for enhanced sensitivity.

### Example 8

### Combination of a Protein (e.g., Autoantibody) and Methylation Markers to Improve Lung Cancer Detection Sensitivity

Tumor-associated antigens in lung and other solid tumors can provoke a humoral immune response in the form of autoantibodies, and these antibodies have been observed to be present very early in the disease course, e.g., prior to the presentation of symptoms. (see Chapman C J, Murray A, McElveen J E, et al. Thorax 2008; 63:228-233, which is incorporated herein by reference in its entirety for all purposes). However, the sensitivity of autoantibody detection for detecting lung carcinomas is relatively low. For example, autoantibodies to tumor antigen NY-ESO-1 (Accession #P78358, sequence shown as SEQ ID NO: 442; also known as CTAGIB) has been shown in the literature to be a good marker for non small-cell lung cancer (NSCLC; Chapman, supra), but it is not sufficiently sensitive to be useful alone. The detection of one or more tumor-associated autoantibodies in combination with the detection of one or more methylation markers provides an assay with greater sensitivity.

Blood samples are collected, and autoantibodies are detected using standard methods, e.g., ELISA detection, as described by Chapman, supra. Detecting methylation and/or mutation markers in DNA isolated the samples is done as described in Example 1, above. Detection of NY-ESO-1 autoantibody alone results in a sensitivity of 40% at 95% specificity (Türeci, et al., Cancer Letters 236(1):64 (2006). As discussed above, the assaying the methylation of the combination of BARX1, FAM59B, HOXA9, SOBP, and IFFO1 markers results in a sensitivity of 77.2% at 92.3% specificity. Combining analysis of this autoantibody marker with the assay for this combination of methylation markers results in a combined theoretical sensitivity of 86.3%, with at specificity of 87.7%.

This analysis shows that combined assays of levels of autoantibodies with analysis of one or more methylation markers results in an assay having improved sensitivity compared to either method alone. A cancer detection assay that combines different classes of markers has the advantage of being able to detect the biological differences between early and late diseases stages as well as different biological responses or sources of cancer.

### Example 9

### Combination of mRNA, Methylation Marker(s), and Protein (e.g., Autoantibody) to Improve Lung Cancer Detection Sensitivity

Analysis of combinations of one or more RNAs, marker DNAs, and autoantibodies in a sample or samples from a subject may be performed for enhanced detection of lung and other cancers in the subject. Methods for sample preparation and DNA, RNA, and protein detection are as discussed above.

As discussed in Example 7, analysis of the FPR1 mRNA ratio relative to a housekeeping gene (HNRNPA1) as reported by Morris, et al. resulted in a sensitivity of 68% at a specificity of 89% (Morris, supra); detection of NY-ESO-1 autoantibody alone as reported by Chapman resulted in a sensitivity of 40% at 95% specificity; and assaying the methylation of the combination of BARX1·—FAM59B, HOXA9, SOBP, and IFFO1 markers results in a sensitivity of 77.2% at 92.3% specificity. Combining analysis of the mRNA, the autoantibody marker, and the assay for this combination of methylation markers results in a combined theoretical sensitivity of 95.6%, with a specificity of 77.9%, showing that combined assays of levels of mRNA and levels of autoantibodies with analysis of one or more methylation markers results in an assay having improved sensitivity compared to any one of these methods alone.

Assays as described above may be further enhanced by the addition of an assay to detect one or more antigens. Those of skill in the art will appreciate that detection of an antigen may be added to the detection of any of: RNA(s), methylation marker gene(s), and/or autoantibody(ies), individually or in any combination, and will further enhance overall sensitivity.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

What is claimed is:

1. A method of characterizing a sample, comprising:
a) measuring an amount of at least one methylation marker gene in DNA from the sample, wherein the at least one methylation marker gene comprises at least one of IFFO1 and HOPX;
b) measuring the amount of at least one reference marker in the DNA; and
c) calculating a value for the amount of the at least one methylation marker measured in the DNA as a percentage of the amount of the reference marker measured in the DNA, wherein the value indicates the amount of the at least one methylation marker gene measured in the sample.

2. The method of claim 1, wherein said at least one methylation marker gene consists of one to fifteen methylation marker genes.

3. The method of claim 1, wherein the at least one methylation marker gene comprises one or more marker genes selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145·MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1 and ZNF32.

4. The method of claim 1, wherein the at least one methylation marker gene consists of at least one of IFFO1 and HOPX, and further comprises one or more of BARX1, FLJ45983, HOXA9, ZNF781, HOXB2, SOBP, TRH, and FAM59B.

5. The method of claim 4, wherein the at least one methylation marker gene consists of:
at least one of IFFO1 and HOPX; and
the group consisting of BARX1, FLJ45983, HOXA9, ZNF781, HOXB2, SOBP, TRH, and FAM59B.

6. The method of any one of claims 1 to 5, wherein the at least one reference marker comprises one or more reference marker selected from B3GALT6 DNA and β-actin DNA.

7. The method of any one of claims 1 to 6, wherein the DNA is treated with a reagent that selectively modifies DNA in a manner specific to the methylation status of the DNA.

8. The method of claim 7, wherein the reagent comprises a bisulfite reagent, a methylation-sensitive restriction enzyme, or a methylation-dependent restriction enzyme.

9. The method of any one of claims 1 to 8, wherein the sample comprises one or more of tissue, blood, serum, plasma, and sputum.

10. The method of any one of claims 1 to 9, wherein the DNA is extracted from the sample.

11. The method of any one of claims 1 to 10, wherein the DNA is treated with a bisulfite reagent to produce bisulfite-treated DNA.

12. The method of any one of claims 1 to 11 wherein measuring amounts of a methylation marker gene comprises using one or more of polymerase chain reaction, nucleic acid sequencing, mass spectrometry, methylation-specific nuclease, mass-based separation, and target capture.

13. The method of claim 12, wherein the measuring comprises multiplex amplification.

14. The method of any one of claims 1 to 13, wherein measuring the amount of at least one methylation marker gene comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

15. A method of characterizing at least one sample from a subject, comprising
a) measuring an amount of at least one methylation marker gene in DNA from a sample obtained from a subject, the method comprising:
i) measuring an amount of at least one reference marker in the DNA; and
iii) calculating a value for the amount of the at least one methylation marker gene measured in the DNA as a percentage of the amount of the reference marker measured in the DNA, wherein the value indicates the amount of the at least one methylation marker gene measured in the sample;
and one or more of
b) measuring an amount of at least one RNA marker in a sample obtained from the subject;
and
c) assaying for the presence or absence of at least one protein marker in a sample obtained from the subject.

16. The method of claim 15, wherein measuring an amount of at least one RNA marker in a sample comprises:
i) measuring an amount of a reference RNA in the sample; and
ii) calculating a value for the amount of the at least one RNA marker measured in the sample as a percentage of the amount of reference RNA measured in the sample, wherein the value indicates the amount of the at least one RNA marker measured in the sample, wherein the amount of the at least one RNA marker in the sample is indicative of a level of expression for a gene for said at least one RNA marker.

17. The method of claim 15 or claim 16, wherein the at least one RNA marker comprises mRNA.

18. The method of claim 17, wherein the at least one RNA marker comprises mRNA selected from the group consisting of GAGE12D, FAM83A, LRG1, XAGE-1 d, MAGEA4, SFTPB, AKAP4, and CYP24A1.

19. The method of any one of claims 15 to 18, wherein said reference RNA is selected from the group consisting of CASC3 mRNA, β-actin mRNA, U1 snRNA and U6 snRNA.

20. The method of any one of claims 15 to 19, wherein the at least one methylation marker gene comprises one or more marker genes selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERMT3, NFIX, SIPR4, SKI, SUCLG2, TBX15, ZDHHC1, ZNF32, IFFO1 and HOPX.

21. The method of any one of claims 15 to 20, wherein the protein is an autoantibody.

22. The method of claim 21, wherein the autoantibody is an antibody to a cancer-associated antigen.

23. The method any one of claims 15 to 20, wherein the protein is a cancer-associated antigen.

24. The method of any one of claims 15 to 23, comprising measuring an amount of a methylation marker gene, measuring an amount of an RNA, and assaying for the presence or absence of a protein.

25. The method of any one of claims 15 to 24, wherein said measuring and assaying are conducted on a single sample from the subject.

26. A kit, comprising:
a) at least one marker oligonucleotide, wherein at least a portion of said oligonucleotide specifically hybridizes to a methylation marker selected from the group consisting of IFFO1 and HOPX, and
b) at least one reference oligonucleotide, wherein at least a portion of said reference oligonucleotide specifically hybridizes to a reference nucleic acid.

27. The kit of claim 26, further comprising one or more additional marker oligonucleotides, wherein each of the one or more additional marker oligonucleotides specifically hybridizes to a methylation marker gene selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12A8, BHLHE23, CAPN2, FGF14, FLJ34208, B3GALT6, BIN2_Z, DNMT3A, FERAMT3, NFIX, SIPR4, SKI, SUCLG2, TBX15, ZDHHC1 and ZNF32.

28. The kit of any one of claims 26 to 27, wherein said portion of said marker oligonucleotide specifically hybridizes to a bisulfite-treated DNA comprising said methylation marker.

29. The kit of any one of claims 26 to 28, wherein said kit comprises at least two additional marker oligonucleotides.

30. The kit of any one of claims 26 to 29, wherein said kit further comprises one or more of a methylation-specific restriction enzyme and a bisulfite reagent.

31. The kit of any one of claims 26 to 30, wherein said at least one methylation marker comprises at least one of IFFO1 and HOPX, and further comprises one or more methylation markers selected from the group consisting of BARX1, FLJ45983, HOXA9, ZNF781, HOXB2, SOBP, TRH, and FAM59B.

32. The kit of any one of claims 26 to 31, wherein said at least one methylation marker consists of:
at least one of IFFO1 and HOPX; and
the group consisting of BARX1, FLJ45983, HOXA9, ZNF781, HOXB2, SOBP, TRH, and FAM59B.

33. The kit of any one of claims 26 to 32, wherein said at least one marker oligonucleotide is selected from one or more of a capture oligonucleotide, a pair of nucleic acid primers, a nucleic acid probe, and an invasive oligonucleotide.

34. The kit of any one of claims 26 to 33, wherein said kit further comprises a solid support.

35. The kit of claim 34, wherein said solid support is a magnetic bead.

36. The kit of claim 34 or 35, wherein said solid support comprises one or more capture reagents.

37. The kit of claim 36, wherein said capture reagents are oligonucleotides complementary said one or more methylation markers.

38. A composition comprising a reaction mixture comprising at least one complex comprising a methylation marker DNA and a marker oligonucleotide specifically hybridized to the methylation marker DNA, wherein the methylation marker DNA is selected from IFFO1 and HOPX, and an additional complex comprising an additional methylation marker DNA and an additional marker oligonucleotide specifically hybridized to the additional methylation marker DNA, wherein the additional methylation marker DNA is selected from said the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, ANKRD13B, DOCK2, MAX_chr19.163, ZNFJ32, MAX chr19.372, HOXA9, TRH, SP9, DMRTA2, ARHGEF4, CYP26C1, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12a, BHLHE23, CAPN2, FGF14, FLJ34208, B3GALT6, BIN2_Z, DNMT3A, FERMT3, NFIX, SIPR4, SKI, SUCLG2, TBX15, ZDHHC1, and ZNF32.

39. The composition of claim 38, wherein said methylation marker DNAs are bisulfite-converted methylation marker DNA.

40. The composition of claim 38 or claim 39, wherein said marker oligonucleotides comprise one or more of a capture oligonucleotide, a pair of nucleic acid primers, a hybridization probe, a hydrolysis probe, a flap assay probe, and an invasive oligonucleotide.

41. The composition of any one of claims 38 or 40, comprising a methylation marker DNA comprising a nucleic acid sequence selected from SEQ ID NOS: 412 and 426 and complements thereof, wherein the additional methylation marker DNA comprises a nucleic acid sequence selected from the group consisting of SEQ ID NOS: 1, 6, 11, 16, 21, 28, 33, 38, 43, 48, 53, 58, 63, 68, 73, 78, 86, 91, 96, 101, 106, 111, 116, 121, 126, 131, 136, 141, 146, 151, 156, 161, 166, 171, 176, 181, 186, 191, 196, 201, 214, 219, 224, 229, 234, 239, 247, 252, 257, 262, 267, 272, 277, 282, 287, 292, 298, 303, 308, 313, 319, 327, 336, 341, 346, 351, 356, 361, 366, 371, 384, and 403, and complements thereof.

42. The composition of any one of claims 39 to 40, comprising a methylation marker DNA comprising a nucleic acid sequence selected from SEQ ID NOS: 413 and 427 and complements thereof, wherein the additional methylation marker DNA comprises a nucleic acid sequence selected from the group consisting of SEQ ID NOS: SEQ ID NOS: 2, 7, 12, 17, 22, 29, 34, 39, 44, 49, 54, 59, 64, 69, 74, 79, 87, 92, 97, 102, 107, 112, 117, 122, 127, 132, 137, 142, 147, 152, 157, 162, 167, 172, 177, 182, 187, 192, 197, 202, 210, 215, 220, 225, 230, 235, 240, 248, 253, 258, 263, 268, 273, 278, 283, 288, 293, 299, 304, 309, 314, 320, 328, 337, 342, 347, 352, 357, 362, 367, 372, 385, and 404, and complements thereof.

43. The composition of any one of claims 38 to 42, wherein each of said marker oligonucleotides comprises a reporter molecule.

44. The composition of claim 43, where said reporter molecule comprises a fluorophore.

45. The composition of claim any one of claims 38 to 44, wherein one or more of said marker oligonucleotides comprises a flap sequence.

46. The composition of any one of claims 38 to 45, further comprising one or more of a FRET cassette; a FEN-1 endonuclease and a thermostable DNA polymerase.
